(19) United States
(12) Patent Application Publication
(10) Pub. No.: US 2023/0044602 A1
(43) Pub. Date: Feb. 9, 2023

(54) CANCER SIGNATURES, METHODS OF GENERATING CANCER SIGNATURES, AND USES THEREOF

(71) Applicant: EDWARD VIA COLLEGE OF OSTEOPATHIC MEDICINE, Blacksburg, VA (US)

(72) Inventors: Robin T. VARGHESE, Christianburg, VA (US); Kevin SHENG, Roanoke, VA (US)

(21) Appl. No.: 17/786,373

(22) PCT Filed: Dec. 20, 2020
(86) PCT No.: PCT/US2020/066282

§ 371 Date: Jun. 16, 2022

Related U.S. Application Data
(60) Provisional application No. 62/951,084, filed on Dec. 20, 2019.

(51) Int. Cl. C12Q 1/6886; C12Q 1/686; G06K 9/62; G06K 9/00; G16H 50/30; G16H 50/20

(52) U.S. Cl. CPC C12Q 1/6886; C12Q 1/686; G06K 9/6247; G06K 9/00536; G16H 50/30; G16H 50/20; C12Q 2600/112; C12Q 2600/118; C12Q 2600/154; C12Q 2600/158

(57) ABSTRACT

Described herein are compositions, methods, and techniques to generate a cancer signature and uses thereof. The cancer signature can be used to determine a cancer progression risk of a subject based upon expression levels of genes of a progression gene signature in a sample. The methods can be used to predict a prognosis, to select an appropriate treatment regimen, to identify or screen for an agent effective against a cancer, or a combination thereof. Computer implemented methods and systems that implement those methods are also provided. This abstract is intended as a scanning tool for purposes of searching in the particular art and is not intended to be limiting of the present disclosure.

### CROSS-REFERENCE TO RELATED APPLICATIONS

This Application claims the benefit of U.S. Provisional Application No. 62/951,084, filed on Dec. 20, 2019, which is incorporated herein by reference in its entirety.

### REFERENCE TO A SEQUENCE LISTING SUBMITTED AS A TEXT FILE VIA EFS-WEB

The Sequence Listing submitted Dec. 20, 2020 as a text file named “2020-12-18_Sequence_Listing_VCOM-00001-U-PCT-01_ST25.K” created on Dec. 19, 2020, and having a size 236,295 bytes is hereby incorporated by reference pursuant to 37 C.F.R. § 1.52(e)(5).

### TECHNICAL FIELD

The subject matter disclosed herein is generally directed to compositions, methods, and techniques for diagnosing and/or prognosing cancer.

### BACKGROUND

Cancer is a leading cause of morbidity and mortality worldwide. Further, cancer can be heterogenous in presentation across any given patient population. Some of the heterogeneity can be attributed to an incomplete characterization of any given type of cancer. However, a larger factor contributing to the heterogeneity is the interaction between any given individual patient and the cancer. The heterogeneity of cancer, particularly when considered at the individual patient level, has inhibited the development of robustly effective therapeutic options. Thus, there is an urgent and unmet need for methods and techniques that can be effective to characterize a cancer at the individual patient level and/or stratify patients in a patient population with an improved granularity to facilitate appropriate treatment at the individual patient level and/or patient subpopulation level.

Citation or identification of any document in this application is not an admission that such a document is available as prior art to the present invention.

### SUMMARY

In accordance with the purpose(s) of the disclosure, as embodied and broadly described herein, the disclosure, in one aspect, relates to methods of determining a cancer progression risk score of a subject. The methods can include detecting expression levels of genes of a progression gene signature in a sample; and calculating the cancer progression risk score of the subject using the expression levels of genes associated with a progression gene signature in the sample. In some aspects, the sample obtained from a subject, e.g. a human subject. In some aspects, the sample is obtained from a tumor, tissue, bodily fluid, or a combination thereof.

In some aspects, the progression gene signature includes a glioblastoma progression gene signature, a non-small cell lung squamous cell carcinoma progression gene signature, a non-small cell lung adenocarcinoma progression gene signature, or combinations thereof.

In some aspects, the cancer progression risk score is high risk progression or low risk progression. For example, in some aspects a low risk progression indicates that the patient will be more responsive to chemotherapeutics. In some aspects, the high risk progression indicates the patient will be more resistant to chemotherapeutic treatment and a more aggressive or non-standard treatment regimen should be considered.

In some aspects, the progression gene signature includes a glioblastoma progression gene signature; wherein the glioblastoma progression gene signature comprises one or more genes selected from RPS11, UBB, TUBB, RPS6, EEF1A1, EEF2, PKM, C3, ENO1, HSP90AB1, FTL, CFL1, YWHAE, CKB, TUBA1A, FLNA, APP, CD63, ACTB, VIM, CTSB, MME, GLUL, MT3, ACTG1, HLA-C, B2M, CRYAB, LRP1, S100B, and FN1.

In some aspects, the progression gene signature includes a non-small cell lung squamous cell carcinoma progression gene signature; and wherein the non-small cell lung squamous cell carcinoma progression gene signature comprises one or more genes selected from GAPDH, KRT5, ACTG1, ENO1, PKM, CTSB, PSAP, MYH9, KRT14, RPS4X, CALR, FLNA, HSPA8, SFTPA2, RPS11, HSP90B1, HSPB1, SDC1, HLA-C, APP, ATP1A1, HSPA5, and RPL37.

In some aspects, the progression gene signature includes a non-small cell lung adenocarcinoma progression gene signature; and wherein the non-small cell lung adenocarcinoma progression gene signature comprises one or more genes selected from ACTB, FTL, SFTPA2, CD74, FN1, B2M, CTSD, CEACAM6, EEF2, PGC, UBC, HSP90AB1, SERPINA1, HSPA8, HSP90AA1, GNB2L1 (RACK1), CEACAM5, CD63, PIGR, KRT18, GLUL, and KRT19.

The methods can include stratifying the subjects using a classification method selected from the group consisting of a profile similarity; an artificial neural network; a support vector machine (SVM); a logic regression, a linear or quadratic discriminant analysis, a decision trees, a clustering, a principal component analysis, a nearest neighbor classifier analysis, a nearest shrunken centroid, a random forest, and a combination thereof. random

In some aspects, the classification method is trained on a subset of components from a set of components generated using a reduced dimensionality representation such as from principal component analysis, the subset of components being more highly correlated to the risk of progression as compared to a correlation of the unselected components.

Methods of detecting cancer, methods of treating cancer, and methods of screening an agent effective against a cancer are also provided based on the progression gene signatures.

Systems (e.g. computer systems) and computer-implemented methods for generating a progression gene signature for a cancer are also provided.

These and other aspects, objects, features, and advantages of the example aspects will become apparent to those having ordinary skill in the art upon consideration of the following detailed description of example aspects.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A is a schematic flowchart illustrating step-wise a biomarker discovery pipeline. The log₂ fold change values of shRNA depletion for the top 100 most ubiquitously expressed genes in LUAD (FIG. 1B), LUSC (FIG. 1C), and GBM (FIG. 1D) were calculated from Project Achilles and shown in the left panels. 29, 26, and 22 genes were undetected in Project Achilles for LUAD, LUSC, and GBM, respectively, and excluded from downstream analyses. A fold change cutoff of <0 (red line, left panels) was used to select genes essential for cancer cell survival. One-sample one-tailed t-tests and Fisher's method determined the significance of fold change <0 (Supplementary Table S5). Survival genes were then entered into a backward stepwise variable regression model and selected to form PGSs using an arbitrary p-value threshold of 0.25 (red line, right panels) to select for interacting variables. Each survival gene and their corresponding stepwise P-values are shown in the right and middle panels, respectively.

FIG. 2A shows a schematic flowchart illustrating a risk score algorithm to quantify patient risk for disease progression. ROC curves trained on PGS risk scores were used to calculate AUC values for LUAD-PGS (FIG. 2B), LUSC-PGS (FIG. 2C), and GBM-PGS (FIG. 2D) describing the overall accuracy of the model. Pair-wise comparisons were used to determine significance of PGS performance compared to current clinical biomarkers independently and in conjunction.

FIGS. 3A-3C show the risk score and patient stratification for LUAD (FIG. 3A), LUSC (FIG. 3B), and GBM (FIG. 3C) demonstration the accurately stratify patients into risk groups correlating with tumor progression. Patients were stratified as high-risk progression (risk score >0) or low-risk progression (risk score <0) and analyzed for correlations with tumor progression incidence. Fisher's Exact Tests determined significance of correlation. (FIGS. 3D-3F for LUAD, LUSC, and GBM respectively) Kaplan-Meier survival curves of disease-free survival (DFS) time between high- and low-risk patients. Median DFS times for each risk group are shown in months. P-values were calculated using log-rank tests. C.C.B—combined current biomarkers, DFS—disease-free survival.

FIG. 4. High-risk patients stratified by PGSs do not benefit from chemotherapy. FIG. 4A shows the disease free survival time of patients receiving ACT in NSCLC or TMZ in GBM demonstrating that high-risk patients stratified by PGSs do not benefit from chemotherapy. Average DFS times for each risk group are shown in months. P-values were calculated using student t tests. (FIGS. 4B-4C) Correlation of PGS risk stratification with patient response to ACT. Significance was determined using Fisher's Exact Tests. (FIG. 4D) Buffa tumor hypoxia scores between PGS risk groups. Higher scores indicate hypoxia, while lower scores indicate normoxia. P-values were calculated using two-tailed t-tests on unequal variances. ***P<0.0001, NS—not significant.

FIGS. 5A-5B show patient risk stratification by LUAD-PGS (FIG. 5A) and LUSC-PGS (FIG. 5B) in a 246-patient and 207-patient validation cohort compiled from four independent microarray datasets from Gene Expression Omnibus (GEO). P-values were calculated via Fisher's Exact Tests. FIGS. 5C-5D show patient risk stratification by GBM-PGS in a 126-patient TCGA validation cohort excluded from training (FIG. 5C) and a 200-patient external validation cohort from Rembrandt (FIG. 5D). Overall survival (OS) status was used in Rembrandt due to a lack of progression data. P-values were calculated via Fisher's Exact Tests. FIGS. 5E-5F show Kaplan-Meier survival curves of survival time between high- and low-risk NSCLC patients for LUAD (FIG. 5E) and LUSC (FIG. 5F). P-values were calculated using log-rank tests. FIGS. 5G-5H show Kaplan-Meier survival curves of DFS time (FIG. 5G) or OS time (FIG. 5H) between high- and low-risk GBM patients. P-values were calculated using log-rank tests. FIG. 5I shows primary cells were established from GBM tumor samples collected from Carilion Clinic. Expression of GBM-PGS genes were determined by RT-qPCR and analyzed using the GBM-PGS risk algorithm, stratifying five patients as high-risk (red) and one patient as low-risk (blue).

FIGS. 6A-6H show Kaplan-Meier survival curves of all patients in training (FIGS. 6A, 6C, and 6E) and validation (FIGS. 6B, 6D, and 6F-6H) cohorts for LUAD (FIGS. 6A-6B), LUSC (FIGS. 6C-6D), and GBM (FIGS. 6E-6H) are shown. Median DFS or OS times are shown in months. DFS—disease-free survival, OS—overall survival.

FIGS. 7A-7E show Kaplan-Meier survival curves of disease-free survival (DFS) time in patients with mutant or wild-type EEF2 in LUAD (FIG. 7A), CTSB (FIG. 7B) or HSP90B1 (FIG. 7C) in LUSC, and APP (FIG. 7D) or MME (FIG. 7E) in GBM. Median DFS times are shown in months. P-values were calculated using log-rank tests.

FIG. 8 shows frequencies of high-risk progression (HR) or low-risk progression (LR) stratification in patients with mutant PGS genes are shown. The DFS status of patients in each risk group are shown as gray (disease-free) or black (progressed).

FIGS. 9A-9C show patients stratified as high-risk progression (risk score >0) or low-risk progression (risk score <0) by GBM-PGS were analyzed for correlations with tumor progression incidence. Fisher's Exact Tests determined significance of correlation. FIGS. 9D-9F show Kaplan-Meier survival curves of disease-free survival (DFS) time between high- and low-risk patients. Median DFS times for each risk group are shown in months. P-values were calculated using log-rank tests. DFS—disease-free survival.

FIGS. 10A-10C show patient risk stratification by GBM-PGS in each GBM subtype in the 126-patient TOGA GBM validation cohort. P-values were calculated via Fisher's Exact Tests. FIGS. 10D-10F show Kaplan-Meier survival curves of disease-free survival (DFS) time between high- and low-risk patients. Median DFS times for each risk group are shown in months. P-values were calculated using log-rank tests. DFS—disease-free survival.

FIG. 11A is a schematic showing how to perform a quadruplex ddPCR, primers and different probes (1×FAM-probe A, 0.5×FAM-probe B, 1×HEX-probe C, and 0.5×HEX-probe D) are mixed with the template cDNA. 0.5× and 1× Probes will have 2-fold difference of amplitude. After compartmentalization using the droplet generator, 20,000 droplets are generated from 20 μl reaction. Then PCR amplification is done in a thermocycler. FIG. 11B shows how the amplicons with different fluorescence intensities are quantified at FAM or HEX channel and plotted. Difference populations with A, B, C, and/or D amplicons are analyzed using QuantaSoft.

FIG. 12 shows a flow diagram of an example process for processing biological information.

FIG. 13 shows an exemplary computer system that can be used for processing biological information.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

An understanding of the features and advantages of the present invention will be obtained by reference to the following detailed description that sets forth illustrative aspects, in which the principles of the invention may be utilized, and the accompanying drawings.

### DETAILED DESCRIPTION

Glioblastoma (GBM) is the most common and aggressive malignancy of the central nervous system. The average length of survival for GBM patients is approximately 12-15 months, with only 3-5% of patients surviving for longer than 5 years after diagnosis even with aggressive treatment including surgical resection and chemotherapy. Therefore, identification of underlying molecular mechanisms associated with poorer patient prognosis may reveal novel therapeutic avenues for GBM.

Lung cancer is the most common malignant neoplasm and leading cause of cancer-associated mortality worldwide, with a five-year survival rate of 17.8%. Tumors are broadly stratified into two subtypes—non-small cell lung carcinoma (NSCLC), comprising of 85% of all lung cancer cases, and small cell lung carcinoma. NSCLC can be further classified into three histological subtypes: large cell carcinoma, adenocarcinoma (LUAD), and squamous cell carcinoma (LUSC). LUAD and LUSC account for approximately 50% and 35% of NSCLC diagnoses, respectively.

With that said, aspects disclosed herein can provide signatures, such as gene signatures, methods and techniques that can be useful in at least the diagnosis, prognosis, and/or patient stratification of a cancer, such as glioblastoma or a lung cancer (e.g. NSCLC). Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

In some aspects, a method is provided for determining a cancer progression risk score of a subject. The method can include detecting expression levels of genes of a progression gene signature in a sample; and calculating the cancer progression risk score of the subject using the expression levels of genes associated with a progression gene signature in the sample; wherein the progression gene signature includes one or more of a glioblastoma progression gene signature, a non-small cell lung squamous cell carcinoma progression gene signature, a non-small cell lung adenocarcinoma progression gene signature, or combinations thereof. The progression risk score can be used to stratify subjects or samples therefrom into high risk progression or low risk progression.

In some aspects, the genes are selected from RPS11, UBB, TUBB, RPS6, EEF1A1, EEF2, PKM, C3, ENO1, HSP90AB1, FTL, CFL1, YWHAE, CKB, TUBA1A, FLNA, APP, CD63, ACTB, VIM, CTSB, MME, GLUL, MT3, ACTG1, HLA-C, B2M, CRYAB, LRP1, S100B, and FN1. In some aspects, the genes are selected from GAPDH, KRT5, ACTG1, ENO1, PKM, CTSB, PSAP, MYH9, KRT14, RPS4X, CALR, FLNA, HSPA8, SFTPA2, RPS11, HSP90B1, HSPB1, SDC1, HLA-C, APP, ATP1A1, HSPA5, and RPL37. In some aspects, the genes are selected from ACTB, FTL, SFTPA2, CD74, FN1, B2M, CTSD, CEACAM6, EEF2, PGC, UBC, HSP90AB1, SERPINA1, HSPA8, HSP90AA1, GNB2L1 (RACK1), CEACAM5, CD63, PIGR, KRT18, GLUL, and KRT19.

In some aspects, the cancer progression risk score is determined based upon a classification method selected from the group consisting of a profile similarity; an artificial neural network; a support vector machine (SVM); a logic regression, a linear or quadratic discriminant analysis, a decision trees, a clustering, a principal component analysis, a nearest neighbor classifier analysis, a nearest shrunken centroid, a random forest, and a combination thereof. Systems and methods are also provided, e.g. computer-implemented methods and computer systems for carrying out the methods, for constructing and/or computing the cancer progression risk score.

Before the present disclosure is described in greater detail, it is to be understood that this disclosure is not limited to particular aspects described, and as such may, of course, vary. It is also to be understood that the terminology used herein is for the purpose of describing particular aspects only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual aspects described and illustrated herein has discrete components and features which may be readily separated from or combined with the features of any of the other several aspects without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboraotry Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” may contain whole cells and/or live cells and/or cell debris. The biological sample may contain (or be derived from) a “bodily fluid”. The present invention encompasses aspects wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein “cancer” can refer to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basa cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodyspastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sézary syndrome, skin cancer, small intestine cancer, large intestine cancer (colon cancer), soft tissue sarcoma, T-cell lymphoma, throat cancer, oropharyngeal cancer, nasopharyngeal cancer, hypoharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein, “administering” refers to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated.

As used herein, “cell identity” is the outcome of the instantaneous intersection of all factors that affect it. Wagner et al., 2016. Nat Biotechnol. 34(111): 1145-1160. A cell's identity can be affected by temporal and/or spatial elements. A cell's identity is also affected by its spatial context that includes the cell's absolute location, defined as its position in the tissue (for example, the location of a cell along the dorsal ventral axis determines its exposure to a morphogen gradient), and the cell's neighborhood, which is the identity of neighboring cells. The cell's identity is manifested in its molecular contents. Genomic experiments measure these in molecular profiles, and computational methods infer information on the cell's identity from the measured molecular profiles (inevitably, the molecular profile also reflects allele-intrinsic and technical variation that must be handled properly by computational methods before any analysis is done). This is referred to herein as inferring facets of the cell's identity (or the factors that created it) to stress that none describes it fully, but each is an important, distinguishable aspect. The facets relate to vectors that span the space of cell identities Computational analysis methods can be used of finds such basis vectors directly (Wagner et al., 2016).

As used herein, “cell type” refers to the more permanent aspects (e.g. a hepatocyte typically can't on its own turn into a neuron) of a cell's identity. Cell state can be thought of as the permanent characteristic profile or phenotype of a cell. Cell types are often organized in a hierarchical taxonomy, types may be further divided into finer subtypes; such taxonomies are often related to a cell fate map, which reflect key steps in differentiation or other points along a development process. Wagner et al., 2016. Nat Biotechnol. 34(111): 1145-1160

As used herein, “agent” refers to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “cell state” are used to describe transient elements of a cell's identity. Cell state can be thought of as the transient characteristic profile or phenotype of a cell. Cell states arise transiently during time-dependent processes, either in a temporal progression that is unidirectional (e.g., during differentiation, or following an environmental stimulus) or in a state vacillation that is not necessarily unidirectional and in which the cell may return to the origin state. Vacillating processes can be oscillatory (e.g., cell-cycle or circadian rhythm) or can transition between states with no predefined order (e.g., due to stochastic, or environmentally controlled, molecular events). These time-dependent processes may occur transiently within a stable cell type (as in a transient environmental response), or may lead to a new, distinct type (as in differentiation). Wagner et al., 2016. Nat Biotechnol. 34(111): 1145-1160.

As used herein, “cellular phenotype” refers to the configuration of observable traits in a single cell or a population of cells.

As used herein, “chemotherapeutic agent” or “chemotherapeutic” refers to a therapeutic agent utilized to prevent or treat cancer.

As used herein, “control” can refer to an alternative subject or sample used in an experiment for comparison purpose and included to minimize or distinguish the effect of variables other than an independent variable.

As used herein, “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, in aspects modulation may encompass an increase in the value of the measured variable by about 10 to 500 percent or more. In aspects, modulation can encompass an increase in the value of at least 10%, 20%, 30%, 40%, 50%, 75%, 100%, 150%, 200%, 250%, 300%, 400% to 500% or more, compared to a reference situation or suitable control without said modulation. In aspects, modulation may encompass a decrease or reduction in the value of the measured variable by about 5 to about 100%. In some aspects, the decrease can be about 5%, 10%, 20%, 30%, 40%, 50%, 60%, 70%, 80%, 90%, 95%, 96%, 97%, 98%, 99% to about 100%, compared to a reference situation or suitable control without said modulation. In aspects, modulation may be specific or selective, hence, one or more desired phenotypic aspects of a cell or cell population may be modulated without substantially altering other (unintended, undesired) phenotypic aspect(s).

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, a “progression gene signature” and “PGS” can be used interchangeably and refer to a gene that is highly associated with cancer progression as disclosed herein. A PGS, as disclosed, herein may be associated with at least one cancer type. However, a given PGS can be associated with more than one cancer type.

Various aspects are described hereinafter. It should be noted that the specific aspects are not intended as an exhaustive description or as a limitation to the broader aspects discussed herein. One aspect described in conjunction with a particular aspect is not necessarily limited to that aspect and can be practiced with any other aspect(s). Reference throughout this specification to “one aspect”, “an aspect,” “an example aspect,” means that a particular feature, structure or characteristic described in connection with the aspect is included in at least one aspect of the present invention. Thus, appearances of the phrases “in one aspect,” “in an aspect,” or “an example aspect” in various places throughout this specification are not necessarily all referring to the same aspect, but may. Furthermore, the particular features, structures or characteristics may be combined in any suitable manner, as would be apparent to a person skilled in the art from this disclosure, in one or more aspects. Furthermore, while some aspects described herein include some but not other features included in other aspects, combinations of features of different aspects are meant to be within the scope of the invention. For example, in the appended claims, any of the claimed aspects can be used in any combination.

All publications, published patent documents, and patent applications cited herein are hereby incorporated by reference to the same extent as though each individual publication, published patent document, or patent application was specifically and individually indicated as being incorporated by reference.

### Signatures.

Various gene signatures are described for determining a risk of cancer progression, e.g. for determining if a subject or a sample (e.g. obtained from a tumor, tissue, bodily fluid, or a combination thereof.) from a subject presents a high risk progression or a low risk progression. Methods of determining gene signatures for cancers are also described.

In some aspects the signature is a glioblastoma progression gene signature; and wherein the glioblastoma progression gene signature includes one, two, three, four, five, six, seven, eight, nine, ten, or more genes selected from RPS11, UBB, TUBB, RPS6, EEF1A1, EEF2, PKM, C3, ENO1, HSP90AB1, FTL, CFL1, YWHAE, CKB, TUBA1A, FLNA, APP, CD63, ACTB, VIM, CTSB, MME, GLUL, MT3, ACTG1, HLA-C, B2M, CRYAB, LRP1, S100B, and FN1. In some aspects, the signature includes detecting expression levels of each of the genes RPS11, UBB, TUBB, RPS6, EEF1A1, EEF2, PKM, C3, ENO1, HSP90AB1, FTL, CFL1, YWHAE, CKB, TUBA1A, FLNA, APP, CD63, ACTB, VIM, CTSB, MME, GLUL, MT3, ACTG1, HLA-C, B2M, CRYAB, LRP1, S100B, and FN1.

In some aspects the signature is a non-small cell lung squamous cell carcinoma progression gene signature; and the non-small cell lung squamous cell carcinoma progression gene signature includes one, two, three, four, five, six, seven, eight, nine, ten, or more genes selected from GAPDH, KRT5, ACTG1, ENO1, PKM, CTSB, PSAP, MYH9, KRT14, RPS4X, CALR, FLNA, HSPA8, SFTPA2, RPS11, HSP90B1, HSPB1, SDC1, HLA-C, APP, ATP1A1, HSPA5, and RPL37. In some aspects, the signature includes detecting expression levels of each of the genes GAPDH, KRT5, ACTG1, ENO1, PKM, CTSB, PSAP, MYH9, KRT14, RPS4X, CALR, FLNA, HSPA8, SFTPA2, RPS11, HSP90B1, HSPB1, SDC1, HLA-C, APP, ATP1A1, HSPA5, and RPL37.

In some aspects the signature is a non-small cell lung adenocarcinoma progression gene signature; wherein the non-small cell lung adenocarcinoma progression gene signature includes one, two, three, four, five, six, seven, eight, nine, ten, or more genes selected from ACTB, FTL, SFTPA2, CD74, FN1, B2M, CTSD, CEACAM6, EEF2, PGC, UBC, HSP90AB1, SERPINA1, HSPA8, HSP90AA1, GNB2L1 (RACK1), CEACAM5, CD63, PIGR, KRT18, GLUL, and KRT19. In some aspects, the signature includes detecting expression levels of each of the genes ACTB, FTL, SFTPA2, CD74, FN1, B2M, CTSD, CEACAM6, EEF2, PGC, UBC, HSP90AB1, SERPINA1, HSPA8, HSP90AA1, GNB2L1 (RACK1), CEACAM5, CD63, PIGR, KRT18, GLUL, and KRT19.

Methods of Modulating, Inhibiting, and/or Killing a Cancer Cell.

Described herein are methods of modulating a cancer cell from one cell state to another. In some aspects, the method can include modulating a cell or population thereof that is in a first cancer cell state to a second cancer cell state and/or non-diseased or normal cell state. Described herein are methods of inhibiting an activity and/or function of a cancer cell. Described herein are methods of killing a cancer cell. In some aspects, the method of inhibiting an activity and/or function of a cancer cell and/or method of killing a cancer cell can include a method of modulating a cancer cell. In some aspects, the method can include modulating a cell or population thereof that is in a first cancer cell state to a second cancer cell state and/or non-diseased or normal cell state.

The methods of modulating astrocytes described herein can be used, for example, to engineer cancer cells having a particular cell state and corresponding characteristics and attributes, to screen and identify agents capable of inducing a particular cell state, inhibiting a function and/or activity of a cancer cell and/or killing a cancer cell, and/or for the treatment of cancer (such as glioblastoma and/or NSCLC) among others. These and other applications, features, and advantages for/of the methods of modulating, inhibiting, and/or killing a cancer cell (such as glioblastoma and/or NSCLC) are described in greater detail elsewhere herein.

In some aspects the method of modulating cancer cells, inhibiting a function and/or activity of a cancer cell, and/or killing a cancer cell can include administering an active agent to a subject having or suspected of having cancer or cell population that can include one or more cancer cells. In some aspects, the active agent can directly (e.g. directly act on or affect a cancer cell) or indirectly (e.g. by stimulating an immune response or other pathway in a subject that subsequently affects the cancer cell or population thereof) to modulate the cancer cell(s), inhibit a function and/or activity of the cancer cell(s), and/or kill the cancer cell(s). Modulation of the cancer cell(s) can include a shift from one cancer cell state to another cancer cell state or normal or non-diseased cell state. Signatures that are characteristic of these cell states are described elsewhere herein.

Methods of screening for one or more agents effective to modulate the cancer cell(s), inhibit a function and/or activity of the cancer cell(s), and/or kill the cancer cell(s) are also described herein. In some aspects, the method of screening for one or more agents can include contacting a cell population composed of one or more cancer or cancer-associated cells having an initial cell state, activity, and/or function with a test agent or library of agents, detecting and/or determining a cell state, activity, function, and/or death of the cancer and/or cancer-associated cell(s), and selecting an agent that is effective to shift the state of one or more cancer cell(s) or otherwise modulate a signature of a cell(s), inhibit a function and/or activity of the cancer cell(s), and/or kill the cancer cell(s).

### Methods of Using a Gene Signature.

Generally, the methods described herein can be effective to analyze the cellular landscape and determine the particular cell states of various cells present in a cancer or as the result of the presence of a cancer, such as glioblastoma or NSCLC. In aspects, the methods described herein can stratify cell identities, types, and/or states with a greater granularity that current methods, which can allow for identification of previously unrecognized and unrealized cell identities, types, and/or states and/or the translation of these cell states into diagnostics and therapies for cancers such as glioblastoma or NSCLC. Described herein are methods and assays capable of detecting various cell-states in various cell types, including cancer cells, methods of diagnosing and/or prognosing a cancer (such as glioblastoma or NSCLC) in a subject based on a cellular landscape of a sample tested and/or signature of one or more cells of a subject. Also described herein are methods of treating a cancer, such as glioblastoma or NSCLC. Also described herein are methods of assays capable of identifying agents effective against a specific cancer cell or population thereof.

Aspects disclosed herein provide methods of detecting and identifying cell states in cancer cells. The cell state can correspond to a cell state in a progression of cell states in the development and progression of a cancer such as glioblastoma or NSCLC. In various aspects, the methods described herein can be used to detect an activated cell state in an astrocyte. Cancer cell states/types can be characterized by a specific and unique cancer signature and/or expression profile. Cancer signatures and expression profiles, including glioblastoma and NSCLC signatures that can be detected via these and other aspects are described in greater detail elsewhere herein.

Aspects disclosed herein, provide methods of diagnosing a cell or tissue in a subject having or being suspected of having a cancer, such as glioblastoma or NSCLC. In some aspects, the sample can be obtained from a subject. In some aspects, the subject suffers from a cancer, such as glioblastoma or NSCLC.

The methods described here and elsewhere herein can be used to stratify a patient population into previously unknown patient pools, which then can be applied to unexpectedly alter and/or improve patient treatment. The methods described here and elsewhere herein can be used to stratify a patient population into previously unknown patient pools, which then can be applied to unexpectedly alter and/or improve patient treatment for a cancer, such as glioblastoma or NSCLC.

Aspects disclosed herein, provide methods of diagnosing and/or prognosing a cancer, where the method comprises the step of detecting a signature, such as gene signature/gene expression profile in one or more cancer cells or tissues and/or cells and/or tissues associated with and/or affected by the cancer. In some aspects, the cancer can be glioblastoma or NSCLC. The order of steps provided herein is exemplary, certain steps may be carried out simultaneously or in a different order. Cancer signatures and expression profiles, including glioblastoma and NSCLC signatures that can be detected via these and other aspects are described in greater detail elsewhere herein.

Aspects disclosed herein provide methods of detecting a cancer, which can include determining a fraction of cells having a particular signature and/or expression profile in a sample from a subject; and diagnosing and/or prognosing the cancer in the subject when the fraction of cells having the particular signature and/or expression profile in the sample is modulated (e.g. either increased or decreased) relative to a fraction of homeostatic or non-diseased control cells or has crossed a predetermined threshold value. Suitable homeostatic of non-diseased controls will be appreciated by those of ordinary skill in the art. Cancer signatures and expression profiles, including glioblastoma and NSCLC signatures that can be detected via these and other aspects are described in greater detail elsewhere herein.

Aspects disclosed herein provide methods of treating a patient having or suspected of having a cancer or a symptom thereof, such as one with a particular signature, by administering an agent effective to modulate the signature of a cancer (e.g. glioblastoma or NSCLC), modulate a function or activity of a cancer cell, kill a cancer cell, increase the sensitivity of a cancer cell to a chemotherapeutic agent or a subject's own immune cell, increase the activity of a subject's own immune system against the cancer cell, or any combination thereof. The method of treating can include exposing of a cell, such as a cancer cell, to an agent capable of killing, inhibiting an activity or function, and/or modulating a signature of a cancer (such as glioblastoma or NSCLC) cell. Exposure of the cells to the agent can occur in vitro, ex vivo, or in vivo. In some aspects, the method of treating a patient described herein can include administering an agent capable of killing, inhibiting an activity or function, and/or modulating a signature of a cancer (such as glioblastoma or NSCLC) cell to the patient.

Aspects disclosed herein provide methods of screening agents to identify agents capable of inhibiting an activity or function, and/or modulating a signature of a cancer (such as glioblastoma or NSCLC) cell. In some aspects, the cell or cells can be isolated from a patient having or suspected of having a cancer such as glioblastoma or NSCLC. Cancer signatures and expression profiles, including glioblastoma and NSCLC signatures that can be detected via these and other aspects are described in greater detail elsewhere herein.

In any of aspects of the methods described above a sample to be processed and/or analyzed using one or more of the methods described herein can contain a population of cells. The population of cells can contain cancer cells, and/or normal non-diseased cells. In some aspects, the population of cells can include a single cell type and/or subtype, a combination of cell types/subtypes, a cell-based therapeutic, an explant, and/or an organoid. The sample can be any biological sample. In some aspects, the sample is obtained from brain tissue, cerebrospinal fluid, or blood. The sample can be obtained from a subject. The subject can have or be suspected of having a cancer, such as glioblastoma and/or NSCLC.

As previously discussed, the method can include detecting and/or measuring a signature and/or expression profile of a cell or cell population. A suitable method and/or technique can be used to detect and/or measure a signature and/or expression profile of a cell or cell population. Suitable techniques include, but are not limited to, an RNA-seq method or technique, an immunoaffinity-based method or technique (e.g. immunohistochemistry, immunocytochemistry, immunoseparation assay, Western analysis, and the like), a polynucleotide sequencing method or technique (e.g. Maxium-Gilbert sequencing, chain-termination sequencing (e.g. Sanger sequencing), shotgun sequencing methods and techniques, bridge PCR, massively parallel signature sequencing, polony sequencing, pyrosequencing, Solexa sequencing, combinatorial probe anchor synthesis, SOLiD sequencing, Ion torrent semiconductor sequencing, nanoball sequencing, heliscope single molecule sequencing, single molecule real time sequencing, nanopore sequencing, microfluidic system-based sequencing, tunneling currents sequencing, sequencing by hybridization, sequencing with mass spectrometry, a RNA polymerase based-sequencing method, an in vitro virus high-throughput method, a bisulfite sequencing technique, or a combination thereof), a PCR based method or technique (e.g. PCR, RT-PCR, qPCR, RT-qPCR, etc.), a protein analysis technique (e.g. mass spectrometry, polypeptide sequencing, an immunoaffinity method or technique, and the like), an epigenome analysis technique, and combinations thereof. Other suitable methods and techniques will be appreciated by those of ordinary skill in the art. In some aspects, the technique or method may be able to measure the expression at the single-cell level. In some aspects, the technique may be a single-cell RNA-seq method or technique.

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (1¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

In certain aspects, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain aspects, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain aspects, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain aspects, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain aspects, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

In some aspects, differences between cell-state between a cancer cell and a normal or non-cancer can include comparing a gene expression distribution of a cancer cell(s) with a gene expression distribution of normal or non-diseased cells as determined by a single-cell gene expression method (e.g. single-cell RNA-seq) or another suitable method described herein.

In certain example aspects, assessing the cell (sub)types and states present in the in sample may comprise analysis of expression matrices from expression data, performing dimensionality reduction, graph-based clustering and deriving list of cluster-specific genes in order to identify cell types and/or states present in the in vivo system. These marker genes may then be used throughout to relate one cell state to another. For example, these marker genes can be used to relate a cancer cell (sub)types and/or states to the non-diseased or normal cell (sub(types) and/or states. The same analysis may then be applied to the source material for the sample or a control. From both sets of the expression analysis an initial distribution of gene expression data is obtained. In certain aspects, the distribution may be a count-based metric for the number of transcripts of each gene present in a cell. Further the clustering and gene expression matrix analysis allow for the identification of key genes in the homeostatic cell-state and the DAA cell state, such as differences in the expression of key transcription factors. In certain example aspects, this may be done conducting differential expression analysis. Other analytic methods can be included or performed on their own. Such additional methods are discussed in Examples herein. For example, in the Examples below, differential gene expression analysis can be conducted and/or data therefrom be processed according to a method described there and/or elsewhere herein to determine a cancer cell state and/or type or the presence thereof, as well as diagnose, prognose, and/or otherwise identify a cancer in a subject. In some aspects, the cancer is glioblastoma and/or NSCLC.

In some aspects, identification of a cancer cell or cell population can include detecting a shift, such as a statistically significant shift, in the cell-state as indicated by a modulation (e.g. an increased distance) in the gene expression space between a first cancer cell-state and a second cancer cell state and/or a normal or non-diseased cell. In certain aspects, the distance is measured by a Euclidean distance, Pearson coefficient, Spearman coefficient, or combination thereof.

In certain aspects, the gene expression space comprises 10 or more genes, 20 or more genes, 30 or more genes, 40 or more genes, 50 or more genes, 100 or more genes, 500 or more genes, or 1000 or more genes. In certain aspects, the expression space defines one or more cell pathways.

The statistically significant shift may be at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%. The statistical shift may include the overall transcriptional identity or the transcriptional identity of one or more genes, gene expression cassettes, or gene expression signatures of the a first cancer cell state compared to a second cancer cell state and/or a normal or non-diseased state (i.e., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% of the genes, gene expression cassettes, or gene expression signatures are statistically shifted in a gene expression distribution). A shift of 0% means that there is no difference to the homeostatic and/or activated cell state.

A gene distribution may be the average or range of expression of particular genes, gene expression cassettes, or gene expression signatures in a first cancer cell-state, a second cancer cell state, and/or a normal or non-diseased cell state (e.g., a plurality of a cell of interest from a subject may be sequenced and a distribution is determined for the expression of genes, gene expression cassettes, or gene expression signatures). In certain aspects, the distribution is a count-based metric for the number of transcripts of each gene present in a cell. A statistical difference between the distributions indicates a shift. The one or more genes, gene expression cassettes, or gene expression signatures may be selected to compare transcriptional identity based on the one or more genes, gene expression cassettes, or gene expression signatures having the most variance as determined by methods of dimension reduction (e.g., tSNE analysis).

In certain aspects, comparing a gene expression distribution comprises comparing the initial cells with the lowest statistically significant shift as compared to the a second cell state or a normal or non-diseased cell (e.g., determining shifts when comparing only the cancer cells with a shift of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10% to the homeostatic cell state). In certain example aspects, statistical shifts may be determined by defining a normal or non-diseased cell and/or cancer cell state score.

For example, a gene list of key genes enriched in a homeostatic/activated model may be defined. To determine the fractional contribution to a cell's transcriptome to that gene list, the total log (scaled UMI+1) expression values for gene with the list of interest are summed and then divided by the total amount of scaled UMI detected in that cell giving a proportion of a cell's transcriptome dedicated to producing those genes. Thus, statistically significant shifts may be shifts in an initial score for the normal or non-diseased score towards the cancer cell state score.

The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein may refer to a single mRNA or target nucleic acid to be sequenced. The UMI may also be used to determine the number of transcripts that gave rise to an amplified product, or in the case of target barcodes as described herein, the number of binding events. In preferred aspects, the amplification is by PCR or multiple displacement amplification (MDA). Unique molecular identifiers can be used, for example, to normalize samples for variable amplification efficiency. For example, in various aspects, featuring a solid or semisolid support (for example a hydrogel bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support. Design and construction of UMIs are generally known in the art and can be used with the methods herein. See e.g., Islam S. et al., 2014. Nature Methods No:11, 163-166, International Patent Publication No. WO 2014/047561. Other barcoding and tagging methods can be used with the invention herein, which are also known in the art. See e.g. Kress et al., “Use of DNA barcodes to identify flowering plants” Proc. Natl. Acad. Sci. U.S.A. 102(23):8369-8374 (2005), Koch H., “Combining morphology and DNA barcoding resolves the taxonomy of Western Malagasy Liotrigona Moure, 1961” African Invertebrates 51(2): 413-421 (2010); and Seberg et al., “How many loci does it take to DNA barcode a crocus?” PLoS One 4(2):e4598 (2009), CBOL Plant Working Group, “A DNA barcode for land plants” PNAS 106(31):12794-12797 (2009), Kress et al., “DNA barcodes: Genes, genomics, and bioinformatics” PNAS 105(8):2761-2762 (2008), Lahaye et al., “DNA barcoding the floras of biodiversity hotspots” Proc Natl Acad Sci USA 105(8):2923-2928 (2008), Ausubel, J., “A botanical macroscope” Proceedings of the National Academy of Sciences 106(31):12569 (2009), Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106(7):2289-94).

In some aspects, the method can include generating a sequencing library. Methods of generating such a library are generally known in the art and can be used with the invention described herein.

Other methods for assessing differences in the normal or non-diseased and cancer cells may be employed. In certain example aspects, an assessment of differences in the cancer and normal or non-diseased proteome may be used to further identify key differences in cell type and sub-types or cells. states. For example, isobaric mass tag labeling and liquid chromatography mass spectroscopy may be used to determine relative protein abundances in the ex vivo and in vivo systems. Description provided elsewhere herein further disclosure on leveraging proteome analysis within the context of the methods disclosed herein.

The invention provides biomarkers (e.g., phenotype specific or cell type) for the identification, diagnosis, prognosis and manipulation of cell properties, for use in a variety of diagnostic and/or therapeutic indications, particularly for cancer (e.g. glioblastoma and/or NSCLC). Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain aspects, biomarkers include the signature genes or signature gene products, and/or cells as described herein.

Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations at risk or suffering from an immune response based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Suitably, an altered quantity or phenotype of the immune cells in the subject compared to a control subject having normal immune status or not having a disease comprising an immune component indicates that the subject has an impaired immune status or has a disease comprising an immune component or would benefit from an immune therapy.

Hence, the methods may rely on comparing the quantity of immune cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further aspect, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In one aspect, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

In certain aspects, signature genes and biomarkers related to the disease may be a cancer (e.g. a glioblastoma or a NSCLC), such as by comparing single cell expression profiles obtained from healthy or normal cells and diseased (e.g. cancer) cells.

In one particular aspect, signature genes and biomarkers related to the cancer may be identified by comparing single cell expression profiles obtained from normal or non-diseased cells and diseased (or cancer) cells.

Various aspects and aspects of the invention may involve analyzing gene signatures, protein signature, and/or other genetic or epigenetic signature based on single cell analyses (e.g. single cell RNA sequencing) or alternatively based on cell population analyses, as is defined and described herein elsewhere.

A gene profile can be a gene signature, or expression profile. In one aspect, the gene expression profile measures upregulation or down regulation of particular genes or pathways and is further defined and described elsewhere herein. In particular instances, the gene expression profile comprises one or more genes from genes of a first cancer cell state signature, a second gene signature, and/or a normal or non-diseased cell gene signature.

The methods described herein can be used to isolate a cell or population thereof from a sample where the isolated cells have a desired signature, such as a cancer signature as described herein. Methods of physically isolating cells (e.g. flow cytometry, immunoseparation, based on the expression of one or more genes and/or proteins are generally known in the art and can be used to detect and/or isolate cells having an inventive signature described herein. Thus, also described herein are cells and populations thereof that can have a unique cancer signature such as any described herein. In some aspects, the cancer signature is a signature described herein. The cell(s) can be isolated from a sample that was obtained from a subject having or suspected of having cancer and/or in need of treatment. The cells can be used in a screening method, such as a screening method to identify an agent effective against the isolated cancer cells. Exemplary screening methods are described in greater detail elsewhere herein.

### Computer Systems for Processing Biological Information.

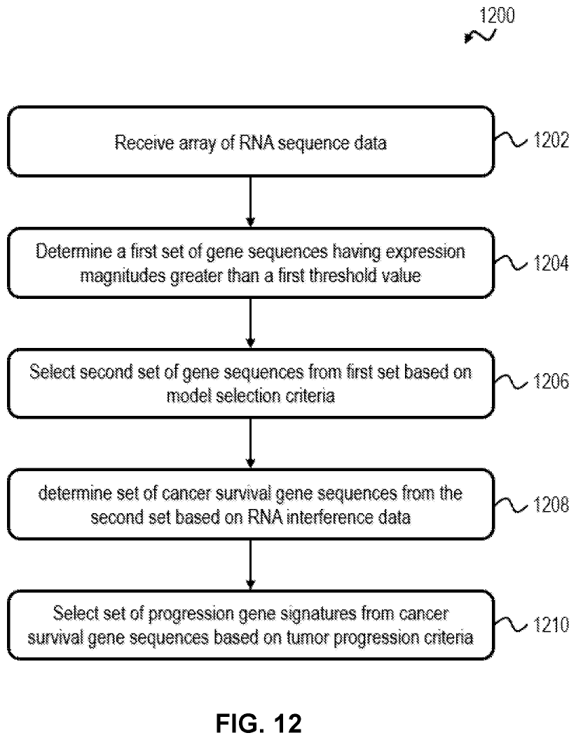

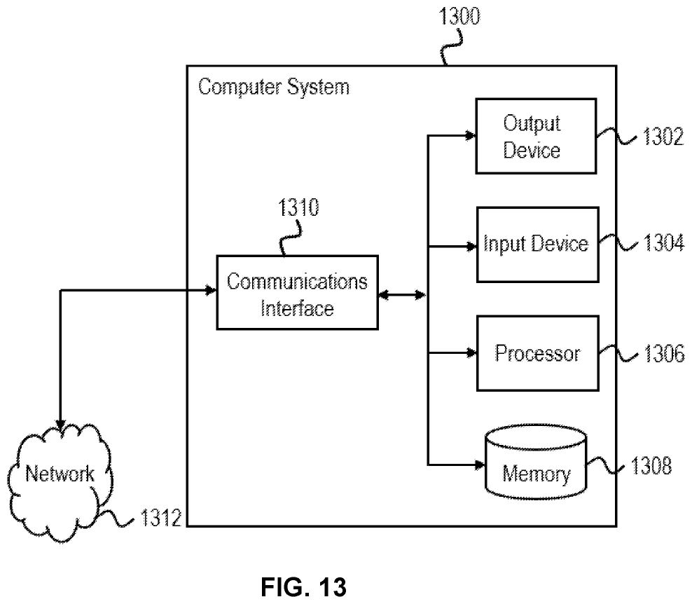

FIG. 12 shows a flow diagram of an example process 1200 for processing biological information. The process 1200 can be executed, for example, by a computer server, such as an example computer system discussed below in relation to FIG. 13. The process 1200 can be used to execute a biomarker pipeline that integrates genome-wide RNAi screens with comprehensive RNA-seq and clinical data to identify survival gene-based progression gene signatures. The biomarkers can be utilized for cancer diagnosis and therapeutic intervention in patients. The process 1200 includes receiving an array of RNA sequence data associated with a group of patients (1202). The RNA sequence data can include, for example, RNA-seq data or microarray data. As another example, the RNA sequence data can include RNA-seq data or clinical data from databases such as, for example, the cancer genome atlas (TOGA), which contains publicly accessible RSEM-processed RNA-seq data for more than 500 quality-controlled primary tumor samples in LUAD and LUSC, and genome-wide microarray profiling for 528 quality-controlled primary GBM samples. However, RNA-seq or microarray data from other databases for various other cancers can also be utilized.

The process 1200 further includes determining a first set of gene sequences having expression magnitudes greater than a first threshold value (1202). In particular, the process 1200 includes identifying the most ubiquitous gene expressions in the in at least one subtype form the array of RNA sequence data. The first threshold value can be set such that only those gene sequences that show large expression magnitudes are determined to be in the first set of gene sequences. The first threshold value can be used to indicate the desired expression magnitude. In some examples, the first threshold value can be between 95^{th} percentile to 99^{th} percentile. In some other examples, the first threshold value can be equal to 99^{th} percentile. The process 1200 also includes selecting a second set of gene sequences from the first set of gene sequences based on a model selection criteria (1206). The first set of gene sequences can include gene sequences, which while having associated gene expression magnitude above the first threshold, may not contribute to survival of the cancer cells. It would be efficient to remove such gene sequences from further analysis. To remove such gene sequences, the system can execute statistical model selection criteria, such as, for example, Bayesian information criterion (BIC), Akaike information criterion (AIC), and other likelihood based metrics, to remove such gene sequences from the first set of gene sequences. The resulting second set of gene sequences can be a subset of the first set of gene sequences and can include those gene sequences from the first set of gene sequences having the lowest BIC score. Once example of selecting a subset of genes is indicated in Table 2, which show a total number of gene sequences that result in the lowest BIC scores. Of course, the data shown in Table 2 is only an example.

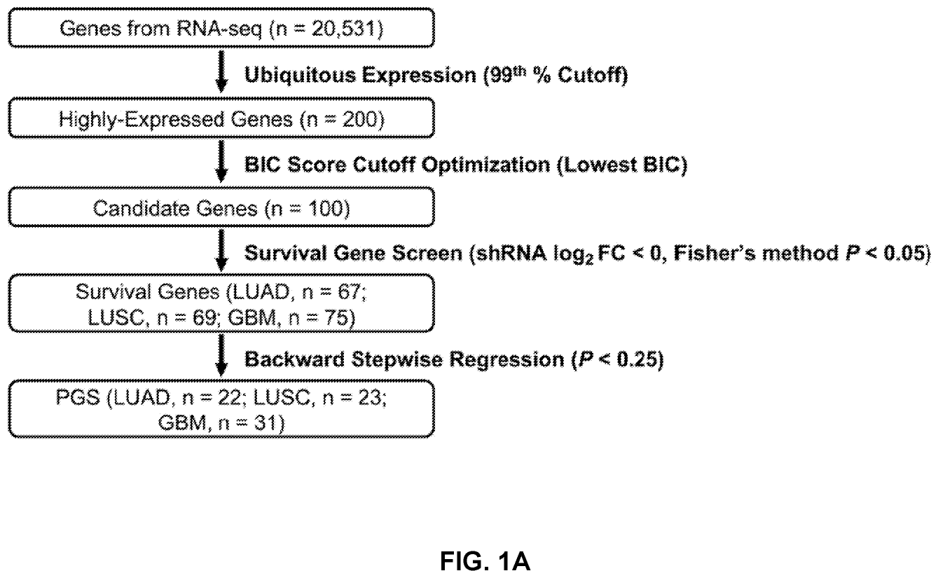

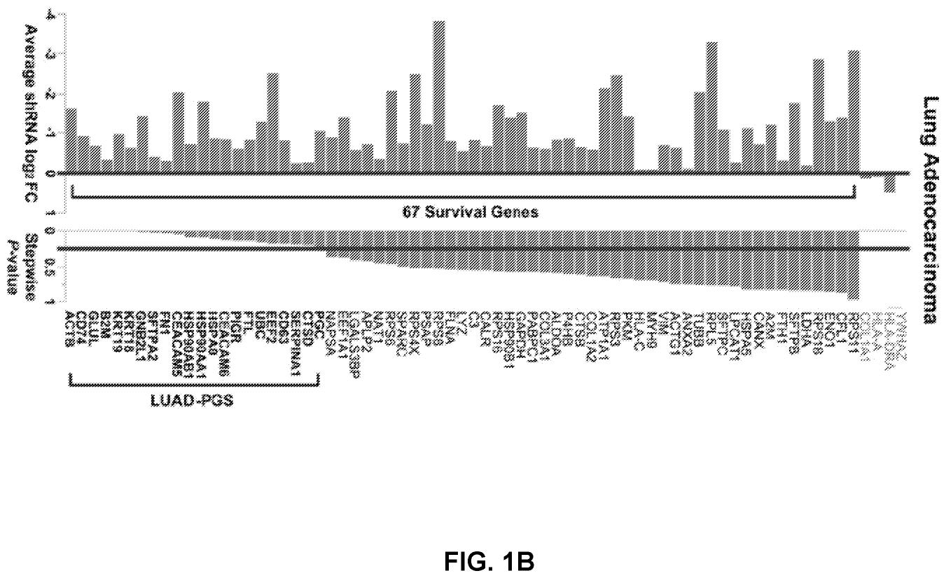

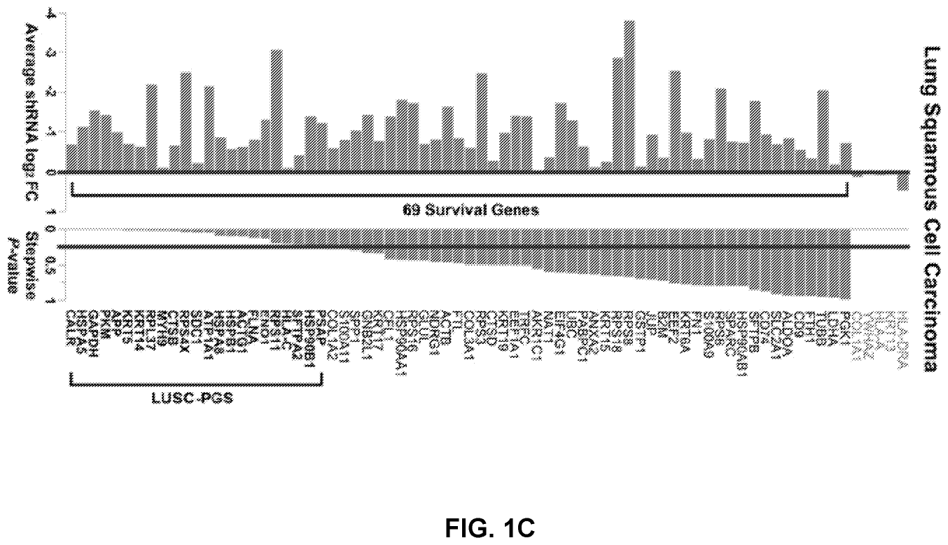

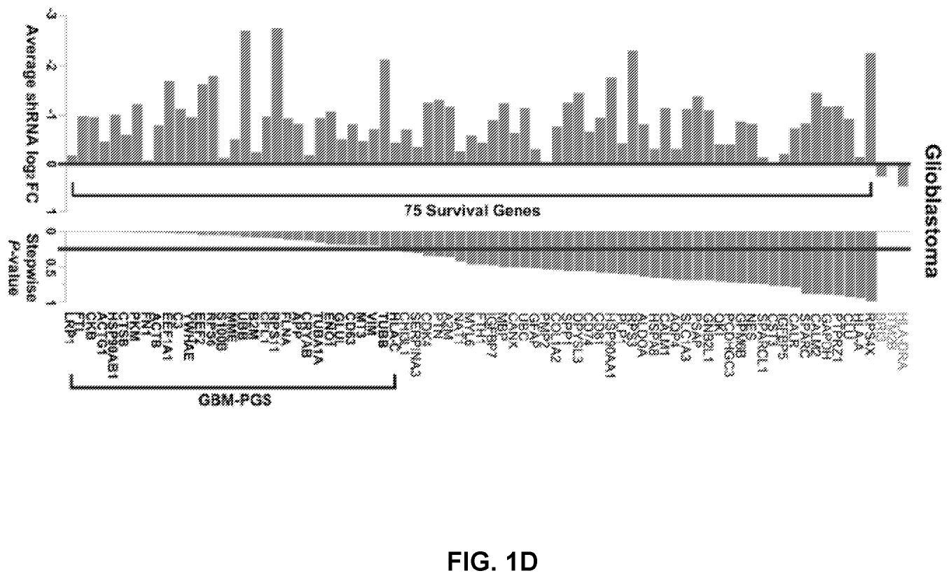

The process 1200 further includes determining a set of cancer survival gene sequences from the second set of gene sequences based on cross-referencing each gene sequence from the second set of gene sequences with RNA interference data (1208). In particular, the gene sequences can be cross-referenced one or more cancer cell lines with genome-wide RNAi screen data. In some examples, the RNAi screen data can be obtained from the Cancer Dependency Map (DepMap), which includes Project Achilles form Broad Institute. However, other sources of RNAi screen data can also be utilized. The cell lines can be associated with a cancer subtype, such as, for example, LUAD, LUSC, and GBM. In some examples, the process can include determining the set of cancer survival gene sequences based in part on selection of those gene sequences from the second set of gene sequences having corresponding fold change of less than zero in the RNAi data. For example, some RNAi results are presented in log₂ fold changes that are indicative of shRNA loss. Thus, lower fold change values indicate a stronger depletion of shRNAs, and thus a larger reduction in cell viability when the corresponding gene sequence is removed. As an example, a shRNA fold change of less than zero can be selected to determine those gene sequences that are associated with cancer cell survival. FIG. 1B shows an example list of 67 survival genes that have an average shRNA fold change of less than zero. All those gene sequences that have an average shRNA fold change of equal to or greater than zero are not included in the set of cancer survival gene sequences. In some examples, the threshold value of zero, in relation to the fold change, can be different. For example, the process can include determining the fold change threshold value based on one-tailed one-sample t-test to determine the significance of the fold change threshold value. In some other examples, the process can include utilizing the Fisher's combined probability test to determine a false discovery rate (FDR)-adjusted significance of average shRNA fold change.

The process 1200 also includes selecting from the set of cancer survival gene sequences as set of progression gene signatures based on a tumor progression criteria (1210). In particular, the process 1200 includes applying a tumor progression criteria to the set of cancer survival gene sequences to select a subset of gene sequences that can serve as progression gene signatures. The tumor progression criteria can include, for example, a backward stepwise regression model with a predetermined p-value. The tumor progression criteria can include forward stepwise regression with a predetermined p-value, bidirectional stepwise regression with a predetermined p-value, forward stepwise regression minimizing Bayesian Information Criterion (BIC) value, backward stepwise regression minimizing BIC value, bidirectional stepwise regression minimizing BIC value, or a combination thereof. In some aspects, the predetermined p-value can be about 0.10 to about 0.35, or about 0.20 to about 0.35.

In particular aspects, the process can enter the set of survival genes into the backward stepwise variable regression model trained on a yes/no indicator of tumor progression with a p-value of 0.25 to determine the set of PGSs. FIG. 1B shows an example list of 22 PGSs selected from the set of cancer survival gene sequences based on a predetermined p-value of 0.25, and indicated by the label “LUAD-PGS.” The p-value of 0.25 shown in FIG. 1B is only an example, and other predetermined p-values can also be selected. In some aspects, the predetermined p-value can be about 0.10 to about 0.35, or about 0.20 to about 0.35.

In some aspects, the stepwise regression using a p-value threshold of 0.25 results in the PGS with optimal accuracy in stratifying patient risk for cancer progression. Not wishing to be bound by any particular theory, it is believed the optimal results may have been due to the production of suppressor effects that can occur from forward/bidirectional approaches. The process of adding predictors to the model based on a criterion may result in the inclusion of predictors that are only significant when all other predictors are held constant. In addition, these approaches may add predictors that render other predictors already included in the model insignificant. Both drawbacks may be avoided by using a backward stepwise regression approach. Also, using the p-value threshold of 0.25 as the criterion resulted in the optimal model since minimizing the BIC value is a very strict criterion that did not take into account interactions between the candidate genes.

Selecting a higher p-value results in a more accurate model but also a greater chance for overfitting while selecting a lower p-value results in less chance for overfitting but also a less accurate model. Using higher p-values also generally result in more complex models while lower p-values construct oversimplified models. Not wishing to be bound by any particular theory, it is believed that selecting a predetermined p-value of about 0.10 to about 0.35, or about 0.20 to about 0.35, or about 0.25 can provide optimal results.

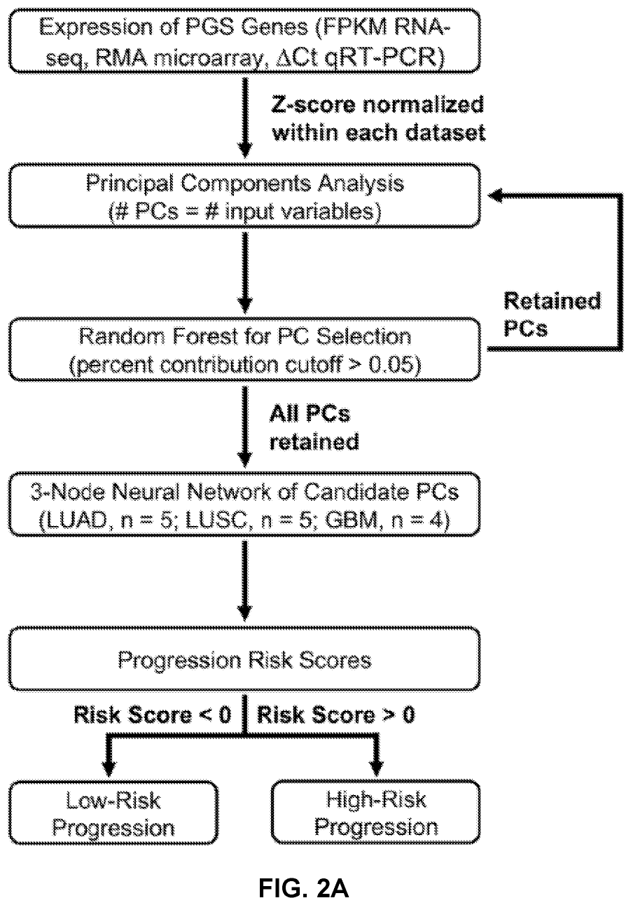

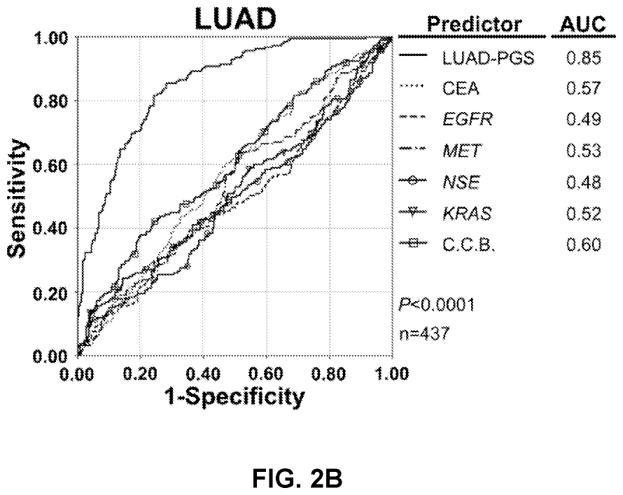

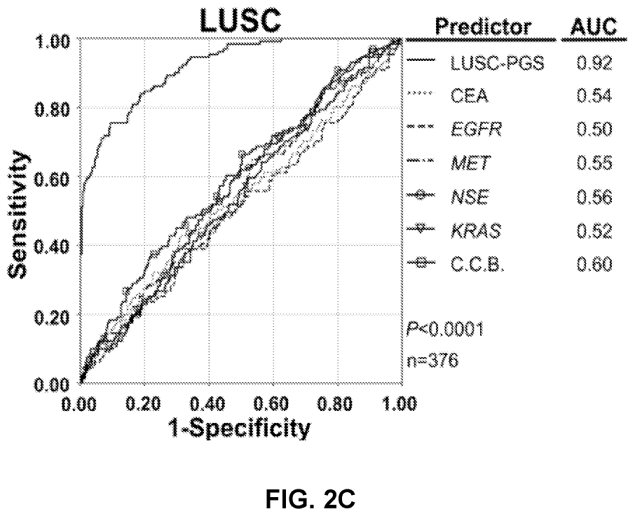

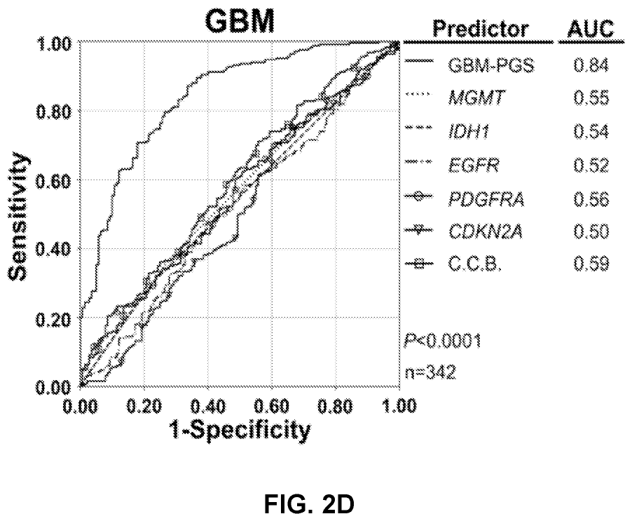

The PGSs determined by the process 1200 discussed above can be utilized as biomarkers of cancer progression. In some examples, the process 1200 can include ranking patients for cancer risk based on the biomarkers including one or more PGSs associated with the cancer. The process 1200 can include displaying the list of patients according to the rank. In some examples, the process 1200 can determine the cancer risk associated with one or more patients based on the biomarkers including one or more PGSs associated with the cancer, and provide the cancer risk on an output device. In some examples, the process 1200 can include instructions to execute the process shown in FIG. 2A for derivation of PGS risk scores and patient risk stratification, and output the results to the patient or a health provider.

FIG. 13 shows the general architecture of an illustrative computer system 1300 that may be employed to implement any of the computer systems discussed herein in accordance with some implementations. The computer system 1300 comprises one or more processors 1306 communicatively coupled to memory 1308, one or more communications interfaces 1310, and one or more output devices 1302 (e.g., one or more display units) and one or more input devices 1304.

In the computer system 1300, the memory 1308 may comprise any computer-readable storage media, and may store computer instructions such as processor-executable instructions for implementing the various functionalities described herein for respective systems, as well as any data relating thereto, generated thereby, or received via the communications interface(s) or input device(s) (if present). In particular, the memory 1308 can store instructions related to the process 1200 discussed above in relation to FIG. 12. Furthermore, the memory 1308 can store the array of RNA sequence data associated with patients, the first set of gene sequences, the second set of gene sequences, the model selection criteria, the set of cancer survival gene sequences, RNA interference data, the set of progression gene signatures, and tumor progression criteria. Furthermore, the memory 1308 can store RNA-seq or microarray data associated with one or more types or subtypes of cancers, such as for example, LUAD, LUSC, and GBM. The memory 1308 can also store the at least one of the BIC, AIC, and any other likelihood based metrics. The memory 1308 may also store data related to cancer cell lines and genome-wide RNAi screen data. The memory 1308 may also store the threshold value for determining the first set of gene sequences, and one or more predetermined p-values. In some examples, one or more data or instructions discussed above in relation to the memory 1308 can be stored in whole or in part in a remote memory that can be accessed over the network 1312.

The processor(s) 1306 may be used to execute instructions stored in the memory 1308 and, in so doing, also may read from or write to the memory various information processed and or generated pursuant to execution of the instructions. The processor 1306 of the computer system 1300 also may be communicatively coupled to or control the communications interface(s) 1310 to transmit or receive various information pursuant to execution of instructions. For example, the communications interface(s) 1310 may be coupled to a wired or wireless network, bus, or other communication means and may therefore allow the computer system 1300 to transmit information to or receive information from other devices (e.g., other computer systems). While not shown explicitly in the computer system 1300, one or more communications interfaces facilitate information flow between the components of the system 1300. In some implementations, the communications interface(s) may be configured (e.g., via various hardware components or software components) to provide a website as an access portal to at least some aspects of the computer system 1300. Examples of communications interfaces 1310 include user interfaces (e.g., web pages), through which the user can communicate with the computer system 1300.

The output devices 1302 of the computer system 1300 may be provided, for example, to allow various information to be viewed or otherwise perceived in connection with execution of the instructions. The input device(s) 1304 may be provided, for example, to allow a user to make manual adjustments, make selections, enter data, or interact in any of a variety of manners with the processor during execution of the instructions. Additional information relating to a general computer system architecture that may be employed for various systems discussed herein is provided further herein.

Implementations of the subject matter and the operations described in this specification can be implemented in digital electronic circuitry, or in computer software embodied on a tangible medium, firmware, or hardware, including the structures disclosed in this specification and their structural equivalents, or in combinations of one or more of them. Implementations of the subject matter described in this specification can be implemented as one or more computer programs, i.e., one or more components of computer program instructions, encoded on computer storage medium for execution by, or to control the operation of, data processing apparatus. The program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can include a source or destination of computer program instructions encoded in an artificially-generated propagated signal. The computer storage medium can also be, or be included in, one or more separate physical components or media (e.g., multiple CDs, disks, or other storage devices).

### REFERENCES

References are cited herein throughout using the format of reference number(s) enclosed by parentheses corresponding to one or more of the following numbered references. For example, citation of references numbers 1 and 2 immediately herein below would be indicated in the disclosure as (Refs. 1 and 2).
(1) Siegel, R. L., Miller, K. D. & Jemal, A. Cancer statistics, 2019. CA Cancer J. Clin. 69, 7-34. (2019).
(2) Ostrom, Q. T. et al. CBTRUS statistical report: primary brain and other central nervous system tumors diagnosed in the United States in 2009-2013. Neuro Oncol. 18, v1-v75. (2016).
(3) Wick, W., Osswald, M., Wick, A. & Winkler, F. Treatment of glioblastoma in adults. Ther. Adv. Neurol. Disord. 11, 1756286418790452. (2018).
(4) Davis, M. E. Glioblastoma: overview of disease and treatment. Clin. J. Oncol. Nurs. 20, S2-8. (2016).
(5) Torre, L. A., Siegel, R. L. &Jemal, A. Lung cancer statistics. Adv. Exp. Med. Biol. 893, 1-19. (2016).
(6) Testa, U., Castelli, G. & Pelosi, E. Lung cancers: molecular characterization, clonal heterogeneity and evolution, and cancer stem cells. Cancers (Basel). (2018)
(7) Karnofsky, D. A. & Burchenal, J. H. in Evaluation of Chemotherapeutic Agents (ed C. M MacLeod) 196-196 (Columbia University Press, 1949).
(8) Amin, M. B. et al. (2017) The Eighth Edition AJCC Cancer Staging Manual: Continuing to build a bridge from a population-based to a more “personalized” approach to cancer staging. CA Cancer J Clin 67, 93-99. (2017).
(9) Kelly, C. M. & Shahrokni, A. Moving beyond Karnofsky and ECOG performance status assessments with new technologies. J. Oncol. 2016, U.S. Pat. No. 6,186,543. (2016).
(10) Ludwig, J. A. & Weinstein, J. N. Biomarkers in cancer staging, prognosis and treatment selection. Nat. Rev. Cancer 5, 845-856. (2005).
(11) Lee, S. Y. Temozolomide resistance in glioblastoma multiforme. Genes Dis. 3, 198-210. (2016).
(12) Riihimaki, M. et al. Metastatic sites and survival in lung cancer. Lung Cancer 86, 78-84. (2014).
(13) Duma, N., Santana-Davila, R. & Molina, J. R. Non-small cell lung cancer: epidemiology, screening, diagnosis, and treatment. Mayo Clin. Proc. 94, 1623-1640. (2019).
(14) Villalobos, P. & Wistuba, I. I. Lung cancer biomarkers. Hematol. Oncol. Clin. North Am. 31, 13-29. (2017).
(15) Hegi, M. E. et al. Correlation of O⁶-methylguanine methyltransferase (MGMT) promoter methylation with clinical outcomes in glioblastoma and clinical strategies to modulate MGMT activity. J. Clin. Oncol. 26, 4189-4199. (2008).
(16) Finocchiaro, G., Toschi, L., Gianoncelli, L., Baretti, M. & Santoro, A. Prognostic and predictive value of MET deregulation in non-small cell lung cancer. Ann. Transl. Med. 3, 83. (2015).
(17) Murphy, S. F. et al. Connexin 43 inhibition sensitizes chemoresistant glioblastoma cells to temozolomide. Cancer Res. 76, 139-149. (2016).
(18) Nakamura, H., Kawasaki, N., Taguchi, M. & Kabasawa, K. Survival impact of epidermal growth factor receptor overexpression in patients with non-small cell lung cancer: a meta-analysis. Thorax 61, 140-145. (2006).
(19) Meert, A. P. et al. The role of EGF-R expression on patient survival in lung cancer: a systematic review with meta-analysis. Eur. Respir. J. 20, 975-981. (2002).
(20) Martin, P., Leighl, N. B., Tsao, M. S. & Shepherd, F. A. KRAS mutations as prognostic and predictive markers in non-small cell lung cancer. J. Thorac. Oncol. 8, 530-542. (2013).
(21) Roman, M. et al. KRAS oncogene in non-small cell lung cancer: clinical perspectives on the treatment of an old target. Mol. Cancer 17, 33. (2018).
(22) Binabaj, M. M. et al. The prognostic value of MGMT promoter methylation in glioblastoma: a meta-analysis of clinical trials. J. Cell Physiol. 233, 378-386. (2018).
(23) Bailey, A. M. et al. Implementation of biomarker-driven cancer therapy: existing tools and remaining gaps. Discov. Med. 17, 101-114 (2014).
(24) Vogelstein, B. et al. Cancer genome landscapes. Science 339, 1546-1558. (2013).
(25) Lawrence, M. S. et al. Discovery and saturation analysis of cancer genes across 21 tumour types. Nature 505, 495-501. (2014).
(26) Nevins, J. R. & Potti, A. Mining gene expression profiles: expression signatures as cancer phenotypes. Nat. Rev. Genet. 8, 601-609. (2007).
(27) Larsen, J. E. et al. Gene expression signature predicts recurrence in lung adenocarcinoma. Clin. Cancer Res. 13, 2946-2954. (2007).
(28) Larsen, J. E. et al. Expression profiling defines a recurrence signature in lung squamous cell carcinoma. Carcinogenesis 28, 760-766. (2007).
(29) Chen, W., Yu, Q., Chen, B., Lu, X. & Li, Q. The prognostic value of a seven-microRNA classifier as a novel biomarker for the pre-diction and detection of recurrence in glioma patients. Oncotarget 7, 53392-53413. (2016).
(30) Chen, H. Y. et al. A five-gene signature and clinical outcome in non-small-cell lung cancer. N. Engl. J. Med. 356, 11-20. (2007).
(31) Lu, Y., Wang, L., Liu, P., Yang, P. & You, M. Gene-expression signature predicts postoperative recurrence in stage I non-small cell lung cancer patients. PLoS ONE 7, e30880. (2012).
(32) Fatai, A. A. & Gamieldien, J. A 35-gene signature discriminates between rapidly- and slowly-progressing glioblastoma multiforme and predicts survival in known subtypes of the cancer. BMC Cancer 18, 377. (2018).
(33) Alkhateeb, A. et al. Transcriptomics signature from next-generation sequencing data reveals new transcriptomic biomarkers related to prostate cancer. Cancer Inform. 18, 1176935119835522. (2019).
(34) Hamzeh, O. et al. A hierarchical machine learning model to discover gleason grade-specific biomarkers in prostate cancer. Diagnostics (Basel). (2019).
(35) Director's Challenge Consortium for the Molecular Classification of Lung, A. et al. Gene expression-based survival prediction in lung adenocarcinoma: a multi-site, blinded validation study. Nat. Med. 14, 822-827, (2008).
(36) Sun, Z., Wigle, D. A. & Yang, P. Non-overlapping and non-cell-type-specific gene expression signatures predict lung cancer survival. J. Clin. Oncol. 26, 877-883. (2008).
(37) Drucker, E. & Krapfenbauer, K. Pitfalls and limitations in translation from biomarker discovery to clinical utility in predictive and personalised medicine. EPMA J. 4, 7. (2013).
(38) McDermott, J. E. et al. Challenges in biomarker discovery: combining expert insights with statistical analysis of Complex Omics Data. Expert Opin. Med. Diagn 7, 37-51. (2013).
(39) Zhang, L., Yoder, S. J. & Enkemann, S. A. Identical probes on different high-density oligonucleotide microarrays can produce different measurements of gene expression. BMC Genom. 7, 153 (2006).
(40) Mohr, S. E., Smith, J. A., Shamu, C. E., Neumuller, R. A. & Perrimon, N. RNAi screening comes of age: improved techniques and complementary approaches. Nat. Rev. Mol. Cell Biol. 15, 591-600. (2014).
(41) Sheng, K. L., Pridham, K. J., Sheng, Z., Lamouille, S. & Varghese, R. T. Functional blockade of small GTPase RAN inhibits glio-blastoma cell viability. Front. Oncol. 8, 662. (2018).
(42) Varghese, R. T. et al. Survival kinase genes present prognostic significance in glioblastoma. Oncotarget 7, 20140-20151. (2016).
(43) Goidts, V. et al. RNAi screening in glioma stem-like cells identifies PFKFB4 as a key molecule important for cancer cell survival. Oncogene 31, 3235-3243. (2012).
(44) D'Alesio, C. et al. RNAi screens identify CHD4 as an essential gene in breast cancer growth. Oncotarget 7, 80901-80915. (2016).
(45) Luo, C. W. et al. CHD4-mediated loss of E-cadherin determines metastatic ability in triple-negative breast cancer cells. Exp. Cell Res. 363, 65-72. (2018).
(46) Tsherniak, A. et al. Defining a cancer dependency map. Cell 170, 564-576. (2017).
(47) Cerami, E. et al. The cBio cancer genomics portal: an open platform for exploring multidimensional cancer genomics data. Cancer Discov. 2, 401-404. (2012).
(48) Gao, J. et al. Integrative analysis of complex cancer genomics and clinical profiles using the cBioPortal. Sci. Signal. (2013).
(49) Bild, A. H. et al. Oncogenic pathway signatures in human cancers as a guide to targeted therapies. Nature 439, 353-357 (2006).
(50) Lee, E. S. et al. Prediction of recurrence-free survival in postoperative non-small cell lung cancer patients by using an integrated model of clinical information and gene expression. Clin. Cancer Res. 14, 7397-7404. (2008).
(51) Hou, J. et al. Gene expression-based classification of non-small cell lung carcinomas and survival prediction. PLoS ONE 5, e10312. (2010).
(52) Rousseaux, S. et al. Ectopic activation of germline and placental genes identifies aggressive metastasis-prone lung cancers. Sci. Transl. Med. (2013).
(53) Gusev, Y. et al. The REMBRANDT study, a large collection of genomic data from brain cancer patients. Sci. Data 5, 180158. (2018).
(54) Buffa, F. M., Harris, A. L., West, C. M. & Miller, C. J. Large meta-analysis of multiple cancers reveals a common, compact and highly prognostic hypoxia metagene. Br. J. Cancer 102, 428-435. (2010).
(55) Xie, F., Xiao, P., Chen, D., Xu, L. & Zhang, B. miRDeepFinder: a miRNA analysis tool for deep sequencing of plant small RNAs. Plant Mol. Biol. (2012).
(56) 56R Core Team. R: A Language and Environment for Statistical Computing. Software version 3.6.1. R Foundation for Statistical Computing. Vienna, Austria. (2019).
(57) Fabregat, A. et al. Reactome pathway analysis: a high-performance in-memory approach. BMC Bioinform. 18, 142. (2017).
(58) Mollinedo, F. Neutrophil degranulation, plasticity, and cancer metastasis. Trends Immunol. 40, 228-242. (2019).
(59) Lee, M. & Rhee, I. Cytokine signaling in tumor progression. Immune Netw. 17, 214-227. (2017).
(60) Szklarczyk, D. et al. STRING v11: protein-protein association networks with increased coverage, supporting functional discovery in genome-wide experimental datasets. Nucl. Acids Res. 47, D607-D613. (2019).
(61) Grunnet, M. & Sorensen, J. B. Carcinoembryonic antigen (CEA) as tumor marker in lung cancer. Lung Cancer 76, 138-143. (2012).
(62) Isgro, M. A., Bottoni, P. & Scatena, R. Neuron-specific enolase as a biomarker:
biochemical and clinical aspects. Adv. Exp. Med. Biol. 867, 125-143. (2015).
(63) Szopa, W., Burley, T. A., Kramer-Marek, G. & Kaspera, W. Diagnostic and therapeutic biomarkers in glioblastoma: current status and future perspectives. Biomed. Res. Int 2017, U.S. Pat. No. 8,013,575. (2017).
(64) Pirker, R. Adjuvant chemotherapy in patients with completely resected non-small cell lung cancer. Transl. Lung Cancer Res. 3, 305-310. (2014).
(65) Cosse, J. P. & Michiels, C. Tumour hypoxia affects the responsiveness of cancer cells to chemotherapy and promotes cancer pro-gression. Anticancer Agents Med. Chem. 8, 790-797. (2008).
(66) Bhandari, V. et al. Molecular landmarks of tumor hypoxia across cancer types. Nat. Genet. 51, 308-318. (2019).
(67) Wu, J. et al. Heat shock proteins and cancer. Trends Pharmacol. Sci. 38, 226-256. (2017).
(68) Ciocca, D. R. & Calderwood, S. K. Heat shock proteins in cancer: diagnostic, prognostic, predictive, and treatment implications. Cell Stress Chaperones 10, 86-103. (2005).
(69) Fife, C. M., McCarroll, J. A. & Kavallaris, M. Movers and shakers: cell cytoskeleton in cancer metastasis. Br. J. Pharmacol. 171, 5507-5523. (2014).
(70) Jerhammar, F. et al. Fibronectin 1 is a potential biomarker for radioresistance in head and neck squamous cell carcinoma. Cancer Biol. Ther. 10, 1244-1251. (2010).
(71) Jin, Y. & Yang, Y. Identification and analysis of genes associated with head and neck squamous cell carcinoma by integrated bio-informatics methods. Mol. Genet. Genom. Med. 7, e857. (2019).
(72) Cao, X. X. et al. RACK1: A superior independent predictor for poor clinical outcome in breast cancer. Int. J. Cancer 127, 1172-1179. (2010).
(73) Han, H., Wang, D., Yang, M. & Wang, S. High expression of RACK1 is associated with poor prognosis in patients with pancreatic ductal adenocarcinoma. Oncol. Lett. 15, 2073-2078. (2018).
(74) Qian, X. et al. Enolase 1 stimulates glycolysis to promote chemoresistance in gastric cancer. Oncotarget 8, 47691-47708. (2017).
(75) Zhu, W. et al. Enolase-1 serves as a biomarker of diagnosis and prognosis in hepatocellular carcinoma patients. Cancer Manag. Res. 10, 5735-5745. (2018).
(76) Yang, W. E. et al. Cathepsin B expression and the correlation with clinical aspects of oral squamous cell carcinoma. PLoS ONE 11, e0152165. (2016).
(77) Zhang, J., Pavlova, N. N. & Thompson, C. B. Cancer cell metabolism: the essential role of the nonessential amino acid, glutamine. EMBO J. 36, 1302-1315. (2017).
(78) Altman, B. J., Stine, Z. E. & Dang, C. V. From Krebs to clinic: glutamine metabolism to cancer therapy. Nat. Rev. Cancer 16, 619-634. (2016).
(79) Jeitner, T. M. & Cooper, A. J. Inhibition of human glutamine synthetase by L-methionine-S, R-sulfoximine-relevance to the treat-ment of neurological diseases. Metab. Brain Dis. 29, 983-989. (2014).
(80) Olson, O. C. & Joyce, J. A. Cysteine cathepsin proteases: regulators of cancer progression and therapeutic response. Nat. Rev. Cancer 15, 712-729. (2015).
(81) Ruan, H., Hao, S., Young, P. & Zhang, H. Targeting Cathepsin B for cancer therapies. Horiz. Cancer Res. 56, 23-40 (2015).
(82) Budhwani, M., Mazzieri, R. & Dolcetti, R. Plasticity of Type I interferon-mediated responses in cancer therapy: from anti-tumor immunity to resistance. Front. Oncol. 8, 322. (2018).
(83) McFarland, B. C. et al. Therapeutic potential of AZD1480 for the treatment of human glioblastoma. Mol. Cancer Ther. 10, 2384-2393. (2011).
(84) Nie, Y., Li, Y. & Hu, S. A novel small inhibitor, LLL12, targets STAT3 in non-small cell lung cancer in vitro and in vivo. Oncol. Lett. 16, 5349-5354. (2018).
(85) Ball, S., Li, C., Li, P. K. & Lin, J. The small molecule, LLL12, inhibits STAT3 phosphorylation and induces apoptosis in medulloblastoma and glioblastoma cells. PLoS ONE 6, e18820. (2011).
(86) Hu, Y. et al. Inhibition of the JAK/STAT pathway with ruxolitinib overcomes cisplatin resistance in non-small-cell lung cancer NSCLC. Apoptosis 19, 1627-1636. (2014).

### Aspects

The following listing of exemplary aspects supports and is supported by the disclosure provided herein.
Aspect 1. A method of determining a cancer progression risk score of a subject, the method comprising: detecting expression levels of genes of a progression gene signature in a sample; and calculating the cancer progression risk score of the subject using the expression levels of genes associated with a progression gene signature in the sample; wherein the progression gene signature comprises a glioblastoma progression gene signature, a non-small cell lung squamous cell carcinoma progression gene signature, a non-small cell lung adenocarcinoma progression gene signature, or combinations thereof; and wherein the cancer progression risk score is high risk progression or low risk progression.
Aspect 2. The method of any one of Aspect 1-Aspect 28, wherein the sample is obtained from the subject.
Aspect 3. The method of any one of Aspect 1-Aspect 28, wherein the sample is obtained from a tumor, tissue, bodily fluid, or a combination thereof.
Aspect 4. The method of any one of Aspect 1-Aspect 28, wherein the subject is a human.
Aspect 5. The method of any one of Aspect 1-Aspect 28, wherein the subject is diagnosed with a cancer.
Aspect 6. The method of any one of Aspect 1-Aspect 28, wherein the cancer is non-small cell lung cancer.
Aspect 7. The method of any one of Aspect 1-Aspect 28, wherein the cancer is a glioblastoma.
Aspect 8. The method of any one of Aspect 1-Aspect 28, wherein the detecting expression levels of genes of the progression gene signature comprises detecting expression levels of a glioblastoma progression gene signature; and wherein the glioblastoma progression gene signature comprises one or more genes selected from RPS11, UBB, TUBB, RPS6, EEF1A1, EEF2, PKM, C3, ENO1, HSP90AB1, FTL, CFL1, YWHAE, CKB, TUBA1A, FLNA, APP, CD63, ACTB, VIM, CTSB, MME, GLUL, MT3, ACTG1, HLA-C, B2M, CRYAB, LRP1, S100B, and FN1.
Aspect 9. The method of any one of Aspect 1-Aspect 28, wherein the detecting comprises detecting expression levels of five genes selected from RPS11, UBB, TUBB, RPS6, EEF1A1, EEF2, PKM, C3, ENO1, HSP90AB1, FTL, CFL1, YWHAE, CKB, TUBA1A, FLNA, APP, CD63, ACTB, VIM, CTSB, MME, GLUL, MT3, ACTG1, HLA-C, B2M, CRYAB, LRP1, S100B, and FN1.
Aspect 10. The method of any one of Aspect 1-Aspect 28, wherein the detecting comprises detecting expression levels of ten genes selected from RPS11, UBB, TUBB, RPS6, EEF1A1, EEF2, PKM, C3, ENO1, HSP90AB1, FTL, CFL1, YWHAE, CKB, TUBA1A, FLNA, APP, CD63, ACTB, VIM, CTSB, MME, GLUL, MT3, ACTG1, HLA-C, B2M, CRYAB, LRP1, S100B, and FN1.
Aspect 11. The method of any one of Aspect 1-Aspect 28, wherein the detecting comprises detecting expression levels of each of the genes RPS11, UBB, TUBB, RPS6, EEF1A1, EEF2, PKM, C3, ENO1, HSP90AB1, FTL, CFL1, YWHAE, CKB, TUBA1A, FLNA, APP, CD63, ACTB, VIM, CTSB, MME, GLUL, MT3, ACTG1, HLA-C, B2M, CRYAB, LRP1, S100B, and FN1.
Aspect 12. The method of any one of Aspect 1-Aspect 28, wherein the detecting expression levels of genes of the progression gene signature comprising detecting expression levels of a non-small cell lung squamous cell carcinoma progression gene signature; and wherein the non-small cell lung squamous cell carcinoma progression gene signature comprises one or more genes selected from GAPDH, KRT5, ACTG1, ENO1, PKM, CTSB, PSAP, MYH9, KRT14, RPS4X, CALR, FLNA, HSPA8, SFTPA2, RPS11, HSP90B1, HSPB1, SDC1, HLA-C, APP, ATP1A1, HSPA5, and RPL37.
Aspect 13. The method of any one of Aspect 1-Aspect 28, wherein the detecting comprises detecting expression levels of five genes selected from GAPDH, KRT5, ACTG1, ENO1, PKM, CTSB, PSAP, MYH9, KRT14, RPS4X, CALR, FLNA, HSPA8, SFTPA2, RPS11, HSP90B1, HSPB1, SDC1, HLA-C, APP, ATP1A1, HSPA5, and RPL37.
Aspect 14. The method of any one of Aspect 1-Aspect 28, wherein the detecting comprises detecting expression levels of ten genes selected from GAPDH, KRT5, ACTG1, ENO1, PKM, CTSB, PSAP, MYH9, KRT14, RPS4X, CALR, FLNA, HSPA8, SFTPA2, RPS11, HSP90B1, HSPB1, SDC1, HLA-C, APP, ATP1A1, HSPA5, and RPL37.
Aspect 15. The method of any one of Aspect 1-Aspect 28, wherein the detecting comprises detecting expression levels of each of the genes GAPDH, KRT5, ACTG1, ENO1, PKM, CTSB, PSAP, MYH9, KRT14, RPS4X, CALR, FLNA, HSPA8, SFTPA2, RPS11, HSP90B1, HSPB1, SDC1, HLA-C, APP, ATP1A1, HSPA5, and RPL37.
Aspect 16. The method of any one of Aspect 1-Aspect 28, wherein the detecting expression levels of genes of the progression gene signature comprising detecting expression levels of a non-small cell lung adenocarcinoma progression gene signature; and wherein the non-small cell lung adenocarcinoma progression gene signature comprises one or more genes selected from ACTB, FTL, SFTPA2, CD74, FN1, B2M, CTSD, CEACAM6, EEF2, PGC, UBC, HSP90AB1, SERPINA1, HSPA8, HSP90AA1, GNB2L1 (RACK1), CEACAM5, CD63, PIGR, KRT18, GLUL, and KRT19.
Aspect 17. The method of any one of Aspect 1-Aspect 28, wherein the detecting comprises detecting expression levels of five genes selected from ACTB, FTL, SFTPA2, CD74, FN1, B2M, CTSD, CEACAM6, EEF2, PGC, UBC, HSP90AB1, SERPINA1, HSPA8, HSP90AA1, GNB2L1 (RACK1), CEACAM5, CD63, PIGR, KRT18, GLUL, and KRT19.
Aspect 18. The method of any one of Aspect 1-Aspect 28, wherein the detecting comprises detecting expression levels of ten genes selected from ACTB, FTL, SFTPA2, CD74, FN1, B2M, CTSD, CEACAM6, EEF2, PGC, UBC, HSP90AB1, SERPINA1, HSPA8, HSP90AA1, GNB2L1 (RACK1), CEACAM5, CD63, PIGR, KRT18, GLUL, and KRT19.
Aspect 19. The method of any one of Aspect 1-Aspect 28, wherein the detecting comprises detecting expression levels of each of the genes ACTB, FTL, SFTPA2, CD74, FN1, B2M, CTSD, CEACAM6, EEF2, PGC, UBC, HSP90AB1, SERPINA1, HSPA8, HSP90AA1, GNB2L1 (RACK1), CEACAM5, CD63, PIGR, KRT18, GLUL, and KRT19.
Aspect 20. The method of any one of Aspect 1-Aspect 28, wherein the detecting expression levels of genes of a progression gene signature in a sample comprises detecting using a method selected from a PCR method, a RNASeq method, and combinations thereof.
Aspect 21. The method of any one of Aspect 1-Aspect 28, wherein the detecting expression levels of genes of a progression gene signature in a sample comprises detecting using a PCR method selected from ddPCR, digital droplet PCR, qPCR, and combinations thereof.
Aspect 22. The method of any one of Aspect 1-Aspect 28, wherein the PCR method utilizes one or more primers selected from SEQ ID NOs. 1-62.
Aspect 23. The method of any one of Aspect 1-Aspect 28, wherein the calculating the cancer progression risk of the subject using the expression levels of genes associated with a progression gene signature in the sample comprises: deriving a cancer progression risk score model comprising: carrying our principal component analysis of a set of principal components (PCs) linearizing z-score-normalized gene expression values across the progression gene signature for a dataset comprising at least 100 patient samples with known tumor progression outcome; wherein the number principal components generated was equal to the number of genes in the progression gene signature; screening the principal components using random forests of 1000 trees trained on a yes/no indicator of tumor progression and selecting principal components correlated with incidence of the tumor progression, and implementing a percent contribution cutoff of >0.05; selecting principal components and repeating the carrying our principal component analysis and screening the principal components until random forests retained all principal components; subjecting the end principal component set into a neural network with three tan H nodes boosted 100 times at a 0.1 learning rate with tenfold cross validation; providing the formula output as a probability of the tumor progression on a scale of 0 to 1, and then transposing to a scale of −50 to 50; wherein a cutoff of 0 stratified the tumor progression as high risk and <0 stratified the tumor progression as low risk; providing data for the expression levels of genes associated with a progression gene signature in the sample as input to the cancer progression risk score model to determine the cancer progression risk score of the subject.
Aspect 24. The method of any one of Aspect 1-Aspect 28, wherein the calculating the cancer progression risk of the subject using the expression levels of genes associated with a progression gene signature in the sample comprises using a classifier trained with a training data set comprising measured expression levels of the genes from training subjects having a high risk of progression and training subjects having a low risk of progression.
Aspect 25. The method of any one of Aspect 1-Aspect 28, wherein the calculating the cancer progression risk of the subject using the expression levels of genes associated with a progression gene signature in the sample comprises a classification method selected from the group consisting of a profile similarity; an artificial neural network; a support vector machine (SVM); a logic regression, a linear or quadratic discriminant analysis, a decision trees, a clustering, a principal component analysis, a nearest neighbor classifier analysis, a nearest shrunken centroid, a random forest, and a combination thereof.
Aspect 26. The method of any one of Aspect 1-Aspect 28, wherein the calculating the cancer progression risk of the subject using the expression levels of genes associated with a progression gene signature in the sample comprises using a classification method, the classification method constructed by: (a) generating a set of components by dimensionality reduction of expression levels of the genes in a training data set, the training data set comprising gene expression levels from training subjects having a high risk of progression and training subjects having a low risk of progression; (b) training a machine learning model to select a subset of components from the set of components, the subset of components being more highly correlated to the risk of progression as compared to a correlation of the unselected components; (c) repeating steps (a) and (b) with the selected subset of components from the set of components until there are no unselected components from the machine learning model of step (b); and (d) constructing the classification method from the subset of components.
Aspect 27. The method of any one of Aspect 1-Aspect 28, wherein the classification method is a neural network.
Aspect 28. The method of any one of Aspect 1-Aspect 28, wherein the subset of components being more highly correlated comprises having a percent contribution cutoff of about 0.05 or more
Aspect 29. A method of detecting a cancer in a subject or a sample therefrom containing cells comprising: determining a cancer progression risk score of a subject as in any of claims 1-23; and diagnosing the cancer in the subject when a cancer signature is detected.
Aspect 30. The method of any one of Aspect 29-Aspect 31, wherein the cancer is glioblastoma and/or non-small cell cancer.
Aspect 31. The method of any one of Aspect 29-Aspect 31, further comprising administering a chemotherapy agent or modality to the subject.
Aspect 32. A method of treating a cancer in a subject, comprising: determining a cancer progression risk score of a subject as in any of Aspect 1-Aspect 28; and administering an effective amount of an agent effective to modulate, inhibit a function and/or activity of a cancer cell, and/or kill a cancer cell, or a combination thereof to the subject.
Aspect 33. The method of any one of Aspect 32-Aspect 40, wherein the progression signature is indicative of the subject having a high risk of progression or a low risk of progression; and treating the subject with a more aggressive cancer treatment based upon the subject having a high risk of progression or a less aggressive cancer treatment based upon the subject having a low risk of progression.
Aspect 34. The method of any one of Aspect 32-Aspect 40, wherein the more aggressive cancer treatment comprises a more aggressive traditional therapy, a non-standard treatment regimen, or a combination thereof.
Aspect 35. The method of any one of Aspect 32-Aspect 40, wherein the less aggressive cancer treatment comprises a less aggressive traditional therapy.
Aspect 36. The method of any one of Aspect 32-Aspect 40, wherein the cancer is a lung cancer, and wherein the more aggressive cancer treatment comprises chemotherapy, a combination of chemotherapy and radiation therapy, or a non-standard treatment regimen.
Aspect 37. The method of any one of Aspect 32-Aspect 40, wherein the cancer is a lung cancer, and wherein the less aggressive cancer treatment comprises surgery, chemotherapy, radiation therapy, or a combination thereof.
Aspect 38. The method of any one of Aspect 32-Aspect 40, wherein the less aggressive cancer treatment comprises observing and monitoring a progression of the cancer.
Aspect 39. The method of any one of Aspect 32-Aspect 40, wherein the cancer is a glioblastoma, and wherein the less aggressive cancer treatment comprises surgical resection, an abbreviated radiation therapy, administering adjuvant chemotherapy such as Temozolomide, or a combination thereof.
Aspect 40. The method of any one of Aspect 32-Aspect 40, wherein the cancer is a glioblastoma, and wherein the more aggressive cancer treatment comprises surgical resection, a combination of surgical resection and adjuvant chemotherapy, or a non-standard treatment regimen.
Aspect 41. A method of screening for an agent effective against a cancer comprising: contacting a cancer cell or population thereof having an initial cell signature and/or cell state with a test agent; determining a change in the initial cell signature and/or shift in initial cell state, wherein a change in the initial cell signature and/or shift in initial cell state identifies an effective agent and wherein determining a change in the initial cell signature and/or shift in initial cell state comprises a method of determining a cancer progression risk score of a subject as in any one of Aspect 1-Aspect 28.
Aspect 42. A system to process biological information, comprising: one or more processors; and one or more memory elements including instructions, which when executed cause the one or more processors to: receive an array of ribonucleic acid (RNA) sequence data associated with a group of patients; determine a first set of gene sequences having respective expression magnitudes greater than a threshold value in at least one subtype from the array of RNA sequence data; select, from the first set of gene sequences, a second set of gene sequences based on a model selection criteria; determine, from the second set of gene sequences, a set of cancer survival gene sequences based on cross-referencing each gene sequence from the second set of gene sequences with RNA interference data; and select, from the set of cancer survival gene sequences, a set of progression gene signatures, based on a tumor progression criteria.
Aspect 43. The system of any one of Aspect 42-Aspect 49, wherein the array of RNA sequence data includes at least one of RNA-seq data and microarray data.
Aspect 44. The system of any one of Aspect 42-Aspect 49, wherein the threshold includes a 99^{th} percentile cut-off.
Aspect 45. The system of any one of Aspect 42-Aspect 49, wherein the at least one subtype includes at least one of lung adenocarcinoma, lung squamous cell carcinoma, and glioblastoma.
Aspect 46. The system of any one of Aspect 42-Aspect 49, wherein the model selection criteria includes at least one of Bayesian information criterion and Akaike information criterion.
Aspect 47. The system of any one of Aspect 42-Aspect 49, wherein the RNA interference data includes at least one cell line associated with the at least one subtype.
Aspect 48. The system of any one of Aspect 42-Aspect 49, wherein the one or more memory elements include instructions which when executed cause the one or more processors to: determine the set of cancer survival gene sequences based in part on selection of those gene sequences from the second set of gene sequences having corresponding fold change of less than zero in the RNA interference data.
Aspect 49. The system of any one of Aspect 42-Aspect 49, wherein the tumor progression criteria includes a backward stepwise regression model with a predetermine p-value.
Aspect 50. A computer-implemented method for processing biological information, comprising: receiving, by a computer server including one or more processors, an array of ribonucleic acid (RNA) sequence data associated with a group of patients; determining, by the computer server, a first set of gene sequences having respective expression magnitudes greater than a threshold value in at least one subtype from the array of RNA sequence data; selecting, by the computer server, from the first set of gene sequences, a second set of gene sequences based on a model selection criteria; determining, by the computer server, from the second set of gene sequences, a set of cancer survival gene sequences based on cross-referencing each gene sequence from the second set of gene sequences with RNA interference data; and selecting, by the computer server, from the set of cancer survival gene sequences, a set of progression gene signatures, based on a tumor progression criteria.
Aspect 51. The method of any one of Aspect 50-Aspect 57, wherein the array of RNA sequence data includes at least one of RNA-seq data and microarray data.
Aspect 52. The method of any one of Aspect 50-Aspect 57, wherein the threshold includes a 99^{th} percentile cut-off.
Aspect 53. The method of any one of Aspect 50-Aspect 57, wherein the at least one subtype includes at least one of lung adenocarcinoma, lung squamous cell carcinoma, and glioblastoma.
Aspect 54. The method of any one of Aspect 50-Aspect 57, wherein the model selection criteria includes at least one of Bayesian information criterion and Akaike information criterion.
Aspect 55. The method of any one of Aspect 50-Aspect 57, wherein the RNA interference data includes at least one cell line associated with the at least one subtype.
Aspect 56. The method of any one of Aspect 50-Aspect 57, further comprising: determining, by the computer server, the set of cancer survival gene sequences based in part on selection of those gene sequences from the second set of gene sequences having corresponding fold change of less than zero in the RNA interference data.
Aspect 57. The method of any one of Aspect 50-Aspect 57, wherein the tumor progression criteria includes a backward stepwise regression model with a predetermine p-value.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of aspects of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example Methods

The following exemplary methods were employed for purposes of the specific examples described herein. In some aspects the methods may be preferred. In other aspects, however, those skilled in the art may recognize suitable alternatives or variations to the methods. Such suitable alternatives and variations are intended to be covered by the instant disclosure to the extent they do not deviate from the claimed aspects.

### Retrieval and Analysis of Patient Gene Expression and Clinical Data.

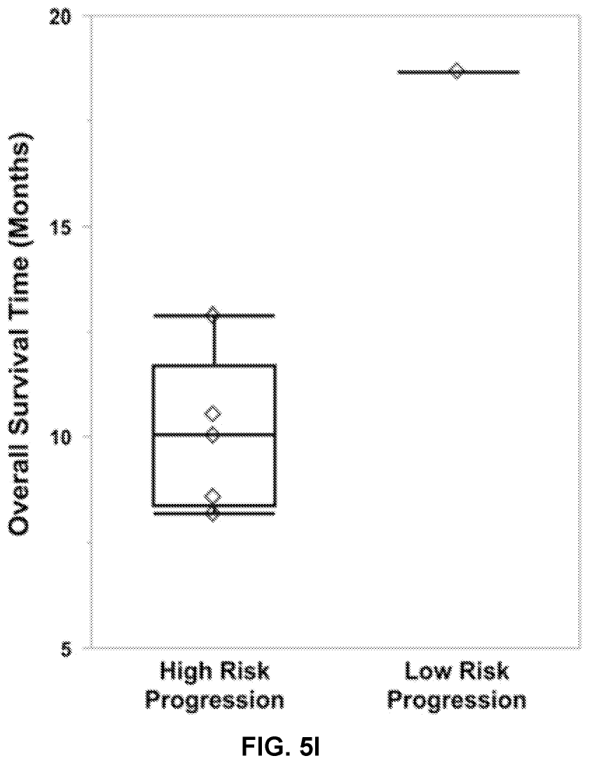

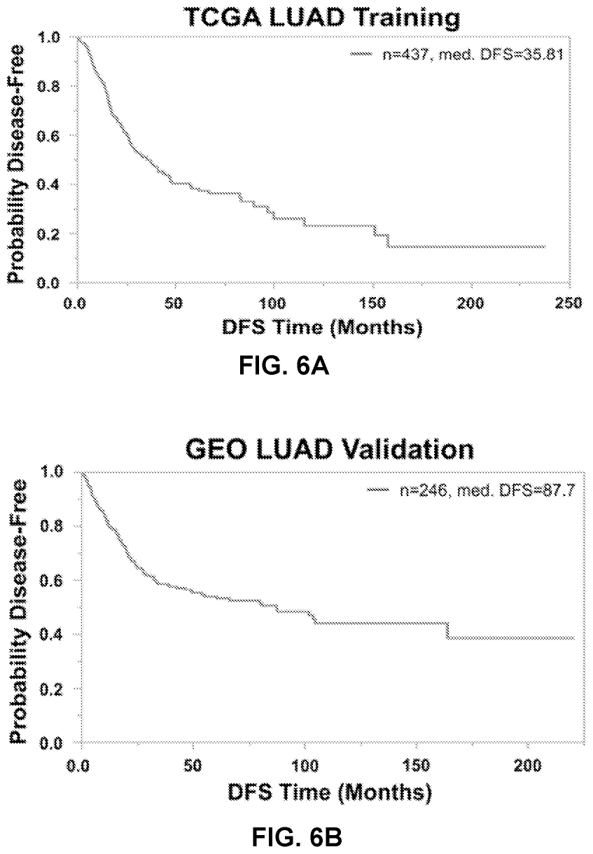

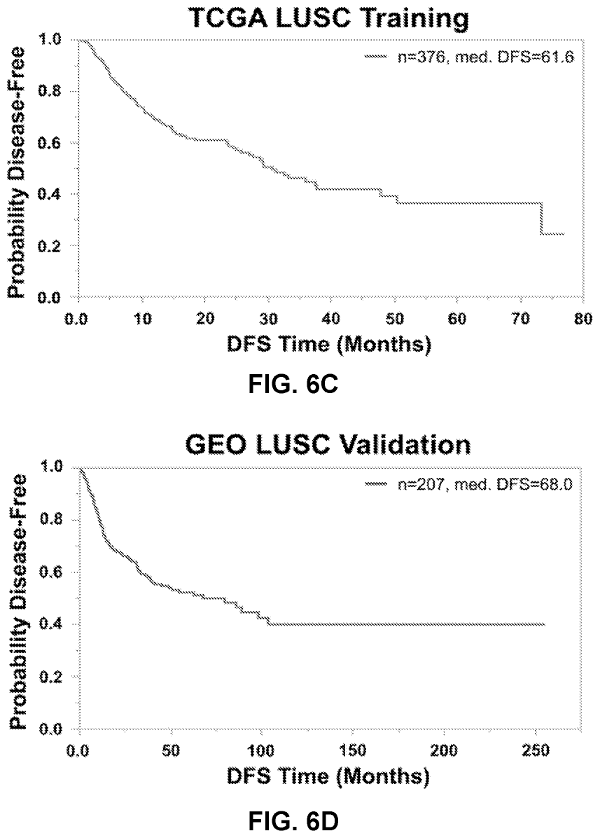

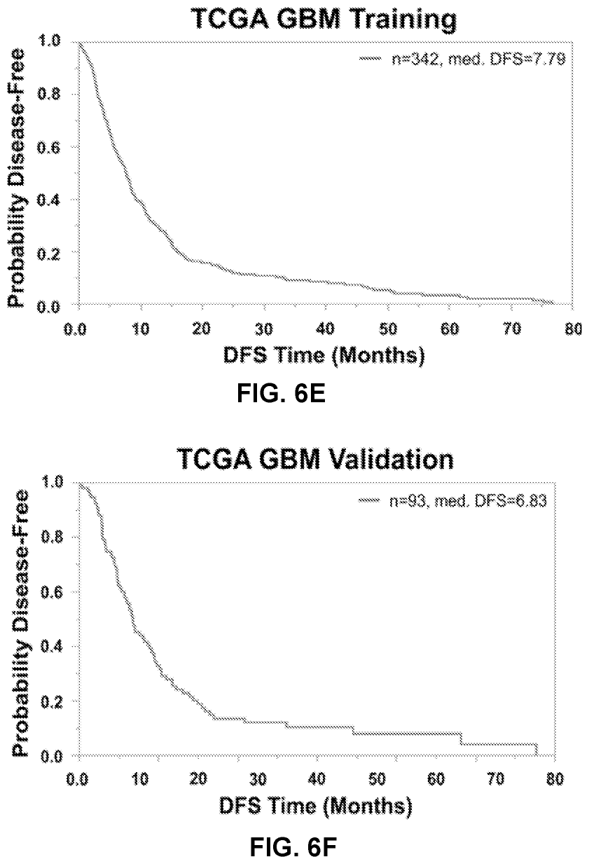

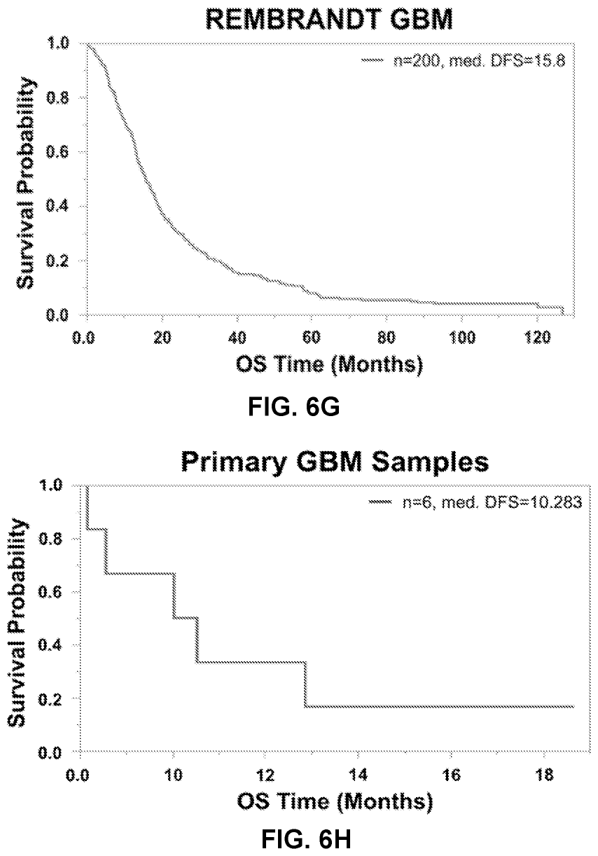

The TOGA database contains publicly-accessible, RSEM-processed RNA sequencing (RNA-seq) data for 500+ quality-controlled primary tumor samples in LUAD and LUSC and genome-wide microarray profiling for 528 quality-controlled primary GBM samples. Gene expression and corresponding clinical data for 517 LUAD, 501 LUSC, and 528 GBM patients were retrieved from cBioPortal(Refs. 47-48) and used as the training set. To compile the NSCLC validation cohort, datasets from the NCBI Gene Expression Omnibus repository were screened for microarray chip type (Affymetrix U133 Plus 2.0, GPL570), availability of LUAD and LUSC samples, and availability of overall survival (OS) or disease-free survival (DFS) status and time-to-event data. Raw data from four selected microarray data-sets (GSE3141 from Ref. 49, GSE8894 from Ref. 50, GSE19188 from Ref. 51, and GSE30219 from Ref. 52) were downloaded and pre-processed using robust multiarray averaging for normalization, then compiled to form validation cohorts that include 246 LUAD or 207 LUSC patients, respectively. In GBM, a random sampling technique stratified on age and gender was used to separate the TOGA cohort into a 396-patient training and 132-patient validation cohort due to the limited avail-ability of external datasets. Microarray profiling and clinical data for 200 GBM patients from Rembrandt (Ref. 53) were retrieved to use as an independent validation cohort. Additionally, OS status and time-to-event data for six primary GBM samples obtained from patients who underwent surgical resection at Carilion Clinic were retrieved for experimental validation. These patients were de-identified and the IRB protocol was approved by Carilion Clinic IRB office. Available clinical characteristics for each cohort are summarized in Table 1. Unstratified survival of all training and validation cohorts are shown in FIG. 6.

TABLE 1
Clinical characteristics of the training and validation cohorts.

| | Training | | | Validation | | | |
|---|---|---|---|---|---|---|---|
| | TCGA | TCGA | TCGA | GEO | GEO | TCGA | REMBRT |
| Variables | LUAD | LUSC | GBM (T) | LUAD | LUSC | GBM (V) | GBM |
| No. of Patients | 517 | 504 | 389 | 246 | 207 | 126 | 200 |
| Gender | | | | | | | |
| Male | 240 | 131 | 236 | 100 | 142 | 75 | 99 |
| Female | 277 | 373 | 153 | 48 | 12 | 51 | 55 |
| N/A | 0 | 0 | 0 | 98 | 53 | 0 | 46 |
| Med. Age (Years) | 66 | 68 | 59 | 60.5 | 63.5 | 60 | 57 |
| Race | | | | | | | |
| Caucasian | 393 | 351 | 341 | | | 112 | 139 |
| African American | 53 | 31 | 22 | | | 8 | 2 |
| Asian | 8 | 9 | 11 | | | 0 | 1 |
| N/A | 63 | 113 | 15 | 246 | 207 | 6 | 58 |
| AJCC TNM Stage | | | | | | | |
| Stage I | 279 | 245 | | 81 | 48 | | |
| Stage II | 124 | 163 | | 3 | 5 | | |
| Stage III | 85 | 85 | | 1 | 2 | | |
| Stage IV | 26 | 7 | | 0 | 0 | | |
| N/A | 3 | 4 | | 161 | 152 | | |
| Smoking History | | | | | | | |
| Non-Smoker | 75 | 18 | | | | | |
| Current Smoker | 122 | 134 | | | | | |
| Reformed Smoker | 307 | 335 | | | | | |
| N/A | 13 | 17 | | 246 | 207 | | |
| Subtype | | | | | | | |
| Classical | | | 107 | | | 36 | |
| Mesenchymal | | | 112 | | | 41 | |
| Proneural | | | 170 | | | 49 | |
| | | | | | | | 200 |
| N/A | | | 0 | | | 0 | |

Analysis of RNAi Screen Data from the Cancer Dependency Map Database.

The DepMap data-base contains data from the Project Achilles initiative by Broad Institute. This database contains publicly accessible, genome-wide RNAi screen results across 501 cancer cell lines, including 18 NSCLC and 20 GBM cell lines (Ref. 46). The screens include over 50,000 short hairpin RNAs (shRNAs) targeting the human genome and present results as log₂ fold change of shRNA depletion. RNAi results from the Achilles 2.20.2 release were retrieved from DepMap and pre-processed to calculate the average log 2 fold change across all shRNAs targeting each gene in each cell line.

### Isolation and Culture of Primary GBM Cells.

The use of human GBM patient specimens has been approved by the Institutional Review Board at Carilion Clinic and we confirm that informed consent was obtained from all participants and/or their legal guardians as required in the IRB. Freshly resected human GBM tumors (pathologically confirmed) were minced into small pieces. Single cells were prepared using Liberase (Roche Diagnostics) according to the manufacturer's instructions. Red blood cells were removed using Red Blood Cell Lysis Solution purchased from Miltenyi Biotec Inc. Isolated cells were cultured in DMEM (Life Technologies) supplemented with 15% FBS (Peak Serum, Inc.), streptomycin (100 μg/mL), and penicillin (100 IU/ml), (Life Technologies Corporation). Primary GBM cells were kept at no more than 10 passages.

### Identification of PGSs.

Comprehensive RNA-seq or microarray data for over 500 patients in the TCGA training cohort were first used to identify the most ubiquitously expressed genes in two predominant NSCLC subtypes, LUAD and LUSC, and in GBM. A 99th-percentile cutoff was initially employed to ensure mRNA detection in other gene expression profiling platforms, resulting in the selection of 200 genes. This cutoff was further refined to 100 genes after downstream Bayesian Information Criterion (BIC) score optimization of the resulting gene signatures (Table 2). Genes from this primary candidate pool were subsequently cross-referenced in 18 NSCLC or 20 GBM cell lines with available genome-wide RNAi screen data through Project Achilles. Since Project Achilles presents RNAi results as log 2 fold changes indicative of shRNA loss, lower fold change values confer a stronger depletion of shRNAs and, thus, a larger reduction in cell viability following target gene knockdown. An average shRNA fold change cutoff of <0 was implemented to select survival genes associated with cancer cell survival. One-tailed one-sample t-tests determined the significance of fold change <0 for each shRNA, and Fisher's combined probability test confirmed the false discovery rate (FDR)-adjusted significance of average shRNA fold change <0. Genes not present in the Project Achilles database were excluded from further analyses. All survival genes were then entered into a backward stepwise variable regression model trained on a yes/no indicator of tumor progression incidence with a p-value threshold of 0.25 for PGS assembly.

TABLE 2
BIC score optimization to determine the cutoff for
highly-expressed genes. PGSs were identified using
the working pipeline described in the Methods section.
The cutoffs returning a PGS with the lowest BIC score,
which corresponds to the lowest overfitting potential,
are highlighted in grey.

| | Cutoff Value | Genes in PGS | BIC |
|---|---|---|---|
| LUAD | 50 | 19 | 671.251 |
| | 100 | 22 | 662.856 |
| | 150 | 30 | 684.074 |
| | 200 | 59 | 785.492 |
| LUSC | 50 | 20 | 567.156 |
| | 100 | 23 | 559.984 |
| | 150 | 34 | 580.935 |
| | 200 | 53 | 635.626 |
| GBM | 50 | 28 | 778.526 |
| | 100 | 31 | 767.708 |
| | 150 | 48 | 819.793 |
| | 200 | 48 | 802.173 |

### Derivation of PGS Risk Scores for Patient Risk Stratification.

Tumor progression risk scores were derived by a combination of statistical and machine-learning approaches. Principal component analysis (PCA) was first used to generate a set of principal components (PCs) linearizing z-score-normalized gene expression values across each PGS for each patient. The number of PCs generated was equal to the number of genes in each PGS. Each PC set was then screened using random forests of 1000 trees trained on a yes/no indicator of tumor progression incidence to select PCs highly correlated with progression incidence, implementing a per-cent contribution cutoff of >0.05. Selected PCs were entered into a second PCA, and the process was iterated until random forests retained all PCs. The end PC set was entered into a neural network with three tan H nodes boosted 100 times at a 0.1 learning rate with tenfold cross validation. The resulting formula output the predicted probability of tumor progression on a scale of 0 to 1, which were then transposed to a scale of −50 to 50 for ease of interpretation. A cutoff at 0 stratified patients as high-risk progression (>0) or low-risk progression (<0).

### Assessment of PGS Risk Score Accuracy.

The accuracy of patient risk stratification determined by each PGS was evaluated using various statistical methods. The frequency of tumor progression events within each risk group were calculated within confusion matrices, and significance testing of correlations were evaluated with Fisher's Exact Tests. The area under the receiver operating characteristic (ROC) curve (AUC) values were interpreted as the fraction of accurately predicted cases. Pair-wise comparison of ROC curves fit using PGS-derived risk scores or current progression biomarkers determined significance of accuracy improvement. Kaplan-Meier survival analyses and Cox proportional hazards models determined association of patient risk groups with DFS time.

### Correlation Analysis of PGS-Stratified Risk and Treatment Response.

Clinical data on adjuvant chemotherapy (ACT) or TMZ administration for the TOGA training cohorts were retrieved from the NCI GDC data portal, and the Buffa hypoxia scores for each patient were retrieved from TOGA PanCancer Atlas through cBioPortal (Ref. 54). Differences in patient benefit from treatment across risk groups were assessed using one-tailed two-sample t-tests on unequal variances and Fisher's Exact Tests. Two-tailed two-sample t-tests on unequal variances assessed the correlation of PGS risk stratification with tumor hypoxia in NSCLC.

### Validation of PGS and Risk Algorithm.

The validation of both NSCLC PGSs was accomplished via a retrospectively-compiled cohort of four independent microarray datasets, while GBM-PGS was validated in both an internal TOGA validation cohort and the external Rembrandt cohort. Gene expression data from each study were z-score normalized prior to risk algorithm application. NSCLC clinical data were processed as follows for cross-study compatibility: (1) Relapsed patients were categorized as “progressed” and non-relapsed patients “disease-free” in GS8894 and GSE30219; (2) Deceased patients were categorized as “progressed” and living patients as “disease-free” in GSE3141 and GSE19188, where relapse incidence data were unavailable. Accuracy of risk classification and characterization of risk groups were assessed using Fisher's Exact Tests and Kaplan-Meier survival curves as described previously.

Quantitative Reverse Transcription Polymerase Chain Reaction (gRT-PCR).

Passage numbers for the six primary GBM cells are shown in Table 3. Total RNA was isolated from frozen primary GBM cells using TriZol (Invitrogen), and cDNA was synthesized using reverse transcriptase (New England Biolabs). Primers (Sigma) were retrieved from literature search or PrimerBank and verified in Primer-BLAST (Table 4). mRNA expression levels of GBM-PGS in six patient samples were measured by qRT-PCR using a StepOnePlus™ Real-Time PCR system. Glyceraldehyde-3-phosphate dehydrogenase (GAPDH) demonstrated the most stable expression compared to beta actin (ACTB) or beta 2 microglobulin (B2M) using RefFinder (Ref. 55) and was used as the control (Table 5). ΔCt values were calculated by subtracting Ct values of genes of interest from the Ct value of GAPDH and z-score-normalized within the six GBM primary cells. The GBM-PGS risk algorithm was applied to the z-score-normalized ΔCt values of each gene to calculate risk scores for each sample using the PCs and neural network trained on the GBM training cohort. Patients were stratified as high- or low-risk progression as described previously.

TABLE 3
Passage numbers for primary GBM cells.

| Primary GBM Cell | Passage Number |
|---|---|
| VTC-001 | 3 |
| VTC-004 | 3 |
| VTC-010 | 5 |
| VTC-037 | 3 |
| VTC-058 | 5 |
| VTC-093 | 6 |

TABLE 4
qRT-PCR primers used for detecting mRNA levels
of PGS genes in GBM.

| | Primers Sequence 5′-3′ |
|---|---|
| Gene | (forward, reverse) |
| RPS11 | AGCAGCCGACCATCTTTC |
| | ATAGCCTCCTTGGGTGTCTTG |
| UBB | AAGCCTAAACTGCCTCTC |
| | GTTGCGTCACTTATCACC |
| TUBB | GCAGTCACCTTCATTGGCAAT |
| | GCGGAACATGGCAGTGAACT |
| RPS6 | CGCCAAGTATGTTGTAAGAAAGCCCT |
| | GCTGCAGGACACGTGGAGTAACA |
| EEF1A1 | CAGGACACAGAGACTTTATC |
| | AGTGTGTAAGCCAGAAGGG |
| EEF2 | AGAAGCTGTGGGGTGACAG |
| | GATCAGCTGGCAGAAGGTG |
| PKM | GTGCGAGCCTCAAGTCACTCCACA |
| | TATAAGAAGCCTCCACGCTGCCCA |
| C3 | GCTGAAGCACCTCATTGTGA |
| | CTGGGTGTACCCCTTCTTGA |
| ENO1 | GAGCTCCGGGACAATGATAA |
| | CTGTTCCATCCATCTCGATC |
| HSP90AB1 | TTTGGGAACCATTGCCAAGTC |
| | CACCAAACTGCCCAATCATGG |
| FTL | GCGTCTCCTGAAGATGCAAA |
| | AGGAAGTGAGTCTCCAGGAAGT |
| CFL1 | GAAGGAGGATCTGGTGTTTATCTTCT |
| | CCTTGGAGCTGGCATAAATCAT |
| YWHAE | GGATACGCTGAGTGAAGAAAGC |
| | TATTCTGCTCTTCACCGTCACC |
| CKB | GCTGCGACTTCAGAAGCGA |
| | GGCATGAGGTCGTCGATGG |
| TUBA1A | GCAACAACCTCTCCTCTTCG |
| | GAATCATCTCCTCCCCCAAT |
| FLNA | GCACTTACAGCTGCTCCTACG |
| | CCAGCTCCCACATTCACC |
| APP | AACCAGTGACCATCCAGAAC |
| | ACTTGTCAGGAACGAGAAGG |
| CD63 | AGCAGATGGAGAATTACCC |
| | CTCCCAATCTGTGTAGTTAG |
| ACTB | TTCCTGGGCATGGAGTC |
| | CAGGTCTTTGCGGATGTC |
| VIM | AGCCGAAAACACCCTGCAAT |
| | CGTTCAAGGTCAAGACGTGC |
| CTSB | GGCCCCCTGCATCTATCG |
| | AGGTCTCCCGCTGTTCCACTG |
| MME | CATCGGCATGGTCATAGGACA |
| | TGTTGAGTCCACCAGTCAACGA |
| GLUL | CACACAATCTTGGCATTTCC |
| | ACTCAGGGGAGCAAAGGAAG |
| MT3 | ATGGACCCTGAGACCTGCC |
| | TTGCACACACAGTCCTTGGC |
| ACTG1 | TGTTTCCTTCCATCGTCGGG |
| | CATGTCGTCCCAGTTGGTGA |
| HLA-C | TCCTGGTTGTCCTAGCTGTC |
| | CAGGCTTTACAAGTGATGAG |
| B2M | GATGAGTATGCCTGCCGTGT |
| | TGCGGCATCTTCAAACCTCC |
| CRYAB | AGGTGTTGGGAGATGTGATTGA |
| | GGATGAAGTAATGGTGAGAGGGT |
| LRP1 | ACATATAGCCTCCATCCTAATC |
| | TTCCAATCTCCACGTTCAT |
| S100B | AGACGGTCATGCAAGAAAGC |
| | GCTACAACACGGCTGGAAAG |
| FN1 | AGATCTACCTGTACACCTTGAATGACA |
| | CATGATACCAGCAAGGAATTGG |

TABLE 5
Stability of GAPDH, ACTB, and B2M expression in six primary GBM
cell lines. RefFinder assigns weights to reference gene stability rankings
from four algorithms and calculates their geometric mean. Lower
geometric means confer greater expression stability.

| | | Comparative | | | |
|---|---|---|---|---|---|
| Gene | RefFinder | ΔCt | BestKeeper | NormFinder | GeNorm |
| GAPDH | 1.00 | 1.22 | 0.61 | 0.211 | 0.680 |
| ACTB | 1.68 | 1.27 | 0.87 | 0.647 | 0.680 |
| B2M | 3.00 | 1.80 | 1.32 | 1.738 | 1.429 |

### Software and Programs.

Data preprocessing were performed in Microsoft Excel and R statistical software (Ref. 56). All statistical analyses and machine learning were conducted in JMP Pro 14.3 and Python 3.8.1.

### Results

### Biomarker Identification Pipeline Reveals PGSs in Lung Cancer and GBM.

To address challenges in identifying reliable cancer biomarkers, we developed a working pipeline (FIG. 1A) for the identification of cancer progression biomarkers. First, comprehensive RNA-seq or microarray data in TOGA were used to identify the most ubiquitously expressed genes in two predominant NSCLC subtypes, LUAD and LUSC, and in GBM. A 99th-percentile cutoff resulted in a candidate pool of 200 genes. This cutoff was further refined to 100 genes after using Bayesian Information Criterion (BIC) score optimization of the resulting gene signatures (Table 2). These 100 genes were subsequently cross-referenced in 18 NSCLC or 20 GBM cell lines with available genome-wide RNAi screen data through DepMap. Since DepMap presents RNAi results as log 2 fold changes indicative of shRNA loss, lower fold change values confer a stronger depletion of shRNAs and, thus, a larger reduction in cell viability following target gene knockdown. An shRNA fold change cut-off of <0 was implemented to select survival genes associated with cancer cell survival. One-tailed one-sample t-tests and Fisher's combined probability test confirmed the FDR-adjusted significance of shRNA fold change <0 (Table 6). Genes not present in the DepMap database were excluded from further analyses. All survival genes were then entered into a backward stepwise variable regression model trained on a yes/no indica-tor of tumor progression incidence with a p-value threshold of 0.25 for PGS assembly. This new pipeline allows us to develop gene signatures indicative for the survival of cancer cells and as biomarkers for predicting disease progression.

TABLE 6
Significance of shRNA log₂ fold change <0 for survival genes. One-tailed one-
sample t-tests first analyzed the significance of fold change <0 for each shRNA
targeting each candidate survival gene. False discovery rate-adjusted P-values
(Q-values) were then calculated using Fisher's combined probability test to
determine the combined significance of all shRNA log₂ fold change <0.
Genes with average shRNA fold change >0 are shown in bold.

| LUAD | | LUSC | | GBM | |
|---|---|---|---|---|---|
| Gene | Q-value | Gene | Q-value | Gene | Q-value |
| RPS8 | 4.247E−43 | RPS8 | 5.382E−43 | RPS11 | 1.561E−61 |
| RPL5 | 2.955E−55 | RPS11 | 1.068E−55 | UBB | 5.165E−43 |
| RPS11 | 1.054E−55 | RPS18 | 1.442E−51 | RPS3 | 1.870E−51 |
| RPS18 | 9.482E−52 | EEF2 | 3.447E−38 | RPS4X | 1.433E−38 |
| EEF2 | 3.401E−38 | RPS4X | 2.489E−34 | TUBB | 4.876E−36 |
| RPS4X | 2.455E−34 | RPS3 | 1.167E−43 | RPS6 | 1.774E−39 |
| RPS3 | 8.635E−44 | RPL37 | 1.820E−38 | HSP90AA1 | 7.630E−22 |
| ATP1A1 | 1.896E−34 | ATP1A1 | 1.922E−34 | EEF1A1 | 4.590E−31 |
| RPS6 | 6.508E−41 | RPS6 | 7.917E−41 | EEF2 | 2.763E−26 |
| TUBB | 1.127E−28 | TUBB | 1.142E−28 | CALM2 | 3.081E−25 |
| CEACAM5 | 5.806E−26 | HSP90AA1 | 2.686E−26 | DPYSL3 | 3.397E−25 |
| HSP90AA1 | 2.650E−26 | SFTPB | 3.156E−26 | PSAP | 1.151E−17 |
| SFTPB | 3.113E−26 | EIF4G1 | 4.571E−21 | PTN | 2.892E−16 |
| RPS16 | 4.335E−27 | RPS16 | 4.395E−27 | CDK4 | 1.249E−26 |
| ACTB | 5.152E−17 | ACTB | 5.432E−17 | SPP1 | 6.570E−19 |
| GAPDH | 1.312E−24 | GAPDH | 1.425E−24 | MBP | 5.758E−17 |
| GNB2L1 | 9.111E−23 | GNB2L1 | 9.852E−23 | PKM | 1.152E−17 |
| PKM | 1.012E−17 | PKM | 1.070E−17 | PTPRZ1 | 5.770E−27 |
| EEF1A1 | 1.451E−18 | EEF1A1 | 1.471E−18 | GAPDH | 2.041E−19 |
| CFL1 | 1.868E−22 | TFRC | 7.954E−16 | A2M | 1.674E−18 |
| HSP90B1 | 2.593E−17 | CFL1 | 2.012E−22 | UBC | 5.444E−19 |
| ENO1 | 3.071E−18 | HSP90B1 | 2.738E−17 | CALM1 | 9.975E−13 |
| UBC | 7.652E−16 | ENO1 | 3.113E−18 | C3 | 5.265E−22 |
| PSAP | 7.165E−20 | UBC | 7.757E−16 | SLC1A3 | 7.348E−21 |
| A2M | 4.804E−15 | PSAP | 7.263E−20 | GNB2L1 | 8.158E−17 |
| HSPA5 | 1.989E−04 | HSPA5 | 1.989E−04 | ENO1 | 6.686E−17 |
| SFTPC | 4.079E−12 | SPP1 | 1.688E−13 | HSP90AB1 | 4.738E−19 |
| PGC | 4.227E−19 | APP | 4.062E−07 | FTL | 5.026E−21 |
| KRT19 | 3.572E−14 | KRT6A | 3.805E−19 | CFL1 | 6.756E−19 |
| CD74 | 4.827E−15 | KRT19 | 4.205E−14 | YWHAE | 4.947E−15 |
| NAPSA | 9.726E−15 | CD74 | 5.231E−15 | CKB | 1.484E−08 |
| HSPA8 | 1.839E−13 | JUP | 2.079E−09 | CD81 | 4.035E−18 |
| P4HB | 3.351E−15 | HSPA8 | 2.029E−13 | TUBA1A | 4.360E−29 |
| CEACAM6 | 6.077E−13 | FTL | 1.619E−20 | FLNA | 1.836E−10 |
| FTL | 1.597E−20 | ALDOA | 3.376E−15 | CLU | 3.944E−17 |
| ALDOA | 3.216E−15 | S100A9 | 9.933E−13 | IGFBP7 | 2.989E−16 |
| C3 | 2.176E−13 | NDRG1 | 9.728E−13 | GPM6B | 4.439E−08 |
| CD63 | 9.534E−18 | FLNA | 1.343E−07 | SPARC | 1.298E−11 |
| FLNA | 1.325E−07 | S100A11 | 1.839E−13 | NES | 1.480E−13 |
| SPARC | 4.750E−10 | KRT17 | 2.327E−12 | APP | 1.595E−06 |
| HSP90AB1 | 1.694E−09 | SPARC | 5.029E−10 | CD63 | 9.474E−19 |
| APLP2 | 2.557E−09 | HSP90AB1 | 1.792E−09 | ALDOA | 1.208E−12 |
| CANX | 1.740E−15 | PGK1 | 1.622E−10 | ACTB | 1.091E−13 |
| VIM | 1.001E−12 | KRT5 | 1.323E−07 | COL1A2 | 2.962E−14 |
| GLUL | 2.978E−09 | GLUL | 3.145E−09 | CALR | 2.163E−15 |
| CALR | 8.774E−13 | SLC2A1 | 6.172E−08 | VIM | 1.798E−13 |
| CTSB | 1.862E−14 | CALR | 9.856E−13 | CHI3L1 | 2.054E−15 |
| PABPC1 | 3.860E−12 | CTSB | 2.202E−14 | CD74 | 1.243E−12 |
| KRT18 | 2.481E−10 | PABPC1 | 4.104E−12 | CANX | 1.277E−11 |
| ACTG1 | 6.504E−13 | KRT14 | 5.620E−13 | CTSB | 1.083E−15 |
| PIGR | 1.136E−14 | ACTG1 | 7.326E−13 | MYL6 | 1.340E−07 |
| COL3A1 | 3.334E−10 | COL3A1 | 3.533E−10 | MME | 5.566E−11 |
| COL1A2 | 1.238E−08 | COL1A2 | 1.306E−08 | GLUL | 2.343E−10 |
| LGALS3BP | 1.596E−08 | HSPB1 | 5.633E−08 | MT3 | 1.176E−13 |
| LYZ | 4.403E−08 | CD9 | 3.188E−10 | ACTG1 | 4.093E−14 |
| SFTPA2 | 6.670E−07 | SFTPA2 | 6.761E−07 | FTH1 | 3.312E−09 |
| SFTPA1 | 2.748E−07 | NAT1 | 7.000E−05 | HLA-C | 3.859E−10 |
| NAT1 | 6.686E−05 | B2M | 2.868E−05 | PLP1 | 6.797E−11 |
| B2M | 2.738E−05 | FTH1 | 2.663E−05 | OKI | 2.088E−08 |
| FTH1 | 2.541E−05 | FN1 | 8.189E−04 | PCDHGC3 | 8.640E−07 |
| FN1 | 7.717E−04 | CTSD | 2.598E−04 | SERPINA3 | 2.780E−02 |
| CTSD | 2.446E−04 | KRT15 | 2.935E−02 | BRI3 | 2.782E−08 |
| LPCAT1 | 2.506E−06 | SDC1 | 2.798E−08 | HSPA8 | 1.357E−09 |
| SLC34A2 | 7.538E−05 | LDHA | 2.833E−07 | AQP4 | 3.333E−07 |
| SERPINA1 | 3.826E−06 | GSTP1 | 1.246E−02 | GFAP | 1.795E−06 |
| LDHA | 2.794E−07 | ANXA2 | 8.001E−05 | NAT1 | 1.356E−07 |
| ANXA2 | 7.646E−05 | MYH9 | 4.997E−02 | B2M | 6.925E−04 |
| MYH9 | 4.997E−02 | HLA-C | 9.233E−06 | IGFBP5 | 1.804E−14 |
| HLA-C | 8.805E−06 | AKR1C1 | 1.819E−02 | CRYAB | 9.417E−04 |
| YWHAZ | 4.506E−02 | YWHAZ | 4.502E−02 | LRP1 | 1.732E−08 |
| HLA-A | 6.246E−03 | KRT13 | 1.886E−03 | HLA-A | 3.333E−07 |
| COL1A1 | 1.304E−01 | HLA-A | 6.523E−03 | SPARCL1 | 1.083E−04 |
| HLA-DRA | 6.860E−01 | COL1A1 | 1.303E−01 | S100B | 1.872E−04 |
| | | HLA-DRA | 6.859E−01 | FN1 | 4.728E−02 |
| | | | | CST3 | 6.713E−04 |
| | | | | PMP2 | 4.811E−02 |
| | | | | ITM2B | 1.148E−01 |
| | | | | HLA-DRA | 6.206E−01 |

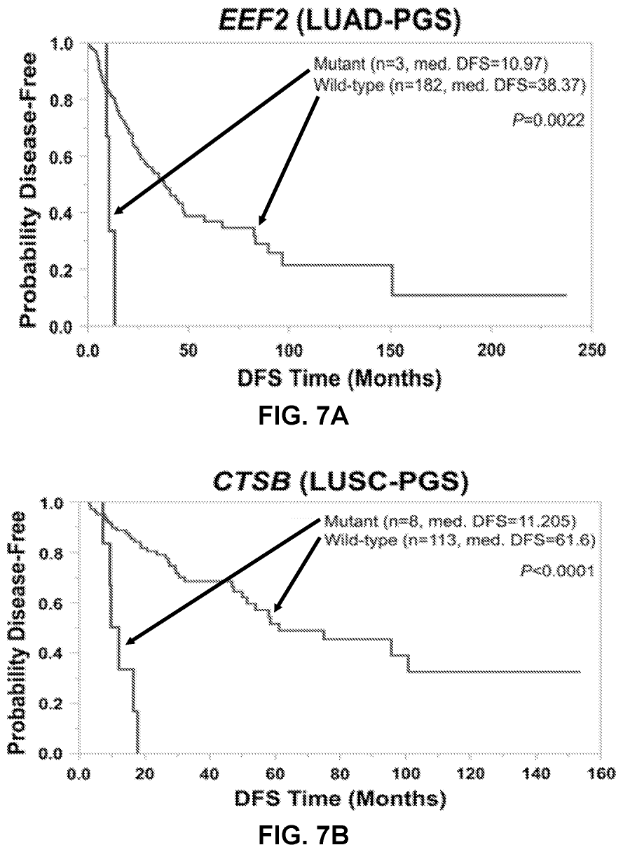

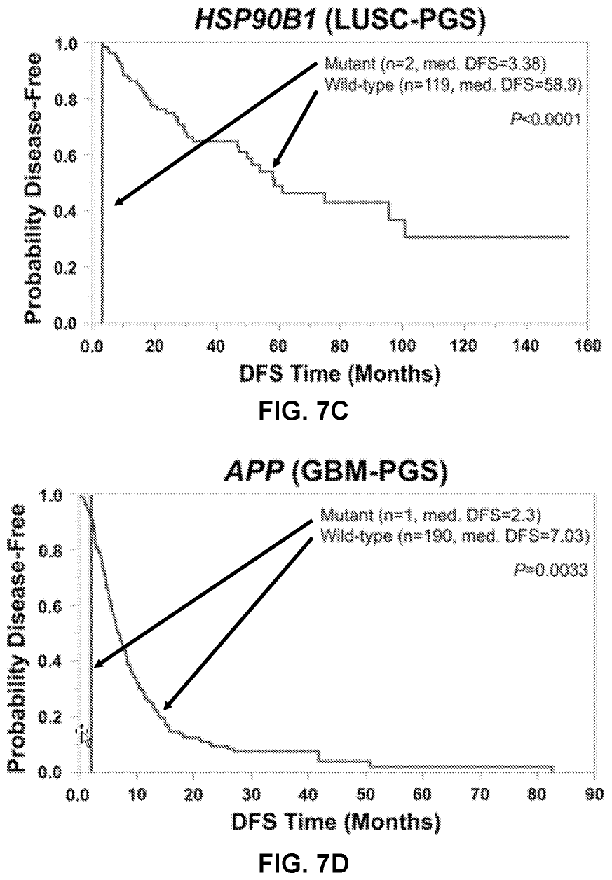

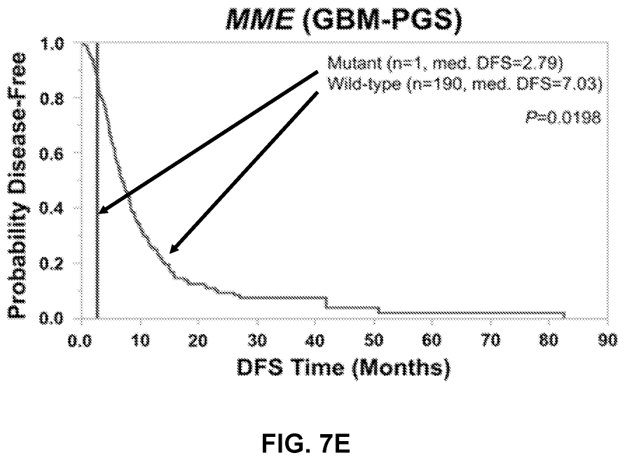

By using the pipeline described in FIG. 1A and a cutoff of average shRNA log 2 fold change <0 (lines), 67, 69, and 75 survival genes were identified in LUAD, LUSC and GBM, respectively (FIG. 1B-D, left panels). These highly expressed survival genes were then collectively assessed for their correlation with tumor progression incidence to assemble PGSs as biomarkers. Using backwards stepwise variable regression, P-values indicating the significance of candidate genes as predictor variables of tumor progression incidence in the model were calculated (FIG. 1B-D, right panel). By employing a P-value threshold of 0.25 (red lines), which allows us to select potential interacting variables that increase performance, a 22-gene LUAD-PGS, 23-gene LUSC-PGS, and 31-gene GBM-PGS were revealed (FIG. 1B-D, highlighted in bold, and Table 7-Table 9). Interestingly, there was only a 2-gene overlap between PGSs identified in LUAD and LUSC. To further characterize these distinct signatures, we investigated the mutation frequency of PGS genes in the TOGA cohorts. Almost all genes in the NSCLC PGSs were mutated in at least one patient (Table 10 and Table 11). Kaplan-Meier survival analyses revealed that mutations in eukaryotic translation elongation factor 2 (EEF2) in LUAD-PGS or cathepsin B (CTSB) and heat shock protein 90 beta family member 1 (HSP90B1) in LUSC-PGS correlated with shorter disease-free survival (DFS) time (FIGS. 7A-7C). These results demonstrate the difference of molecular profiles among NSCLC subtypes and a critical need for biomarkers to monitor disease progression in these subtypes. In GBM-PGS, signature genes were less frequently mutated compared to the NSCLC PGSs (Table 12). Despite the low mutation frequency, mutations in amyloid beta precursor protein (APP) and membrane metalloendopeptidase (MME) significantly correlated with shorter DFS time (FIGS. 7D-7E).

TABLE 7
Genes in LUAD-PGS.

| | | RefSeq Accession | |
|---|---|---|---|
| Gene Symbol | Full Gene Name | No. | SEQ ID NO. |
| ACTB | Actin Beta | NM_001101.5 | SEQ ID NO. 63 |
| FTL | Ferritin Light Chain | NM_000146.4 | SEQ ID NO. 83 |
| SFTPA2 | Surfactant Protein A2 | NM_001098668.4 | SEQ ID NO. 113 |
| CD74 | Cluster of Differentiation 74 Molecule | NM_001025159.2 | SEQ ID NO. 71 |
| FN1 | Fibronectin 1 | NM_212482.4 | SEQ ID NO. 82 |
| B2M | Beta-2-Microglobulin | NM_004048.4 | SEQ ID NO. 67 |
| CTSD | Cathepsin D | NM_001909.5 | SEQ ID NO. 77 |
| CEACAM6 | Carcinoembryonic Antigen-Related | NM_004363.6 | SEQ ID NO. 120 |
| | Cell Adhesion Molecule 6 | | |
| EEF2 | Eukaryotic Translation Elongation | NM_001961.4 | SEQ ID NO. 79 |
| | Factor 2 | | |
| PGC | Progastricsin | NM_002630.4 | SEQ ID NO. 102 |
| UBC | Ubiquitin C | NM_021009.7 | SEQ ID NO. 117 |
| HSP90AB1 | Heat Shock Protein 90 Alpha Family | NM_007355.4 | SEQ ID NO. 89 |
| | Class B Member 1 | | |
| SERPINA1 | Serpin Family A Member 1 | NM_000295.5 | SEQ ID NO. 112 |
| HSPA8 | Heat Shock Protein Family A (Hsp70) | NM_006597.6 | SEQ ID NO. 92 |
| | Member 8 | | |
| HSP90AA1 | Heat Shock Protein 90 Alpha Family | NM_005348.4 | SEQ ID NO. 88 |
| | Class A Member 1 | | |
| GNB2L1 | Receptor For Activated C Kinase 1 | NM_006098.5 | SEQ ID NO. 86 |
| (RACK1) | | | |
| CEACAM5 | Carcinoembryonic Antigen-Related | NM_004363.6 | SEQ ID NO. 72 |
| | Cell Adhesion Molecule 5 | | |
| CD63 | Cluster of Differentiation 63 Molecule | NM_001780.6 | SEQ ID NO. 70 |
| PIGR | Polymeric Immunoglobulin Receptor | NM_002644.4 | SEQ ID NO. 103 |
| KRT18 | Keratin 18 | NM_000224.3 | SEQ ID NO. 95 |
| GLUL | Glutamate-Ammonia Ligase | NM_002065.7 | SEQ ID NO. 85 |
| KRT19 | Keratin 19 | NM_002276.5 | SEQ ID NO. 96 |

TABLE 8
Genes in LUSC-PGS.

| | | RefSeq | |
|---|---|---|---|
| Gene Symbol | Full Gene Name | Accession No. | SEQ ID NO. |
| GAPDH | Glyceraldehyde-3-Phosphate | NM_002046.7 | SEQ ID NO. 84 |
| | Dehydrogenase | | |
| KRT5 | Keratin 5 | NM_000424.4 | SEQ ID NO. 97 |
| ACTG1 | Actin Gamma 1 | NM_001614.5 | SEQ ID NO. 64 |
| ENO1 | Enolase 1 | NM_001428 | SEQ ID NO. 80 |
| PKM | Pyruvate Kinase M1/2 | NM_002654 | SEQ ID NO. 104 |
| CTSB | Cathepsin B | NM_001908 | SEQ ID NO. 76 |
| PSAP | Prosaposin | NM_002778 | SEQ ID NO. 105 |
| MYH9 | Myosin Heavy Chain 9 | NM_002473.6 | SEQ ID NO. 101 |
| KRT14 | Keratin 14 | NM_000526.5 | SEQ ID NO. 94 |
| RPS4X | Ribosomal Protein S4 X-Linked | NM_001007 | SEQ ID NO. 108 |
| CALR | Calreticulin | NM_004343 | SEQ ID NO. 69 |
| FLNA | Filamin A | NM_001456 | SEQ ID NO. 81 |
| HSPA8 | Heat Shock Protein Family A (Hsp70) | NM_006597.6 | SEQ ID NO. 92 |
| | Member 8 | | |
| SFTPA2 | Surfactant Protein A2 | NM_001098668.4 | SEQ ID NO. 113 |
| RPS11 | Ribosomal Protein S11 | NM_001015 | SEQ ID NO. 107 |
| HSP90B1 | Heat Shock Protein 90 Beta Family | NM_003299 | SEQ ID NO. 90 |
| | Member 1 | | |
| HSPB1 | Heat Shock Protein Family B (Small) | NM_001540.5 | SEQ ID NO. 93 |
| | Member 1 | | |
| SDC1 | Syndecan 1 | NM_002997 | SEQ ID NO. 111 |
| HLA-C | Major Histocompatibility Complex, | NM_001243042.1 | SEQ ID NO. 87 |
| | Class I, C | | |
| APP | Amyloid Beta Precursor Protein | NM_000484 | SEQ ID NO. 65 |
| ATP1A1 | ATPase Na+/K+ Transporting Subunit | NM_000701 | SEQ ID NO. 66 |
| | Alpha 1 | | |
| HSPA5 | Heat Shock Protein Family A (Hsp70) | NM_005347.5 | SEQ ID NO. 91 |
| | Member 5 | | |
| RPL37 | Ribosomal Protein L37 | NM_000997.5 | SEQ ID NO. 106 |

TABLE 9
Genes in GBM-PGS.

| | | RefSeq | |
|---|---|---|---|
| Gene Symbol | Full Gene Name | Accession No. | SEQ ID NO. |
| RPS11 | Ribosomal Protein S11 | NM_001015.5 | SEQ ID NO. 107 |
| UBB | Ubiquitin B | NM_018955 | SEQ ID NO. 116 |
| TUBB | Tubulin Beta Class I | NM_178014.4 | SEQ ID NO. 115 |
| RPS6 | Ribosomal Protein S6 | NM_001010 | SEQ ID NO. 109 |
| EEF1A1 | Eukaryotic Translation Elongation | NM_001402 | SEQ ID NO. 78 |
| | Factor 1 Alpha 1 | | |
| EEF2 | Eukaryotic Translation Elongation | NM_001961.4 | SEQ ID NO. 79 |
| | Factor 2 | | |
| PKM | Pyruvate Kinase M1/2 | NM_002654.6 | SEQ ID NO. 104 |
| C3 | Complement C3 | NM_000064 | SEQ ID NO. 68 |
| ENO1 | Enolase 1 | NM_001428.5 | SEQ ID NO. 80 |
| HSP90AB1 | Heat Shock Protein 90 Alpha | NM_007355.4 | SEQ ID NO. 89 |
| | Family Class B Member 1 | | |
| FTL | Ferritin Light Chain | NM_000146.4 | SEQ ID NO. 83 |
| CFL1 | Cofilin 1 | NM_005507 | SEQ ID NO. 73 |
| YWHAE | Tyrosine | NM_006761.5 | SEQ ID NO. 119 |
| | 3-Monooxygenase/Tryptophan | | |
| | 5-Monooxygenase Activation | | |
| | Protein Epsilon | | |
| CKB | Creatine Kinase B | NM_001823 | SEQ ID NO. 74 |
| TUBA1A | Tubulin Alpha 1A | NM_006009.4 | SEQ ID NO. 114 |
| FLNA | Filamin A | NM_001456.4 | SEQ ID NO. 81 |
| APP | Amyloid Beta Precursor Protein | NM_000484.4 | SEQ ID NO. 65 |
| CD63 | Cluster of Differentiation 63 | NM_001780.6 | SEQ ID NO. 70 |
| | Molecule | | |
| ACTB | Actin Beta | NM_001101.5 | SEQ ID NO. 63 |
| VIM | Vimentin | NM_003380.5 | SEQ ID NO. 118 |
| CTSB | Cathepsin B | NM_001908 | SEQ ID NO. 76 |
| MME | Membrane Metalloendopeptidase | NM_007287 | SEQ ID NO. 99 |
| GLUL | Glutamate-Ammonia Ligase | NM_002065.7 | SEQ ID NO. 85 |
| MT3 | Metallothionein 3 | NM_005954 | SEQ ID NO. 100 |
| ACTG1 | Actin Gamma 1 | NM_001614.5 | SEQ ID NO. 64 |
| HLA-C | Major Histocompatibility Complex, | NM_001243042.1 | SEQ ID NO. 87 |
| | Class I, C | | |
| B2M | Beta-2-Microglobulin | NM_004048.4 | SEQ ID NO. 67 |
| CRYAB | Crystallin Alpha B | NM_001885.3 | SEQ ID NO. 75 |
| LRP1 | Low-Density Lipoprotein Receptor | NM_002332 | SEQ ID NO. 98 |
| | Related Protein 1 | | |
| S100B | S100 Calcium Binding Protein B | NM_006272.3 | SEQ ID NO. 110 |
| FN1 | Fibronectin 1 | NM_212482.4 | SEQ ID NO. 82 |

TABLE 10
Frequency and prognostic significance of mutations in LUAD-PGS genes
in the TCGA LUAD cohort. Kaplan-Meier survival curves analyzed
disease-free survival times between mutant or wild-type patients for each
PGS gene. P-values calculated using log-rank tests are shown. Genes with
mutations significantly correlated with patient prognosis are in bold.

| | Mutant | Wild-Type | KM log-rank |
|---|---|---|---|
| Gene | Patients | Patients | P-value |
| ACTB | 12 | 173 | 0.2126 |
| FTL | 2 | 183 | 0.5284 |
| SFTPA2 | 1 | 184 | 0.4086 |
| CD74 | 1 | 184 | 0.8161 |
| FN1 | 7 | 178 | 0.3636 |
| B2M | 6 | 179 | 0.3174 |
| CTSD | 0 | 185 | N/A |
| CEACAM6 | 3 | 182 | 0.3841 |
| EEF2 | 3 | 182 | 0.0022 |
| PGC | 12 | 173 | 0.6639 |
| UBC | 8 | 177 | 0.1792 |
| HSP90AB1 | 12 | 173 | 0.2348 |
| SERPINA1 | 3 | 182 | 0.1377 |
| HSPA8 | 7 | 178 | 0.8296 |
| HSP90AA1 | 7 | 178 | 0.1986 |
| GNB2L1 (RACK1) | 1 | 184 | 0.3223 |
| CEACAM5 | 6 | 179 | 0.1066 |
| CD63 | 4 | 181 | 0.7312 |
| PIGR | 15 | 170 | 0.8551 |
| KRT18 | 3 | 182 | 0.9135 |
| GLUL | 15 | 170 | 0.7759 |
| KRT19 | 1 | 184 | 0.2955 |

TABLE 11
Frequency and prognostic significance of mutations in LUSC-PGS genes
in the TCGA LUSC cohort. Kaplan-Meier survival curves analyzed
disease-free survival times between mutant or wild-type patients
for each PGS gene. P-values calculated using log-rank tests are
shown. Genes with mutations significantly correlated
with patient prognosis are highlighted in grey.

| | Mutant | Wild-Type | KM log-rank |
|---|---|---|---|
| Gene | Patients | Patients | P-value |
| GAPDH | 9 | 112 | 0.6864 |
| KRT5 | 0 | 121 | N/A |
| ACTG1 | 8 | 113 | 0.2008 |
| ENO1 | 1 | 120 | 0.8653 |
| PKM | 5 | 116 | 0.1453 |
| CTSB | 8 | 113 | <0.0001 |
| PSAP | 4 | 117 | 0.1688 |
| MYH9 | 8 | 113 | 0.5038 |
| KRT14 | 6 | 115 | 0.4735 |
| RPS4X | 2 | 119 | 0.5029 |
| CALR | 2 | 119 | 0.3733 |
| FLNA | 12 | 109 | 0.0689 |
| HSPA8 | 3 | 118 | 0.5524 |
| SFTPA2 | 8 | 113 | 0.5463 |
| RPS11 | 3 | 118 | 0.9702 |
| HSP90B1 | 2 | 119 | <0.0001 |
| HSPB1 | 1 | 120 | 0.6272 |
| SDC1 | 4 | 117 | 0.7084 |
| HLA-C | 1 | 120 | N/A |
| APP | 5 | 116 | 0.9992 |
| ATP1A1 | 5 | 116 | 0.2789 |
| HSPA5 | 2 | 119 | 0.3398 |
| RPL37 | 21 | 100 | 0.1694 |

TABLE 12
Frequency and prognostic significance of mutations in GBM-PGS genes
in the TCGA GBM cohort. Kaplan-Meier survival curves analyzed
disease-free survival times between mutant or wild-type patients for
each PGS gene. P-values calculated using log-rank tests are shown.
Genes with mutations significantly correlated with patient
prognosis are highlighted in grey.

| | Mutant | Wild-Type | KM log-rank |
|---|---|---|---|
| Gene | Patients | Patients | P-value |
| RPS11 | 0 | 191 | N/A |
| UBB | 0 | 191 | N/A |
| TUBB | 0 | 191 | N/A |
| RPS6 | 2 | 189 | 0.7997 |
| EEF1A1 | 2 | 189 | 0.6429 |
| EEF2 | 2 | 189 | 0.2193 |
| PKM | 1 | 190 | 0.1026 |
| C3 | 4 | 187 | 0.9628 |
| ENO1 | 2 | 189 | 0.2802 |
| HSP90AB1 | 2 | 189 | 0.6302 |
| FTL | 0 | 191 | N/A |
| CFL1 | 1 | 190 | 0.5808 |
| YWHAE | 1 | 190 | 0.1983 |
| CKB | 1 | 190 | 0.5163 |
| TUBA1A | 2 | 189 | 0.1250 |
| FLNA | 8 | 183 | 0.6733 |
| APP | 1 | 190 | 0.0033 |
| CD63 | 1 | 190 | 0.5421 |
| ACTB | 3 | 188 | 0.1451 |
| VIM | 0 | 191 | N/A |
| CTSB | 1 | 190 | 0.1589 |
| MME | 1 | 190 | 0.0198 |
| GLUL | 1 | 190 | 0.6717 |
| MT3 | 0 | 191 | N/A |
| ACTG1 | 0 | 191 | N/A |
| HLA-C | 0 | 191 | N/A |
| B2M | 0 | 191 | N/A |
| CRYAB | 2 | 189 | 0.9699 |
| LRP1 | 9 | 182 | 0.7616 |
| S100B | 0 | 191 | N/A |
| FN1 | 2 | 189 | 0.4096 |

The above PGSs were selected from genes essential for cancer cell survival; hence, it is likely that they are closely associated with cancer-related signaling pathways that control cancer cell proliferation and survival. To determine the functional relevance among these PGSs and validate their roles in tumor growth and progression, we queried the Reactome program (Ref. 57) to assess the enrichment of PGSs in molecular pathways. As summarized in Table 13, PGSs were heavily enriched in various immune response pathways associated with cancer development and progression. Genes in LUAD-PGS were highly involved in neutrophil degranulation, a process known to be associated with tumor plasticity and cancer metastasis (Ref. 58). In contrast, signature genes in LUSC-PGS or GBM-PGS were associated with cytokine signaling, which is implicated in regulating cellular proliferation and survival (Ref. 59). We next queried STRING, a program that determines potential protein-protein interactions (PPI) (Ref. 60). The number of edges, which describes the interconnectivity among a specified gene set, were 59, 66, and 123 in PPI networks of LUAD-PGS (22 genes), LUSC-PGS (23 genes), and GBM-PGS (31 genes), respectively, demonstrating significant interconnectivity between signature genes (Table 13, P<0.0001). Taken together, these results demonstrate the functional and physical connections among PGSs that are important for cancer growth and progression.

TABLE 13
PGSs are highly enriched in cancer-associated pathways and form significant protein-
protein interaction networks. The three most relevant pathways from Reactome pathway
analysis are shown in the left panel. Protein-protein interaction (PPI) networks
were constructed using STRING and summarized in the right panel. The number of edges
describes the level of interconnectivity of the networks and is expected to be equal
to the number of genes in the network. P-values indicating whether the observed
interactions were due to chance (PPI enrichment) were calculated by STRING.

| | | | | | STRING | |
|---|---|---|---|---|---|---|
| | Reactome | | | | | PPI |
| | | Number in | Total genes | FDRp- | Number of | enrichment |
| | Pathway | pathway | in pathway | value | edges | p-value |
| LUAD | Neutrophil degranulation | 11 | 480 | 4.38e−6 | 59 | 7.36e−11 |
| | Immune system | 22 | 2803 | 4.38e−6 | | |
| | Interleukin-4 and | 6 | 211 | 0.001 | | |
| | Interleukin-13 signaling | | | | | |
| LUSC | Interferon signaling | 16 | 392 | 2.70e−11 | 66 | 1.81e−13 |
| | Cytokine signaling in | 23 | 1245 | 6.39e−10 | | |
| | immune system | | | | | |
| | Cell-cell communication | 7 | 133 | 1.24e−05 | | |
| GBM | Interferon Signaling | 19 | 392 | 1.83e−13 | 123 | <1.00e−16 |
| | Cytokine signaling in | 29 | 1245 | 2.67e−13 | | |
| | immune system | | | | | |
| | Gap junction trafficking | 4 | 52 | 2.58e−03 | | |

### PGS Performance Exceeds Established Biomarkers.

To determine the prognostic significance of PGSs, we developed a risk score algorithm linearizing patient expression levels of each PGS to quantify patient risk for disease progression. Risk scores for each patient in the TOGA training cohorts were calculated on a scale of −50 to +50 representing lowest (−50) to highest (+50) risk of progression. tenfold cross validation in the training cohorts resulted in AUC values of 0.85, 0.92, and 0.84 for LUAD-PGS (A), LUSC-PGS (B), and GBM-PGS (C), respectively (FIG. 2, gray curves). We next determined the performance of established biomarkers such as the carcinoembryonic antigen (CEA) family, EGFR, tyrosine-protein kinase Met (MET), neuron-specific enolase (NSE), and KRAS for NSCLC (Refs. 13,14,61, and 62) and promoter methylation of MGMT, mutation of isocitrate dehydrogenase 1 (IDH1), EGFR, platelet-derived growth factor receptor alpha (PDGFRA), and cyclin-dependent kinase inhibitor 2A (CDKN2A) for GBM (Refs. 15 and 63). The AUC values of these established biomarkers ranged from 0.48 to 0.57 (FIG. 2, curves in different colors) and did not exceed 0.60 when assessed together (shown as combined current biomarkers; C.C.B.). These AUC values from established biomarkers were significantly lower than those of PGSs (P<0.0001).

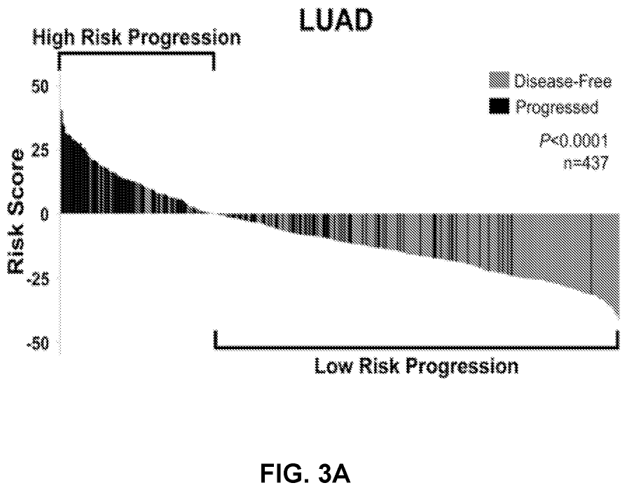

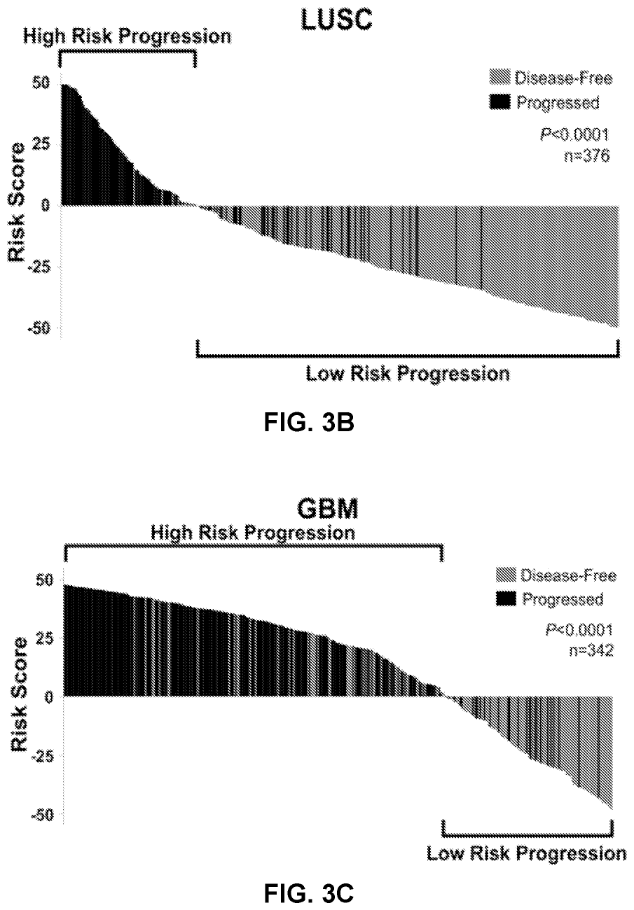

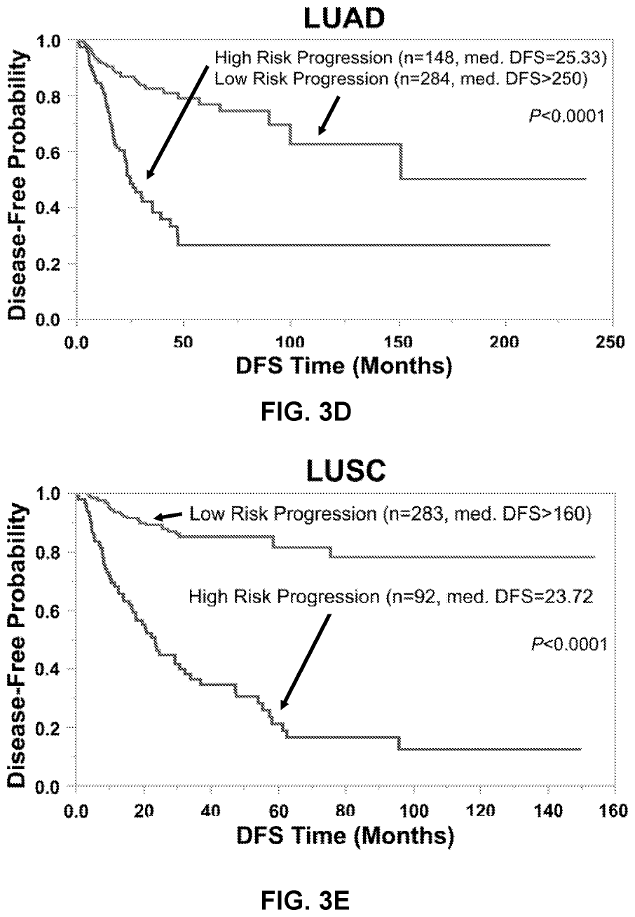

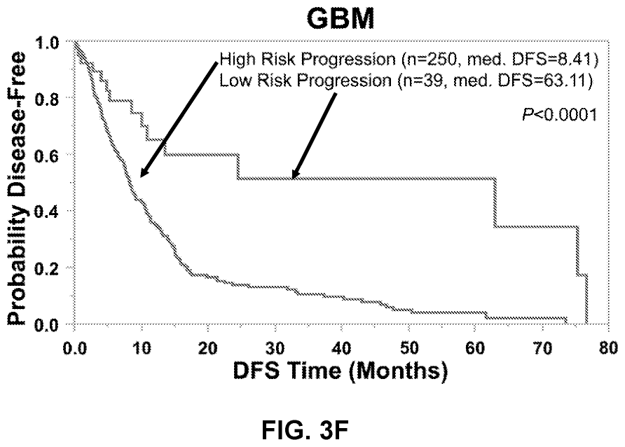

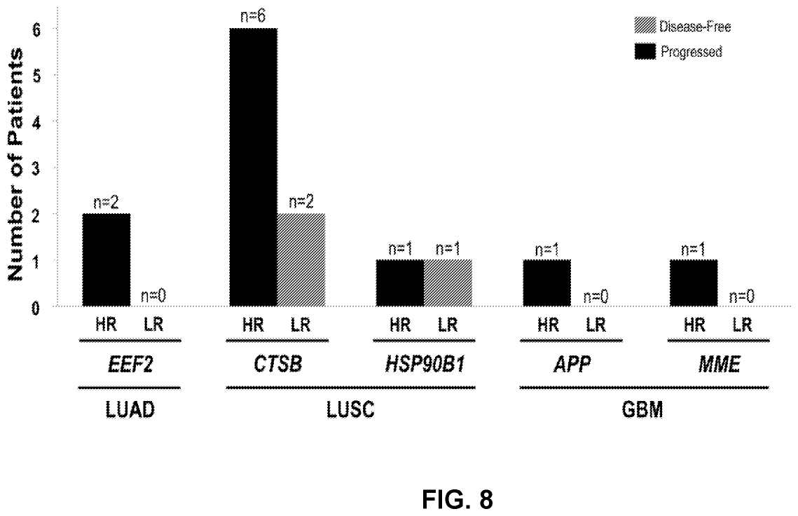

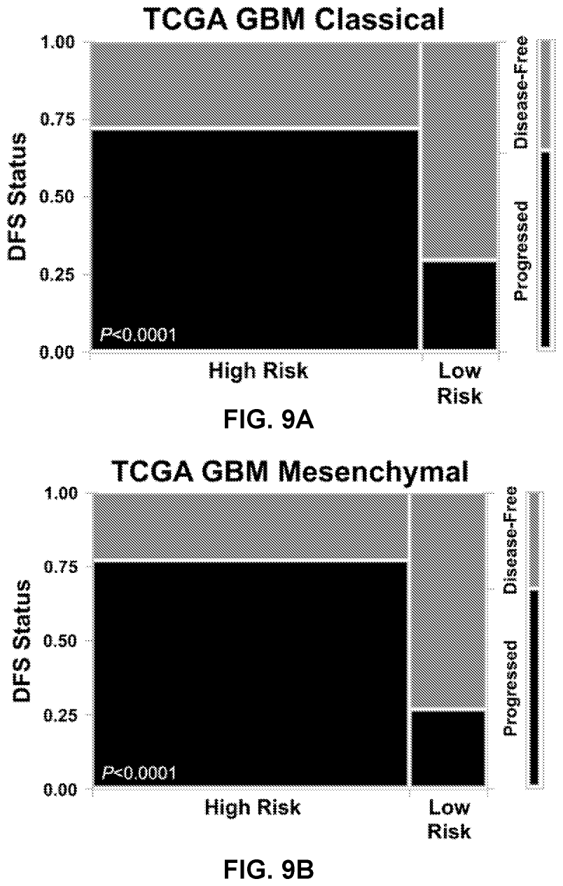

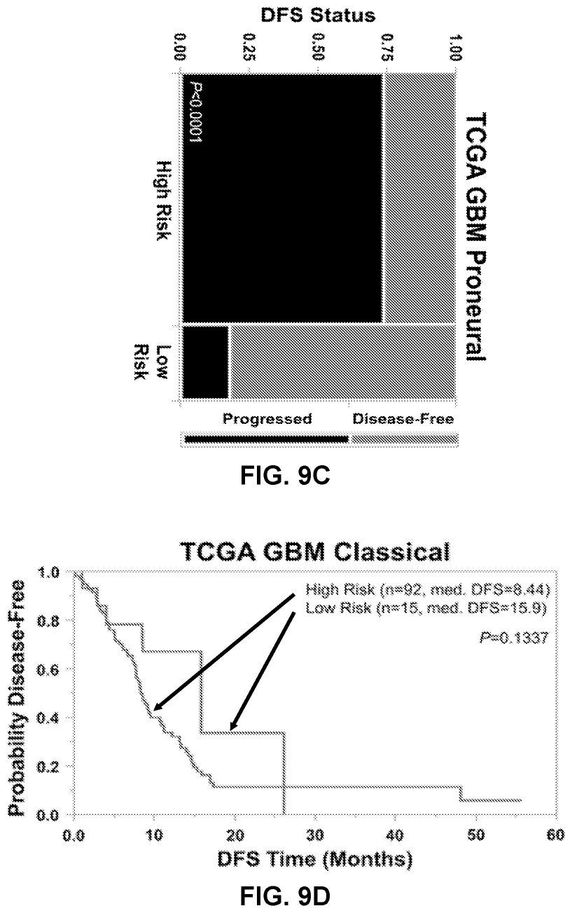

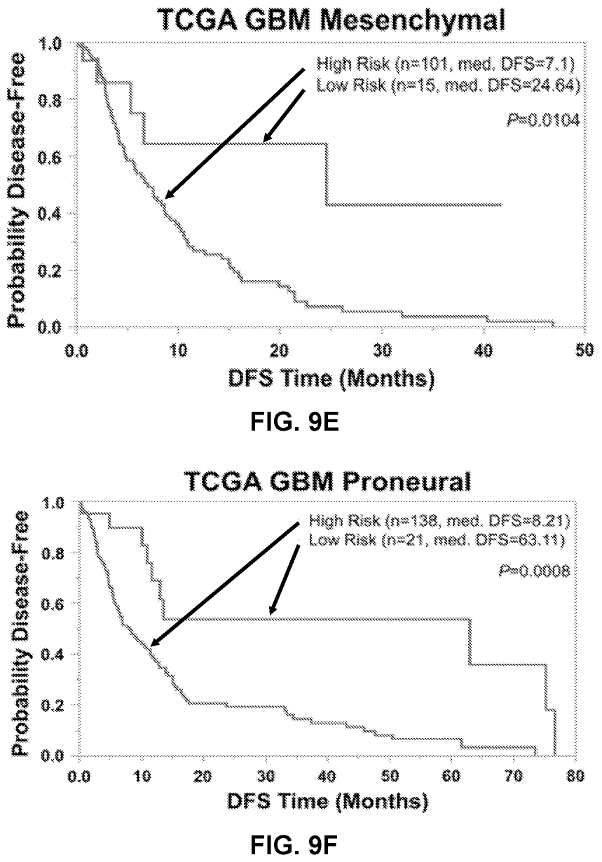

Next, we applied risk scores to stratify patients into high- or low-risk progression groups. A median risk score of 0 was used as the cutoff. As shown in FIG. 3, high-risk progression (risk score >0) patients diagnosed with LUAD (A), LUSC (B) or GBM (C) exhibited significantly increased frequency of tumor progression (highlighted in red), whereas low-risk progression patients (risk score <0) were mostly disease-free (blue). The P-value of this difference was less than 0.0001 in all cancers tested. Interestingly, patients harboring mutations in PGS genes that were prognostically significant were mostly classified as high-risk progression by all PGSs (FIG. 8). Similar results were also observed in the classical (n=107), mesenchymal (n=112), and proneural (n=170) GBM subtypes for high- and low-risk progression patients stratified by GBM-PGS (FIGS. 9A-9C). Kaplan-Meier survival analyses revealed that LUAD (D), LUSC (E), or GBM (F) patients in the high-risk progression group presented much shorter life spans than patients in the low-risk progression group (FIG. 3D, P <0.0001). The median DFS time in high-risk progression groups was 25.33 (FIG. 3D, LUAD), 23.72 (FIG. 3E, LUSC), or 8.41 (FIG. 3F, GBM) months. In stark contrast, median DFS times in low-risk progression groups were >250 (LUAD), >160 (LUSC), or 63.11 (GBM) months. We also analyzed DFS times of GBM-PGS risk groups in the three GBM subtypes to find that high-risk progression groups significantly correlated with worse patient prognosis in the mesenchymal (FIG. 9E, P=0.0104) and proneural (FIG. 9F, P=0.0008) subtypes but not the classical subtype (FIG. 9D, P=0.1337). The median DFS time in high-risk progression groups were 8.44 (classical), 7.1 (mesenchymal), and 8.21 (proneural) months com-pared to 15.9 (classical), 24.64 (mesenchymal), and 63.11 (proneural) months in low-risk progression patients. To further determine the performance of PGSs in patient prognosis, we used Cox proportional hazards models. The hazard ratios (HRs), which indicate risk of death, of LUAD-PGS or LUSC-PGS were 5.07 or 6.91, respectively (Table 14, univariate). In contrast, HRs of Tumor-Node-Metastasis (TNM) stage, age, gender, or smoking history ranged from 0.57 to 2.34, which were significantly lower than the HRs of PGSs. Similarly, GBM-PGS was more significantly associated with tumor progression (HR=3.02) than age or gender (HR=1.02 or 1.04, respectively). To determine whether the prognostic potential of PGSs depends upon other factors, we performed Cox multivariate analysis. LUAD-PGS and LUSC-PGS presented prognostic significance independent of TNM stage, age, gender, or smoking history, and GBM-PGS was unrelated to age or gender in predicting patient prognosis (Table 14) because there was no significant difference between HRs of univariate (HR=5.07, 6.91, or 3.02 for LUAD-PGS, LUSC-PGS, or GBM, respectively) and multivariate analyses (HR=5.06, 6.57, or 2.90, respectively).

TABLE 14
PGSs are independent prognostic factors. Cox univariate and multivariate regression models
were run using TNM stage, age, gender, and smoking history as additional clinicopathologic
predictors for NSCLC and age and gender for GBM. Stage I-II patients were categorized as
early-stage and stage III-IV patients were categorized as late−stage. The hazard ratios
(HR), 95% confidence intervals (CI), and P-values are shown. TNM—Tumor-Node-Metastasis.

| | | Univariate | | | Multivariate | | |
|---|---|---|---|---|---|---|---|
| | | HR | 95% CI | P | HR | 95% CI | P |
| LUAD | LUAD-PGS | 5.07 | [3.42-7.58] | <0.0001 | 5.06 | [3.36-7.68] | <0.0001 |
| | TNM stage | 2.34 | [1.50-3.56] | 0.0003 | 2.36 | [1.50-3.62] | 0.0004 |
| | (Late vs. Early) | | | | | | |
| | Age | 1.00 | [0.98-1.02] | 0.933 | 1.00 | [0.98-1.02] | 0.977 |
| | Gender | 0.92 | [0.62-1.35] | 0.664 | 0.99 | [0.66-1.46] | 0.955 |
| | (Male vs. Female) | | | | | | |
| | Smoking History | 0.99 | [0.59-1.79] | 0.982 | 0.99 | [0.58-1.84] | 0.992 |
| | (Smoker vs. None) | | | | | | |
| LUSC | LUSC-PGS | 6.91 | [4.51-10.80] | <0.0001 | 6.57 | [4.23-10.41] | <0.0001 |
| | TNM stage | 2.23 | [1.39-3.48] | 0.001 | 1.69 | [1.04-2.68] | 0.034 |
| | (Late vs. Early) | | | | | | |
| | Age | 1.02 | [0.99-1.04] | 0.07 | 1.02 | [0.99-1.04] | 0.07 |
| | Gender | 1.27 | [0.79-2.11] | 0.335 | 0.99 | [0.61-1.67] | 0.962 |
| | (Male vs. Female) | | | | | | |
| | Smoking History | 0.57 | [0.21-2.31] | 0.375 | 0.34 | [0.12-1.40] | 0.119 |
| | (Smoker vs. None) | | | | | | |
| GBM | GBM-PGS | 3.02 | [1.78-5.63] | <0.0001 | 2.90 | [1.70-5.42] | <0.0001 |
| | Age | 1.02 | [1.01-1.03] | <0.0001 | 1.02 | [1.01-1.03] | 0.0002 |
| | Gender | 1.04 | [0.79-1.39] | 0.778 | 1.00 | [0.75-1.34] | 0.991 |
| | (Male vs. Female) | | | | | | |

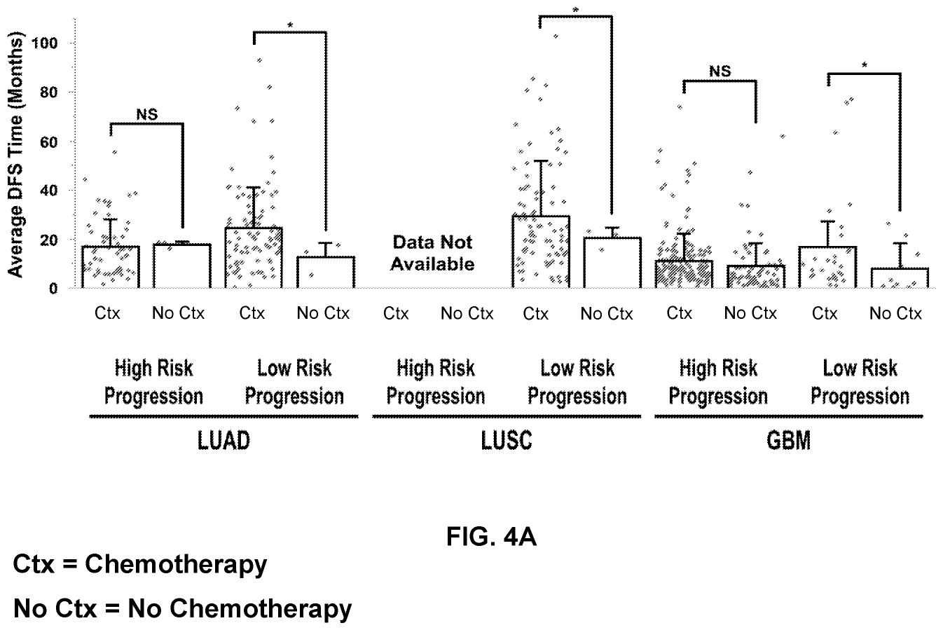

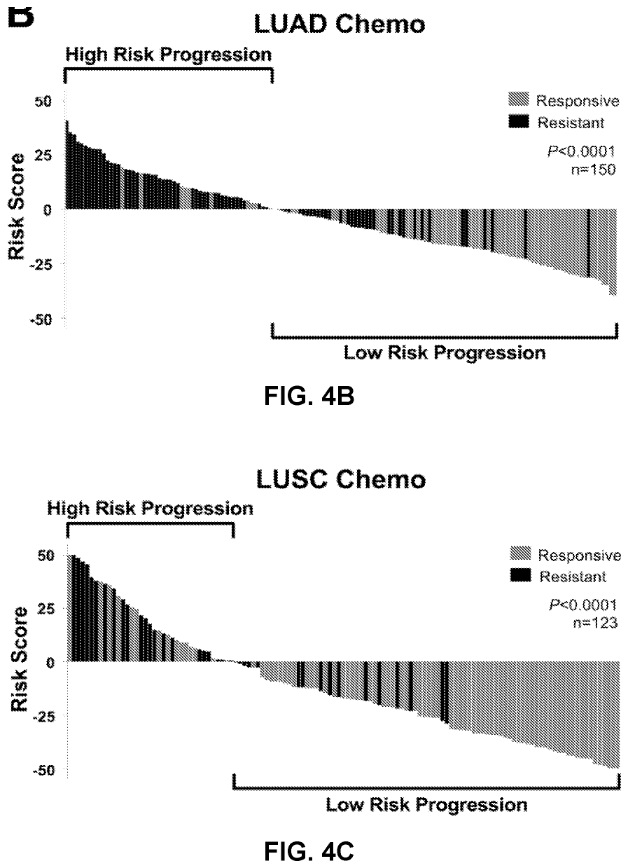

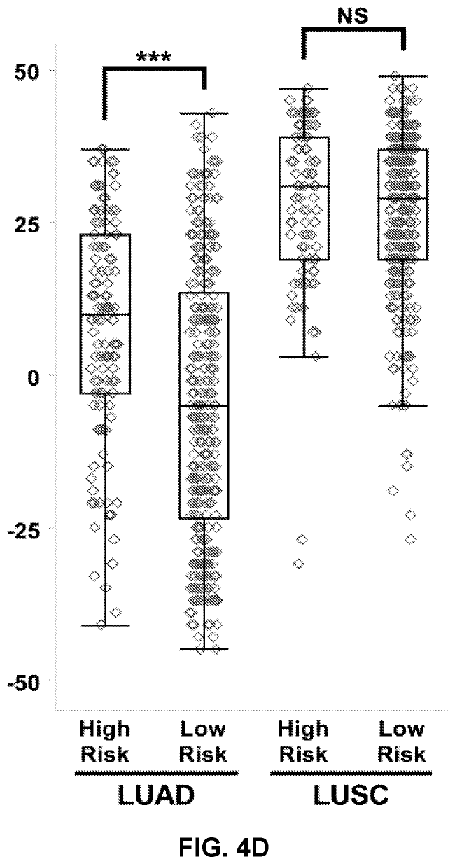

Treatment responses are often associated with tumor progression. ACT is the first-line therapy for NSCLC patients (Refs. 6 and 13), and TMZ is the only alkylating chemotherapeutic agent for GBM because of its efficient penetration through the blood-brain barrier (Refs. 3 and 11). However, ACT only presents a 4-15% survival advantage at 5 years post-treatment in early-stage NSCLC patients (Ref. 64), and around 50% of GBM patients develop resistance to TMZ and present poor prognosis (Ref. 11). To determine whether PGS-defined risk of poor prognosis correlates with treatment response, we analyzed the DFS times of high- and low-risk progression NSCLC patients treated with or without ACT or GBM patients treated with or without TMZ. The DFS times for high-risk progression patients treated with ACT or TMZ did not significantly differ compared to those treated without ACT or TMZ (FIG. 4A, P>0.05). Of note, however, only three LUAD patients were treated without ACT in the high-risk progression group and included in these analyses. The average DFS times for high-risk progression patients treated with ACT or TMZ was 16.40 (LUAD) or 10.80 (GBM) months compared to 18.18 (LUAD) or 8.44 (GBM) months in patients treated without ACT or TMZ. Data were unavailable for LUSC due to a lack of high-risk progression patients treated without ACT. In contrast, DFS times for low-risk progression patients were significantly higher in patients treated with ACT or TMZ (FIG. 4A, P<0.05). The average DFS times were 23.99 (LUAD), 28.86 (LUSC), and 16.52 (GBM) months in low-risk progression patients treated with ACT or TMZ compared to 12.28 (LUAD), 19.95 (LUSC), and 7.61 (GBM) months in patients treated without ACT or TMZ. While the sample sizes for LUAD and LUSC patients treated without ACT were small, these results suggest that patients with high risk of poor prognosis defined by PGSs may be resistant to chemotherapy. To further explore this observation, we retrieved ACT response information from the TCGA NSCLC cohorts. As expected, high-risk progression patients defined by PGSs were resistant to ACT, whereas low-risk progression patients were responsive to ACT (FIG. 4B-C, P<0.0001). Next, we determined tumor hypoxia levels in high- or low-risk progression patients because hypoxia often induces ACT resistance (Ref. 65). Based upon hypoxia scores determined by the Buffa mRNA abundance signature, LUAD patients in the high-risk progression group (red) exhibited a greater incidence of hypoxia than patients in the low-risk progression group (blue) manifested by higher Buffa hypoxia scores (FIG. 4D, P<0.0001). However, no difference in hypoxia score was detected in LUSC patients, possibly because the hypoxia index is already high in LUSC tumors (Ref. 66). Taken together, our results demonstrate that the PGSs identified herein are superior to established biomarkers in prognostic performance and that patients with high risk of poor prognosis, as defined by PGSs, are more likely to have shorter survival spans and develop progressive disease and therapeutic resistance.

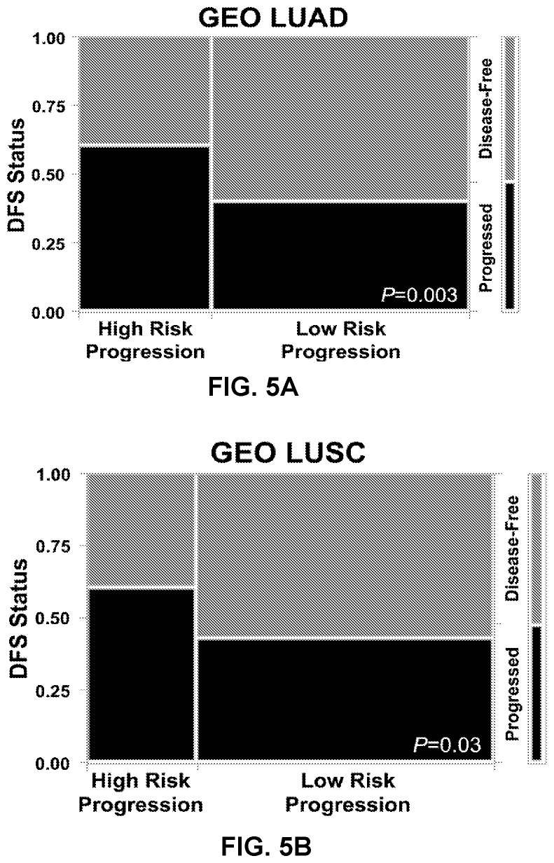

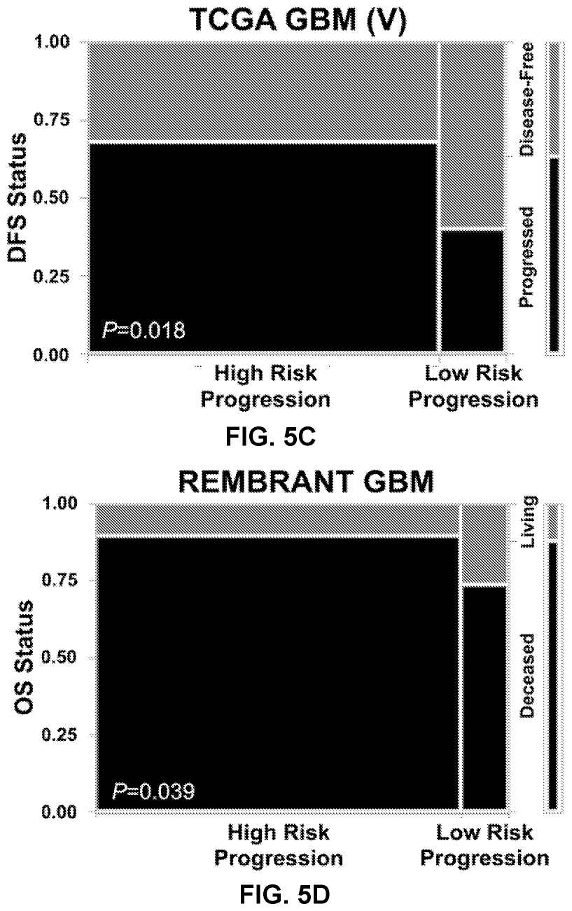

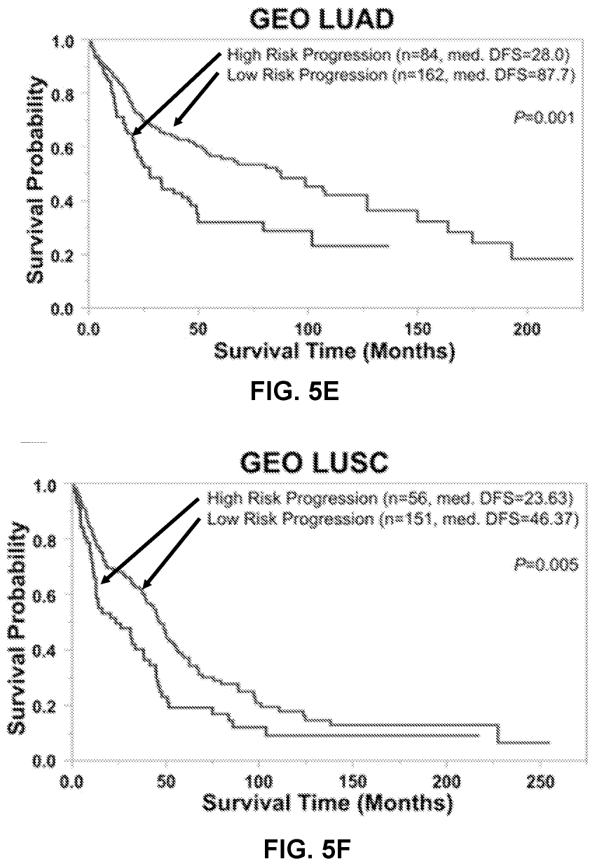

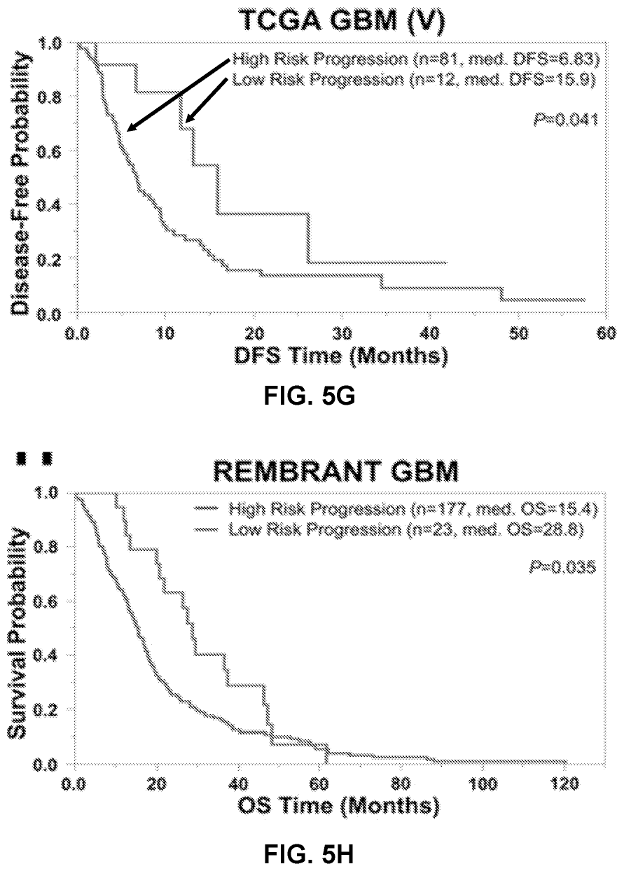

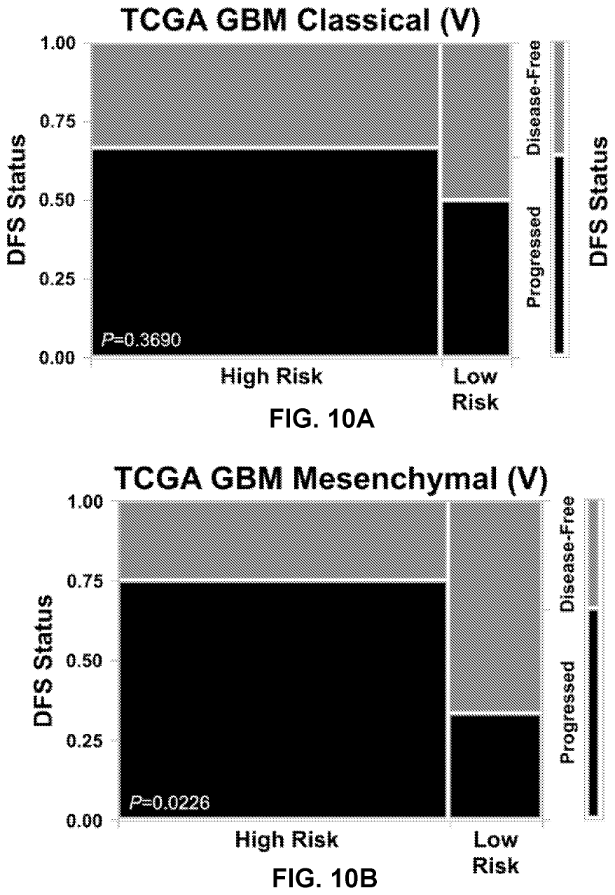

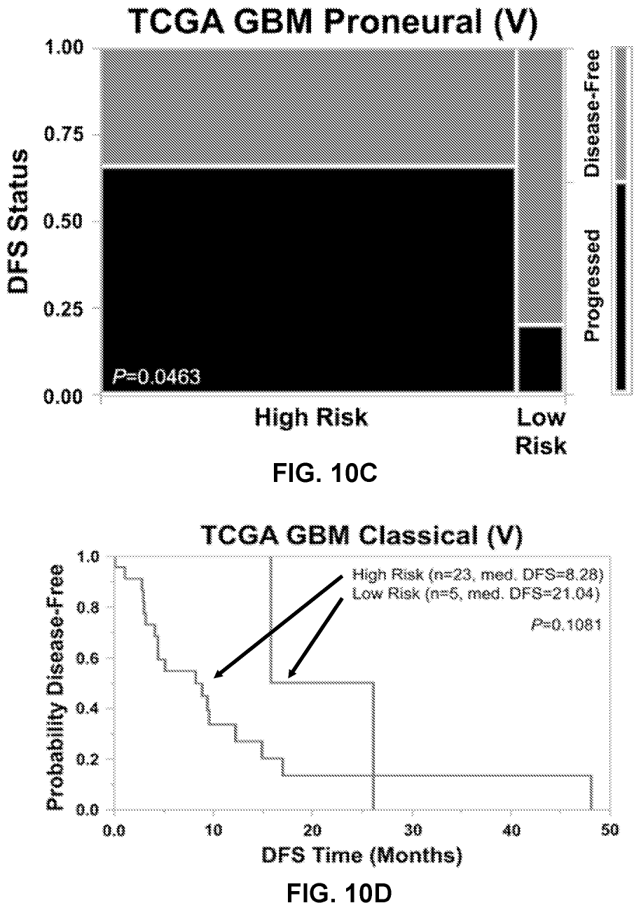

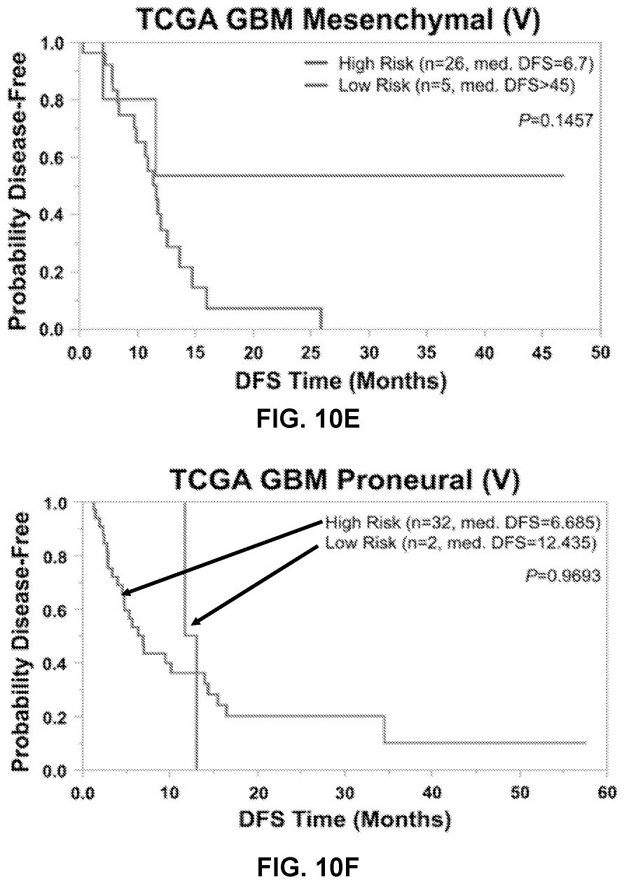

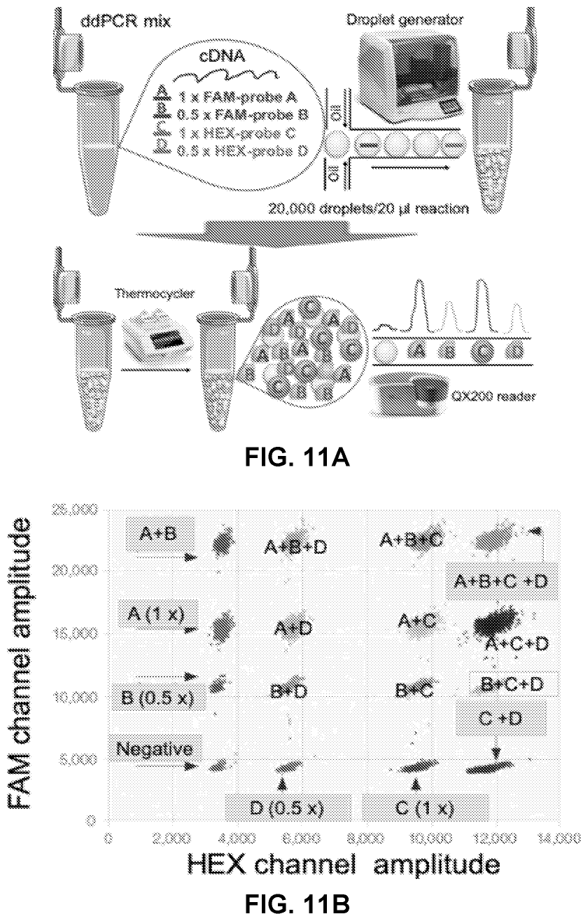

PGSs demonstrate robust performance in prognosis prediction in other patient cohorts and in freshly resected tumors of GBM patients. To validate the potential of PGSs identified herein as prognostic biomarkers, we retrieved four independent NSCLC microarray datasets from the Gene Expression Omni-bus (GEO) database, a TOGA GBM validation cohort comprising 126 samples, and a 200-patient external GBM validation cohort from Rembrandt (Ref. 53). These patient cohorts are thereafter designated as validation cohorts. As expected, high-risk progression patients stratified by PGSs of LUAD (A), LUSC (B), or GBM (C) showed higher levels of tumor progression and lower levels of disease-free survival than low-risk progression patients (FIG. 5, P<0.05). Consistently, data from the Rembrandt validation cohort showed that GBM patients in the high-risk progression group presented a greater chance of death than patients in the low risk progression group (FIG. 5D, P=0.039). When GBM-PGS was analyzed in each GBM subtype, the high-risk progression group correlated with increased tumor progression in the mesenchymal and proneural subtypes (FIGS. 10B-10C, P<0.05) but not the classical subtype (FIG. 10A, P=0.3690). These results were further validated by Kaplan-Meier survival analyses. Median survival times in LUAD (FIG. 5E) or LUSC (FIG. 5F) patients with high risk of poor prognosis were 28 or 23.63 months, respectively. However, median survival times in patients with low risk of poor prognosis were much longer (87.70 months for LUAD and 46.37 months for LUSC).

Similar results were obtained from the TOGA (FIG. 5G) and Rembrandt (FIG. 5H) GBM validation cohorts. The median DFS time in patients with high risk of poor prognosis was 6.83 and 15.4 months, which were significantly shorter than the median DFS time in patients with low risk of poor prognosis (15.2 or 28.8 months; P<0.05). In the GBM subtypes, median DFS times for high-risk progression patients were 8.28 (classical), 6.7 (mesenchymal), and 6.685 (proneural) months compared to 21.04 (classical), >45 (mesenchymal), and 12.435 (proneural) months for low-risk progression patients (FIGS. 10D-10F). However, log-rank tests did not reveal statistical significance for these differences due to the low number of low-risk progression patients (n<6) in all three GBM subtypes.

To prove the concept that PGSs are able to be used in clinical tests, we collaborated with the Fralin Biomedical Research Institute at Virginia Tech Carilion and Carilion Clinic and obtained six GBM primary lines derived from freshly dissected patient tumors. By employing quantitative RT-PCR to quantify mRNA levels of 31 genes in GBM-PGS and applying the risk algorithm defined in this study, five patients were stratified in the high-risk progression group and one patient in the low-risk progression group. As expected, patients in the group with high risk of poor prognosis presented an average OS time of 10.03 months, whereas the patient defined as low risk of poor prognosis survived for 18.68 months (FIG. 5I). While the sample size in this experiment was small, the capability of GBM-PGS in defining patients with high risk of poor prognosis was verified, thereby encouraging us to explore the potential of implementing PGSs into clinical tests. Hence, the results described above demonstrate the robustness of PGS performance in accurately predicting prognosis and highlight the potential of implementing PGSs into clinical tests.

### Discussion

In this report, we developed a biomarker discovery pipeline integrating genome-wide RNAi screens with global mRNA profiling data to identify survival gene-based PGSs in lung cancer and GBM. The importance of PGSs in predicting tumor progression, patient survival, and treatment response was further verified by multiple analyses in training cohorts and validation cohorts obtained from independent studies. Moreover, applying GBM-PGS in a small group of primary GBM samples mimicked a clinical test. Our innovative approach resulted in the identification of gene signatures that can be used as powerful prognostic markers for cancer diagnosis. Tumor staging and performance scoring are two factors often used in the clinic for the prediction of patient outcomes and selection of patients for chemotherapies (Refs. 7-10). However, these two factors are not sufficient. Several recent studies have attempted to apply prospective gene signatures for better prediction of prognosis or therapeutic benefit with or without tumor staging and performance scoring; however, these studies lack a strong translational potential because they only employed gene expression-based approaches, neglecting the functional relevance of candidate genes to the disease. The biomarker pipeline described herein identifies gene signatures based upon the importance of genes to cancer cell survival, which addresses the issue described above.

While we only showed results in lung cancer and GBM, this pipeline could be a powerful tool in identifying biomarkers in other cancers.

The PGSs identified herein presented a robust performance in predicting patient outcomes that was superior to clinically-used biomarkers and molecular prognostic markers established previously, providing a strong support to our hypothesis. More importantly, we found that there was little overlap between the PGSs in this study and gene signatures in other studies (Refs. 27,28, and 30-32). For instance, we identified three heat shock protein (HSP) genes, HSP 90 alpha family class B member 1 (HSP90AB1), HSP family A member 8 (HSPA8), and HSP family A member 5 (HSPA5), as biomarkers in lung cancer and GBM. HSPs are diversely implicated in cell proliferation, invasion, and migration through their roles in controlling cell cycle progression and protecting cells against apoptosis under stress (Ref. 67). Certain HSP genes have been studied for association with patient prognosis and treatment response (Refs. 67 and 68); however, the HSP genes we identified have not been previously reported as lung cancer or GBM biomarkers. We also identified multiple cytoskeleton-associated genes, including keratin 18 (KRT18) in LUAD, keratin 14 (KRT14) in LUSC, and cofilin 1 (CFL1) in GBM as prognostic and predictive biomarkers. Past studies have highlighted the important role of cytoskeletal dynamics in mediating chemotherapy resistance and cancer metastasis (Ref. 69). Taken together, the functional relevance of PGSs to cancer cell survival, proliferation, and drug response further supports the feasibility of using essential survival genes as biomarkers that can accurately predict cancer progression.

The PGSs identified in this study contain some survival genes previously reported as prognostic markers. For example, carcinoembryonic antigen-related cell adhesion molecule 5/6 (CEACAM5/CEACAM6) in LUAD-PGS belongs to the well-known CEA protein family associated with carcinogenesis and progression in multiple cancers61. Fibronectin 1 (FN1) is a prognostic and predictive biomarker in head and neck squamous cell carcinoma (Refs. 70 and 71). Guanine nucleotide-binding protein subunit beta-2-like 1 (GNB2L1), also known as receptor for activated C kinase 1 (RACK1), serves as a prognostic biomarker in pancreatic and breast cancer (Refs. 72 and 73). Enolase 1 (ENO1) and cathepsin B (CTSB), found in both LUSC-PGS and GBM-PGS, are predictive biomarkers for hepatocellular carcinoma, gastric cancer, or oral squamous cell carcinoma (Refs. 74-76). The presence of established biomarkers within PGSs highlights the power and feasibility of our integrated approach to cancer biomarker discovery. It is also noted that the construction of PGSs from genes implicated in cancer cell survival allows for the potential development of targeted therapies as companion therapeutics (Ref. 41). Accordingly, multiple signature genes in PGSs identified herein are appealing therapeutic targets worth further investigation. For instance, glutamate-ammonia ligase (GLUL) in LUAD-PGS and GBM-PGS encodes an enzyme catalyzing the synthesis of glutamine, an essential amino acid for DNA synthesis and repair (Ref. 77). Glutamine metabolism is often remodeled in cancer to increase cell proliferation (Refs. 77 and 78). Given the relatively low expression of GLUL in normal tissues78, the aberrant activity of GLUL in progressive cancer patients can be an appealing therapeutic target for LUAD and GBM. A GLUL inhibitor L-methionine-S,R-sulfoximine is commercially available (Ref. 79), and future studies should investigate the possibility of this inhibitor in treating LUAD or GBM. CTSB is a target candidate in LUSC-PGS and GBM-PGS, encoding a member of the cathepsin protein family which remodel the extracellular matrix to facilitate cancer invasion and metastasis (Ref. 80). A number of CTSB inhibitors have been developed (Ref. 81), but the efficacy of these drugs in lung cancer or GBM has not been explored. Some genes in LUSC-PGS or GBM-PGS were involved in interferon (IFN) signaling pathways. The roles of IFN signaling in tumors are controversial—IFN triggers anti-tumor immunity, but emerging evidence also suggest prolonged activation of IFN signaling leads to therapy resistance through increased JAK/STAT signaling (Ref. 82). As such, a number of JAK/STAT inhibitors including AZD1480 and LLL12 have demonstrated promising efficacy in treating NSCLC and GBM (Refs. 83-85). A recent study by Hu et al. also showed that the JAK2 inhibitor ruxolitinib restored cisplatin sensitivity in NSCLC (Ref. 86). Taken together, our innovative biomarker discovery pipeline identifies PGSs that not only serve as accurate predictors of tumor progression and treatment response, but also help develop effective cancer therapies.

Various modifications and variations of the described methods, pharmaceutical compositions, and kits of the invention will be apparent to those skilled in the art without departing from the scope and spirit of the invention. Although the invention has been described in connection with specific aspects, it will be understood that it is capable of further modifications and that the invention as claimed should not be unduly limited to such specific aspects. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of determining a cancer progression risk score of a subject, the method comprising:
detecting expression levels of genes of a progression gene signature in a sample; and
calculating the cancer progression risk score of the subject using the expression levels of genes associated with a progression gene signature in the sample;
wherein the progression gene signature comprises a glioblastoma progression gene signature;
wherein the cancer progression risk score is high risk progression or low risk progression;
wherein the detecting expression levels of genes of the progression gene signature comprises detecting expression levels of a glioblastoma progression gene signature; and
wherein the detecting comprises detecting expression levels of five genes selected from RPS11, UBB, TUBB, RPS6, EEF1A1, EEF2, PKM, C3, ENO1, HSP90AB1, FTL, CFL1, YWHAE, CKB, TUBA1A, FLNA, APP, CD63, ACTB, VIM, CTSB, MME, GLUL, MT3, ACTG1, HLA-C, B2M, CRYAB, LRP1, S100B, and FN1.

2. The method of claim 1, wherein the sample is obtained from the subject.

3. The method of claim 2, wherein the sample is obtained from a tumor, tissue, bodily fluid, or a combination thereof.

4. The method of claim 1, wherein the subject is a human.

5. The method of claim 4, wherein the subject is diagnosed with a cancer.

6.-9. (canceled)

10. The method of claim 1, wherein the detecting comprises detecting expression levels of ten genes selected from RPS11, UBB, TUBB, RPS6, EEF1A1, EEF2, PKM, C3, ENO1, HSP90AB1, FTL, CFL1, YWHAE, CKB, TUBA1A, FLNA, APP, CD63, ACTB, VIM, CTSB, MME, GLUL, MT3, ACTG1, HLA-C, B2M, CRYAB, LRP1, S100B, and FN1.

11. The method of claim 1, wherein the detecting comprises detecting expression levels of each of the genes RPS11, UBB, TUBB, RPS6, EEF1A1, EEF2, PKM, C3, ENO1, HSP90AB1, FTL, CFL1, YWHAE, CKB, TUBA1A, FLNA, APP, CD63, ACTB, VIM, CTSB, MME, GLUL, MT3, ACTG1, HLA-C, B2M, CRYAB, LRP1, S100B, and FN1.

12.-19. (canceled)

20. The method of claim 1, wherein the detecting expression levels of genes of a progression gene signature in a sample comprises detecting using a method selected from a PCR method, a RNASeq method, and combinations thereof.

21. The method of claim 20, wherein the detecting expression levels of genes of a progression gene signature in a sample comprises detecting using a PCR method selected from ddPCR, digital droplet PCR, qPCR, and combinations thereof.

22. The method of claim 21, wherein the PCR method utilizes one or more primers selected from SEQ ID NOs. 1-62.

23. The method of claim 1, wherein the calculating the cancer progression risk of the subject using the expression levels of genes associated with a progression gene signature in the sample comprises:
deriving a cancer progression risk score model comprising:
carrying our principal component analysis of a set of principal components (PCs) linearizing z-score-normalized gene expression values across the progression gene signature for a dataset comprising at least 100 patient samples with known tumor progression outcome;
wherein the number principal components generated was equal to the number of genes in the progression gene signature;
screening the principal components using random forests of 1000 trees trained on a yes/no indicator of tumor progression and selecting principal components correlated with incidence of the tumor progression, and implementing a percent contribution cutoff of >0.05;
selecting principal components and repeating the carrying our principal component analysis and screening the principal components until random forests retained all principal components;
subjecting the end principal component set into a neural network with three tan H nodes
boosted 100 times at a 0.1 learning rate with tenfold cross validation;
providing the formula output as a probability of the tumor progression on a scale of 0 to 1, and then transposing to a scale of −50 to 50;
wherein a cutoff of 0 stratified the tumor progression as high risk and <0 stratified the tumor progression as low risk;
providing data for the expression levels of genes associated with a progression gene signature in the sample as input to the cancer progression risk score model to determine the cancer progression risk score of the subject.

24.-25. (canceled)

26. The method of claim 1, wherein the calculating the cancer progression risk of the subject using the expression levels of genes associated with a progression gene signature in the sample comprises using a classification method, the classification method constructed by:
a. generating a set of components by dimensionality reduction of expression levels of the genes in a training data set, the training data set comprising gene expression levels from training subjects having a high risk of progression and training subjects having a low risk of progression;
b. training a machine learning model to select a subset of components from the set of components, the subset of components being more highly correlated to the risk of progression as compared to a correlation of the unselected components;
c. repeating steps (a) and (b) with the selected subset of components from the set of components until there are no unselected components from the machine learning model of step (b); and
d. constructing the classification method from the subset of components.

27. The method of claim 26, wherein the classification method is a neural network.

28. The method of claim 26, wherein the subset of components being more highly correlated comprises having a percent contribution cutoff of about 0.05 or more.

29. A method of detecting a cancer in a subject or a sample therefrom containing cells comprising:
determining a cancer progression risk score of a subject as in claim 1; and
diagnosing the cancer in the subject when a cancer signature is detected.

30. (canceled)

31. The method of claim 29, further comprising administering a chemotherapy agent or modality to the subject.

32. A method of treating a cancer in a subject, comprising:
determining a cancer progression risk score of a subject as in claim 1; and
administering an effective amount of an agent effective to modulate, inhibit a function and/or activity of a cancer cell, and/or kill a cancer cell, or a combination thereof to the subject.

33. The method of claim 32, wherein the progression signature is indicative of the subject having a high risk of progression or a low risk of progression; and treating the subject with a more aggressive cancer treatment based upon the subject having a high risk of progression or a less aggressive cancer treatment based upon the subject having a low risk of progression.

34.-41. (canceled)

42. A system to process biological information, comprising:
one or more processors; and
one or more memory elements including instructions, which when executed cause the one or more processors to:
receive an array of ribonucleic acid (RNA) sequence data associated with a group of patients;
determine a first set of gene sequences having respective expression magnitudes greater than a threshold value in at least one subtype from the array of RNA sequence data;
select, from the first set of gene sequences, a second set of gene sequences based on a model selection criteria;
determine, from the second set of gene sequences, a set of cancer survival gene sequences based on cross-referencing each gene sequence from the second set of gene sequences with RNA interference data; and
select, from the set of cancer survival gene sequences, a set of progression gene signatures, based on a tumor progression criteria.

43.-49. (canceled)

50. A computer-implemented method for processing biological information, comprising:
receiving, by a computer server including one or more processors, an array of ribonucleic acid (RNA) sequence data associated with a group of patients;
determining, by the computer server, a first set of gene sequences having respective expression magnitudes greater than a threshold value in at least one subtype from the array of RNA sequence data;
selecting, by the computer server, from the first set of gene sequences, a second set of gene sequences based on a model selection criteria;
determining, by the computer server, from the second set of gene sequences, a set of cancer survival gene sequences based on cross-referencing each gene sequence from the second set of gene sequences with RNA interference data; and
selecting, by the computer server, from the set of cancer survival gene sequences, a set of progression gene signatures, based on a tumor progression criteria.

51.-57. (canceled)


SEQUENCE LISTING (120 sequences)

SEQ ID NO: 1 - DNA, 18 bp, Homo sapiens
agcagccgaccatctttc

SEQ ID NO: 2 - DNA, 21 bp, Homo sapiens
atagcctccttgggtgtcttg

SEQ ID NO: 3 - DNA, 18 bp, Homo sapiens
aagcctaaactgcctctc

SEQ ID NO: 4 - DNA, 18 bp, Homo sapiens
gttgcgtcacttatcacc

SEQ ID NO: 5 - DNA, 21 bp, Homo sapiens
gcagtcaccttcattggcaat

SEQ ID NO: 6 - DNA, 20 bp, Homo sapiens
gcggaacatggcagtgaact

SEQ ID NO: 7 - DNA, 26 bp, Homo sapiens
cgccaagtatgttgtaagaaagccct

SEQ ID NO: 8 - DNA, 23 bp, Homo sapiens
gctgcaggacacgtggagtaaca

SEQ ID NO: 9 - DNA, 20 bp, Homo sapiens
caggacacagagactttatc

SEQ ID NO: 10 - DNA, 19 bp, Homo sapiens
agtgtgtaagccagaaggg

SEQ ID NO: 11 - DNA, 19 bp, Homo sapiens
agaagctgtggggtgacag

SEQ ID NO: 12 - DNA, 19 bp, Homo sapiens
gatcagctggcagaaggtg

SEQ ID NO: 13 - DNA, 24 bp, Homo sapiens
gtgcgagcctcaagtcactccaca

SEQ ID NO: 14 - DNA, 24 bp, Homo sapiens
tataagaagcctccacgctgccca

SEQ ID NO: 15 - DNA, 20 bp, Homo sapiens
gctgaagcacctcattgtga

SEQ ID NO: 16 - DNA, 20 bp, Homo sapiens
ctgggtgtaccccttcttga

SEQ ID NO: 17 - DNA, 20 bp, Homo sapiens
gagctccgggacaatgataa

SEQ ID NO: 18 - DNA, 20 bp, Homo sapiens
ctgttccatccatctcgatc

SEQ ID NO: 19 - DNA, 21 bp, Homo sapiens
tttgggaaccattgccaagtc

SEQ ID NO: 20 - DNA, 21 bp, Homo sapiens
caccaaactgcccaatcatgg

SEQ ID NO: 21 - DNA, 20 bp, Homo sapiens
gcgtctcctgaagatgcaaa

SEQ ID NO: 22 - DNA, 22 bp, Homo sapiens
aggaagtgagtctccaggaagt

SEQ ID NO: 23 - DNA, 26 bp, Homo sapiens
gaaggaggatctggtgtttatcttct

SEQ ID NO: 24 - DNA, 22 bp, Homo sapiens
ccttggagctggcataaatcat

SEQ ID NO: 25 - DNA, 22 bp, Homo sapiens
ggatacgctgagtgaagaaagc

SEQ ID NO: 26 - DNA, 22 bp, Homo sapiens
tattctgctcttcaccgtcacc

SEQ ID NO: 27 - DNA, 19 bp, Homo sapiens
gctgcgacttcagaagcga

SEQ ID NO: 28 - DNA, 19 bp, Homo sapiens
ggcatgaggtcgtcgatgg

SEQ ID NO: 29 - DNA, 20 bp, Homo sapiens
gcaacaacctctcctcttcg

SEQ ID NO: 30 - DNA, 20 bp, Homo sapiens
gaatcatctcctcccccaat

SEQ ID NO: 31 - DNA, 21 bp, Homo sapiens
gcacttacagctgctcctacg

SEQ ID NO: 32 - DNA, 18 bp, Homo sapiens
ccagctcccacattcacc

SEQ ID NO: 33 - DNA, 20 bp, Homo sapiens
aaccagtgaccatccagaac

SEQ ID NO: 34 - DNA, 20 bp, Homo sapiens
acttgtcaggaacgagaagg

SEQ ID NO: 35 - DNA, 19 bp, Homo sapiens
agcagatggagaattaccc

SEQ ID NO: 36 - DNA, 20 bp, Homo sapiens
ctcccaatctgtgtagttag

SEQ ID NO: 37 - DNA, 17 bp, Homo sapiens
ttcctgggcatggagtc

SEQ ID NO: 38 - DNA, 18 bp, Homo sapiens
caggtctttgcggatgtc

SEQ ID NO: 39 - DNA, 20 bp, Homo sapiens
agccgaaaacaccctgcaat

SEQ ID NO: 40 - DNA, 20 bp, Homo sapiens
cgttcaaggtcaagacgtgc

SEQ ID NO: 41 - DNA, 18 bp, Homo sapiens
ggccccctgcatctatcg

SEQ ID NO: 42 - DNA, 21 bp, Homo sapiens
aggtctcccgctgttccactg

SEQ ID NO: 43 - DNA, 21 bp, Homo sapiens
catcggcatggtcataggaca

SEQ ID NO: 44 - DNA, 22 bp, Homo sapiens
tgttgagtccaccagtcaacga

SEQ ID NO: 45 - DNA, 20 bp, Homo sapiens
cacacaatcttggcatttcc

SEQ ID NO: 46 - DNA, 20 bp, Homo sapiens
actcaggggagcaaaggaag

SEQ ID NO: 47 - DNA, 19 bp, Homo sapiens
atggaccctgagacctgcc

SEQ ID NO: 48 - DNA, 20 bp, Homo sapiens
ttgcacacacagtccttggc

SEQ ID NO: 49 - DNA, 20 bp, Homo sapiens
tgtttccttccatcgtcggg

SEQ ID NO: 50 - DNA, 20 bp, Homo sapiens
catgtcgtcccagttggtga

SEQ ID NO: 51 - DNA, 20 bp, Homo sapiens
tcctggttgtcctagctgtc

SEQ ID NO: 52 - DNA, 20 bp, Homo sapiens
caggctttacaagtgatgag

SEQ ID NO: 53 - DNA, 20 bp, Homo sapiens
gatgagtatgcctgccgtgt

SEQ ID NO: 54 - DNA, 20 bp, Homo sapiens
tgcggcatcttcaaacctcc

SEQ ID NO: 55 - DNA, 22 bp, Homo sapiens
aggtgttgggagatgtgattga

SEQ ID NO: 56 - DNA, 23 bp, Homo sapiens
ggatgaagtaatggtgagagggt

SEQ ID NO: 57 - DNA, 22 bp, Homo sapiens
acatatagcctccatcctaatc

SEQ ID NO: 58 - DNA, 19 bp, Homo sapiens
ttccaatctccacgttcat

SEQ ID NO: 59 - DNA, 20 bp, Homo sapiens
agacggtcatgcaagaaagc

SEQ ID NO: 60 - DNA, 20 bp, Homo sapiens
gctacaacacggctggaaag

SEQ ID NO: 61 - DNA, 27 bp, Homo sapiens
agatctacctgtacaccttgaatgaca

SEQ ID NO: 62 - DNA, 22 bp, Homo sapiens
catgataccagcaaggaattgg

SEQ ID NO: 63 - DNA, 1812 bp, Homo sapiens
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatga

SEQ ID NO: 64 - DNA, 1919 bp, Homo sapiens
ctcagtcgccgctgccagctctcgcactctgttcttccgccgctccgccgtcgcgtttctctgccggtcgcaatggaagaagagatcgccgcgctggtcattgacaatggctccggcatgtgcaaagctggttttgctggggacgacgctccccgagccgtgtttccttccatcgtcgggcgccccagacaccagggcgtcatggtgggcatgggccagaaggactcctacgtgggcgacgaggcccagagcaagcgtggcatcctgaccctgaagtaccccattgagcatggcatcgtcaccaactgggacgacatggagaagatctggcaccacaccttctacaacgagctgcgcgtggccccggaggagcacccagtgctgctgaccgaggcccccctgaaccccaaggccaacagagagaagatgactcagattatgtttgagaccttcaacaccccggccatgtacgtggccatccaggccgtgctgtccctctacgcctctgggcgcaccactggcattgtcatggactctggagacggggtcacccacacggtgcccatctacgagggctacgccctcccccacgccatcctgcgtctggacctggctggccgggacctgaccgactacctcatgaagatcctcactgagcgaggctacagcttcaccaccacggccgagcgggaaatcgtgcgcgacatcaaggagaagctgtgctacgtcgccctggacttcgagcaggagatggccaccgccgcatcctcctcttctctggagaagagctacgagctgcccgatggccaggtcatcaccattggcaatgagcggttccggtgtccggaggcgctgttccagccttccttcctgggtatggaatcttgcggcatccacgagaccaccttcaactccatcatgaagtgtgacgtggacatccgcaaagacctgtacgccaacacggtgctgtcgggcggcaccaccatgtacccgggcattgccgacaggatgcagaaggagatcaccgccctggcgcccagcaccatgaagatcaagatcatcgcacccccagagcgcaagtactcggtgtggatcggtggctccatcctggcctcactgtccaccttccagcagatgtggattagcaagcaggagtacgacgagtcgggcccctccatcgtccaccgcaaatgcttctaaacggactcagcagatgcgtagcatttgctgcatgggttaattgagaatagaaatttgcccctggcaaatgcacacacctcatgctagcctcacgaaactggaataagccttcgaaaagaaattgtccttgaagcttgtatctgatatcagcactggattgtagaacttgttgctgattttgaccttgtattgaagttaactgttccccttggtatttgtttaataccctgtacatatctttgagttcaacctttagtacgtgtggcttggtcacttcgtggctaaggtaagaacgtgcttgtggaagacaagtctgtggcttggtgagtctgtgtggccagcagcctctgatctgtgcagggtattaacgtgtcagggctgagtgttctgggatttctctagaggctggcaagaaccagttgttttgtcttgcgggtctgtcagggttggaaagtccaagccgtaggacccagtttcctttcttagctgatgtctttggccagaacaccgtgggctgttacttgctttgagttggaagcggtttgcatttacgcctgtaaatgtattcattcttaatttatgtaaggttttttttgtacgcaattctcgattctttgaagagatgacaacaaattttggttttctactgttatgtgagaacattaggccccagcaacacgtcattgtgtaaggaaaaataaaagtgctgccgtaaccaa

SEQ ID NO: 65 - DNA, 3583 bp, Homo sapiens
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 66 - DNA, 3670 bp, Homo sapiens
ggagctgcggcggggtctggggcgcagagcagcggcgggaggaggcggacacgtggcaacagcggtagcagcccgggcggcggcagcaacagcggcggcggcatcggcccgagccgccggccgccctcccaccctcccgccccgcggcagccctagctccctccacttggctcccctggtcccgctcgctcggccgggagctgctctgtgcttttctctctgattctccagcgacaggacccggcgccgggcactgagcaccgccaccatggggaagggggttggacgtgataagtatgagcctgcagctgtttcagaacaaggtgataaaaagggcaaaaagggcaaaaaagacagggacatggatgaactgaagaaagaagtttctatggatgatcataaacttagccttgatgaacttcatcgtaaatatggaacagacttgagccggggattaacatctgctcgtgcagctgagatcctggcgcgagatggtcccaacgccctcactccccctcccactactcctgaatggatcaagttttgtcggcagctctttggggggttctcaatgttactgtggattggagcgattctttgtttcttggcttatagcatccaagctgctacagaagaggaacctcaaaacgataatctgtacctgggtgtggtgctatcagccgttgtaatcataactggttgcttctcctactatcaagaagctaaaagttcaaagatcatggaatccttcaaaaacatggtccctcagcaagcccttgtgattcgaaatggtgagaaaatgagcataaatgcggaggaagttgtggttggggatctggtggaagtaaaaggaggagaccgaattcctgctgacctcagaatcatatctgcaaatggctgcaaggtggataactcctcgctcactggtgaatcagaaccccagactaggtctccagatttcacaaatgaaaaccccctggagacgaggaacattgccttcttttcaaccaattgtgttgaaggcaccgcacgtggtattgttgtctacactggggatcgcactgtgatgggaagaattgccacacttgcttctgggctggaaggaggccagacccccattgctgcagaaattgaacattttatccacatcatcacgggtgtggctgtgttcctgggtgtgtctttcttcatcctttctctcatccttgagtacacctggcttgaggctgtcatcttcctcatcggtatcatcgtagccaatgtgccggaaggtttgctggccactgtcacggtctgtctgacacttactgccaaacgcatggcaaggaaaaactgcttagtgaagaacttagaagctgtggagaccttggggtccacgtccaccatctgctctgataaaactggaactctgactcagaaccggatgacagtggcccacatgtggtttgacaatcaaatccatgaagctgatacgacagagaatcagagtggtgtctcttttgacaagacttcagctacctggcttgctctgtccagaattgcaggtctttgtaacagggcagtgtttcaggctaaccaggaaaacctacctattcttaagcgggcagttgcaggagatgcctctgagtcagcactcttaaagtgcatagagctgtgctgtggttccgtgaaggagatgagagaaagatacgccaaaatcgtcgagatacccttcaactccaccaacaagtaccagttgtctattcataagaaccccaacacatcggagccccaacacctgttggtgatgaagggcgccccagaaaggatcctagaccgttgcagctctatcctcctccacggcaaggagcagcccctggatgaggagctgaaagacgcctttcagaacgcctatttggagctggggggcctcggagaacgagtcctaggtttctgccacctctttctgccagatgaacagtttcctgaagggttccagtttgacactgacgatgtgaatttccctatcgataatctgtgctttgttgggctcatctccatgattgaccctccacgggcggccgttcctgatgccgtgggcaaatgtcgaagtgctggaattaaggtcatcatggtcacaggagaccatccaatcacagctaaagctattgccaaaggtgtgggcatcatctcagaaggcaatgagaccgtggaagacattgctgcccgcctcaacatcccagtcagccaggtgaaccccagggatgccaaggcctgcgtagtacacggcagtgatctaaaggacatgacctccgagcagctggatgacattttgaagtaccacactgagatagtgtttgccaggacctcccctcagcagaagctcatcattgtggaaggctgccaaagacagggtgctatcgtggctgtgactggtgacggtgtgaatgactctccagctttgaagaaagcagacattggggttgctatggggattgctggctcagatgtgtccaagcaagctgctgacatgattcttctggatgacaactttgcctcaattgtgactggagtagaggaaggtcgtctgatctttgataacttgaagaaatccattgcttataccttaaccagtaacattcccgagatcaccccgttcctgatatttattattgcaaacattccactaccactggggactgtcaccatcctctgcattgacttgggcactgacatggttcctgccatctccctggcttatgagcaggctgagagtgacatcatgaagagacagcccagaaatcccaaaacagacaaacttgtgaatgagcggctgatcagcatggcctatgggcagattggaatgatccaggccctgggaggcttctttacttactttgtgattctggctgagaacggcttcctcccaattcacctgttgggcctccgagtggactgggatgaccgctggatcaacgatgtggaagacagctacgggcagcagtggacctatgagcagaggaaaatcgtggagttcacctgccacacagccttcttcgtcagtatcgtggtggtgcagtgggccgacttggtcatctgtaagaccaggaggaattcggtcttccagcaggggatgaagaacaagatcttgatatttggcctctttgaagagacagccctggctgctttcctttcctactgccctggaatgggtgttgctcttaggatgtatcccctcaaacctacctggtggttctgtgccttcccctactctcttctcatcttcgtatatgacgaagtcagaaaactcatcatcaggcgacgccctggcggctgggtggagaaggaaacctactattagccccccgtcctgcacgccgtggagcatcaggccacacactctgcatccgacacccaccccctctttgtgtacttcagtcttggagtttggaactctaccctggtaggaaagcaccgcagcatgtggggaagcaagacgtcctggaatgaagcatgtagctctatggggggaggggggagggctgcctgaaaaccatccatctgtggaaatgacagcggggaaggtttttatgtgcctttttgtttttgtaaaaaaggaacacccggaaagactgaaagaatacattttatatctggatttttacaaataaagatggctattataatggaa

SEQ ID NO: 67 - DNA, 943 bp, Homo sapiens
attcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttga

SEQ ID NO: 68 - DNA, 5231 bp, Homo sapiens
actcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacccccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtgagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgctgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcccgaggaggacgaatgccaagacgaagagaaccagaaacaatgccaggacctcggcgccttcaccgagagcatggttgtctttgggtgccccaactgaccacacccccattcccccactccagataaagcttcagttatatctcacgtgtctggagttctttgccaagagggagaggctgaaatccccagccgcctcacctgcagctcagctccatcctacttgaaacctcacctgttcccaccgcattttctcctggcgttcgcctgctagtgtg

SEQ ID NO: 69 - DNA, 1901 bp, Homo sapiens
gtccgtactgcagagccgctgccggagggtcgttttaaagggcccgcgcgttgccgccccctcggcccgccatgctgctatccgtgccgctgctgctcggcctcctcggcctggccgtcgccgagcctgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagctggccgcgccaaataatgtctctgtgagactcgagaactttcatttttttccaggctggttcggatttggggtggattttggttttgttcccctcctccactctcccccaccccctccccgcccttttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctc

SEQ ID NO: 70 - DNA, 1023 bp, Homo sapiens
agagagccccggagccgcggcgggagaggaacgcgcagccagccttgggaagcccaggcccggcagccatggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatgtaggggtctggtctcctcagcctcctcatctgggggagtggaatagtatcctccaggtttttcaattaaacggattattttttcagaccgaaaagagatggtctgagtttgtcttagagtgatgcttgattccttcctttccttactgatgttccctgtcctctgggaccttaatgcatgtgtacttcgaggtctattttggggggtgtttgggaaaggaggactttgcgaaggtgttggt

SEQ ID NO: 71 - DNA, 1698 bp, Homo sapiens
ctgcctggggagcccccccgccccacatcctgccccgcaaaaggcagcttcaccaaagtggggtatttccagcctttgtagctttcacttccacatctaccaagtgggcggagtggccttctgtggacgaatcagattcctctccagcaccgactttaagaggcgagccggggggtcagggtcccagatgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagtactgaccaagtgccaggaagaggtcagccacatccctgctgtccacccgggttcattcaggcccaagtgcgacgagaacggcaactatctgccactccagtgctatgggagcatcggctactgctggtgtgtcttccccaacggcacggaggtccccaacaccagaagccgcgggcaccataactgcagtgagtcactggaactggaggacccgtcttctgggctgggtgtgaccaagcaggatctgggcccagtccccatgtgagagcagcagaggcggtcttcaacatcctgccagccccacacagctacagctttcttgctcccttcagcccccagcccctcccccatctcccaccctgtacctcatcccatgagaccctggtgcctggctctttcgtcacccttggacaagacaaaccaagtcggaacagcagataacaatgcagcaaggccctgctgcccaatctccatctgtcaacaggggcgtgaggtcccaggaagtggccaaaagctagacagatccccgttcctgacatcacagcagcctccaacacaaggctccaagacctaggctcatggacgagatgggaaggcacagggagaagggataaccctacacccagaccccaggctggacatgctgactgtcctctcccctccagcctttggccttggcttttctagcctatttacctgcaggctgagccactctcttccctttccccagcatcactccccaaggaagagccaatgttttccacccataatcctttctgccgacccctagttccctctgctcagccaagcttgttatcagctttcagggccatggttcacattagaataaaaggtagtaattagaacaaaaaaaaaaaaaaaaaa

SEQ ID NO: 72 - DNA, 3501 bp, Homo sapiens
agagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcacacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggataatattttcataattttttatttgaaattttgctgattctttaaatgtcttgtttcccagatttcaggaaactttttttcttttaagctatccacagcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtatgacagaatacatttgaaaacattggttatattaccaagactttgactagaatgtcgtatttgaggatataaacccataggtaataaacccacaggtactacaaacaaagtctgaagtcagccttggtttggcttcctagtgtcaattaaacttctaaaagtttaatctgagattccttataaaaacttccagcaaagcaactttaaaaaagtctgtgtgggccgggcgcggtggctcacgcctgtaatcccagcactttgatccgccgaggcgggcggatcacgaggtcaggagatccagaccatcctggctaacacagtgaaaccccgtctctactaaaaatacaaaaaaagttagccgggcgtggtggtgggggcctgtagtcccagctactcaggaggctgaggcaggagaacggcatgaacccgggaggcagggcttgcagtgagccaagatcatgccgctgcactccagcctgggagacaaagtgagactccgtcaaaaaaaaaaaaaa

SEQ ID NO: 73 - DNA, 1245 bp, Homo sapiens
gttcagtcgggtcccggcagcggctgcagcgctctcgtcttctgcggctctcggtgccctctccttttcgtttccggaaacatggcctccggtgtggctgtctctgatggtgtcatcaaggtgttcaacgacatgaaggtgcgtaagtcttcaacgccagaggaggtgaagaagcgcaagaaggcggtgctcttctgcctgagtgaggacaagaagaacatcatcctggaggagggcaaggagatcctggtgggcgatgtgggccagactgtcgacgacccctacgccacctttgtcaagatgctgccagataaggactgccgctatgccctctatgatgcaacctatgagaccaaggagagcaagaaggaggatctggtgtttatcttctgggcccccgagtctgcgccccttaagagcaaaatgatttatgccagctccaaggacgccatcaagaagaagctgacagggatcaagcatgaattgcaagcaaactgctacgaggaggtcaaggaccgctgcaccctggcagagaagctggggggcagtgccgtcatctccctggagggcaagcctttgtgagccccttctggccccctgcctggagcatctggcagccccacacctgcccttgggggttgcaggctgcccccttcctgccagaccggaggggctggggggatcccagcagggggagggcaatcccttcaccccagttgccaaacagaccccccaccccctggattttccttctccctccatcccttgacggttctggccttcccaaactgcttttgatcttttgattcctcttgggctgaagcagaccaagttccccccaggcaccccagttgtgggggagcctgtattttttttaacaacatccccattccccacctggtcctcccccttcccatgctgccaacttctaaccgcaatagtgactctgtgcttgtctgtttagttctgtgtataaatggaatgttgtggagatgacccctccctgtgccggctggttcctctcccttttcccctggtcacggctactcatggaagcaggaccagtaagggaccttcgattaaaaaaaaaaaagacaataataaaaaggctcattaatgggatgtgtttttcaaggttgggacacaggagacttcaggattgggggtgcactgggataatggtgctatctctctcctctgtactccacacccaaccagcccctgcacacccttatctggctccaa

SEQ ID NO: 74 - DNA, 1420 bp, Homo sapiens
gttcgcctgcgtcgctccgggagctgccgacggacggagcgcccccgcccccgcccggccgcccgcccgccgccgccatgcccttctccaacagccacaacgcactgaagctgcgcttcccggccgaggacgagttccccgacctgagcgcccacaacaaccacatggccaaggtgctgacccccgagctgtacgcggagctgcgcgccaagagcacgccgagcggcttcacgctggacgacgtcatccagacaggcgtggacaacccgggccacccgtacatcatgaccgtgggctgcgtggcgggcgacgaggagtcctacgaagtgttcaaggatctcttcgaccccatcatcgaggaccggcacggcggctacaagcccagcgatgagcacaagaccgacctcaaccccgacaacctgcagggcggcgacgacctggaccccaactacgtgctgagctcgcgggtgcgcacgggccgcagcatccgtggcttctgcctccccccgcactgcagccgcggggagcgccgcgccatcgagaagctcgcggtggaagccctgtccagcctggacggcgacctggcgggccgatactacgcgctcaagagcatgacggaggcggagcagcagcagctcatcgacgaccacttcctcttcgacaagcccgtgtcgcccctgctgctggcctcgggcatggcccgcgactggcccgacgcccgcggtatctggcacaatgacaataagaccttcctggtgtgggtcaacgaggaggaccacctgcgggtcatctccatgcagaaggggggcaacatgaaggaggtgttcacccgcttctgcaccggcctcacccagattgaaactctcttcaagtctaaggactatgagttcatgtggaaccctcacctgggctacatcctcacctgcccatccaacctgggcaccgggctgcgggcaggtgtgcatatcaagctgcccaacctgggcaagcatgagaagttctcggaggtgcttaagcggctgcgacttcagaagcgaggcacaggcggtgtggacacggctgcggtgggcggggtcttcgacgtctccaacgctgaccgcctgggcttctcagaggtggagctggtgcagatggtggtggacggagtgaagctgctcatcgagatggagcagcggctggagcagggccaggccatcgacgacctcatgcctgcccagaaatgaagcccggcccacacccgacaccagccctgctgcttcctaacttattgcctgggcagtgcccaccatgcacccctgatgttcgccgtctggcgagcccttagccttgctgtagagacttccgtcacccttggtagagtttatttttttgatggctaagatactgctgatgctgaaataaactagggttttggcctgcc

SEQ ID NO: 75 - DNA, 983 bp, Homo sapiens
acacccaggccggcaaagagcaggtatcagcactgcaagcaccaagtgtgtcttgagctcagtgagtactgggtatgtgtcacattgccaaatcccggatcacaagtctccatgaactgctggtgagctaggataataaaacccctgacatcaccattccagaagcttcacaagactgcatatataaggggctggctgtagctgcagctgaaggagctgaccagccagctgacccctcacactcacctagccaccatggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacaggaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatagatgccctttcttgaattgcattttttaaaacaagaaagtttccccaccagtgaatgaaagtcttgtgactagtgctgaagcttattaatgctaagggcaggcccaaattatcaagctaataaaatatcattcagcaacagataactgtcttgtgtttgaatattccacacacttttaaataaatatacagataccacaga

SEQ ID NO: 76 - DNA, 3733 bp, Homo sapiens
ggctgcagggctccggcaaccgctccggcaacgccaaccgctccgctgcgcgcaggctgggctgcaggctctcggctgcagcgctgggtggatctaggatccggcttccaacatgtggcagctctgggcctccctctgctgcctgctggtgttggccaatgcccggagcaggccctctttccatcccctgtcggatgagctggtcaactatgtcaacaaacggaataccacgtggcaggccgggcacaacttctacaacgtggacatgagctacttgaagaggctatgtggtaccttcctgggtgggcccaagccaccccagagagttatgtttaccgaggacctgaagctgcctgcaagcttcgatgcacgggaacaatggccacagtgtcccaccatcaaagagatcagagaccagggctcctgtggctcctgctgggccttcggggctgtggaagccatctctgaccggatctgcatccacaccaatgcgcacgtcagcgtggaggtgtcggcggaggacctgctcacatgctgtggcagcatgtgtggggacggctgtaatggtggctatcctgctgaagcttggaacttctggacaagaaaaggcctggtttctggtggcctctatgaatcccatgtagggtgcagaccgtactccatccctccctgtgagcaccacgtcaacggctcccggcccccatgcacgggggagggagatacccccaagtgtagcaagatctgtgagcctggctacagcccgacctacaaacaggacaagcactacggatacaattcctacagcgtctccaatagcgagaaggacatcatggccgagatctacaaaaacggccccgtggagggagctttctctgtgtattcggacttcctgctctacaagtcaggagtgtaccaacacgtcaccggagagatgatgggtggccatgccatccgcatcctgggctggggagtggagaatggcacaccctactggctggttgccaactcctggaacactgactggggtgacaatggcttctttaaaatactcagaggacaggatcactgtggaatcgaatcagaagtggtggctggaattccacgcaccgatcagtactgggaaaagatctaatctgccgtgggcctgtcgtgccagtcctgggggcgagatcggggtagaaatgcattttattctttaagttcacgtaagatacaagtttcagacagggtctgaaggactggattggccaaacatcagacctgtcttccaaggagaccaagtcctggctacatcccagcctgtggttacagtgcagacaggccatgtgagccaccgctgccagcacagagcgtccttccccctgtagactagtgccgtagggagtacctgctgccccagctgactgtggccccctccgtgatccatccatctccagggagcaagacagagacgcaggaatggaaagcggagttcctaacaggatgaaagttcccccatcagttcccccagtacctccaagcaagtagctttccacatttgtcacagaaatcagaggagagacggtgttgggagccctttggagaacgccagtctcccaggccccctgcatctatcgagtttgcaatgtcacaacctctctgatcttgtgctcagcatgattctttaatagaagttttattttttcgtgcactctgctaatcatgtgggtgagccagtggaacagcgggagacctgtgctagttttacagattgcctccttatgacgcggctcaaaaggaaaccaagtggtcaggagttgtttctgacccactgatctctactaccacaaggaaaatagtttaggagaaaccagcttttactgtttttgaaaaattacagcttcaccctgtcaagttaacaaggaatgcctgtgccaataaaagttttctccaacttgaagtctactctgatgggatctcagatcctttgtcactgcctatagacttgtagctgctgtctctctttgtccctgcagagaatcacgtcctggaactgcatgttcttgcgactcttgggacttcatcttaacttctcgctgccccagccatgttttcaaccatggcatccctcccccaattagttccctgtcatcctcgtcaaccttctctgtaagtgcctggtaagcttgcccttgcttaagaactcaaaacatagctgtgctctatttttttgttgttgttgtgactgacagagtgagattccgtctcccaggctggagtgcagtggcgccttctcagctcactgcaacctgcagcctcctagattcaagcgattctcctgcttcagccttccgagtagctgggatgacaggcactcaccaatatgcctgggtaatttttgtatttttaagtacatacaggatttcaccatgttggccaggctagtttcaaactcccggcctcaggtggtctgcctgcctcagcctcccaaagtgttgggattacaggcgtgagccactgggccctgcctgtattttttatcagccacaaatccagcaacaagctgaggattcagctcataaaacaggcttggtgtcttggtgatctcacataaccaagatgctaccccgtggggaaccacatccccctggatgccctccagccttggtttgggctggagtcagggcctgtatacagtattttgaatttgtatgccactggtttgcattgctggtcaggaactctagtgctttgcatagccctggtttagaaacatgttatagcagttcttggtatagagcaaactagaagaaccagcaatcattccactgtcctgccaaggtacacctcagtactccccttcccaactgaagtggtatgaggctagctctttccaaaagcattcaagtttggcttctgatgtgactcagaatttaggaaccagatgctagatcaaataagctctgaaaatctgaggaacattgtaggaaaggtttgttaagcatctcttaagtgccatgatgagcataacagccggccgtcgtggctcacgcctgtaatcccagcactttgggaggccaaggtgggaggatgacaaggtcaggagttcaagaccagcctggccaacatgctgaaacctcacctctactaaaaatacaaaaattagctgggcatggtggcacatgcctgtaatcccagctacttgggaggctgaggcaggagaatcgcttgaacccgggaggcggaggttgcagtgagccaagacagtgccagtgcactccagcctcggtgacagcgcaaggctccgtctcaataattaaaaaaaaaaaaaaaaaaaaaaaggccgggcgcagtggctcaagcctgtaatcccagcactttgggaggctgaggcgggcagatcacctgaggtcaggagttttgagatcagccttggcaacacggtgaaaccccatctctactaaaaatacaaaattagccaagcatgctggcacatgcctgtaatcccagctactcgggaggctgaggtacgagaatcgcttgaacctgggaggcagaggatgcagtgagccgagatcacgccattgcactccagcctgggggacaagagtgaatctgtgtctcaccaaaaaaaaaaagaaaaagaaagatgcttaacaaaggttaccataagccacaaattcataaccacttatccttccagtttcaagtagaatatattcataacctcaataaagttctccctgctcccaaa

SEQ ID NO: 77 - DNA, 2055 bp, Homo sapiens
aactgcggcgtcatcccggctataagcgcacggcctcggcgaccctctccgacccggccgccgccgccatgcagccctccagccttctgccgctcgccctctgcctgctggctgcacccgcctccgcgctcgtcaggatcccgctgcacaagttcacgtccatccgccggaccatgtcggaggttgggggctctgtggaggacctgattgccaaaggccccgtctcaaagtactcccaggcggtgccagccgtgaccgaggggcccattcccgaggtgctcaagaactacatggacgcccagtactacggggagattggcatcgggacgcccccccagtgcttcacagtcgtcttcgacacgggctcctccaacctgtgggtcccctccatccactgcaaactgctggacatcgcttgctggatccaccacaagtacaacagcgacaagtccagcacctacgtgaagaatggtacctcgtttgacatccactatggctcgggcagcctctccgggtacctgagccaggacactgtgtcggtgccctgccagtcagcgtcgtcagcctctgccctgggcggtgtcaaagtggagaggcaggtctttggggaggccaccaagcagccaggcatcaccttcatcgcagccaagttcgatggcatcctgggcatggcctacccccgcatctccgtcaacaacgtgctgcccgtcttcgacaacctgatgcagcagaagctggtggaccagaacatcttctccttctacctgagcagggacccagatgcgcagcctgggggtgagctgatgctgggtggcacagactccaagtattacaagggttctctgtcctacctgaatgtcacccgcaaggcctactggcaggtccacctggaccaggtggaggtggccagcgggctgaccctgtgcaaggagggctgtgaggccattgtggacacaggcacttccctcatggtgggcccggtggatgaggtgcgcgagctgcagaaggccatcggggccgtgccgctgattcagggcgagtacatgatcccctgtgagaaggtgtccaccctgcccgcgatcacactgaagctgggaggcaaaggctacaagctgtccccagaggactacacgctcaaggtgtcgcaggccgggaagaccctctgcctgagcggcttcatgggcatggacatcccgccacccagcgggccactctggatcctgggcgacgtcttcatcggccgctactacactgtgtttgaccgtgacaacaacagggtgggcttcgccgaggctgcccgcctctagttcccaaggcgtccgcgcgccagcacagaaacagaggagagtcccagagcaggaggcccctggcccagcggcccctcccacacacacccacacactcgcccgcccactgtcctgggcgccctggaagccggcggcccaagcccgacttgctgttttgttctgtggttttcccctccctgggttcagaaatgctgcctgcctgtctgtctctccatctgtttggtgggggtagagctgatccagagcacagatctgtttcgtgcattggaagaccccacccaagcttggcagccgagctcgtgtatcctggggctcccttcatctccagggagtcccctccccggccctaccagcgcccgctgggctgagcccctaccccacaccaggccgtcctcccgggccctcccttggaaacctgccctgcctgagggcccctctgcccagcttgggcccagctgggctctgccaccctacctgttcagtgtcccgggcccgttgaggatgaggccgctagaggcctgaggatgagctggaaggagtgagaggggacaaaacccaccttgttggagcctgcagggtggtgctgggactgagccagtcccaggggcatgtattggcctggaggtggggttgggattgggggctggtgccagccttcctctgcagctgacctctgttgtcctccccttgggcggctgagagccccagctgacatggaaatacagttgttggcctccggcctcccctc

SEQ ID NO: 78 - DNA, 3512 bp, Homo sapiens
ctttttcgcaacgggtttgccgccagaacacaggtgtcgtgaaaactacccctaaaagccaaaatgggaaaggaaaagactcatatcaacattgtcgtcattggacacgtagattcgggcaagtccaccactactggccatctgatctataaatgcggtggcatcgacaaaagaaccattgaaaaatttgagaaggaggctgctgagatgggaaagggctccttcaagtatgcctgggtcttggataaactgaaagctgagcgtgaacgtggtatcaccattgatatctccttgtggaaatttgagaccagcaagtactatgtgactatcattgatgccccaggacacagagactttatcaaaaacatgattacagggacatctcaggctgactgtgctgtcctgattgttgctgctggtgttggtgaatttgaagctggtatctccaagaatgggcagacccgagagcatgcccttctggcttacacactgggtgtgaaacaactaattgtcggtgttaacaaaatggattccactgagccaccctacagccagaagagatatgaggaaattgttaaggaagtcagcacttacattaagaaaattggctacaaccccgacacagtagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtcatcaaagcagtggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgtgttgcatagaaagcttcatgttgctaaaccaatgttaagtgaatctttggaaacaaaatgtttccaaattactgggatgtgcatgttgaaacgtgggttaaaatgactgggcagtgaaagttgactatttgccatgacataagaaataagtgtagtggctagtgtacaccctatgagtggaagggtccattttgaagtcagtggagtaagctttatgccagtttgatggtttcacaagttctattgagtgctattcagaataggaacaaggttctaatagaaaaagatggcaatttgaagtagctataaaattagactaatctacattgcttttctcctgcagagtctaataccttttatgctttgataattagcagtttgtctacttggtcactaggaatgaaactacatggtaataggcttaacaggtgtaatagcccacttactcctgaatctttaagcatttgtgcatttgaaaaatgcttttcgcgatcttcctgctgggattacaggcatgagccactgtgcctgacctcccatatgtaaaagtgtctaaaggtttttttttggttataaaaggaaaatttttgcttaagtttgaaggataggtaaaattaaaggacatgctttctgtttgtgtgatggtttttaaaaattttttttaagatggagttcttgttgcccaggctagaatgcaatggcaaaatctcactgcaatctcctcctcctgggttcaagcaattctcctacttcagcctcccaagtagctgggattacaggcatgtgctaatttggtgtttttaatagagatgaggtttttccatgttggtcaggctggtctcaaactcctgaccttaggtgatcgcctcggcctcctaaagtgctggaattacaggcatgagccaccatgcctggccaggacatgtgttcttaaggacatgctaagcaggagttaaagcagcccaagagataaggcctcttaaagtgactggcaatgtgtattgctcaagattcaaaggtacttgaattggccatagacaagtctgtaatgaagtgttatcgttttccctcatctgagtctgaattagataaaatgccttcccatcagccagtgctctgaggtatcaagtctaaattgaactagagatttttgtccttagtttctttgctatctaatgtttacacaagtaaatagtctaagatttgctggatgacagaaaaaacaggtaaggcctttaatagatggccaatagatgccctgataatgaaagttgacacctgtaagatttaccagtagagaattcttgacatgcaaggaagcaagatttaactgaaaaattgttcccactggaagcaggaatgagtcagtttacttgcatatactgagattgagattaacttcctgtgaaacccagtgtcttagacaactgtggcttgagcaccacctgctggtattcattacaaacttgctcactacaataaatgaattttaagctttaa

SEQ ID NO: 79 - DNA, 3158 bp, Homo sapiens
ctcttccgccgtcgtcgccgccatcctcggcgcgactcgcttctttcggttctacctgggagaatccaccgccatccgccaccatggtgaacttcacggtagaccagatccgcgccatcatggacaagaaggccaacatccgcaacatgtctgtcatcgcccacgtggaccatggcaagtccacgctgacagactccctggtgtgcaaggcgggcatcatcgcctcggcccgggccggggagacacgcttcactgatacccggaaggacgagcaggagcgttgcatcaccatcaagtcaactgccatctccctcttctacgagctctcggagaatgacttgaacttcatcaagcagagcaaggacggtgccggcttcctcatcaacctcattgactcccccgggcatgtcgacttctcctcggaggtgactgctgccctccgagtcaccgatggcgcattggtggtggtggactgcgtgtcaggcgtgtgcgtgcagacggagacagtgctgcggcaggccattgccgagcgcatcaagcctgtgctgatgatgaacaagatggaccgcgccctgctggagctgcagctggagcccgaggagctctaccagactttccagcgcatcgtggagaacgtgaacgtcatcatctccacctacggcgagggcgagagcggccccatgggcaacatcatgatcgatcctgtcctcggtaccgtgggctttgggtctggcctccacgggtgggccttcaccctgaagcagtttgccgagatgtatgtggccaagttcgccgccaagggggagggccagttggggcctgccgagcgggccaagaaagtagaggacatgatgaagaagctgtggggtgacaggtactttgacccagccaacggcaagttcagcaagtcagccaccagccccgaagggaagaagctgccacgcaccttctgccagctgatcctggaccccatcttcaaggtgtttgatgcgatcatgaatttcaagaaagaggagacagcaaaactgatagagaaactggacatcaaactggacagcgaggacaaggacaaagaaggcaaacccctgctgaaggctgtgatgcgccgctggctgcctgccggagacgccttgttgcagatgatcaccatccacctgccctcccctgtgacggcccagaagtaccgctgcgagctcctgtacgaggggcccccggacgacgaggctgccatgggcattaaaagctgtgaccccaaaggccctcttatgatgtatatttccaaaatggtgccaacctccgacaaaggtcggttctacgcctttggacgagtcttctcggggctggtctccactggcctgaaggtcaggatcatggggcccaactatacccctgggaagaaggaggacctctacctgaagccaatccagagaacaatcttgatgatgggccgctacgtggagcccatcgaggatgtgccttgtgggaacattgtgggcctcgtgggcgtggaccagttcctggtgaagacgggcaccatcaccaccttcgagcacgcgcacaacatgcgggtgatgaagttcagcgtcagccctgttgtcagagtggccgtggaggccaagaacccggctgacctgcccaagctggtggaggggctgaagcggctggccaagtccgaccccatggtgcagtgcatcatcgaggagtcgggagagcatatcatcgcgggcgccggcgagctgcacctggagatctgcctgaaggacctggaggaggaccacgcctgcatccccatcaagaaatctgacccggtcgtctcgtaccgcgagacggtcagtgaagagtcgaacgtgctctgcctctccaagtcccccaacaagcacaaccggctgtacatgaaggcgcggcccttccccgacggcctggccgaggacatcgataaaggcgaggtgtccgcccgtcaggagctcaagcagcgggcgcgctacctggccgagaagtacgagtgggacgtggctgaggcccgcaagatctggtgctttgggcccgacggcaccggccccaacatcctcaccgacatcaccaagggtgtgcagtacctcaacgagatcaaggacagtgtggtggccggcttccagtgggccaccaaggagggcgcactgtgtgaggagaacatgcggggtgtgcgcttcgacgtccacgacgtcaccctgcacgccgacgccatccaccgcggagggggccagatcatccccacagcacggcgctgcctctatgccagtgtgctgaccgcccagccacgcctcatggagcccatctaccttgtggagatccagtgtccagagcaggtggtcggtggcatctacggggttttgaacaggaagcggggccacgtgttcgaggagtcccaggtggccggcacccccatgtttgtggtcaaggcctatctgcccgtcaacgagtcctttggcttcaccgctgacctgaggtccaacacgggcggccaggcgttcccccagtgtgtgtttgaccactggcagatcctgcccggagaccccttcgacaacagcagccgccccagccaggtggtggcggagacccgcaagcgcaagggcctgaaagaaggcatccctgccctggacaacttcctggacaaattgtaggcggcccttcctgcagcgcctgccgccccggggactcgcagcacccacagcaccacgtcctcgaattctcagacgacacctggagactgtcccgacacagcgacgctcccctgagaggtttctggggcccgctgcgtgccatcactcaaccataacacttgatgccgtttctttcaatatttatttccagagtccggaggcagcagacacgccctcttagtagggacttaatgggccggtcggggagggggaggcgggatgggacacccaacactttttccatttcttcagagggaaactcagatgtccaaactaattttaacaaacgcattaagaggtttatttgggtacatggcccgcagtggcttttgccccagaaaggggaaaggaacacgcgggtagatgatttctagcaggcaggaagtcctgtgcggtgtcaccatgagcacctccagctgtactagtgccattggaataataaatttgataaggtggtga

SEQ ID NO: 80 - DNA, 1781 bp, Homo sapiens
agtatctgtgggtacccggagcacggagatctcgccggctttacgttcacctcggtgtctgcagcaccctccgcttcctctcctaggcgacgagacccagtggctagaagttcaccatgtctattctcaagatccatgccagggagatctttgactctcgcgggaatcccactgttgaggttgatctcttcacctcaaaaggtctcttcagagctgctgtgcccagtggtgcttcaactggtatctatgaggccctagagctccgggacaatgataagactcgctatatggggaagggtgtctcaaaggctgttgagcacatcaataaaactattgcgcctgccctggttagcaagaaactgaacgtcacagaacaagagaagattgacaaactgatgatcgagatggatggaacagaaaataaatctaagtttggtgcgaacgccattctgggggtgtcccttgccgtctgcaaagctggtgccgttgagaagggggtccccctgtaccgccacatcgctgacttggctggcaactctgaagtcatcctgccagtcccggcgttcaatgtcatcaatggcggttctcatgctggcaacaagctggccatgcaggagttcatgatcctcccagtcggtgcagcaaacttcagggaagccatgcgcattggagcagaggtttaccacaacctgaagaatgtcatcaaggagaaatatgggaaagatgccaccaatgtgggggatgaaggcgggtttgctcccaacatcctggagaataaagaaggcctggagctgctgaagactgctattgggaaagctggctacactgataaggtggtcatcggcatggacgtagcggcctccgagttcttcaggtctgggaagtatgacctggacttcaagtctcccgatgaccccagcaggtacatctcgcctgaccagctggctgacctgtacaagtccttcatcaaggactacccagtggtgtctatcgaagatccctttgaccaggatgactggggagcttggcagaagttcacagccagtgcaggaatccaggtagtgggggatgatctcacagtgaccaacccaaagaggatcgccaaggccgtgaacgagaagtcctgcaactgcctcctgctcaaagtcaaccagattggctccgtgaccgagtctcttcaggcgtgcaagctggcccaggccaatggttggggcgtcatggtgtctcatcgttcgggggagactgaagataccttcatcgctgacctggttgtggggctgtgcactgggcagatcaagactggtgccccttgccgatctgagcgcttggccaagtacaaccagctcctcagaattgaagaggagctgggcagcaaggctaagtttgccggcaggaacttcagaaaccccttggccaagtaagctgtgggcaggcaagcccttcggtcacctgttggctacacagacccctcccctcgtgtcagctcaggcagctcgaggcccccgaccaacacttgcaggggtccctgctagttagcgccccaccgccgtggagttcgtaccgcttccttagaacttctacagaagccaagctccctggagccctgttggcagctctagctttgcagtcgtgtaattggcccaagtcattgtttttctcgcctcactttccaccaagtgtctagagtcatgtgagcctcgtgtcatctccggggtggccacaggctagatccccggtggttttgtgctcaaaataaaaagcctcagtgacccatgagaata

SEQ ID NO: 81 - DNA, 8483 bp, Homo sapiens
gcgtggaggcgcgtcgcgcgcagcggacgccgacagaatccccgaggcgcctggcgcgggcgcgggcgcgaaggcgatccgggcgccaccccgcggtcatcggtcaccggtcgctctcaggaacagcagcgcaacctctgctccctgcctcgcctcccgcgcgcctaggtgcctgcgactttaattaaagggccgtcccctcgccgaggctgcagcaccgcccccccggcttctcgcgcctcaaaatgagtagctcccactctcgggcgggccagagcgcagcaggcgcggctccgggcggcggcgtcgacacgcgggacgccgagatgccggccaccgagaaggacctggcggaggacgcgccgtggaagaagatccagcagaacactttcacgcgctggtgcaacgagcacctgaagtgcgtgagcaagcgcatcgccaacctgcagacggacctgagcgacgggctgcggcttatcgcgctgttggaggtgctcagccagaagaagatgcaccgcaagcacaaccagcggcccactttccgccaaatgcagcttgagaacgtgtcggtggcgctcgagttcctggaccgcgagagcatcaaactggtgtccatcgacagcaaggccatcgtggacgggaacctgaagctgatcctgggcctcatctggaccctgatcctgcactactccatctccatgcccatgtgggacgaggaggaggatgaggaggccaagaagcagacccccaagcagaggctcctgggctggatccagaacaagctgccgcagctgcccatcaccaacttcagccgggactggcagagcggccgggccctgggcgccctggtggacagctgtgccccgggcctgtgtcctgactgggactcttgggacgccagcaagcccgttaccaatgcgcgagaggccatgcagcaggcggatgactggctgggcatcccccaggtgatcacccccgaggagattgtggaccccaacgtggacgagcactctgtcatgacctacctgtcccagttccccaaggccaagctgaagccaggggctcccttgcggcccaaactgaacccgaagaaagcccgtgcctacgggccaggcatcgagcccacaggcaacatggtgaagaagcgggcagagttcactgtggagaccagaagtgctggccagggagaggtgctggtgtacgtggaggacccggccggacaccaggaggaggcaaaagtgaccgccaataacgacaagaaccgcaccttctccgtctggtacgtccccgaggtgacggggactcataaggttactgtgctctttgctggccagcacatcgccaagagccccttcgaggtgtacgtggataagtcacagggtgacgccagcaaagtgacagcccaaggtcccggcctggagcccagtggcaacatcgccaacaagaccacctactttgagatctttacggcaggagctggcacgggcgaggtcgaggttgtgatccaggaccccatgggacagaagggcacggtagagcctcagctggaggcccggggcgacagcacataccgctgcagctaccagcccaccatggagggcgtccacaccgtgcacgtcacgtttgccggcgtgcccatccctcgcagcccctacactgtcactgttggccaagcctgtaacccgagtgcctgccgggcggttggccggggcctccagcccaagggtgtgcgggtgaaggagacagctgacttcaaggtgtacacaaagggcgctggcagtggggagctgaaggtcaccgtgaagggccccaagggagaggagcgcgtgaagcagaaggacctgggggatggcgtgtatggcttcgagtattaccccatggtccctggaacctatatcgtcaccatcacgtggggtggtcagaacatcgggcgcagtcccttcgaagtgaaggtgggcaccgagtgtggcaatcagaaggtacgggcctggggccctgggctggagggcggcgtcgttggcaagtcagcagactttgtggtggaggctatcggggacgacgtgggcacgctgggcttctcggtggaagggccatcgcaggctaagatcgaatgtgacgacaagggcgacggctcctgtgatgtgcgctactggccgcaggaggctggcgagtatgccgttcacgtgctgtgcaacagcgaagacatccgcctcagccccttcatggctgacatccgtgacgcgccccaggacttccacccagacagggtgaaggcacgtgggcctggattggagaagacaggtgtggccgtcaacaagccagcagagttcacagtggatgccaagcacggtggcaaggccccacttcgggtccaagtccaggacaatgaaggctgccctgtggaggcgttggtcaaggacaacggcaatggcacttacagctgctcctacgtgcccaggaagccggtgaagcacacagccatggtgtcctggggaggcgtcagcatccccaacagccccttcagggtgaatgtgggagctggcagccaccccaacaaggtcaaagtatacggccccggagtagccaagacagggctcaaggcccacgagcccacctacttcactgtggactgcgccgaggctggccagggggacgtcagcatcggcatcaagtgtgcccctggagtggtaggccccgccgaagctgacatcgacttcgacatcatccgcaatgacaatgacaccttcacggtcaagtacacgccccggggggctggcagctacaccattatggtcctctttgctgaccaggccacgcccaccagccccatccgagtcaaggtggagccctctcatgacgccagtaaggtgaaggccgagggccctggcctcagtcgcactggtgtcgagcttggcaagcccacccacttcacagtaaatgccaaagctgctggcaaaggcaagctggacgtccagttctcaggactcaccaagggggatgcagtgcgagatgtggacatcatcgaccaccatgacaacacctacacagtcaagtacacgcctgtccagcagggtccagtaggcgtcaatgtcacttatggaggggatcccatccctaagagccctttctcagtggcagtatctccaagcctggacctcagcaagatcaaggtgtctggcctgggagagaaggtggacgttggcaaagaccaggagttcacagtcaaatcaaagggtgctggtggtcaaggcaaagtggcatccaagattgtgggcccctcgggtgcagcggtgccctgcaaggtggagccaggcctgggggctgacaacagtgtggtgcgcttcctgccccgtgaggaagggccctatgaggtggaggtgacctatgacggcgtgcccgtgcctggcagcccctttcctctggaagctgtggcccccaccaagcctagcaaggtgaaggcgtttgggccggggctgcagggaggcagtgcgggctcccccgcccgcttcaccatcgacaccaagggcgccggcacaggtggcctgggcctgacggtggagggcccctgtgaggcgcagctcgagtgcttggacaatggggatggcacatgttccgtgtcctacgtgcccaccgagcccggggactacaacatcaacatcctcttcgctgacacccacatccctggctccccattcaaggcccacgtggttccctgctttgacgcatccaaagtcaagtgctcaggccccgggctggagcgggccaccgctggggaggtgggccaattccaagtggactgctcgagcgcgggcagcgcggagctgaccattgagatctgctcggaggcggggcttccggccgaggtgtacatccaggaccacggtgatggcacgcacaccattacctacattcccctctgccccggggcctacaccgtcaccatcaagtacggcggccagcccgtgcccaacttccccagcaagctgcaggtggaacctgcggtggacacttccggtgtccagtgctatgggcctggtattgagggccagggtgtcttccgtgaggccaccactgagttcagtgtggacgcccgggctctgacacagaccggagggccgcacgtcaaggcccgtgtggccaacccctcaggcaacctgacggagacctacgttcaggaccgtggcgatggcatgtacaaagtggagtacacgccttacgaggagggactgcactccgtggacgtgacctatgacggcagtcccgtgcccagcagccccttccaggtgcccgtgaccgagggctgcgacccctcccgggtgcgtgtccacgggccaggcatccaaagtggcaccaccaacaagcccaacaagttcactgtggagaccaggggagctggcacgggcggcctgggcctggctgtagagggcccctccgaggccaagatgtcctgcatggataacaaggacggcagctgctcggtcgagtacatcccttatgaggctggcacctacagcctcaacgtcacctatggtggccatcaagtgccaggcagtcctttcaaggtccctgtgcatgatgtgacagatgcgtccaaggtcaagtgctctgggcccggcctgagcccaggcatggttcgtgccaacctccctcagtccttccaggtggacacaagcaaggctggtgtggccccattgcaggtcaaagtgcaagggcccaaaggcctggtggagccagtggacgtggtagacaacgctgatggcacccagaccgtcaattatgtgcccagccgagaagggccctacagcatctcagtactgtatggagatgaagaggtaccccggagccccttcaaggtcaaggtgctgcctactcatgatgccagcaaggtgaaggccagtggccccgggctcaacaccactggcgtgcctgccagcctgcccgtggagttcaccatcgatgcaaaggacgccggggagggcctgctggctgtccagatcacggatcccgaaggcaagccgaagaagacacacatccaagacaaccatgacggcacgtatacagtggcctacgtgccagacgtgacaggtcgctacaccatcctcatcaagtacggtggtgacgagatccccttctccccgtaccgcgtgcgtgccgtgcccaccggggacgccagcaagtgcactgtcacaggtgctggcatcggccccaccattcagattggggaggagacggtgatcactgtggacactaaggcggcaggcaaaggcaaagtgacgtgcaccgtgtgcacgcctgatggctcagaggtggatgtggacgtggtggagaatgaggacggcactttcgacatcttctacacggccccccagccgggcaaatacgtcatctgtgtgcgctttggtggcgagcacgtgcccaacagccccttccaagtgacggctctggctggggaccagccctcggtgcagccccctctacggtctcagcagctggccccacagtacacctacgcccagggcggccagcagacttgggccccggagaggcccctggtgggtgtcaatgggctggatgtgaccagcctgaggccctttgaccttgtcatccccttcaccatcaagaagggcgagatcacaggggaggttcggatgccctcaggcaaggtggcgcagcccaccatcactgacaacaaagacggcaccgtgaccgtgcggtatgcacccagcgaggctggcctgcacgagatggacatccgctatgacaacatgcacatcccaggaagccccttgcagttctatgtggattacgtcaactgtggccatgtcactgcctatgggcctggcctcacccatggagtagtgaacaagcctgccaccttcaccgtcaacaccaaggatgcaggagaggggggcctgtctctggccattgagggcccgtccaaagcagaaatcagctgcactgacaaccaggatgggacatgcagcgtgtcctacctgcctgtgctgccgggggactacagcattctagtcaagtacaatgaacagcacgtcccaggcagccccttcactgctcgggtcacaggtgacgactccatgcgtatgtcccacctaaaggtcggctctgctgccgacatccccatcaacatctcagagacggatctcagcctgctgacggccactgtggtcccgccctcgggccgggaggagccctgtttgctgaagcggctgcgtaatggccacgtggggatttcattcgtgcccaaggagacgggggagcacctggtgcatgtgaagaaaaatggccagcacgtggccagcagccccatcccggtggtgatcagccagtcggaaattggggatgccagtcgtgttcgggtctctggtcagggccttcacgaaggccacacctttgagcctgcagagtttatcattgatacccgcgatgcaggctatggtgggctcagcctgtccattgagggccccagcaaggtggacatcaacacagaggacctggaggacgggacgtgcagggtcacctactgccccacagagccaggcaactacatcatcaacatcaagtttgccgaccagcacgtgcctggcagccccttctctgtgaaggtgacaggcgagggccgggtgaaagagagcatcacccgcaggcgtcgggctccttcagtggccaacgttggtagtcattgtgacctcagcctgaaaatccctgaaattagcatccaggatatgacagcccaggtgaccagcccatcgggcaagacccatgaggccgagatcgtggaaggggagaaccacacctactgcatccgctttgttcccgctgagatgggcacacacacagtcagcgtgaagtacaagggccagcacgtgcctgggagccccttccagttcaccgtggggcccctaggggaagggggagcccacaaggtccgagctgggggccctggcctggagagagctgaagctggagtgccagccgaattcagtatctggacccgggaagctggtgctggaggcctggccattgctgtcgagggccccagcaaggctgagatctcttttgaggaccgcaaggacggctcctgtggtgtggcttatgtggtccaggagccaggtgactacgaagtctcagtcaagttcaacgaggaacacattcccgacagccccttcgtggtgcctgtggcttctccgtctggcgacgcccgccgcctcactgtttctagccttcaggagtcagggctaaaggtcaaccagccagcctcttttgcagtcagcctgaacggggccaagggggcgatcgatgccaaggtgcacagcccctcaggagccctggaggagtgctatgtcacagaaattgaccaagataagtatgctgtgcgcttcatccctcgggagaatggcgtttacctgattgacgtcaagttcaacggcacccacatccctggaagccccttcaagatccgagttggggagcctgggcatggaggggacccaggcttggtgtctgcttacggagcaggtctggaaggcggtgtcacagggaacccagctgagttcgtcgtgaacacgagcaatgcgggagctggtgccctgtcggtgaccattgacggcccctccaaggtgaagatggattgccaggagtgccctgagggctaccgcgtcacctatacccccatggcacctggcagctacctcatctccatcaagtacggcggcccctaccacattgggggcagccccttcaaggccaaagtcacaggcccccgtctcgtcagcaaccacagcctccacgagacatcatcagtgtttgtagactctctgaccaaggccacctgtgccccccagcatggggccccgggtcctgggcctgctgacgccagcaaggtggtggccaagggcctggggctgagcaaggcctacgtaggccagaagagcagcttcacagtagactgcagcaaagcaggcaacaacatgctgctggtgggggttcatggcccaaggaccccctgcgaggagatcctggtgaagcacgtgggcagccggctctacagcgtgtcctacctgctcaaggacaagggggagtacacactggtggtcaaatggggggacgagcacatcccaggcagcccctaccgcgttgtggtgccctgagtctggggcccgtgccagccggcagcccccaagcctgccccgctacccaagcagccccgccctcttcccctcaaccccggcccaggccgccctggccgcccgcctgtcactgcagccgcccctgccctgtgccgtgctgcgctcacctgcctccccagccagccgctgacctctcggctttcacttgggcagagggagccatttggtggcgctgcttgtcttctttggttctgggaggggtgagggatgggggtcctgtacacaaccacccactagttctcttctccagccaagaggaataaagttttgcttccattc

SEQ ID NO: 82 - DNA, 8390 bp, Homo sapiens
gcccgcgccggctgtgctgcacagggggaggagagggaaccccaggcgcgagcgggaagaggggacctgcagccacaacttctctggtcctctgcatcccttctgtccctccacccgtccccttccccaccctctggcccccaccttcttggaggcgacaacccccgggaggcattagaagggatttttcccgcaggttgcgaagggaagcaaacttggtggcaacttgcctcccggtgcgggcgtctctcccccaccgtctcaacatgcttaggggtccggggcccgggctgctgctgctggccgtccagtgcctggggacagcggtgccctccacgggagcctcgaagagcaagaggcaggctcagcaaatggttcagccccagtccccggtggctgtcagtcaaagcaagcccggttgttatgacaatggaaaacactatcagataaatcaacagtgggagcggacctacctaggcaatgcgttggtttgtacttgttatggaggaagccgaggttttaactgcgagagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggacttcctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaattggagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacgttctactcctgcaccacagaagggcgacaggacggacatctttggtgcagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctaaaaattctgtaggccgttggaaggaagctaccataccaggccacttaaactcctacaccatcaaaggcctgaagcctggtgtggtatacgagggccagctcatcagcatccagcagtacggccaccaagaagtgactcgctttgacttcaccaccaccagcaccagcacacctgtgaccagcaacaccgtgacaggagagacgactcccttttctcctcttgtggccacttctgaatctgtgaccgaaatcacagccagtagctttgtggtctcctgggtctcagcttccgacaccgtgtcgggattccgggtggaatatgagctgagtgaggagggagatgagccacagtacctggatcttccaagcacagccacttctgtgaacatccctgacctgcttcctggccgaaaatacattgtaaatgtctatcagatatctgaggatggggagcagagtttgatcctgtctacttcacaaacaacagcgcctgatgcccctcctgacacgactgtggaccaagttgatgacacctcaattgttgttcgctggagcagaccccaggctcccatcacagggtacagaatagtctattcgccatcagtagaaggtagcagcacagaactcaaccttcctgaaactgcaaactccgtcaccctcagtgacttgcaacctggtgttcagtataacatcactatctatgctgtggaagaaaatcaagaaagtacacctgttgtcattcaacaagaaaccactggcaccccacgctcagatacagtgccctctcccagggacctgcagtttgtggaagtgacagacgtgaaggtcaccatcatgtggacaccgcctgagagtgcagtgaccggctaccgtgtggatgtgatccccgtcaacctgcctggcgagcacgggcagaggctgcccatcagcaggaacacctttgcagaagtcaccgggctgtcccctggggtcacctattacttcaaagtctttgcagtgagccatgggagggagagcaagcctctgactgctcaacagacaaccaaactggatgctcccactaacctccagtttgtcaatgaaactgattctactgtcctggtgagatggactccacctcgggcccagataacaggataccgactgaccgtgggccttacccgaagaggacagcccaggcagtacaatgtgggtccctctgtctccaagtacccactgaggaatctgcagcctgcatctgagtacaccgtatccctcgtggccataaagggcaaccaagagagccccaaagccactggagtctttaccacactgcagcctgggagctctattccaccttacaacaccgaggtgactgagaccaccattgtgatcacatggacgcctgctccaagaattggttttaagctgggtgtacgaccaagccagggaggagaggcaccacgagaagtgacttcagactcaggaagcatcgttgtgtccggcttgactccaggagtagaatacgtctacaccatccaagtcctgagagatggacaggaaagagatgcgccaattgtaaacaaagtggtgacaccattgtctccaccaacaaacttgcatctggaggcaaaccctgacactggagtgctcacagtctcctgggagaggagcaccaccccagacattactggttatagaattaccacaacccctacaaacggccagcagggaaattctttggaagaagtggtccatgctgatcagagctcctgcacttttgataacctgagtcccggcctggagtacaatgtcagtgtttacactgtcaaggatgacaaggaaagtgtccctatctctgataccatcatcccagaggtgccccaactcactgacctaagctttgttgatataaccgattcaagcatcggcctgaggtggaccccgctaaactcttccaccattattgggtaccgcatcacagtagttgcggcaggagaaggtatccctatttttgaagattttgtggactcctcagtaggatactacacagtcacagggctggagccgggcattgactatgatatcagcgttatcactctcattaatggcggcgagagtgcccctactacactgacacaacaaacggctgttcctcctcccactgacctgcgattcaccaacattggtccagacaccatgcgtgtcacctgggctccacccccatccattgatttaaccaacttcctggtgcgttactcacctgtgaaaaatgaggaagatgttgcagagttgtcaatttctccttcagacaatgcagtggtcttaacaaatctcctgcctggtacagaatatgtagtgagtgtctccagtgtctacgaacaacatgagagcacacctcttagaggaagacagaaaacaggtcttgattccccaactggcattgacttttctgatattactgccaactcttttactgtgcactggattgctcctcgagccaccatcactggctacaggatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccttattgattggccaacaatcaacagtttctgatgttccgagggacctggaagttgttgctgcgacccccaccagcctactgatcagctgggatgctcctgctgtcacagtgagatattacaggatcacttacggagagacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttagtgtctatgctcagaatccaagcggagagagtcagcctctggttcagactgcagtaaccaacattgatcgccctaaaggactggcattcactgatgtggatgtcgattccatcaaaattgcttgggaaagcccacaggggcaagtttccaggtacagggtgacctactcgagccctgaggatggaatccatgagctattccctgcacctgatggtgaagaagacactgcagagctgcaaggcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggagttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggtgaggaaatccaaattggtcacatccccagggaagatgtagactatcacctgtacccacacggtccgggactcaatccaaatgcctctacaggacaagaagctctctctcagacaaccatctcatgggccccattccaggacacttctgagtacatcatttcatgtcatcctgttggcactgatgaagaacccttacagttcagggttcctggaacttctaccagtgccactctgacaggcctcaccagaggtgccacctacaacgtcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttatgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaaatcatctttccaatccagaggaacaagcatgtctctctgccaagatccatctaaactggagtgatgttagcagacccagcttagagttcttctttctttcttaagccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtggaaagacaactgttttaataaaagatttacattcca

SEQ ID NO: 83 - DNA, 871 bp, Homo sapiens
gcagttcggcggtcccgcgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccatgagctcccagattcgtcagaattattccaccgacgtggaggcagccgtcaacagcctggtcaatttgtacctgcaggcctcctacacctacctctctctgggcttctatttcgaccgcgatgatgtggctctggaaggcgtgagccacttcttccgcgaattggccgaggagaagcgcgagggctacgagcgtctcctgaagatgcaaaaccagcgtggcggccgcgctctcttccaggacatcaagaagccagctgaagatgagtggggtaaaaccccagacgccatgaaagctgccatggccctggagaaaaagctgaaccaggcccttttggatcttcatgccctgggttctgcccgcacggacccccatctctgtgacttcctggagactcacttcctagatgaggaagtgaagcttatcaagaagatgggtgaccacctgaccaacctccacaggctgggtggcccggaggctgggctgggcgagtatctcttcgaaaggctcactctcaagcacgactaagagccttctgagcccagcgacttctgaagggccccttgcaaagtaatagggcttctgcctaagcctctccctccagccaataggcagctttcttaactatcctaacaagccttggaccaaatggaaataaagctttttgatgca

SEQ ID NO: 84 - DNA, 1285 bp, Homo sapiens
gctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacca

SEQ ID NO: 85 - DNA, 7910 bp, Homo sapiens
agagtcgagagtgggagaagagcggagcgtgtgagcagtactgcggcctcctctcctctcctaacctcgctctcgcggcctagctttacccgcccgcctgctcggcgaccagcggggatcctcccccagccgcaagtccacgaagaaagcaacgaatgaaaattatgaagacaacgagaagtcagactcctccgggtcgcgctccagctgcttcggcttcgtcgcctactctgtgaactccggggagagatctcgagtcaagattaagaccttaacccaccaacctgcctgttcggacaccccccgggccggccgctgtctgtccccttctccatcgccctctcccagaaagctccggtgcttggaccagctagagtctgagaaagaggagaggcgcgaacgccactccaaaaagagaagggttaaagagggcaaccctaacgatacgcttgactttctgtggctgggaacaccttccaccatgaccacctcagcaagttcccacttaaataaaggcatcaagcaggtgtacatgtccctgcctcagggtgagaaagtccaggccatgtatatctggatcgatggtactggagaaggactgcgctgcaagacccggaccctggacagtgagcccaagtgtgtggaagagttgcctgagtggaatttcgatggctctagtactttacagtctgagggttccaacagtgacatgtatctcgtgcctgctgccatgtttcgggaccccttccgtaaggaccctaacaagctggtgttatgtgaagttttcaagtacaatcgaaggcctgcagagaccaatttgaggcacacctgtaaacggataatggacatggtgagcaaccagcacccctggtttggcatggagcaggagtataccctcatggggacagatgggcacccctttggttggccttccaacggcttcccagggccccagggtccatattactgtggtgtgggagcagacagagcctatggcagggacatcgtggaggcccattaccgggcctgcttgtatgctggagtcaagattgcggggactaatgccgaggtcatgcctgcccagtgggaatttcagattggaccttgtgaaggaatcagcatgggagatcatctctgggtggcccgtttcatcttgcatcgtgtgtgtgaagactttggagtgatagcaacctttgatcctaagcccattcctgggaactggaatggtgcaggctgccataccaacttcagcaccaaggccatgcgggaggagaatggtctgaagtacatcgaggaggccattgagaaactaagcaagcggcaccagtaccacatccgtgcctatgatcccaagggaggcctggacaatgcccgacgtctaactggattccatgaaacctccaacatcaacgacttttctgctggtgtagccaatcgtagcgccagcatacgcattccccggactgttggccaggagaagaagggttactttgaagatcgtcgcccctctgccaactgcgaccccttttcggtgacagaagccctcatccgcacgtgtcttctcaatgaaaccggcgatgagcccttccagtacaaaaattaagtggactagacctccagctgttgagcccctcctagttcttcatcccactccaactcttccccctctcccagttgtcccgattgtaactcaaagggtggaatatcaaggtcgtttttttcattccatgtgcccagttaatcttgctttctttgtttggctgggatagaggggtcaagttattaatttcttcacacctaccctcctttttttccctatcactgaagctttttagtgcattagtggggaggagggtggggagacataaccactgcttccatttaatggggtgcacctgtccaataggcgtagctatccggacagagcacgtttgcagaagggggtctcttcttccaggtagctgaaaggggaagacctgacgtactctggttaggttaggacttgccctcgtggtggaaacttttcttaaaaagttataaccaacttttctattaaaagtgggaattaggagagaaggtaggggttgggaatcagagagaatggctttggtctcttgcttgtgggactagcctggcttgggactaaatgccctgctctgaacacgaagcttagtataaactgatggatatccctaccttgaaagaagaaaaggttcttactgcttggtccttgatttatcacacaaagcagaatagtatttttatatttaaatgtaaagacaaaaaactatatgtatggttttgtggattatgtgtgttttgctaaaggaaaaaaccatccaggtcacggggcaccaaatttgagacaaatagtcggattagaaataaagcatctcattttgagtagagagcaagggaagtggttcttagatggtgatctgggattaggccctcaagacccttttgggtttctgccctgcccaccctctggagaaggtgggcactggattagttaacagacaacacgttactagcagtcacttgatctccgtggctttggtttaaaagacacacttgtccacataggtttagagataagagttggctggtcaacttgagcatgttactgacagagggggtattggggttattttctggtaggaatagcatgtcactaaagcaggccttttgatattaaattttttaaaaagcaaaattatagaagtttagattttaatcaaatttgtagggtttctaggtaatttttacagaattgcttgtttgcttcaactgtctcctacctctgctcttggaggagatggggacagggctggagtcaaaacacttgtaattttgtatcttgatgtctttgttaagactgctgaagaattattttttttcttttataataaggaataaaccccacctttattccttcatttcatctaccattttctggttcttgtgttggctgtggcaggccagctgtggttttcttttgccatgacaacttctaattgccatgtacagtatgttcaaagtcaaataactcctcattgtaaacaaactgtgtaactgcccaaagcagcacttataaatcagcctaacataagatctctctgatgtgtttgtgattctttcaaatccctatgtgccattatatttctttatttcctaaaacaggcaaaataagctcaagtttatgtactctgagtttttaaaacactggagtgatgttgctgaccagccgtttcctgtacctctctaagttgggtatttgggacttaagggattaagtttttcacctagacttagttacacacaatcttggcatttcctagcctagaggtttgtagcagggtacaagccccactcctcccccttcctttgctcccctgagtttggttttggcttaccataacattgttttgaccattcctagcctaatacaatagcctaacataatgtaagattaactggctttacgatttctattctctgctctcagtgataagaaacaaatattagctaccctgctaccctggttgaagccttccaaggctggctatgccctaggcatgggctcatccttgggtgtatcttgccttgcaggaagaccagtggaccgattgtgattctcaaaagctctgtgttgtcacctgtgcccttgccccttgctcttatcttggtccgtgtatctgggagttcttccaccttatcttggccaattcctaccttcgttcattcctcatgaggttgggtaaaagctccctccggctcccatgatgctgtgcatatacctagcaaaaagcaattattggacacattggagtgcaatattattaatagcattaatactactaataatgtgggcaatagtgattgtttttaaaaggcagtatactcttaccagtgcgaggtagctggggcctgtgatagtttttagagataagttcttcaggcaactgtgtattttacactagtcaagtaatcctagatatccgtggtttttcttaagaaagttggctcgtaatatgatttaatattcaaagtagagtcatctacctattagcttgctggcgtggtcctagtttatgcctgtttcagcatgattgttgagtaccctgtttcatccttagcattttcttgattttgttgttaaatgatgtatacccttatttccattgaatctgtgcttccacccccccaactgaagttgtcttccctttgcttggccacccttacagcctcttggatggtgtatcctacagtgtaagcactaaactgaagaggcagtgacctgagcactttggattttgttcattgtaatcaattccatgacaaaatgattgcatgagaaggaattttaaattcataggatcagaatttaggtgaaaacaaccagcatatttgtttcttcaccctctcacctagaattagctttgacctacaggtcacagtgcaatccccttgtatttctaaggtgttttttatagttcatttgcagacaatgggttatgtgataacttttatcagtgatagattaaacagaataatgaccaagctttcaaccttaaggagtcaggccagtatttacaaaaggaggtctccatgaactccttaaatatgagttcccctaatatcatcttgccaggtactaaataacaactgatagcacaagctatagggaatttgaaagaattccatggatgggtgttgtctagggccttttgttgtttttgagacggggtctgactctcacccaggctggagtatagtgtggcgcaatcttggctcactgcaacttctgcctcccagattcaagcgattctcctgcctcagcctcccaagtagctgagactacaggtgtgcaccaccatgctcagctaatttttgtatttttagtacagatggggtttcaccatgttggccaggctggtcttgaactcctgatctcccaaagtgaggtcttgaactggtcttgaactcctccacctcccaaagtgctgggattacaggcgtgagccactgcacccggcctagggccatgtaaaaagccagatctgtgctgctgtctgtgtagaagggtagacaagtggatgagaagttcctgaactattcttggcccttttaccactaagtgaaagtaacttgctgccccaaagaaagatgtctcatcattcgacaggactttctagttgaacttcatgaaagcaagagatcctgtttttcttgctcaccactgtatcttgagacctgttgtagtgcctgcaatacttatttaataagttatttttaagtatcagttttgtgagctttaactctatgaggtctttgttgtttgactgtattttaactctggccatgacagcaagacaaagttccatttttattgagcttaaaaagaatcaaggccaggtgaagtggcttacgcctgtgatcccaacactttgtgaggctgcagcaggaggatctcttgagcccaggagtttgagaccgttctaggcaatgtagtgaggtccagactccacaaaataatttttttttaaattgcacgcctgtagtctcagctatcaggaggctgagatgggaggatgacttgagcccaggaaattgaagctgcagtgaattgtgattgcaccactgcactccagcctgggtgacagatcaagaccttgcctaaacaaaacaaaacaaacaaaaccccaaaaaacaaattgaaaatgttgattctttttactacaaacattatggcagcactaaaaacttcgtgggagtgtactgtggaaaatagtgtacttaattaattctcattgtaatcaggctaccaagagccttgtgttgctttaagagttataactgccaggcacagtggctcatgcctataatcccagcaccttgagaggccgaggcaggtggatcacctgagatcgggagtttgagaccagccgggccaatatggtgaaacaagctgtgtctctactaaatacaaaaaattagccgggcgtggtggcacatgcctgtaatcccagctgcttgggagactgagacaggagaattgcttgaacctggaaggcggaggttgcagtgagctgagattgcaacattgtactccagcctgggcaacaagagggaaactccatctcaaaaaaaaaaaaaaagttgtaactgaggctgggcatggtggctcatacctgtaatcccagcactttgaaaagccgaggcaggtagatcacttgagctcagaagttcgagactagcctgggcaacatgacaaaaccccatctctacaaaaaatacgaaaaattagctgggcgtggtggcatgcacctgtagtcctagctacctgggaggctgaggtgggaagattacttgaagctgcagtgagccatggttgtgccactgccctccagtctgggcaacaaagtgagaccctgtctcaaaaaaacaaaaaaaaattataactgatgtaaactggcagtttaggctgggtgtggtggctcaagcctgtaatcctagcactttgggaggccaaggcaggtggatcacctgagttcaggagttcgagaccagcgtggccaacatggtgaaaccttgtctctattaaaaataccaaaattagcaagatgtggtggtgggtgcctataattccagctactcaggaggctgaggcaggaggatcgctggagccagggaggcagaggttacagtaagcaaagatcactccacttcactccagcctgggcaaaagagtgagacatatcaaaaaataaacaaataaataaataaataagtggcagttcatcatttaactccaaagactttgcgtacatttctactgaaaacaatctgagctgattagaaccctgccattttatagcctttagctcgatctccgaccgttcatttaaaaaaattctacttcaggccgggcatggtggctcaagcctgtaatcccatcactgtaggaggccaaagtgggcagatcacttaaggtcaggagtttgagaccagcctggccaccatggtgaaaccccatctctactaaaaatacaaaaattagccgggcttggtggtgagcacctgtaatcccaccctgccgagtggcaggctgaggcaggagaatcgcttgagcccaagagccggaggttgcagtgagccaagcttgcaccattgcactccagcctaggcaacagagtgtgactccatctcaagaaaaaaaaaattctatttcattttacaatatgcagatatatgtccatacacatgcataatataaatgtataccatatttgtgagaatatgcatatatgtacacattagatacacaatacaagcacaatacatatgtcttttgcccaagatacagcattttgtaaaggagacaggaatttagtaatatatgttccagaaacagtacacaagagaattcgccgagatgagaaagttgtcactaggaatggggagtggtaagatgtagaaggtataattgttcttaaagttctactgccaactctttccaattaattacccactctgccatgctttatggacaggaggttgtcggacactgtcaattaataaatatttgagcatgatacactgcttggagctcctctaatataggagagtgatatcctagtgcatgttacagagggagtgtccacacagttcctattgtcatttgatgagttacttttcaggggccttgtacctgagcaagttgtcctctttttgatggatttcagattgagttacctgcattgtcttgagattgcagcgtgtttcctccactgtacggcgtagtcagcagatctattagttaaactccagtgggccctcagtcactaaatctatcctctgtgttgaaggctttctgcatttgcctttcaataaaggtttagaataac

SEQ ID NO: 86 - DNA, 1140 bp, Homo sapiens
ctctctttcactgcaaggcggcggcaggagaggttgtggtgctagtttctctaagccatccagtgccatcctcgtcgctgcagcgacacacgctctcgccgccgccatgactgagcagatgacccttcgtggcaccctcaagggccacaacggctgggtaacccagatcgctactaccccgcagttcccggacatgatcctctccgcctctcgagataagaccatcatcatgtggaaactgaccagggatgagaccaactatggaattccacagcgtgctctgcggggtcactcccactttgttagtgatgtggttatctcctcagatggccagtttgccctctcaggctcctgggatggaaccctgcgcctctgggatctcacaacgggcaccaccacgaggcgatttgtgggccataccaaggatgtgctgagtgtggccttctcctctgacaaccggcagattgtctctggatctcgagataaaaccatcaagctatggaataccctgggtgtgtgcaaatacactgtccaggatgagagccactcagagtgggtgtcttgtgtccgcttctcgcccaacagcagcaaccctatcatcgtctcctgtggctgggacaagctggtcaaggtatggaacctggctaactgcaagctgaagaccaaccacattggccacacaggctatctgaacacggtgactgtctctccagatggatccctctgtgcttctggaggcaaggatggccaggccatgttatgggatctcaacgaaggcaaacacctttacacgctagatggtggggacatcatcaacgccctgtgcttcagccctaaccgctactggctgtgtgctgccacaggccccagcatcaagatctgggatttagagggaaagatcattgtagatgaactgaagcaagaagttatcagtaccagcagcaaggcagaaccaccccagtgcacctccctggcctggtctgctgatggccagactctgtttgctggctacacggacaacctggtgcgagtgtggcaggtgaccattggcacacgctagaagtttatggcagagctttacaaataaaaaaaaaactggcttttctgacttttaggtttttttttcttatatgcaaaaaa

SEQ ID NO: 87 - DNA, 1586 bp, Homo sapiens
tccgcagtcccggttctaaagtccccagtcacccacccggactcacattctccccagaggccgagatgcgggtcatggcgccccgagccctcctcctgctgctctcgggaggcctggccctgaccgagacctgggcctgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaactacaagcgccaggcacaggctgaccgagtgagcctgcggaacctgcgcggctactacaaccagagcgaggacgggtctcacaccctccagaggatgtatggctgcgacctggggcccgacgggcgcctcctccgcgggtatgaccagtccgcctacgacggcaaggattacatcgccctgaacgaggacctgcgctcctggaccgccgcggacaccgcggctcagatcacccagcgcaagttggaggcggcccgtgcggcggagcagctgagagcctacctggagggcacgtgcgtggagtggctccgcagatacctggagaacgggaaggagacgctgcagcgcgcagaacccccaaagacacacgtgacccaccaccccctctctgaccatgaggccaccctgaggtgctgggccctgggcttctaccctgcggagatcacactgacctggcagcgggatggggaggaccagacccaggacaccgagcttgtggagaccaggccagcaggagatggaaccttccagaagtgggcagctgtggtggtgccttctggacaagagcagagatacacgtgccatatgcagcacgaggggctgcaagagcccctcaccctgagctgggagccatcttcccagcccaccatccccatcatgggcatcgttgctggcctggctgtcctggttgtcctagctgtccttggagctgtggtcaccgctatgatgtgtaggaggaagagctcaggtggaaaaggagggagctgctctcaggctgcgtgcagcaacagtgcccagggctctgatgagtctctcatcacttgtaaagcctgagacagctgcctgtgtgggactgagatgcaggatttcttcacacctctcctttgtgacttcaagagcctctggcatctctttctgcaaaggcgtctgaatgtgtctgcgttcctgttagcataatgtgaggaggtggagagacagcccacccccgtgtccaccgtgacccctgtccccacactgacctgtgttccctccccgatcatctttcctgttccagagaggtggggctggatgtctccatctctgtctcaaattcatggtgcactgagctgcaacttcttacttccctaatgaagttaagaacctgaatataaatttgtgttctcaaatatttgctatgaagcgttgatggattaattaaataagtcaattcctagaagttgagagagcaaataaagacctgagaaccttccagaa

SEQ ID NO: 88 - DNA, 3228 bp, Homo sapiens
agttgcttcagcgtcccggtgtggctgtgccgttggtcctgtgcggtcacttagccaagatgcctgaggaaacccagacccaagaccaaccgatggaggaggaggaggttgagacgttcgcctttcaggcagaaattgcccagttgatgtcattgatcatcaatactttctactcgaacaaagagatctttctgagagagctcatttcaaattcatcagatgcattggacaaaatccggtatgaaagcttgacagatcccagtaaattagactctgggaaagagctgcatattaaccttataccgaacaaacaagatcgaactctcactattgtggatactggaattggaatgaccaaggctgacttgatcaataaccttggtactatcgccaagtctgggaccaaagcgttcatggaagctttgcaggctggtgcagatatctctatgattggccagttcggtgttggtttttattctgcttatttggttgctgagaaagtaactgtgatcaccaaacataacgatgatgagcagtacgcttgggagtcctcagcagggggatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaagaagaaaaagaaaaagaagagaaagagtcggaagacaaacctgaaattgaagatgttggttctgatgaggaagaagaaaagaaggatggtgacaagaagaagaagaagaagattaaggaaaagtacatcgatcaagaagagctcaacaaaacaaagcccatctggaccagaaatcccgacgatattactaatgaggagtacggagaattctataagagcttgaccaatgactgggaagatcacttggcagtgaagcatttttcagttgaaggacagttggaattcagagcccttctatttgtcccacgacgtgctccttttgatctgtttgaaaacagaaagaaaaagaacaacatcaaattgtatgtacgcagagttttcatcatggataactgtgaggagctaatccctgaatatctgaacttcattagaggggtggtagactcggaggatctccctctaaacatatcccgtgagatgttgcaacaaagcaaaattttgaaagttatcaggaagaatttggtcaaaaaatgcttagaactctttactgaactggcggaagataaagagaactacaagaaattctatgagcagttctctaaaaacataaagcttggaatacacgaagactctcaaaatcggaagaagctttcagagctgttaaggtactacacatctgcctctggtgatgagatggtttctctcaaggactactgcaccagaatgaaggagaaccagaaacatatctattatatcacaggtgagaccaaggaccaggtagctaactcagcctttgtggaacgtcttcggaaacatggcttagaagtgatctatatgattgagcccattgatgagtactgtgtccaacagctgaaggaatttgaggggaagactttagtgtcagtcaccaaagaaggcctggaacttccagaggatgaagaagagaaaaagaagcaggaagagaaaaaaacaaagtttgagaacctctgcaaaatcatgaaagacatattggagaaaaaagttgaaaaggtggttgtgtcaaaccgattggtgacatctccatgctgtattgtcacaagcacatatggctggacagcaaacatggagagaatcatgaaagctcaagccctaagagacaactcaacaatgggttacatggcagcaaagaaacacctggagataaaccctgaccattccattattgagaccttaaggcaaaaggcagaggctgataagaacgacaagtctgtgaaggatctggtcatcttgctttatgaaactgcgctcctgtcttctggcttcagtctggaagatccccagacacatgctaacaggatctacaggatgatcaaacttggtctgggtattgatgaagatgaccctactgctgatgataccagtgctgctgtaactgaagaaatgccaccccttgaaggagatgacgacacatcacgcatggaagaagtagactaatctctggctgagggatgacttacctgttcagtactctacaattcctctgataatatattttcaaggatgtttttctttatttttgttaatattaaaaagtctgtatggcatgacaactactttaaggggaagataagatttctgtctactaagtgatgctgtgataccttaggcactaaagcagagctagtaatgctttttgagtttcatgttggtttattttcacagattggggtaacgtgcactgtaagacgtatgtaacatgatgttaactttgtggtctaaagtgtttagctgtcaagccggatgcctaagtagaccaaatcttgttattgaagtgttctgagctgtatcttgatgtttagaaaagtattcgttacatcttgtaggatctactttttgaacttttcattccctgtagttgacaattctgcatgtactagtcctctagaaataggttaaactgaagcaacttgatggaaggatctctccacagggcttgttttccaaagaaaagtattgtttggaggagcaaagttaaaagcctacctaagcatatcgtaaagctgttcaaaaataactcagacccagtcttgtggatggaaatgtagtgctcgagtcacattctgcttaaagttgtaacaaatacagatgagttaaaagatattgtgtgacagtgtcttatttagggggaaaggggagtatctggatgacagttagtgccaaaatgtaaaacatgaggcgctagcaggagatggttaaacactagctgctccaagggttgacatggtcttcccagcatgtactcagcaggtgtggggtggagcacacgtaggcacagaaaacaggaatgcagacaacatgcatcccctgcgtccatgagttacatgtgttctcttagtgtccacgttgttttgatgttattcatggaataccttctgtgttaaatacagtcacttaattccttggc

SEQ ID NO: 89 - DNA, 2555 bp, Homo sapiens
ctctcgagtcactccggcgcagtgttgggactgtctgggtatcggaaagcaagcctacgttgctcactattacgtataatccttttcttttcaagatgcctgaggaagtgcaccatggagaggaggaggtggagacttttgcctttcaggcagaaattgcccaactcatgtccctcatcatcaataccttctattccaacaaggagattttccttcgggagttgatctctaatgcttctgatgccttggacaagattcgctatgagagcctgacagacccttcgaagttggacagtggtaaagagctgaaaattgacatcatccccaaccctcaggaacgtaccctgactttggtagacacaggcattggcatgaccaaagctgatctcataaataatttgggaaccattgccaagtctggtactaaagcattcatggaggctcttcaggctggtgcagacatctccatgattgggcagtttggtgttggcttttattctgcctacttggtggcagagaaagtggttgtgatcacaaagcacaacgatgatgaacagtatgcttgggagtcttctgctggaggttccttcactgtgcgtgctgaccatggtgagcccattggcaggggtaccaaagtgatcctccatcttaaagaagatcagacagagtacctagaagagaggcgggtcaaagaagtagtgaagaagcattctcagttcataggctatcccatcaccctttatttggagaaggaacgagagaaggaaattagtgatgatgaggcagaggaagagaaaggtgagaaagaagaggaagataaagatgatgaagaaaaacccaagatcgaagatgtgggttcagatgaggaggatgacagcggtaaggataagaagaagaaaactaagaagatcaaagagaaatacattgatcaggaagaactaaacaagaccaagcctatttggaccagaaaccctgatgacatcacccaagaggagtatggagaattctacaagagcctcactaatgactgggaagaccacttggcagtcaagcacttttctgtagaaggtcagttggaattcagggcattgctatttattcctcgtcgggctccctttgacctttttgagaacaagaagaaaaagaacaacatcaaactctatgtccgccgtgtgttcatcatggacagctgtgatgagttgataccagagtatctcaattttatccgtggtgtggttgactctgaggatctgcccctgaacatctcccgagaaatgctccagcagagcaaaatcttgaaagtcattcgcaaaaacattgttaagaagtgccttgagctcttctctgagctggcagaagacaaggagaattacaagaaattctatgaggcattctctaaaaatctcaagcttggaatccacgaagactccactaaccgccgccgcctgtctgagctgctgcgctatcatacctcccagtctggagatgagatgacatctctgtcagagtatgtttctcgcatgaaggagacacagaagtccatctattacatcactggtgagagcaaagagcaggtggccaactcagcttttgtggagcgagtgcggaaacggggcttcgaggtggtatatatgaccgagcccattgacgagtactgtgtgcagcagctcaaggaatttgatgggaagagcctggtctcagttaccaaggagggtctggagctgcctgaggatgaggaggagaagaagaagatggaagagagcaaggcaaagtttgagaacctctgcaagctcatgaaagaaatcttagataagaaggttgagaaggtgacaatctccaatagacttgtgtcttcaccttgctgcattgtgaccagcacctacggctggacagccaatatggagcggatcatgaaagcccaggcacttcgggacaactccaccatgggctatatgatggccaaaaagcacctggagatcaaccctgaccaccccattgtggagacgctgcggcagaaggctgaggccgacaagaatgataaggcagttaaggacctggtggtgctgctgtttgaaaccgccctgctatcttctggcttttcccttgaggatccccagacccactccaaccgcatctatcgcatgatcaagctaggtctaggtattgatgaagatgaagtggcagcagaggaacccaatgctgcagttcctgatgagatcccccctctcgagggcgatgaggatgcgtctcgcatggaagaagtcgattaggttaggagttcatagttggaaaacttgtgcccttgtatagtgtccccatgggctcccactgcagcctcgagtgcccctgtcccacctggctccccctgctggtgtctagtgtttttttccctctcctgtccttgtgttgaaggcagtaaactaagggtgtcaagccccattccctctctactcttgacagcaggattggatgttgtgtattgtggtttattttattttcttcattttgttctgaaattaaagtatgcaaaataaagaatatgccgtttttataca

SEQ ID NO: 90 - DNA, 2782 bp, Homo sapiens
agtgggcggaccgcgcggctggaggtgtgaggatccgaacccaggggtggggggtggaggcggctcctgcgatcgaaggggacttgagactcaccggccgcacgccatgagggccctgtgggtgctgggcctctgctgcgtcctgctgaccttcgggtcggtcagagctgacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaaaaagatgaattgtaaattatactctcaccatttggatcctgtgtggagagggaatgtgaaatttacatcatttctttttgggagagacttgttttggatgccccctaatccccttctcccctgcactgtaaaatgtgggattatgggtcacaggaaaaagtgggttttttagttgaattttttttaacattcctcatgaatgtaaatttgtactatttaactgactattcttgatgtaaaatcttgtcatgtgtataaaaataaaaaagatcccaaata

SEQ ID NO: 91 - DNA, 3921 bp, Homo sapiens
gacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcaagatgaagctctccctggtggccgcgatgctgctgctgctcagcgcggcgcgggccgaggaggaggacaagaaggaggacgtgggcacggtggtcggcatcgacctggggaccacctactcctgcgtcggcgtgttcaagaacggccgcgtggagatcatcgccaacgatcagggcaaccgcatcacgccgtcctatgtcgccttcactcctgaaggggaacgtctgattggcgatgccgccaagaaccagctcacctccaaccccgagaacacggtctttgacgccaagcggctcatcggccgcacgtggaatgacccgtctgtgcagcaggacatcaagttcttgccgttcaaggtggttgaaaagaaaactaaaccatacattcaagttgatattggaggtgggcaaacaaagacatttgctcctgaagaaatttctgccatggttctcactaaaatgaaagaaaccgctgaggcttatttgggaaagaaggttacccatgcagttgttactgtaccagcctattttaatgatgcccaacgccaagcaaccaaagacgctggaactattgctggcctaaatgttatgaggatcatcaacgagcctacggcagctgctattgcttatggcctggataagagggagggggagaagaacatcctggtgtttgacctgggtggcggaaccttcgatgtgtctcttctcaccattgacaatggtgtcttcgaagttgtggccactaatggagatactcatctgggtggagaagactttgaccagcgtgtcatggaacacttcatcaaactgtacaaaaagaagacgggcaaagatgtcaggaaagacaatagagctgtgcagaaactccggcgcgaggtagaaaaggccaaacgggccctgtcttctcagcatcaagcaagaattgaaattgagtccttctatgaaggagaagacttttctgagaccctgactcgggccaaatttgaagagctcaacatggatctgttccggtctactatgaagcccgtccagaaagtgttggaagattctgatttgaagaagtctgatattgatgaaattgttcttgttggtggctcgactcgaattccaaagattcagcaactggttaaagagttcttcaatggcaaggaaccatcccgtggcataaacccagatgaagctgtagcgtatggtgctgctgtccaggctggtgtgctctctggtgatcaagatacaggtgacctggtactgcttgatgtatgtccccttacacttggtattgaaactgtgggaggtgtcatgaccaaactgattccaaggaacacagtggtgcctaccaagaagtctcagatcttttctacagcttctgataatcaaccaactgttacaatcaaggtctatgaaggtgaaagacccctgacaaaagacaatcatcttctgggtacatttgatctgactggaattcctcctgctcctcgtggggtcccacagattgaagtcacctttgagatagatgtgaatggtattcttcgagtgacagctgaagacaagggtacagggaacaaaaataagatcacaatcaccaatgaccagaatcgcctgacacctgaagaaatcgaaaggatggttaatgatgctgagaagtttgctgaggaagacaaaaagctcaaggagcgcattgatactagaaatgagttggaaagctatgcctattctctaaagaatcagattggagataaagaaaagctgggaggtaaactttcctctgaagataaggagaccatggaaaaagctgtagaagaaaagattgaatggctggaaagccaccaagatgctgacattgaagacttcaaagctaagaagaaggaactggaagaaattgttcaaccaattatcagcaaactctatggaagtgcaggccctcccccaactggtgaagaggatacagcagaaaaagatgagttgtagacactgatctgctagtgctgtaatattgtaaatactggactcaggaacttttgttaggaaaaaattgaaagaacttaagtctcgaatgtaattggaatcttcacctcagagtggagttgaaactgctatagcctaagcggctgtttactgcttttcattagcagttgctcacatgtctttgggtgggggggagaagaagaattggccatcttaaaaagcgggtaaaaaacctgggttagggtgtgtgttcaccttcaaaatgttctatttaacaactgggtcatgtgcatctggtgtaggaagttttttctaccataagtgacaccaataaatgtttgttatttacactggtctaatgtttgtgagaagcttctaattagatcaattacttattttaggaaatttaagactagatactcgtgtgtggggtgaggggagggagtatttggtatgttgggataaggaaacacttctatttaatgcttccagggatttttttttttttttttaaccctcctgggcccaagtgatccttccacctcagtctcccagctaattgagaccacaggcttgttaccaccatgctcggcttttgcattaatctaagaaaaggggagagaagttaatccacatctttactcaggcaaggggcatttcacagtgcccaagagtggggttttcttgaacatacttggtttcctatttccccttatctttctaaaactgcctttctggtggctttttttaaaattattactaatgatgcttttatagctgcttggattctctgagaaatgatggggagtgagtgatcactggtattaactttatacacttggatttcatttgtaactttaggatgtaaaggtatattgtgaaccctagctgtgtcagaatctccatccctgaaatttctcattagtggtactggggtgggatcttggatggtgacattgaaactacactaaatcccctcactatgaatgggttgttaaaggcaatggtttgtgtcaaaactggtttaggattacttagattgtgttcctgaagaaaagagtccaggtaaatggtatgatcaataaaggacaggctggtgctaacataaaatccaatattgtaatcctagcactttgggaggccaaggcgggtggatcacaaggtcaagagatagagaccatctttgccaacatggtgaaactccatctctactgaaaatacaaaaattagctgggcgtggtagtgcaagctgaaggctgaggcaggagaatcactcgaacccgggaggcagaggttgcagtgagccgagatcacaccactgtactccagcccggcactccagcctggcgacaagagtgagactccacctcaaaaaaaaaaaaaagaatccaatactgcccaaggataggtattttatagatgggcaactggctgaaaggttaattctctagggctagtagaactggatcccaacaccaaactcttaattagacctaggcctcagctgcactgcccgaaaagcatttgggcagaccctgagcagaatactggtctcaggccaagcccaatacagccattaaagatgacctacagtgctgtgtaccctggggcaatagggttaaatggtagttagcaactagggctagtcttcccttacctcaaaggctctcactaccgtggaccacctagtctgtaactctttctgaggagctgttactgaatattaaaaagatagacttcaa

SEQ ID NO: 92 - DNA, 2264 bp, Homo sapiens
ctcattgaactcgcctgcagctcttgggttttttgtggcttccttcgttattggagccaggcctacaccccagcaaccatgtccaagggacctgcagttggtattgatcttggcaccacctactcttgtgtgggtgttttccagcacggaaaagtcgagataattgccaatgatcagggaaaccgaaccactccaagctatgtcgcctttacggacactgaacggttgatcggtgatgccgcaaagaatcaagttgcaatgaaccccaccaacacagtttttgatgccaaacgtctgattggacgcagatttgatgatgctgttgtccagtctgatatgaaacattggccctttatggtggtgaatgatgctggcaggcccaaggtccaagtagaatacaagggagagaccaaaagcttctatccagaggaggtgtcttctatggttctgacaaagatgaaggaaattgcagaagcctaccttgggaagactgttaccaatgctgtggtcacagtgccagcttactttaatgactctcagcgtcaggctaccaaagatgctggaactattgctggtctcaatgtacttagaattattaatgagccaactgctgctgctattgcttacggcttagacaaaaaggttggagcagaaagaaacgtgctcatctttgacctgggaggtggcacttttgatgtgtcaatcctcactattgaggatggaatctttgaggtcaagtctacagctggagacacccacttgggtggagaagattttgacaaccgaatggtcaaccattttattgctgagtttaagcgcaagcataagaaggacatcagtgagaacaagagagctgtaagacgcctccgtactgcttgtgaacgtgctaagcgtaccctctcttccagcacccaggccagtattgagatcgattctctctatgaaggaatcgacttctatacctccattacccgtgcccgatttgaagaactgaatgctgacctgttccgtggcaccctggacccagtagagaaagcccttcgagatgccaaactagacaagtcacagattcatgatattgtcctggttggtggttctactcgtatccccaagattcagaagcttctccaagacttcttcaatggaaaagaactgaataagagcatcaaccctgatgaagctgttgcttatggtgcagctgtccaggcagccatcttgtctggagacaagtctgagaatgttcaagatttgctgctcttggatgtcactcctctttcccttggtattgaaactgctggtggagtcatgactgtcctcatcaagcgtaataccaccattcctaccaagcagacacagaccttcactacctattctgacaaccagcctggtgtgcttattcaggtttatgaaggcgagcgtgccatgacaaaggataacaacctgcttggcaagtttgaactcacaggcatacctcctgcaccccgaggtgttcctcagattgaagtcacttttgacattgatgccaatggtatactcaatgtctctgctgtggacaagagtacgggaaaagagaacaagattactatcactaatgacaagggccgtttgagcaaggaagacattgaacgtatggtccaggaagctgagaagtacaaagctgaagatgagaagcagagggacaaggtgtcatccaagaattcacttgagtcctatgccttcaacatgaaagcaactgttgaagatgagaaacttcaaggcaagattaacgatgaggacaaacagaagattctggacaagtgtaatgaaattatcaactggcttgataagaatcagactgctgagaaggaagaatttgaacatcaacagaaagagctggagaaagtttgcaaccccatcatcaccaagctgtaccagagtgcaggaggcatgccaggaggaatgcctgggggatttcctggtggtggagctcctccctctggtggtgcttcctcagggcccaccattgaagaggttgattaagccaaccaagtgtagatgtagcattgttccacacatttaaaacatttgaaggacctaaattcgtagcaaattctgtggcagttttaaaaagttaagctgctatagtaagttactgggcattctcaatacttgaatatggaacatatgcacaggggaaggaaataacattgcactttataaacactgtattgtaagtggaaaatgcaatgtcttaaataaaactatttaaaattggcaccatacaa

SEQ ID NO: 93 - DNA, 777 bp, Homo sapiens
gcacttttctgagcagacgtccagagcagagtcagccagcatgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggcccagcttgggggcccagaagctgcaaaatccgatgagactgccgccaagtaaagccttagcccggatgcccacccctgctgccgccactggctgtgcctcccccgccacctgtgtgttcttttgatacatttatcttctgtttttctcaaataaagttcaaagcaaccacc

SEQ ID NO: 94 - DNA, 1636 bp, Homo sapiens
acccgagcaccttctcttcactcagccaactgctcgctcgctcacctccctcctctgcaccatgaccacctgcagccgccagttcacctcctccagctccatgaagggctcctgcggcatcgggggcggcatcgggggcggctccagccgcatctcctccgtcctggccggagggtcctgccgcgcccccagcacctacgggggcggcctgtctgtctcatcctcccgcttctcctctgggggagcctgcgggctggggggcggctatggcggtggcttcagcagcagcagcagcagctttggtagtggctttgggggaggatatggtggtggccttggtgctggcttgggtggtggctttggtggtggctttgctggtggtgatgggcttctggtgggcagtgagaaggtgaccatgcagaacctcaatgaccgcctggcctcctacctggacaaggtgcgtgctctggaggaggccaacgccgacctggaagtgaagatccgtgactggtaccagaggcagcggcctgctgagatcaaagactacagtccctacttcaagaccattgaggacctgaggaacaagattctcacagccacagtggacaatgccaatgtccttctgcagattgacaatgcccgtctggccgcggatgacttccgcaccaagtatgagacagagttgaacctgcgcatgagtgtggaagccgacatcaatggcctgcgcagggtgctggacgaactgaccctggccagagctgacctggagatgcagattgagagcctgaaggaggagctggcctacctgaagaagaaccacgaggaggagatgaatgccctgagaggccaggtgggtggagatgtcaatgtggagatggacgctgcacctggcgtggacctgagccgcattctgaacgagatgcgtgaccagtatgagaagatggcagagaagaaccgcaaggatgccgaggaatggttcttcaccaagacagaggagctgaaccgcgaggtggccaccaacagcgagctggtgcagagcggcaagagcgagatctcggagctccggcgcaccatgcagaacctggagattgagctgcagtcccagctcagcatgaaagcatccctggagaacagcctggaggagaccaaaggtcgctactgcatgcagctggcccagatccaggagatgattggcagcgtggaggagcagctggcccagctccgctgcgagatggagcagcagaaccaggagtacaagatcctgctggacgtgaagacgcggctggagcaggagatcgccacctaccgccgcctgctggagggcgaggacgcccacctctcctcctcccagttctcctctggatcgcagtcatccagagatgtgacctcctccagccgccaaatccgcaccaaggtcatggatgtgcacgatggcaaggtggtgtccacccacgagcaggtccttcgcaccaagaactgaggctgcccagccccgctcaggcctaggaggccccccgtgtggacacagatcccactggaagatcccctctcctgcccaagcacttcacagctggaccctgcttcaccctcaccccctcctggcaatcaatacagcttcattatctgagttgcataa

SEQ ID NO: 95 - DNA, 1413 bp, Homo sapiens
gccgccaccgtcgtccgcaaagcctgagtcctgtcctttctctctccccggacagcatgagcttcaccactcgctccaccttctccaccaactaccggtccctgggctctgtccaggcgcccagctacggcgcccggccggtcagcagcgcggccagcgtctatgcaggcgctgggggctctggttcccggatctccgtgtcccgctccaccagcttcaggggcggcatggggtccgggggcctggccaccgggatagccgggggtctggcaggaatgggaggcatccagaacgagaaggagaccatgcaaagcctgaacgaccgcctggcctcttacctggacagagtgaggagcctggagaccgagaaccggaggctggagagcaaaatccgggagcacttggagaagaagggaccccaggtcagagactggagccattacttcaagatcatcgaggacctgagggctcagatcttcgcaaatactgtggacaatgcccgcatcgttctgcagattgacaatgcccgtcttgctgctgatgactttagagtcaagtatgagacagagctggccatgcgccagtctgtggagaacgacatccatgggctccgcaaggtcattgatgacaccaatatcacacgactgcagctggagacagagatcgaggctctcaaggaggagctgctcttcatgaagaagaaccacgaagaggaagtaaaaggcctacaagcccagattgccagctctgggttgaccgtggaggtagatgcccccaaatctcaggacctcgccaagatcatggcagacatccgggcccaatatgacgagctggctcggaagaaccgagaggagctagacaagtactggtctcagcagattgaggagagcaccacagtggtcaccacacagtctgctgaggttggagctgctgagacgacgctcacagagctgagacgtacagtccagtccttggagatcgacctggactccatgagaaatctgaaggccagcttggagaacagcctgagggaggtggaggcccgctacgccctacagatggagcagctcaacgggatcctgctgcaccttgagtcagagctggcacagacccgggcagagggacagcgccaggcccaggagtatgaggccctgctgaacatcaaggtcaagctggaggctgagatcgccacctaccgccgcctgctggaagatggcgaggactttaatcttggtgatgccttggacagcagcaactccatgcaaaccatccaaaagaccaccacccgccggatagtggatggcaaagtggtgtctgagaccaatgacaccaaagttctgaggcattaagccagcagaagcagggtaccctttggggagcaggaggccaataaaaagttcagagttcattgga

SEQ ID NO: 96 - DNA, 1390 bp, Homo sapiens
gctcctcccgcgaatcgcagcttctgagaccagggttgctccgtccgtgctccgcctcgccatgacttcctacagctatcgccagtcgtcggccacgtcgtccttcggaggcctgggcggcggctccgtgcgttttgggccgggggtcgcctttcgcgcgcccagcattcacgggggctccggcggccgcggcgtatccgtgtcctccgcccgctttgtgtcctcgtcctcctcgggggcctacggcggcggctacggcggcgtcctgaccgcgtccgacgggctgctggcgggcaacgagaagctaaccatgcagaacctcaacgaccgcctggcctcctacctggacaaggtgcgcgccctggaggcggccaacggcgagctagaggtgaagatccgcgactggtaccagaagcaggggcctgggccctcccgcgactacagccactactacacgaccatccaggacctgcgggacaagattcttggtgccaccattgagaactccaggattgtcctgcagatcgacaatgcccgtctggctgcagatgacttccgaaccaagtttgagacggaacaggctctgcgcatgagcgtggaggccgacatcaacggcctgcgcagggtgctggatgagctgaccctggccaggaccgacctggagatgcagatcgaaggcctgaaggaagagctggcctacctgaagaagaaccatgaggaggaaatcagtacgctgaggggccaagtgggaggccaggtcagtgtggaggtggattccgctccgggcaccgatctcgccaagatcctgagtgacatgcgaagccaatatgaggtcatggccgagcagaaccggaaggatgctgaagcctggttcaccagccggactgaagaattgaaccgggaggtcgctggccacacggagcagctccagatgagcaggtccgaggttactgacctgcggcgcacccttcagggtcttgagattgagctgcagtcacagctgagcatgaaagctgccttggaagacacactggcagaaacggaggcgcgctttggagcccagctggcgcatatccaggcgctgatcagcggtattgaagcccagctgggcgatgtgcgagctgatagtgagcggcagaatcaggagtaccagcggctcatggacatcaagtcgcggctggagcaggagattgccacctaccgcagcctgctcgagggacaggaagatcactacaacaatttgtctgcctccaaggtcctctgaggcagcaggctctggggcttctgctgtcctttggagggtgtcttctgggtagagggatgggaaggaagggacccttacccccggctcttctcctgacctgccaataaaaatttatggtccaaggga

SEQ ID NO: 97 - DNA, 2238 bp, Homo sapiens
aacagagccaccttctgcgtcctgctgagctctgttctctccagcacctcccaacccactagtgcctggttctcttgctccaccaggaacaagccaccatgtctcgccagtcaagtgtgtccttccggagcgggggcagtcgtagcttcagcaccgcctctgccatcaccccgtctgtctcccgcaccagcttcacctccgtgtcccggtccgggggtggcggtggtggtggcttcggcagggtcagccttgcgggtgcttgtggagtgggtggctatggcagccggagcctctacaacctggggggctccaagaggatatccatcagcactagtggtggcagcttcaggaaccggtttggtgctggtgctggaggcggctatggctttggaggtggtgccggtagtggatttggtttcggcggtggagctggtggtggctttgggctcggtggcggagctggctttggaggtggcttcggtggccctggctttcctgtctgccctcctggaggtatccaagaggtcactgtcaaccagagtctcctgactcccctcaacctgcaaatcgaccccagcatccagagggtgaggaccgaggagcgcgagcagatcaagaccctcaacaataagtttgcctccttcatcgacaaggtgcggttcctggagcagcagaacaaggttctggacaccaagtggaccctgctgcaggagcagggcaccaagactgtgaggcagaacctggagccgttgttcgagcagtacatcaacaacctcaggaggcagctggacagcatcgtgggggaacggggccgcctggactcagagctgagaaacatgcaggacctggtggaagacttcaagaacaagtatgaggatgaaatcaacaagcgtaccactgctgagaatgagtttgtgatgctgaagaaggatgtagatgctgcctacatgaacaaggtggagctggaggccaaggttgatgcactgatggatgagattaacttcatgaagatgttctttgatgcggagctgtcccagatgcagacgcatgtctctgacacctcagtggtcctctccatggacaacaaccgcaacctggacctggatagcatcatcgctgaggtcaaggcccagtatgaggagattgccaaccgcagccggacagaagccgagtcctggtatcagaccaagtatgaggagctgcagcagacagctggccggcatggcgatgacctccgcaacaccaagcatgagatctctgagatgaaccggatgatccagaggctgagagccgagattgacaatgtcaagaaacagtgcgccaatctgcagaacgccattgcggatgccgagcagcgtggggagctggccctcaaggatgccaggaacaagctggccgagctggaggaggccctgcagaaggccaagcaggacatggcccggctgctgcgtgagtaccaggagctcatgaacaccaagctggccctggacgtggagatcgccacttaccgcaagctgctggagggcgaggaatgcagactcagtggagaaggagttggaccagtcaacatctctgttgtcacaagcagtgtttcctctggatatggcagtggcagtggctatggcggtggcctcggtggaggtcttggcggcggcctcggtggaggtcttgccggaggtagcagtggaagctactactccagcagcagtgggggtgtcggcctaggtggtgggctcagtgtggggggctctggcttcagtgcaagcagtggccgagggctgggggtgggctttggcagtggcgggggtagcagctccagcgtcaaatttgtctccaccacctcctcctcccggaagagcttcaagagctaagaacctgctgcaagtcactgccttccaagtgcagcaacccagcccatggagattgcctcttctaggcagttgctcaagccatgttttatccttttctggagagtagtctagaccaagccaattgcagaaccacattctttggttcccaggagagccccattcccagcccctggtctcccgtgccgcagttctatattctgcttcaaatcagccttcaggtttcccacagcatggcccctgctgacacgagaacccaaagttttcccaaatctaaatcatcaaaacagaatccccaccccaatcccaaattttgttttggttctaactacctccagaatgtgttcaataaaatgcttttataatataa

SEQ ID NO: 98 - DNA, 14923 bp, Homo sapiens
acttcagtccggggaacagcggtgcgagctccaggcccatgcactgaggaggcggaaacaaggggagcccccagagctccatcaagccccctccaaaggctcccctacccggtccacgccccccaccccccctccccgcctcctcccaattgtgcatttttgcagccggaggcggctccgagatggggctgtgagcttcgcccggggagggggaaagagcagcgaggagtgaagcgggggggtggggtgaagggtttggatttcggggcagggggcgcacccccgtcagcaggccctccccaaggggctcggaactctacctcttcacccacgcccctggtgcgctttgccgaaggaaagaataagaacagagaaggaggagggggaaaggaggaaaagggggaccccccaactggggggggtgaaggagagaagtagcaggaccagaggggaaggggctgctgcttgcatcagcccacaccatgctgaccccgccgttgctcctgctgctgcccctgctctcagctctggtcgcggcggctatcgacgcccctaagacttgcagccccaagcagtttgcctgcagagatcaaataacctgtatctcaaagggctggcggtgcgacggtgagagggactgcccagacggatctgacgaggcccctgagatttgtccacagagtaaggcccagcgatgccagccaaacgagcataactgcctgggtactgagctgtgtgttcccatgtcccgcctctgcaatggggtccaggactgcatggacggctcagatgaggggccccactgccgagagctccaaggcaactgctctcgcctgggctgccagcaccattgtgtccccacactcgatgggcccacctgctactgcaacagcagctttcagcttcaggcagatggcaagacctgcaaagattttgatgagtgctcagtgtacggcacctgcagccagctatgcaccaacacagacggctccttcatatgtggctgtgttgaaggatacctcctgcagccggataaccgctcctgcaaggccaagaacgagccagtagaccggccccctgtgctgttgatagccaactcccagaacatcttggccacgtacctgagtggggcccaggtgtctaccatcacacctacgagcacgcggcagaccacagccatggacttcagctatgccaacgagaccgtatgctgggtgcatgttggggacagtgctgctcagacgcagctcaagtgtgcccgcatgcctggcctaaagggcttcgtggatgagcacaccatcaacatctccctcagtctgcaccacgtggaacagatggccatcgactggctgacaggcaacttctactttgtggatgacatcgatgataggatctttgtctgcaacagaaatggggacacatgtgtcacattgctagacctggaactctacaaccccaagggcattgccctggaccctgccatggggaaggtgtttttcactgactatgggcagatcccaaaggtggaacgctgtgacatggatgggcagaaccgcaccaagctcgtcgacagcaagattgtgtttcctcatggcatcacgctggacctggtcagccgccttgtctactgggcagatgcctatctggactatattgaagtggtggactatgagggcaagggccgccagaccatcatccagggcatcctgattgagcacctgtacggcctgactgtgtttgagaattatctctatgccaccaactcggacaatgccaatgcccagcagaagacgagtgtgatccgtgtgaaccgctttaacagcaccgagtaccaggttgtcacccgggtggacaagggtggtgccctccacatctaccaccagaggcgtcagccccgagtgaggagccatgcctgtgaaaacgaccagtatgggaagccgggtggctgctctgacatctgcctgctggccaacagccacaaggcgcggacctgccgctgccgttccggcttcagcctgggcagtgacgggaagtcatgcaagaagccggagcatgagctgttcctcgtgtatggcaagggccggccaggcatcatccggggcatggatatgggggccaaggtcccggatgagcacatgatccccattgaaaacctcatgaacccccgagccctggacttccacgctgagaccggcttcatctactttgccgacaccaccagctacctcattggccgccagaagattgatggcactgagcgggagaccatcctgaaggacggcatccacaatgtggagggtgtggccgtggactggatgggagacaatctgtactggacggacgatgggcccaaaaagacaatcagcgtggccaggctggagaaagctgctcagacccgcaagactttaatcgagggcaaaatgacacaccccagggctattgtggtggatccactcaatgggtggatgtactggacagactgggaggaggaccccaaggacagtcggcgtgggcggctggagagggcgtggatggatggctcacaccgagacatctttgtcacctccaagacagtgctttggcccaatgggctaagcctggacatcccggctgggcgcctctactgggtggatgccttctacgaccgcatcgagacgatactgctcaatggcacagaccggaagattgtgtatgaaggtcctgagctgaaccacgcctttggcctgtgtcaccatggcaactacctcttctggactgagtatcggagtggcagtgtctaccgcttggaacggggtgtaggaggcgcaccccccactgtgacccttctgcgcagtgagcggccccccatctttgagatccgaatgtatgatgcccagcagcagcaagttggcaccaacaaatgccgggtgaacaatggcggctgcagcagcctgtgcttggccacccctgggagccgccagtgcgcctgtgctgaggaccaggtgttggacgcagacggcgtcacttgcttggcgaacccatcctacgtgcctccaccccagtgccagccaggcgagtttgcctgtgccaacagccgctgcatccaggagcgctggaagtgtgacggagacaacgattgcctggacaacagtgatgaggccccagccctctgccatcagcacacctgcccctcggaccgattcaagtgcgagaacaaccggtgcatccccaaccgctggctctgcgacggggacaatgactgtgggaacagtgaagatgagtccaatgccacttgttcagcccgcacctgcccccccaaccagttctcctgtgccagtggccgctgcatccccatctcctggacgtgtgatctggatgacgactgtggggaccgctctgatgagtctgcttcgtgtgcctatcccacctgcttccccctgactcagtttacctgcaacaatggcagatgtatcaacatcaactggagatgcgacaatgacaatgactgtggggacaacagtgacgaagccggctgcagccactcctgttctagcacccagttcaagtgcaacagcgggcgttgcatccccgagcactggacctgcgatggggacaatgactgcggagactacagtgatgagacacacgccaactgcaccaaccaggccacgaggccccctggtggctgccacactgatgagttccagtgccggctggatggactatgcatccccctgcggtggcgctgcgatggggacactgactgcatggactccagcgatgagaagagctgtgagggagtgacccacgtctgcgatcccagtgtcaagtttggctgcaaggactcagctcggtgcatcagcaaagcgtgggtgtgtgatggcgacaatgactgtgaggataactcggacgaggagaactgcgagtccctggcctgcaggccaccctcgcacccttgtgccaacaacacctcagtctgcctgccccctgacaagctgtgtgatggcaacgacgactgtggcgacggctcagatgagggcgagctctgcgaccagtgctctctgaataacggtggctgcagccacaactgctcagtggcacctggcgaaggcattgtgtgttcctgccctctgggcatggagctggggcccgacaaccacacctgccagatccagagctactgtgccaagcatctcaaatgcagccaaaagtgcgaccagaacaagttcagcgtgaagtgctcctgctacgagggctgggtcctggaacctgacggcgagagctgccgcagcctggaccccttcaagccgttcatcattttctccaaccgccatgaaatccggcgcatcgatcttcacaaaggagactacagcgtcctggtgcccggcctgcgcaacaccatcgccctggacttccacctcagccagagcgccctctactggaccgacgtggtggaggacaagatctaccgcgggaagctgctggacaacggagccctgactagtttcgaggtggtgattcagtatggcctggccacacccgagggcctggctgtagactggattgcaggcaacatctactgggtggagagtaacctggatcagatcgaggtggccaagctggatgggaccctccggaccaccctgctggccggtgacattgagcacccaagggcaatcgcactggatccccgggatgggatcctgttttggacagactgggatgccagcctgccccgcattgaggcagcctccatgagtggggctgggcgccgcaccgtgcaccgggagaccggctctgggggctggcccaacgggctcaccgtggactacctggagaagcgcatcctttggattgacgccaggtcagatgccatttactcagcccgttacgacggctctggccacatggaggtgcttcggggacacgagttcctgtcgcacccgtttgcagtgacgctgtacgggggggaggtctactggactgactggcgaacaaacacactggctaaggccaacaagtggaccggccacaatgtcaccgtggtacagaggaccaacacccagccctttgacctgcaggtgtaccacccctcccgccagcccatggctcccaatccctgtgaggccaatgggggccagggcccctgctcccacctgtgtctcatcaactacaaccggaccgtgtcctgcgcctgcccccacctcatgaagctccacaaggacaacaccacctgctatgagtttaagaagttcctgctgtacgcacgtcagatggagatccgaggtgtggacctggatgctccctactacaactacatcatctccttcacggtgcccgacatcgacaacgtcacagtgctagactacgatgcccgcgagcagcgtgtgtactggtctgacgtgcggacacaggccatcaagcgggccttcatcaacggcacaggcgtggagacagtcgtctctgcagacttgccaaatgcccacgggctggctgtggactgggtctcccgaaacctgttctggacaagctatgacaccaataagaagcagatcaatgtggcccggctggatggctccttcaagaacgcagtggtgcagggcctggagcagccccatggccttgtcgtccaccctctgcgtgggaagctctactggaccgatggtgacaacatcagcatggccaacatggatggcagcaatcgcaccctgctcttcagtggccagaagggccccgtgggcctggctattgacttccctgaaagcaaactctactggatcagctccgggaaccataccatcaaccgctgcaacctggatgggagtgggctggaggtcatcgatgccatgcggagccagctgggcaaggccaccgccctggccatcatgggggacaagctgtggtgggctgatcaggtgtcggaaaagatgggcacatgcagcaaggctgacggctcgggctccgtggtccttcggaacagcaccaccctggtgatgcacatgaaggtctatgacgagagcatccagctggaccataagggcaccaacccctgcagtgtcaacaacggtgactgctcccagctctgcctgcccacgtcagagacgacccgctcctgcatgtgcacagccggctatagcctccggagtggccagcaggcctgcgagggcgtaggttcctttctcctgtactctgtgcatgagggaatcaggggaattcccctggatcccaatgacaagtcagatgccctggtcccagtgtccgggacctcgctggctgtcggcatcgacttccacgctgaaaatgacaccatctactgggtggacatgggcctgagcacgatcagccgggccaagcgggaccagacgtggcgtgaagacgtggtgaccaatggcattggccgtgtggagggcattgcagtggactggatcgcaggcaacatctactggacagaccagggctttgatgtcatcgaggtcgcccggctcaatggctccttccgctacgtggtgatctcccagggtctagacaagccccgggccatcaccgtccacccggagaaagggtacttgttctggactgagtggggtcagtatccgcgtattgagcggtctcggctagatggcacggagcgtgtggtgctggtcaacgtcagcatcagctggcccaacggcatctcagtggactaccaggatgggaagctgtactggtgcgatgcacggacagacaagattgaacggatcgacctggagacaggtgagaaccgcgaggtggttctgtccagcaacaacatggacatgttttcagtgtctgtgtttgaggatttcatctactggagtgacaggactcatgccaacggctctatcaagcgcgggagcaaagacaatgccacagactccgtgcccctgcgaaccggcatcggcgtccagcttaaagacatcaaagtcttcaaccgggaccggcagaaaggcaccaacgtgtgcgcggtggccaatggcgggtgccagcagctgtgcctgtaccggggccgtgggcagcgggcctgcgcctgtgcccacgggatgctggctgaagacggagcatcgtgccgcgagtatgccggctacctgctctactcagagcgcaccattctcaagagtatccacctgtcggatgagcgcaacctcaatgcgcccgtgcagcccttcgaggaccctgagcacatgaagaacgtcatcgccctggcctttgactaccgggcaggcacctctccgggcacccccaatcgcatcttcttcagcgacatccactttgggaacatccaacagatcaacgacgatggctccaggaggatcaccattgtggaaaacgtgggctccgtggaaggcctggcctatcaccgtggctgggacactctctattggacaagctacacgacatccaccatcacgcgccacacagtggaccagacccgcccaggggccttcgagcgtgagaccgtcatcactatgtctggagatgaccacccacgggccttcgttttggacgagtgccagaacctcatgttctggaccaactggaatgagcagcatcccagcatcatgcgggcggcgctctcgggagccaatgtcctgacccttatcgagaaggacatccgtacccccaatggcctggccatcgaccaccgtgccgagaagctctacttctctgacgccaccctggacaagatcgagcggtgcgagtatgacggctcccaccgctatgtgatcctaaagtcagagcctgtccaccccttcgggctggccgtgtatggggagcacattttctggactgactgggtgcggcgggcagtgcagcgggccaacaagcacgtgggcagcaacatgaagctgctgcgcgtggacatcccccagcagcccatgggcatcatcgccgtggccaacgacaccaacagctgtgaactctctccatgccgaatcaacaacggtggctgccaggacctgtgtctgctcactcaccagggccatgtcaactgctcatgccgagggggccgaatcctccaggatgacctcacctgccgagcggtgaattcctcttgccgagcacaagatgagtttgagtgtgccaatggcgagtgcatcaacttcagcctgacctgcgacggcgtcccccactgcaaggacaagtccgatgagaagccatcctactgcaactcccgccgctgcaagaagactttccggcagtgcagcaatgggcgctgtgtgtccaacatgctgtggtgcaacggggccgacgactgtggggatggctctgacgagatcccttgcaacaagacagcctgtggtgtgggcgagttccgctgccgggacgggacctgcatcgggaactccagccgctgcaaccagtttgtggattgtgaggacgcctcagatgagatgaactgcagtgccaccgactgcagcagctacttccgcctgggcgtgaagggcgtgctcttccagccctgcgagcggacctcactctgctacgcacccagctgggtgtgtgatggcgccaatgactgtggggactacagtgatgagcgcgactgcccaggtgtgaaacgccccagatgccctctgaattacttcgcctgccctagtgggcgctgcatccccatgagctggacgtgtgacaaagaggatgactgtgaacatggcgaggacgagacccactgcaacaagttctgctcagaggcccagtttgagtgccagaaccatcgctgcatctccaagcagtggctgtgtgacggcagcgatgactgtggggatggctcagacgaggctgctcactgtgaaggcaagacgtgcggcccctcctccttctcctgccctggcacccacgtgtgcgtccccgagcgctggctctgtgacggtgacaaagactgtgctgatggtgcagacgagagcatcgcagctggttgcttgtacaacagcacttgtgacgaccgtgagttcatgtgccagaaccgccagtgcatccccaagcacttcgtgtgtgaccacgaccgtgactgtgcagatggctctgatgagtcccccgagtgtgagtacccgacctgcggccccagtgagttccgctgtgccaatgggcgctgtctgagctcccgccagtgggagtgtgatggcgagaatgactgccacgaccagagtgacgaggctcccaagaacccacactgcaccagccaagagcacaagtgcaatgcctcgtcacagttcctgtgcagcagtgggcgctgtgtggctgaggcactgctctgcaacggccaggatgactgtggcgacagctcggacgagcgtggctgccacatcaatgagtgtctcagccgcaagctcagtggctgcagccaggactgtgaggacctcaagatcggcttcaagtgccgctgtcgccctggcttccggctgaaggacgacggccggacgtgtgctgatgtggacgagtgcagcaccaccttcccctgcagccagcgctgcatcaacactcatggcagctataagtgtctgtgtgtggagggctatgcaccccgcggcggcgacccccacagctgcaaggctgtgactgacgaggaaccgtttctgatcttcgccaaccggtactacctgcgcaagctcaacctggacgggtccaactacacgttacttaagcagggcctgaacaacgccgttgccttggattttgactaccgagagcagatgatctactggacagatgtgaccacccagggcagcatgatccgaaggatgcaccttaacgggagcaatgtgcaggtcctacaccgtacaggcctcagcaaccccgatgggctggctgtggactgggtgggtggcaacctgtactggtgcgacaaaggccgggacaccatcgaggtgtccaagctcaatggggcctatcggacggtgctggtcagctctggcctccgtgagcccagggctctggtggtggatgtgcagaatgggtacctgtactggacagactggggtgaccattcactgatcggccgcatcggcatggatgggtccagccgcagcgtcatcgtggacaccaagatcacatggcccaatggcctgacgctggactatgtcactgagcgcatctactgggccgacgcccgcgaggactacattgaatttgccagcctggatggctccaatcgccacgttgtgctgagccaggacatcccgcacatctttgcactgaccctgtttgaggactacgtctactggaccgactgggaaacaaagtccattaaccgagcccacaagaccacgggcaccaacaaaacgctcctcatcagcacgctgcaccggcccatggacctgcatgtcttccatgccctgcgccagccagacgtgcccaatcacccctgcaaggtcaacaatggtggctgcagcaacctgtgcctgctgtcccccgggggagggcacaaatgtgcctgccccaccaacttctacctgggcagcgatgggcgcacctgtgtgtccaactgcacggctagccagtttgtatgcaagaacgacaagtgcatccccttctggtggaagtgtgacaccgaggacgactgcggggaccactcagacgagcccccggactgccctgagttcaagtgccggcccggacagttccagtgctccacaggtatctgcacaaaccctgccttcatctgcgatggcgacaatgactgccaggacaacagtgacgaggccaactgtgacatccacgtctgcttgcccagtcagttcaaatgcaccaacaccaaccgctgtattcccggcatcttccgctgcaatgggcaggacaactgcggagatggggaggatgagagggactgccccgaggtgacctgcgcccccaaccagttccagtgctccattaccaaacggtgcatcccccgggtctgggtctgcgaccgggacaatgactgtgtggatggcagtgatgagcccgccaactgcacccagatgacctgtggtgtggacgagttccgctgcaaggattcgggccgctgcatcccagcgcgttggaagtgtgacggagaggatgactgtggggatggctcggatgagcccaaggaagagtgtgatgaacgcacctgtgagccataccagttccgctgcaagaacaaccgctgcgtgcccggccgctggcagtgcgactacgacaacgattgcggtgacaactccgatgaagagagctgcacccctcggccctgctccgagagtgagttctcctgtgccaacggccgctgcatcgcggggcgctggaaatgcgatggagaccacgactgcgcggacggctcggacgagaaagactgcaccccccgctgtgacatggaccagttccagtgcaagagcggccactgcatccccctgcgctggcgctgtgacgcagacgccgactgcatggacggcagcgacgaggaggcctgcggcactggcgtgcggacctgccccctggacgagttccagtgcaacaacaccttgtgcaagccgctggcctggaagtgcgatggcgaggatgactgtggggacaactcagatgagaaccccgaggagtgtgcccggttcgtgtgccctcccaaccggcccttccgttgcaagaatgaccgcgtctgtctgtggatcgggcgccaatgcgatggcacggacaactgtggggatgggactgatgaagaggactgtgagccccccacagcccacaccacccactgcaaagacaagaaggagtttctgtgccggaaccagcgctgcctctcctcctccctgcgctgcaacatgttcgatgactgcggggacggctctgacgaggaggactgcagcatcgaccccaagctgaccagctgcgccaccaatgccagcatctgtggggacgaggcacgctgcgtgcgcaccgagaaagcggcctactgtgcctgccgctcgggcttccacaccgtgcccggccagcccggatgccaagacatcaacgagtgcctgcgcttcggcacctgctcccagctctgcaacaacaccaagggcggccacctctgcagctgcgctcggaacttcatgaagacgcacaacacctgcaaggccgaaggctctgagtaccaggtcctgtacatcgctgatgacaatgagatccgcagcctgttccccggccacccccattcggcttacgagcaggcattccagggtgacgagagtgtccgcattgatgctatggatgtccatgtcaaggctggccgtgtctattggaccaactggcacacgggcaccatctcctaccgcagcctgccacctgctgcgcctcctaccacttccaaccgccaccggcgacagattgaccggggtgtcacccacctcaacatttcagggctgaagatgcccagaggcatcgccatcgactgggtggccggaaacgtgtactggaccgactcgggccgagatgtgattgaggtggcgcagatgaagggcgagaaccgcaagacgctcatctcgggcatgattgacgagccccacgccattgtggtggacccactgagggggaccatgtactggtcagactggggcaaccaccccaagattgagacggcagcgatggatgggacgcttcgggagacactggtgcaggacaacattcagtggcccacaggcctggccgtggattatcacaatgagcggctgtactgggcagacgccaagctttcagtcatcggcagcatccggctcaatggcacggaccccattgtggctgctgacagcaaacgaggcctaagtcaccccttcagcatcgacgtctttgaggattacatctatggtgtcacctacatcaataatcgtgtcttcaagatccataagtttggccacagccccttggtcaacctgacagggggcctgagccacgcctctgacgtggtcctttaccatcagcacaagcagcccgaagtgaccaacccatgtgaccgcaagaaatgcgagtggctctgcctgctgagccccagtgggcctgtctgcacctgtcccaatgggaagcggctggacaacggcacatgcgtgcctgtgccctctccaacgccccccccagatgctccccggcctggaacctgtaacctgcagtgcttcaacggtggcagctgtttcctcaatgcacggaggcagcccaagtgccgctgccaaccccgctacacgggtgacaagtgtgaactggaccagtgctgggagcactgtcgcaatgggggcacctgtgctgcctccccctctggcatgcccacgtgccggtgccccacgggcttcacgggccccaaatgcacccagcaggtgtgtgcgggctactgtgccaacaacagcacctgcactgtcaaccagggcaaccagccccagtgccgatgcctacccggcttcctgggcgaccgctgccagtaccggcagtgctctggctactgtgagaactttggcacatgccagatggctgctgatggctcccgacaatgccgctgcactgcctactttgagggatcgaggtgtgaggtgaacaagtgcagccgctgtctcgaaggggcctgtgtggtcaacaagcagagtggggatgtcacctgcaactgcacggatggccgggtggcccccagctgtctgacctgcgtcggccactgcagcaatggcggctcctgtaccatgaacagcaaaatgatgcctgagtgccagtgcccaccccacatgacagggccccggtgtgaggagcacgtcttcagccagcagcagccaggacatatagcctccatcctaatccctctgctgttgctgctgctgctggttctggtggccggagtggtattctggtataagcggcgagtccaaggggctaagggcttccagcaccaacggatgaccaacggggccatgaacgtggagattggaaaccccacctacaagatgtacgaaggcggagagcctgatgatgtgggaggcctactggacgctgactttgccctggaccctgacaagcccaccaacttcaccaaccccgtgtatgccacactctacatggggggccatggcagtcgccactccctggccagcacggacgagaagcgagaactcctgggccggggccctgaggacgagataggggaccccttggcatagggccctgccccgtcggactgcccccagaaagcctcctgccccctgccagtgaagtccttcagtgagcccctccccagccagcccttccctggccccgccggatgtataaatgtaaaaatgaaggaattacattttatatgtgagcgagcaagccggcaagcgagcacagtattatttctccatcccctccctgcctgctccttggcacccccatgctgccttcagggagacaggcagggagggcttggggctgcacctcctaccctcccaccagaacgcaccccactgggagagctggtggtgcagccttcccctccctgtataagacactttgccaaggctctcccctctcgccccatccctgcttgcccgctcccacagcttcctgagggctaattctgggaagggagagttctttgctgcccctgtctggaagacgtggctctgggtgaggtaggcgggaaaggatggagtgttttagttcttgggggaggccaccccaaaccccagccccaactccaggggcacctatgagatggccatgctcaacccccctcccagacaggccctccctgtctccagggcccccaccgaggttcccagggctggagacttcctctggtaaacattcctccagcctcccctcccctggggacgccaaggaggtgggccacacccaggaagggaaagcgggcagccccgttttggggacgtgaacgttttaataatttttgctgaattcctttacaactaaataacacagatattgttataaataaaattgtaaaaaaaaaaaaaaaaaaaaa

SEQ ID NO: 99 - DNA, 5640 bp, Homo sapiens
gcgccttgggcggatgcacggactgagaggcgcttggctgggctctcagcccgccactgccacccggccccgtgcgctcattggtcgggatgtgtcggctcagcagcctccaacttctcccgaatcccactgattttaggtgatgggcaagtcagaaagtcagatggatataactgatatcaacactccaaagccaaagaagaaacagcgatggactccactggagatcagcctctcggtccttgtcctgctcctcaccatcatagctgtgacaatgatcgcactctatgcaacctacgatgatggtatttgcaagtcatcagactgcataaaatcagctgctcgactgatccaaaacatggatgccaccactgagccttgtacagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtataaatgaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgccaatgctacggctaaacctgaagatcgaaatgatccaatgcttctgtataacaagatgacattggcccagatccaaaataacttttcactagagatcaatgggaagccattcagctggttgaatttcacaaatgaaatcatgtcaactgtgaatattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacctcgagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggaaacttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttggtgatcttcaaaagaagcattgcagcccttggctagacttgccaacaccacagaaatggggaattctctaatcgaaagaaaatgggccctaggggtcactgtactgacttgagggtgattaacagagagggcaccatcacaatacagataacattaggttgtcctagaaagggtgtggagggaggaagggggtctaaggtctatcaagtcaatcatttctcactgtgtacataatgcttaatttctaaagataatattactgtttatttctgtttctcatatggtctaccagtttgctgatgtccctagaaaacaatgcaaaacctttgaggtagaccaggatttctaatcaaaagggaaaagaagatgttgaagaatacagttaggcaccagaagaacagtaggtgacactatagtttaaaacacattgcctaactactagtttttacttttatttgcaacatttacagtccttcaaaatccttccaaagaattcttatacacattggggccttggagcttacatagttttaaactcatttttgccatacatcagttattcattctgtgatcatttattttaagcactcttaaagcaaaaaatgaatgtctaaaattgttttttgttgtacctgctttgactgatgctgagattcttcaggcttcctgcaattttctaagcaatttcttgctctatctctcaaaacttggtatttttcagagatttatataaatgtaaaaataataatttttatatttaattattaactacatttatgagtaactattattataggtaatcaatgaatattgaagtttcagcttaaaataaacagttgtgaaccaagatctataaagcgatatacagatgaaaatttgagactatttaaacttataaatcatattgatgaaaagatttaagcacaaactttagggtaaaaattgccattggacagttgtctagagatatatatacttgtggttttcaaattggactttcaaaattaaatctgtccctgagagtgtctctgataaaagggcaaatctgcacctatgtagctctgcatctcctgtcttttcaggtttgtcatcagatggaaatattttgataataaattgaaattgtgaactcattgctccctaagactgtgacaactgtctaactttagaagtgcatttctgaatagaaatgggaggcctctgatggaccttctagaattataagtcacaaagagttctggaaaagaactgtttactgcttgataggaattcatcttttgaggcttctgttcctctcttttcctgttgtattgactattttcgttcattacttgattaagattttacaaaagaggagcacttccaaaattcttatttttcctaacaaaagatgaaagcagggaatttctatctaaatgatgagtattagttccctgtctcttgaaaaatgcccatttgcctttaaaaaaaaaagttacagaaatactataacatatgtacataaattgcataaagcataagtatacagttcaataaacttaactttaactgaacaatggccctgtagccagcacctgtaagaaacagagcagtaccagcgctctaaaagcacctccttgtcactttattactcccagaacaacaactatcctgacttctaatatcattcactagctttgcctggttttgtcttttatgcagatagaatcaatcagtatgtattcttttgtgcctggcttctttctctcagccttacatttgtgagattcctctgtattgtgctgattgtggatcttttcattctcattgcagaataatgttctattgtgggacttattacaatttgttcatcctattgttgatgggcacttgagaactttccattttggcgctattacaaatagtgcaactatgaatgtactgcatgttaccatcttacttgagcctttaatggacttatttcttcaaatccttccaaaaattattataagcattgaaattatagtttcaagccaactgtggatacccttaccctttcctcctttatcacaaccaccgttacaagtatacttatatttccctaaaatacatttaaaacttacctaagtgacatttgtagttggagtaataggagcttccagctctaataaaacagctgtctctaacttattttatttccatcatgtcagagcaggtgaagagccagaagtgaagagtgactagtacaaattataaaaagccactagactcttcactgttagctttttaaaacattaggctcccatccctatggaggaacaactctccagtgcctggatcccctctgtctacaaatataagattttctgggcctaaaggatagatcaaagtcaaaaatagcaatgcctccctatccctcacacatccagacatcatgaattttacatggtactcttgttgagttctgtagagccttctgatgtctctaaagcactaccgattctttggagttgtcacatcagataagacatatctctaattccatccataaatccagttctactatggctgagttctggtcaaagaaagaaagtttagaagctgagacacaaagggttgggagctgatgaaactcacaaatgatggtaggaagaagctctcgacaatacccgttggcaaggagtctgcctccatgctgcagtgttcgagtggattgtaggtgcaagatggaaaggattgtaggtgcaagctgtccagagaaaagagtccttgttccagccctattctgccactcctgacagggtgaccttgggtatttgcaatattcctttgggcctctgcttctctcacctaaaaaaagagaattagattatattggtggttctcagcaagagaaggagtatgtgtccaatgctgccttcccatgaatctgtctcccagttatgaatcagtgggcaggataaactgaaaactcccatttacgtgtctgaatcgagtgagacaaaattttagtccaaataacaagtaccaaagttttatcaagtttgggtctgtgctgctgttactgttaaccatttaagtggggcaaaaccttgctaattttctcaaaagcatttatcattcttgttgccacagctggagctctcaaactaaaagacatttgttattttggaaagaagaaagactctattctcaaagtttcctaatcagaaatttttatcagtttccagtctcaaaaatacaaaataaaaacaaacgtttttaatacta

SEQ ID NO: 100 - DNA, 406 bp, Homo sapiens
aagccgcgcgtccagttgcttggagaagcccgttcaccgcctccagctgctgctctcctcgacatggaccctgagacctgcccctgcccttctggtggctcctgcacctgcgcggactcctgcaagtgcgagggatgcaaatgcacctcctgcaagaagagctgctgctcctgctgccctgcggagtgtgagaagtgtgccaaggactgtgtgtgcaaaggcggagaggcagctgaggcagaagcagagaagtgcagctgctgccagtgagaaggcacccctccgtgtggagcacgtggagatagtgccaggtggctcagtgccacctatgcctgtggtgaagtgtggctggtgtccccttcccctgctgaccttggaggaatgacaataaatcccatgaacagca

SEQ ID NO: 101 - DNA, 7451 bp, Homo sapiens
gcagatcaccgcggttcctgggcagggcacggaaggctaagcaaggctgacctgctgcagctcccgcctcgtgcgctcgccccacccggccgccgcccgagcgctcgagaaagtcctctcgggagaagcagcgcctgttcccggggcagatccaggttcaggtcctggctataagtcaccatggcacagcaagctgccgataagtatctctatgtggataaaaacttcatcaacaatccgctggcccaggccgactgggctgccaagaagctggtatgggtgccttccgacaagagtggctttgagccagccagcctcaaggaggaggtgggcgaagaggccatcgtggagctggtggagaatgggaagaaggtgaaggtgaacaaggatgacatccagaagatgaacccgcccaagttctccaaggtggaggacatggcagagctcacgtgcctcaacgaagcctcggtgctgcacaacctcaaggagcgttactactcagggctcatctacacctattcaggcctgttctgtgtggtcatcaatccttacaagaacctgcccatctactctgaagagattgtggaaatgtacaagggcaagaagaggcacgagatgccccctcacatctatgccatcacagacaccgcctacaggagtatgatgcaagaccgagaagatcaatccatcttgtgcactggtgaatctggagctggcaagacggagaacaccaagaaggtcatccagtatctggcgtacgtggcgtcctcgcacaagagcaagaaggaccagggcgagctggagcggcagctgctgcaggccaaccccatcctggaggccttcgggaacgccaagaccgtgaagaatgacaactcctcccgcttcggcaaattcattcgcatcaactttgatgtcaatggctacattgttggagccaacattgagacttatcttttggagaaatctcgtgctatccgccaagccaaggaagaacggaccttccacatcttctattatctcctgtctggggctggagagcacctgaagaccgatctcctgttggagccgtacaacaaataccgcttcctgtccaatggacacgtcaccatccccgggcagcaggacaaggacatgttccaggagaccatggaggccatgaggattatgggcatcccagaagaggagcaaatgggcctgctgcgggtcatctcaggggttcttcagctcggcaacatcgtcttcaagaaggagcggaacactgaccaggcgtccatgcccgacaacacagctgcccaaaaggtgtcccatctcttgggtatcaatgtgaccgatttcaccagaggaatcctcaccccgcgcatcaaggtgggacgggattacgtccagaaggcgcagactaaagagcaggctgactttgccatcgaggccttggccaaggcgacctatgagcggatgttccgctggctggtgctgcgcatcaacaaggctctggacaagaccaagaggcagggcgcctccttcatcgggatcctggacattgccggcttcgagatctttgatctgaactcgtttgagcagctgtgcatcaattacaccaatgagaagctgcagcagctcttcaaccacaccatgttcatcctggagcaggaggagtaccagcgcgagggcatcgagtggaacttcatcgactttggcctcgacctgcagccctgcatcgacctcattgagaagccagcaggccccccgggcattctggccctgctggacgaggagtgctggttccccaaagccaccgacaagagcttcgtggagaaggtgatgcaggagcagggcacccaccccaagttccagaagcccaagcagctgaaggacaaagctgatttctgcattatccactatgccggcaaggtggattacaaagctgacgagtggctgatgaagaacatggatcccctgaatgacaacatcgccacactgctccaccagtcctctgacaagtttgtctcggagctgtggaaggatgtggaccgcatcatcggcctggaccaggtggccggcatgtcggagaccgcactgcccggggccttcaagacgcggaagggcatgttccgcactgtggggcagctttacaaggagcagctggccaagctgatggctacgctgaggaacacgaaccccaactttgtccgctgcatcatccccaaccacgagaagaaggccggcaagctggacccgcatctcgtgctggaccagctgcgctgcaacggtgttctcgagggcatccgtatctgccgccagggcttccccaacagggtggtcttccaggagtttcggcagagatatgagatcctgactccaaactccattcccaagggtttcatggacgggaagcaggcgtgcgtgctcatgataaaagccctggagctcgacagcaatctgtaccgcattggccagagcaaagtcttcttccgtgccggtgtgctggcccacctggaggaggagcgagacctgaagatcaccgacgtcatcatagggttccaggcctgctgcaggggctacctggccaggaaagcatttgccaagcggcagcagcagcttaccgccatgaaggtcctccagcggaactgcgctgcctacctgaagctgcggaactggcagtggtggcggctcttcaccaaggtcaagccgctgctgcaggtgagccggcaggaggaggagatgatggccaaggaggaggagctggtgaaggtcagagagaagcagctggctgcggagaacaggctcacggagatggagacgctgcagtctcagctcatggcagagaaattgcagctgcaggagcagctccaggcagaaaccgagctgtgtgccgaggctgaggagctccgggcccgcctgaccgccaagaagcaggaattagaagagatctgccatgacctagaggccagggtggaggaggaggaggagcgctgccagcacctgcaggcggagaagaagaagatgcagcagaacatccaggagcttgaggagcagctggaggaggaggagagcgcccggcagaagctgcagctggagaaggtgaccaccgaggcgaagctgaaaaagctggaggaggagcagatcatcctggaggaccagaactgcaagctggccaaggaaaagaaactgctggaagacagaatagctgagttcaccaccaacctcacagaagaggaggagaaatctaagagcctcgccaagctcaagaacaagcatgaggcaatgatcactgacttggaagagcgcctccgcagggaggagaagcagcgacaggagctggagaagacccgccggaagctggagggagactccacagacctcagcgaccagatcgccgagctccaggcccagatcgcggagctcaagatgcagctggccaagaaagaggaggagctccaggccgccctggccagagtggaagaggaagctgcccagaagaacatggccctcaagaagatccgggagctggaatctcagatctctgaactccaggaagacctggagtctgagcgtgcttccaggaataaagctgagaagcagaaacgggaccttggggaagagctagaggctctgaaaacagagttggaggacacgctggattccacagctgcccagcaggagctcaggtcaaaacgtgagcaggaggtgaacatcctgaagaagaccctggaggaggaggccaagacccacgaggcccagatccaggagatgaggcagaagcactcacaggccgtggaggagctggcggagcagctggagcagacgaagcgggtgaaagcaaacctcgagaaggcaaagcagactctggagaacgagcggggggagctggccaacgaggtgaaggtgctgctgcagggcaaaggggactcggagcacaagcgcaagaaagtggaggcgcagctgcaggagctgcaggtcaagttcaacgagggagagcgcgtgcgcacagagctggccgacaaggtcaccaagctgcaggtggagctggacaacgtgaccgggcttctcagccagtccgacagcaagtccagcaagctcaccaaggacttctccgcgctggagtcccagctgcaggacactcaggagctgctgcaggaggagaaccggcagaagctgagcctgagcaccaagctcaagcaggtggaggacgagaagaattccttccgggagcagctggaggaggaggaggaggccaagcacaacctggagaagcagatcgccaccctccatgcccaggtggccgacatgaaaaagaagatggaggacagtgtggggtgcctggaaactgctgaggaggtgaagaggaagctccagaaggacctggagggcctgagccagcggcacgaggagaaggtggccgcctacgacaagctggagaagaccaagacgcggctgcagcaggagctggacgacctgctggtggacctggaccaccagcgccagagcgcgtgcaacctggagaagaagcagaagaagtttgaccagctcctggcggaggagaagaccatctctgccaagtatgcagaggagcgcgaccgggctgaggcggaggcccgagagaaggagaccaaggctctgtcgctggcccgggccctggaggaagccatggagcagaaggcggagctggagcggctcaacaagcagttccgcacggagatggaggaccttatgagctccaaggatgatgtgggcaagagtgtccacgagctggagaagtccaagcgggccctagagcagcaggtggaggagatgaagacgcagctggaagagctggaggacgagctgcaggccaccgaagatgccaagctgcggttggaggtcaacctgcaggccatgaaggcccagttcgagcgggacctgcagggccgggacgagcagagcgaggagaagaagaagcagctggtcagacaggtgcgggagatggaggcagagctggaggacgagaggaagcagcgctcgatggcagtggccgcccggaagaagctggagatggacctgaaggacctggaggcgcacatcgactcggccaacaagaaccgggacgaagccatcaaacagctgcggaagctgcaggcccagatgaaggactgcatgcgcgagctggatgacacccgcgcctctcgtgaggagatcctggcccaggccaaagagaacgagaagaagctgaagagcatggaggccgagatgatccagttgcaggaggaactggcagccgcggagcgtgccaagcgccaggcccagcaggagcgggatgagctggctgacgagatcgccaacagcagcggcaaaggagccctggcgttagaggagaagcggcgtctggaggcccgcatcgcccagctggaggaggagctggaggaggagcagggcaacacggagctgatcaacgaccggctgaagaaggccaacctgcagatcgaccagatcaacaccgacctgaacctggagcgcagccacgcccagaagaacgagaatgctcggcagcagctggaacgccagaacaaggagcttaaggtcaagctgcaggagatggagggcactgtcaagtccaagtacaaggcctccatcaccgccctcgaggccaagattgcacagctggaggagcagctggacaacgagaccaaggagcgccaggcagcctgcaaacaggtgcgtcggaccgagaagaagctgaaggatgtgctgctgcaggtggatgacgagcggaggaacgccgagcagtacaaggaccaggccgacaaggcatctacccgcctgaagcagctcaagcggcagctggaggaggccgaagaggaggcccagcgggccaacgcctcccgccggaaactgcagcgcgagctggaggacgccactgagacggccgatgccatgaaccgcgaagtcagctccctaaagaacaagctcaggcgcggggacctgccgtttgtcgtgccccgccgaatggcccggaaaggcgccggggatggctccgacgaagaggtagatggcaaagcggatggggctgaggccaaacctgccgaataagcctcttctcctgcagcctgagatggatggacagacagacaccacagcctccccttcccagaccccgcagcacgcctctccccaccttcttgggactgctgtgaacatgcctcctcctgccctccgccccgtccccccatcccgtttccctccaggtgttgttgagggcatttggcttcctctgctgcatccccttccagctccctcccctgctcagaatctgataccaaagagacagggcccgggcccaggcagagagcgaccagcaggctcctcagccctctcttgccaaaaagcacaagatgttgaggcgagcagggcaggcccccggggaggggccagagttttctatgaatctatttttcttcagactgaggccttttggtagtcggagcccccgcagtcgtcagcctccctgacgtctgccaccagcgcccccactcctcctcctttctttgctgtttgcaatcacacgtggtgacctcacacacctctgccccttgggcctcccactcccatggctctgggcggtccagaaggagcaggccctgggcctccacctctgtgcagggcacagaaggctggggtggggggaggagtggattcctccccaccctgtcccaggcagcgccactgtccgctgtctccctcctgattctaaaatgtctcaagtgcaatgccccctcccctcctttaccgaggacagcctgcctctgccacagcaaggctgtcggggtcaagctggaaaggccagcagccttccagtggcttctcccaacactcttggggaccaaatatatttaatggttaagggacttgtcccaagtctgacagccagagcgttagaggggccagcggccctcccaggcgatcttgtgtctactctaggactgggcccgagggtggtttacctgcaccgttgactcagtatagtttaaaaatctgccacctgcacaggtatttttgaaagcaaaataaggttttcttttttcccctttcttgtaataaatgataaaattccgagtctttctcactgcctttgtttagaagagagtagctcgtcctcactggtctacactggttgccgaatttacttgtattcctaactgttttgtatatgctgcattgagacttacggcaagaaggcattttttttttttaaaggaaacaaactctcaaatcatgaagtgatataaaagctgcatatgcctacaaagctctgaattcaggtcccagttgctgtcacaaaggagtgagtgaaactcccaccctacccccttttttatataataaaagtgccttagcatgtgttgcagctgtcaccactacagtaagctggtttacagatgttttccactgagcatcacaataaagagaaccatgtgcta

SEQ ID NO: 102 - DNA, 1371 bp, Homo sapiens
actccactgcgactgcagaactcagagctgctcttcctctgtggccagttggggaccagcatcatgaagtggatggtggtggtcttggtctgcctccagctcttggaggcagcagtggtcaaagtgcccctgaagaaatttaagtctatccgtgagaccatgaaggagaagggcttgctgggggagttcctgaggacccacaagtatgatcctgcttggaagtaccgctttggtgacctcagcgtgacctacgagcccatggcctacatggatgctgcctactttggtgagatcagcatcgggactccaccccagaacttcctggtcctttttgacaccggctcctccaacttgtgggtgccctctgtctactgccagagccaggcctgcaccagtcactcccgcttcaaccccagcgagtcgtccacctactccaccaatgggcagaccttctccctgcagtatggcagtggcagcctcaccggcttctttggctatgacaccctgactgtccagagcatccaggtccccaaccaggagttcggcttgagtgagaatgagcctggtaccaacttcgtctatgcgcagtttgatggcatcatgggcctggcctaccctgctctgtccgtggatgaggccaccacagctatgcagggcatggtgcaggagggcgccctcaccagccccgtcttcagcgtctacctcagcaaccagcagggctccagcgggggagcggttgtctttgggggtgtggatagcagcctgtacacggggcagatctactgggcgcctgtcacccaggaactctactggcagattggcattgaagagttcctcatcggcggccaggcctccggctggtgttctgagggttgccaggccatcgtggacacaggcacctctctgctcactgtgccccagcagtacatgagtgctcttctgcaggccacaggggcccaggaggatgagtatggacagtttctcgtgaactgtaacagcattcagaatctgcccagcttgaccttcatcatcaatggtgtggagttccctctgccaccttcctcctatatcctcagtaacaacggctactgcaccgtgggagtcgagcccacctacctgtcctcccagaacggccagcccctgtggatcctcggggatgtcttcctcaggtcctactattccgtctacgacttgggcaacaacagagtaggctttgccactgccgcctagacttgctgcctcgacacgtgggctcccctcttcctcttgaccctgcaccctcctagggcattgtatctgtctttccactctggattcagccttctttttctggactctggactttctctaataataaatagttcttcttta

SEQ ID NO: 103 - DNA, 4275 bp, Homo sapiens
gagcagagtttcagttttggcagcagcgtccagtgccctgccagtagctcctagagaggcaggggttaccaactggccagcaggctgtgtccctgaagtcagatcaacgggagagaaggaagtggctaaaacattgcacaggagaagtcggcctgagtggtgcggcgctcgggacccaccagcaatgctgctcttcgtgctcacctgcctgctggcggtcttcccagccatctccacgaagagtcccatatttggtcccgaggaggtgaatagtgtggaaggtaactcagtgtccatcacgtgctactacccacccacctctgtcaaccggcacacccggaagtactggtgccggcagggagctagaggtggctgcataaccctcatctcctcggagggctacgtctccagcaaatatgcaggcagggctaacctcaccaacttcccggagaacggcacatttgtggtgaacattgcccagctgagccaggatgactccgggcgctacaagtgtggcctgggcatcaatagccgaggcctgtcctttgatgtcagcctggaggtcagccagggtcctgggctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagagcgctggtctccaccctggtgcccctgggcctggtgctggcagtgggagccgtggctgtgggggtggccagagcccggcacaggaagaacgtcgaccgagtttcaatcagaagctacaggacagacattagcatgtcagacttcgagaactccagggaatttggagccaatgacaacatgggagcctcttcgatcactcaggagacatccctcggaggaaaagaagagtttgttgccaccactgagagcaccacagagaccaaagaacccaagaaggcaaaaaggtcatccaaggaggaagccgagatggcctacaaagacttcctgctccagtccagcaccgtggccgccgaggcccaggacggcccccaggaagcctagacggtgtcgccgcctgctccctgcacccatgacaatcaccttcagaatcatgtcgatcctggggccctcagctcctggggaccccactccctgctctaacacctgcctaggtttttcctactgtcctcagaggcgtgctggtcccctcctcagtgacatcaaagcctggcctaattgttcctattggggatgagggtggcatgaggaggtcccacttgcaacttctttctgttgagagaacctcaggtacggagaagaatagaggtcctcatgggtcccttgaaggaagagggaccagggtgggagagctgattgcagaaaggagagacgtgcagcgcccctctgcacccttatcatgggatgtcaacagaatttttccctccactccatccctccctcccgtccttcccctcttcttctttccttccatcaaaagatgtatttgaattcatactagaattcaggtgctttgctagatgctgtgacaggtatgccaccaacactgctcacagcctttctgaggacaccagtgaaagaagccacagctcttcttggcgtatttatactcactgagtcttaacttttcaccaggggtgctcacctctgcccctattgggagaggtcataaaatgtctcgagtcctaaggccttaggggtcatgtatgatgagcatacacacaggtaattataaacccacattcttaccatttcacacataagaaaattgaggtttggaagagtgaagcgtttttctttttcttttttttttttgagacggagtctctcactgtcgcccaggctggagtgcagtggcgcaatctcggctcactgcaacctccgcctcccaggttgacaccattctcctgcctcaccctcccaagtagctgggactacaggcgcctgccagcacgcctggctaattttttgtatttttagtagagacagggtttcaccgtgttagccaggatggtctcgatctcctgacctcgtgatccgcctgcctctgcctcccaaagtgctgggattacaggcgtgagccaccgcgtccggcctctttttttcttttcttttttttgagacaaagtctcactgtgtcacccagactggaatgcagtgacacaatctcggctcactgaaacctctgccttccaggttcaagctattctcatgcctcagcctctcaagtagctgggactacagatgtgggccaccatgtctggctaattttttttttttttttttttttttgtagagacagggtttcgccatgttgacgagactggtctcgaactcctggcctcaagtgatctgccgcctcagcttctcaaagtactgggattatataggcatgagccactgagcctggccctgaagcgtttttctcaaaggccctcagtgagataaattagatttggcatctcctgtcctgggccagggatctctctacaagagcccctgcccctctgttggaggcacagttttagaataaggaggaggagggagaagagaaaatgtaaaggagggagatctttcccaggccgcaccatttctgtcactcacatggacccaagataaaagaatggccaaaccctcacaacccctgatgtttgaagagttccaagttgaagggaaacaaagaagtgtttgatggtgccagagaggggctgctctccagaaagctaaaatttaatttcttttttcctctgagttctgtacttcaaccagcctacaagctggcacttgctaacaaatcagaaatatgacaattaatgattaaagactgtgattgccaccaa

SEQ ID NO: 104 - DNA, 2305 bp, Homo sapiens
gcacagcagctgcacgcgccgtggctccggatctcttcgtctttgcagcgtagcccgagtcggtcagcgccggaggacctcagcagccatgtcgaagccccatagtgaagccgggactgccttcattcagacccagcagctgcacgcagccatggctgacacattcctggagcacatgtgccgcctggacattgattcaccacccatcacagcccggaacactggcatcatctgtaccattggcccagcttcccgatcagtggagacgttgaaggagatgattaagtctggaatgaatgtggctcgtctgaacttctctcatggaactcatgagtaccatgcggagaccatcaagaatgtgcgcacagccacggaaagctttgcttctgaccccatcctctaccggcccgttgctgtggctctagacactaaaggacctgagatccgaactgggctcatcaagggcagcggcactgcagaggtggagctgaagaagggagccactctcaaaatcacgctggataacgcctacatggaaaagtgtgacgagaacatcctgtggctggactacaagaacatctgcaaggtggtggaagtgggcagcaagatctacgtggatgatgggcttatttctctccaggtgaagcagaaaggtgccgacttcctggtgacggaggtggaaaatggtggctccttgggcagcaagaagggtgtgaaccttcctggggctgctgtggacttgcctgctgtgtcggagaaggacatccaggatctgaagtttggggtcgagcaggatgttgatatggtgtttgcgtcattcatccgcaaggcatctgatgtccatgaagttaggaaggtcctgggagagaagggaaagaacatcaagattatcagcaaaatcgagaatcatgagggggttcggaggtttgatgaaatcctggaggccagtgatgggatcatggtggctcgtggtgatctaggcattgagattcctgcagagaaggtcttccttgctcagaagatgatgattggacggtgcaaccgagctgggaagcctgtcatctgtgctactcagatgctggagagcatgatcaagaagccccgccccactcgggctgaaggcagtgatgtggccaatgcagtcctggatggagccgactgcatcatgctgtctggagaaacagccaaaggggactatcctctggaggctgtgcgcatgcagcacctgattgcccgtgaggcagaggctgccatctaccacttgcaattatttgaggaactccgccgcctggcgcccattaccagcgaccccacagaagccaccgccgtgggtgccgtggaggcctccttcaagtgctgcagtggggccataatcgtcctcaccaagtctggcaggtctgctcaccaggtggccagataccgcccacgtgcccccatcattgctgtgacccggaatccccagacagctcgtcaggcccacctgtaccgtggcatcttccctgtgctgtgcaaggacccagtccaggaggcctgggctgaggacgtggacctccgggtgaactttgccatgaatgttggcaaggcccgaggcttcttcaagaagggagatgtggtcattgtgctgaccggatggcgccctggctccggcttcaccaacaccatgcgtgttgttcctgtgccgtgatggaccccagagcccctcctccagcccctgtcccacccccttcccccagcccatccattaggccagcaacgcttgtagaactcactctgggctgtaacgtggcactggtaggttgggacaccagggaagaagatcaacgcctcactgaaacatggctgtgtttgcagcctgctctagtgggacagcccagagcctggctgcccatcatgtggccccacccaatcaagggaagaaggaggaatgctggactggaggcccctggagccagatggcaagagggtgacagcttcctttcctgtgtgtactctgtccagttcctttagaaaaaatggatgcccagaggactcccaaccctggcttggggtcaagaaacagccagcaagagttaggggccttagggcactgggctgttgttccattgaagccgactctggccctggcccttacttgcttctctagctctctaggcctctccagtttgcacctgtccccaccctccactcagctgtcctgcagcaaacactccaccctccaccttccattttcccccactactgcagcacctccaggcctgttgctatagagcctacctgtatgtcaataaacaacagctgaagcacc

SEQ ID NO: 105 - DNA, 2748 bp, Homo sapiens
gcattgcagactgcggagtcagacggcgctatgtacgccctcttcctcctggccagcctcctgggcgcggctctagccggcccggtccttggactgaaagaatgcaccaggggctcggcagtgtggtgccagaatgtgaagacggcgtccgactgcggggcagtgaagcactgcctgcagaccgtttggaacaagccaacagtgaaatcccttccctgcgacatatgcaaagacgttgtcaccgcagctggtgatatgctgaaggacaatgccactgaggaggagatccttgtttacttggagaagacctgtgactggcttccgaaaccgaacatgtctgcttcatgcaaggagatagtggactcctacctccctgtcatcctggacatcattaaaggagaaatgagccgtcctggggaggtgtgctctgctctcaacctctgcgagtctctccagaagcacctagcagagctgaatcaccagaagcagctggagtccaataagatcccagagctggacatgactgaggtggtggcccccttcatggccaacatccctctcctcctctaccctcaggacggcccccgcagcaagccccagccaaaggataatggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaacccaaggagatctgtgcgctggttgggttctgtgatgaggtgaaagagatgcccatgcagactctggtccccgccaaagtggcctccaagaatgtcatccctgccctggaactggtggagcccattaagaagcacgaggtcccagcaaagtctgatgtttactgtgaggtgtgtgaattcctggtgaaggaggtgaccaagctgattgacaacaacaagactgagaaagaaatactcgacgcttttgacaaaatgtgctcgaagctgccgaagtccctgtcggaagagtgccaggaggtggtggacacgtacggcagctccatcctgtccatcctgctggaggaggtcagccctgagctggtgtgcagcatgctgcacctctgctctggcacgcggctgcctgcactgaccgttcacgtgactcagccaaaggacggtggcttctgcgaagtgtgcaagaagctggtgggttatttggatcgcaacctggagaaaaacagcaccaagcaggagatcctggctgctcttgagaaaggctgcagcttcctgccagacccttaccagaagcagtgtgatcagtttgtggcagagtacgagcccgtgctgatcgagatcctggtggaggtgatggatccttccttcgtgtgcttgaaaattggagcctgcccctcggcccataagcccttgttgggaactgagaagtgtatatggggcccaagctactggtgccagaacacagagacagcagcccagtgcaatgctgtcgagcattgcaaacgccatgtgtggaactaggaggaggaatattccatcttggcagaaaccacagcattggtttttttctacttgtgtgtctgggggaatgaacgcacagatctgtttgactttgttataaaaatagggctcccccacctcccccatttctgtgtcctttattgtagcattgctgtctgcaagggagcccctagcccctggcagacatagctgcttcagtgccccttttctctctgctagatggatgttgatgcactggaggtcttttagcctgcccttgcatggcgcctgctggaggaggagagagctctgctggcatgagccacagtttcttgactggaggccatcaaccctcttggttgaggccttgttctgagccctgacatgtgcttgggcactggtgggcctgggcttctgaggtggcctcctgccctgatcagggaccctccccgctttcctgggcctctcagttgaacaaagcagcaaaacaaaggcagttttatatgaaagattagaagcctggaataatcaggctttttaaatgatgtaattcccactgtaatagcatagggattttggaagcagctgctggtggcttgggacatcagtggggccaagggttctctgtccctggttcaactgtgatttggctttcccgtgtctttcctggtgatgccttgtttggggttctgtgggtttgggtgggaagagggccatctgcctgaatgtaacctgctagctctccgaaggccctgcgggcctggcttgtgtgagcgtgtggacagtggtggccgcgctgtgcctgctcgtgttgcctacatgtccctggctgttgaggcgctgcttcagcctgcacccctcccttgtctcatagatgctccttttgaccttttcaaataaatatggatggcgagctcctaggcctctggcttcctggtagagggcggcatgccgaagggtctgctgggtgtggattggatgctggggtgtgggggttggaagctgtctgtggcccacttgggcacccacgcttctgtccacttctggttgccaggagacagcaagcaaagccagcaggacatgaagttgctattaaatggacttcgtgatttttgttttgcactaaagtttctgtgatttaacaataaaattctgttagccaga

SEQ ID NO: 106 - DNA, 7573 bp, Homo sapiens
ctcttccggtctttctggtctcggccgcagaagcgagatgacgaagggaacgtcatcgtttggaaagcgtcgcaataagacgcacacgttgtgccgccgctgtggctctaaggcctaccaccttcagaagtcgacctgtggcaaatgtggctaccctgccaagcgcaagagaaagtataactggagtgccaaggctaaaagacgaaataccaccggaactggtcgaatgaggcacctaaaaattgtataccgcagattcaggcatggattccgtgaaggaacaacacctaaacccaagagggcagctgttgcagcatccagttcatcttaagaatgtcaacgattagtcatgcaataaatgttctggttttaaaaaatacatatctggttttggtaaggtatttttaatcaattaggcttgtagtatcagtgaaatactgtaggtttagggactgggctagcttcatatcagatttacttgttaagtgactgttttggaatgtttacttttggactgggtttgtaacacggttaaaggcaatgagaaacaagcagaattccaggagtccttgaagcagagggcactggaagacaatatagcagattaaaatagcacagctcatgtggcataggtgggtattttagatgtttgagtaaatttgaaagagtatgatgtttaaattacctttagcaacatgttcatctgctatgctgtcatgactagggggatgattattagtcacatagagcttgggagtaccactggaaacgtatgggtaggagtttaggtggcttctgtttttcaaaagatgatcttatcctagtatctgtaatgctcacttggcacacctgacttgtgggctgtgtgtaaggtggctagctaagtgaaaaaagcctgctaggtgtgagtcaacttaagaatatgtaaataggtttgagaaaaagtagggcttgggtgcaagtaaagattgagcaggaaataaaggaaaatcaagtataatccctgagatttgtagactaaaggcaatgatgtgggactacttggtcgaatttttttagccctcaacttggtaattgggtgtttctgtgttaaagcactgaaacttgctgtcgtgccttcctagttttcgtggtttattgacagggttgggggttttttttgtttttttaaaatgaagggacaaagtcaactggactgctgagtgagagggcaggggcagttgaagggaacatgaattgctggaacagctacataaaatagtgatgtagccaagtcatgctatttaaattataattctccactgtgtttagaataacatctgaggttcttaacctggccttggaagggtatcacttttacttgtaacctggaatggctttataatgtgctagctaattgctactctcatcttgtattttaactcctaatttacccttcaggtctcagcttcagaacattcacttataaagaaaccctgctgattaaatctctcttgggcttcctcccgaaatgtgagactatactttaaagatgtatggttagagtccaattgccattgcctttcttgtttacagatagtgcaatggcgcaatcctggctcactgcagcctctaattcctgggctcaagtcaagtggttctcctgcctcagccttctgagtagctgggactacagatgcacaccaccacacctggctaatttttgtgttttttgtagagatggagattcactcgactaattctttttgtattcttagtagagactgggtttcaacatgttgaccaggctggtctcgaactcgacctcgtgatccgccgcctcggcctcccaaagtgctaggattacaggcgtgagccaccactcccggctgtcatcatcaaattttcaagtgaagatagtctgttgaagattgaacaatgaccttgaaagacagctgagttgctgtgggtataatgtaaagctggtgaagtttggccagtttgggcttcagaagtctaagtctagtgaaggtaccctgacccccatataaacaacccttgaggccggtgtggtggctcacggctgtaatcctggcactttgggaggccgaggtgggcggatcacaaggtcaagagatcgagatcaccctggcccacatggtgagaccccaactctactaaaaatacaaaaattagctgggtgtggtggcatgtgcctgtagtcttagctacttgagaggctgaggcaggagaatcgcttgaacctgggaggcagaggttgcagtgagctgagatcatgccactgtgctccagcctggtggcacagtgagactccacctcaaaaaaaaaaaaaaatccttgaactaatgactcaaattttcaaatgaaacaaaataagcagtggatcttgcattggagatggagttaaactatgttgcccaggctggtcttggactcctggtttcaagtgatcctctcatcttggcctcccaaagtgctgggattacagggacgagccaccacaccccacctattgtctatatttctatctttaacagcacttcagtcctgccttaagttatagttatgtatagatacccattatactttaaatttttcagcagaaattatgcttttatcttctctgcagtgttatatgttggtgtgcaaaaatgttaaatttatttttcctaagtaccccatctgcttttcaactctgtcctctgcctgaaaagcctccctccagcccctacttccctcccatcttagttcacaaagtcaggttgatttgcccccagctgtcaaagcagactacctgtttccacatgtaactggttatgttctgtaaagttacaaaaatagaaaggttgaatctgtggcggccgggtacggtagctcacgtcctatagtcgcagcactttgggaggccaaggtgggcagttcacgtgaggtggggagttcgagaccagcctgaccaacatggagaaaccccgtctctactaaaaatacaaaaatattagccgggcgtggtggtgcatgcctgtaatcccagctactagggaggctgaggcaggagaattgcttgaacctgggagacggaggttgcggtgagctgcagaggcctcacctctaattgagacacaattatatattgttgatatatatatatatatatatacacacacacacacacactatgatggataaatgcatgagtttctgtgagagcattggaaaggagtttgtcactcaataggtgaagccaggctaagatttaagctgagccagggaggacttgaaggaatcatgatgagagagaaggtaagtggctttgccagcaatgaaacagctgacataatggtaaccagtcagaggagggcataactatgaaactggacaccttggttgtcaggttagaaggatggggtgtagggttggtaagaaaagaattcagggaagagcagcgatcagattatgaagaatttgtcttgagaaattacagaggatttaaaccagaatgttaggaatagttattctagcaagatgaatgtggaaagtgttagtgtgcatgtgatgagtcttgaagctggaaactaggtaacaggttcttaaatagttcatgtgaaaatcatgacagactaaggcagtggctgtggggctgtccgggagttctctacagaaaacatctaaaacttgaatgtgcaagtgagtagctaacttccaagcttcccatttctgtataatttaagcatgaaaatgagaacactgagatttgataggcatgtagaagtcagagtaagcaagagggcttgagttcatcgatgaacttcagtaactatccttgacttagtattggtggaaaccatttgtgaatttacagactccaaaaacaaaaagataatttagagtctcaaagtaagatttggggagatgtccattggagacagaagtggagagagggagaagttcatttgttgacatatttatgtatggtgcacctactgtgtgccaagccatgttagagatacagtgggagcaaaaccagatgtgttttctgccctctgactccagtggttgcagaattagcccaggaactagaattggatggagtcacacaaaccaaggaaggggattgtttgaggggggcactcagtcacatgtggctgaggccaaagagaatgaagatatattttaaaattctggatttggcaaaatttaggctagtctttacttttcataaacctctctcatgttagagcagcaaagaaaaaccagtttgccaagaggtcacgagattgggcagtgaggaatggggagtttagtttaaaagacaacactagatacttttggaagtttacctattctgtggttcccttttattgactaaatggtgcccttcattcatgaagcaagtatctatttagtgactactatgtgtcaaaagctatctaggcccagaaacttttaagtggaaaacaaaacaaagaccctgccctgacagatttcaatcatgtatatactgtatgtatgtttgtggaaggtgacataatagacaattgcagatagtgataagtgtgtaagacaaggataccacgagcgtaatagacagtggggatggaggaacctttagaaggttggttaagttgaatgataaggagccattcttttgtctttacctgaatgacttatcattcagccctttttgagttttgggttgcttgcagagtttaaccttgcctgtaattgaaacttaattttgtaatagcataacttcatgtaagaaaagcaaagcaatacattacagaattattttactgaagaacttgtttcagagaaagaggctgtttcacatttattagcacatttggattatatttaggtgttttatttttttttaaacaaaggagtttggatcataatacaagagaagcacagggcaaagacactgcataacctcaagaactaagaatggaaggactggccaggcctggtggtgcacacctgtaatcccagctactcaggaggctgaggcgagagaatcgcttgagcctggggggcagaggttgcagtgagctgagagaacggcgggcggcagagcgagactccatctcaaaaaaaaaaaaggataaaggaaggacttaagcaaaatcttccttgtaagtagaaggatgttttgacaagaaaagttgcaatggaaaaatggttctcatgtacacgagtatgtagaataagcatcgtgtgtggattggattcagatcaaaacattgcttttatgtttgtgtctttatacggtgggagtataccctggtgccccaggatgaagacttgacctgacccatgtatttttagattactcacagataacaaaaagtattttcatcatgattagttgcgaaaacagttttatttcaataggtaaaacgtgcagtcctatgtaatcgtcagaaggtaatcttaattatagcttgggtgtgctttaaactgcaagctggcagtggagggcacgattcctctgatttcagctttctccttatacttttctggagctgtgagctgcaagttaactcagtgggattaaagtgtagactggaggtacaaaaggtgaggagtgaggagatagggtagttcttccttggctggctggcttcataatccctgggccccgcagataattaaatcgactttttctgtctcaggcatttgtatgacctctttggaggttccctgctgggtagttatccttgtatctgatggacccatctcaatttaaaatactctgccaggttcggaggttcatgcttgtcatcccagcactttgggaggctcagaggtgccattggcttgagcccaagagtttgagaccagcctgggcaacctggtgaaacctcttctccattaaaaatacaaaaaattagccaggcatggtggtatgtgcctgtagtcccagctactcagggagctgaggtggaaggattgcttgagcctgggaggtcaaggctgcagtgaaccttgattgtgccactgcactccagcctggatgacaaagaccctgtctcaagaaaagtctgtaattctttcctaacccttagtatccagcctcagtcctgaggttttctttacctctgggggctattttatgccggctctctcctgagtgtcacacatctggtcctcaggaaacattctcacatccctggcctgaaaaaaacaatttcagggagatcgcatggcagcagccctctctgggctccccagctaaatggttgtacgaaacacatttcaaagctctctgaagggcttccttgaagttcctttcactgggtttcaaagtagaaggtagtaactcctttgtccaagaaggctgaattgagcacttaacaattctccaaagaaatttcttcattggtctctatcctagaccccttctgtatccttgatatggctgaggattctaaaaaatgaccagtcttacatgggagggctgggatataaaaaataaaataataaataatgataaaaactgaaaactgaccacgttcttggatatgttttctttgggttgtgtgtgtgtgtgagacttttgatagttacccaaagtaggaaaaatcccattctaataaggttatatttatgtagctctgcaaataaacatctagcaaatgtaaaaagtattttctttgccttaaaaatgattaaaattatttgaactcctgaggagtgttatatgaataaaattagtaagttatttggaggaaagttattttttaaaaagacaactggtaaaacagtacaggagaaaggccagcttcctcaagtgaggacagttgtttagaattgactgaggagcggccgggtgcggaggctcacatctgtaatcccacacgccttgggaggctctgaggcgggtagatcacctgaggtcaggagtttgagaccagcctggccaacatggcgaaaccccgtctccactaaaaatacaaaaattagccaggtgcagtggcacacacctgtaatcccagctactccgaaggctgaggcaggaggatcacctgagcccaggaagttgagactgcagtgagctgagattgcaccactgcactccagcctcagtgacagcgagactgtctcaaaaaaagaaaaaagtgactgaggaggaagaggccaggtggcaaatggaacagaatcaccaaagggtgaacaggactaaggcaatgtagtgtatggctcagctacgtcagagtggaaaaggtgttattagagcagaaactatggtccctgcgtcacagggaagcaacctacagagaagcagcagctccccaagagaggagagataagaagccagaagcctcagagtgaacaattgtcctattagggattgctctagagagagaaacctctgggaacgtacccctgtgaggcagcacagcacaatgcttttagaattgtatgagagttgatatatctccatttgttttgcaaaggcaaaaactaagatacagagattatctgttaaagttatgtatttctttggtaataaagatgctgacagtgttgctggaatgcatttctttaataaagatattgtacaatgaaa

SEQ ID NO: 107 - DNA, 573 bp, Homo sapiens
ctttttttcaggcggccgggaagatggcggacattcagactgagcgtgcctaccaaaagcagccgaccatctttcaaaacaagaagagggtcctgctgggagaaactggcaaggagaagctcccgcggtactacaagaacatcggtctgggcttcaagacacccaaggaggctattgagggcacctacattgacaagaaatgccccttcactggtaatgtgtccattcgagggcggatcctctctggcgtggtgaccaagatgaagatgcagaggaccattgtcatccgccgagactatctgcactacatccgcaagtacaaccgcttcgagaagcgccacaagaacatgtctgtacacctgtccccctgcttcagggacgtccagatcggtgacatcgtcacagtgggcgagtgccggcctctgagcaagacagtgcgcttcaacgtgctcaaggtcaccaaggctgccggcaccaagaagcagttccagaagttctgaggctggacatcggcccgctccccacaatgaaataaagttattttctcattcccaggccagacttgggatcttc

SEQ ID NO: 108 - DNA, 1474 bp, Homo sapiens
aatttctacgcgcaccggaagacggaggtcctctttccttgcctaacgcagccatggctcgtggtcccaagaagcatctgaagcgggtggcagctccaaagcattggatgctggataaattgaccggtgtgtttgctcctcgtccatccaccggtccccacaagttgagagagtgtctccccctcatcattttcctgaggaacagacttaagtatgccctgacaggagatgaagtaaagaagatttgcatgcagcggttcattaaaatcgatggcaaggtccgaactgatataacctaccctgctggattcatggatgtcatcagcattgacaagacgggagagaatttccgtctgatctatgacaccaagggtcgctttgctgtacatcgtattacacctgaggaggccaagtacaagttgtgcaaagtgagaaagatctttgtgggcacaaaaggaatccctcatctggtgactcatgatgcccgcaccatccgctaccccgatcccctcatcaaggtgaatgataccattcagattgatttggagactggcaagattactgatttcatcaagttcgacactggtaacctgtgtatggtgactggaggtgctaacctaggaagaattggtgtgatcaccaacagagagaggcaccctggatcttttgacgtggttcacgtgaaagatgccaatggcaacagctttgccactcgactttccaacatttttgttattggcaagggcaacaaaccatggatttctcttccccgaggaaagggtatccgcctcaccattgctgaagagagagacaaaagactggcggccaaacagagcagtgggtgaaatgggtccctgggtgacatgtcagatctttgtacgtaattaaaaatattgtggcaggattaatagcattttggttttgtgggttgtttttttttttaattcagtttcctgctattttactgtataacatgaacagtgattaagttctcaggcaggcttaggcagtctgttattggctgggcttagttagtggtgggaagtagaagataccagacttgggaaaggaacagcaactaaccaaggagtagaaagagccaagaaatcccggagtatttatctcccccgccttgacttggccaagaacttctctacacagtggacccctctgccctgttaagttcccttggctttctaaaaatctgccctcattttcttctccagtctgtattgcactagcccatccacaagcctggctcatctgtttttcccactcctgtgcccatcttgccaaacgcaggatgaaaatctccagttgccattaaaggtggtcataaatgggctgttgatgttgcaaagcattgcagttccttggacttagtgcccttattctccagctgccgctgcagtcctcaactctgtttttcagaggacagagatgtagaacataaacttgttcttgcttgtccaaa

SEQ ID NO: 109 - DNA, 1369 bp, Homo sapiens
ctcttttccgtggcgcctcggaggcgttcagctgcttcaagatgaagctgaacatctccttcccagccactggctgccagaaactcattgaagtggacgatgaacgcaaacttcgtactttctatgagaagcgtatggccacagaagttgctgctgacgctctgggtgaagaatggaagggttatgtggtccgaatcagtggtgggaacgacaaacaaggtttccccatgaagcagggtgtcttgacccatggccgtgtccgcctgctactgagtaaggggcattcctgttacagaccaaggagaactggagaaagaaagagaaaatcagttcgtggttgcattgtggatgcaaatctgagcgttctcaacttggttattgtaaaaaaaggagagaaggatattcctggactgactgatactacagtgcctcgccgcctgggccccaaaagagctagcagaatccgcaaacttttcaatctctctaaagaagatgatgtccgccagtatgttgtaagaaagcccttaaataaagaaggtaagaaacctaggaccaaagcacccaagattcagcgtcttgttactccacgtgtcctgcagcacaaacggcggcgtattgctctgaagaagcagcgtaccaagaaaaataaagaagaggctgcagaatatgctaaacttttggccaagagaatgaaggaggctaaggagaagcgccaggaacaaattgcgaagagacgcagactttcctctctgcgagcttctacttctaagtctgaatccagtcagaaataagattttttgagtaacaaataaataagatcagactctgatctctgttgacttttcattgctgatagaaaactacataaatgaagagagggaaagttaggtcatagaaaatggggatatgtatatatggtattccaatggtggataaagtcagacctattgtgtgtggtagtggaagacaaggtcagtgtacaggggttcctttcaggggatgggtggcacaggaagggattctgagcataagacacctgcctcttcagaatggcatggaacttaggggaaatgatgaggactattaatcttttttatatagtttatttttgaggaatatagctctttaaagcactatacacaatttaatacatgaaaattgaccttgtgaagatgaacatgtcttttttaaaaggtaacttttaaggcatagttttagtggatacctagttttagtggataccatccactaaatggtttttaagccatttcaaatttgttttaacaaagcttttcaactccagtgaattgttaggctgttcacacactgaaaaggaaatgactaaaaacattcttggatcagtgaaaaa

SEQ ID NO: 110 - DNA, 1109 bp, Homo sapiens
gccgcccaggacccgcagcagagacgacgcctgcagcaaggagaccaggaaggggtgagacaaggaagaggatgtctgagctggagaaggccatggtggccctcatcgacgttttccaccaatattctggaagggagggagacaagcacaagctgaagaaatccgaactgaaggagctcatcaacaatgagctttcccatttcttagaggaaatcaaagagcaggaggttgtggacaaagtcatggaaacactggacaatgatggagacggcgaatgtgacttccaggaattcatggcctttgttgccatggttactactgcctgccacgagttctttgaacatgagtgagattagaaagcagccaaacctttcctgtaacagagacggtcatgcaagaaagcagacagcaagggcttgcagcctagtaggagctgagctttccagccgtgttgtagctaattaggaagcttgatttgctttgtgattgaaaaattgaaaacctctttccaaaggctgttttaacggcctgcatcattctttctgctatattaggcctgtgtgtaagctgactggccccagggactcttgttaacagtaacttaggagtcaggtctcagtgataaagcgtgcaccgtgcagcccgccatggccgtgtagaccctaacccggagggaaccctgactacagaaattaccccggggcacccttaaaacttccactacctttaaaaaacaaagccttatccagcattatttgaaaacactgctgttctttaaatgcgttcctcatccatgcagataacagctggttggccggtgtggccctgcaagggcgtggtggcttcggcctgcttcccgggatgcgcctgatcaccaggtgaacgctcagcgctggcagcgctcctggaaaaagcaactccatcagaactcgcaatccgagccagctctgggggctccagcgtggcctccgtgacccatgcgattcaagtcgcggctgcaggatccttgcctccaacgtgcctccagcacatgcggcttccgagggcactaccgggggctctgagccaccgcgagggcctgcgttcaataaaaagagcgcagcttggga

SEQ ID NO: 111 - DNA, 3165 bp, Homo sapiens
agtttttgcaacggctaaggaagggcctgtgggtttattataaggcggagctcggcgggagaggtgcgggccgaatccgagccgagcggagaggaatccggcagtagagagcggactccagccggcggaccctgcagccctcgcctgggacagcggcgcgctgggcaggcgcccaagagagcatcgagcagcggaacccgcgaagccggcccgcagccgcgacccgcgcagcctgccgctctcccgccgccggtccgggcagcatgaggcgcgcggcgctctggctctggctgtgcgcgctggcgctgagcctgcagccggccctgccgcaaattgtggctactaatttgccccctgaagatcaagatggctctggggatgactctgacaacttctccggctcaggtgcaggtgctttgcaagatatcaccttgtcacagcagaccccctccacttggaaggacacgcagctcctgacggctattcccacgtctccagaacccaccggcctggaggctacagctgcctccacctccaccctgccggctggagaggggcccaaggagggagaggctgtagtcctgccagaagtggagcctggcctcaccgcccgggagcaggaggccaccccccgacccagggagaccacacagctcccgaccactcatctggcctcaacgaccacagccaccacggcccaggagcccgccacctcccacccccacagggacatgcagcctggccaccatgagacctcaacccctgcaggacccagccaagctgaccttcacactccccacacagaggatggaggtccttctgccaccgagagggctgctgaggatggagcctccagtcagctcccagcagcagagggctctggggagcaggacttcacctttgaaacctcgggggagaatacggctgtagtggccgtggagcctgaccgccggaaccagtccccagtggatcagggggccacgggggcctcacagggcctcctggacaggaaagaggtgctgggaggggtcattgccggaggcctcgtggggctcatctttgctgtgtgcctggtgggtttcatgctgtaccgcatgaagaagaaggacgaaggcagctactccttggaggagccgaaacaagccaacggcggggcctaccagaagcccaccaaacaggaggaattctatgcctgacgcgggagccatgcgccccctccgccctgccactcactaggcccccacttgcctcttccttgaagaactgcaggccctggcctcccctgccaccaggccacctccccagcattccagcccctctggtcgctcctgcccacggagtcgtggggtgtgctgggagctccactctgcttctctgacttctgcctggagacttagggcaccaggggtttctcgcataggacctttccaccacagccagcacctggcatcgcaccattctgactcggtttctccaaactgaagcagcctctccccaggtccagctctggaggggagggggatccgactgctttggacctaaatggcctcatgtggctggaagatcctgcgggtggggcttggggctcacacacctgtagcacttactggtaggaccaagcatcttgggggggtggccgctgagtggcaggggacaggagtccactttgtttcgtggggaggtctaatctagatatcgacttgtttttgcacatgtttcctctagttctttgttcatagcccagtagaccttgttacttctgaggtaagttaagtaagttgattcggtatccccccatcttgcttccctaatctatggtcgggagacagcatcagggttaagaagactttttttttttttttttaaactaggagaaccaaatctggaagccaaaatgtaggcttagtttgtgtgttgtctcttgagtttgtcgctcatgtgtgcaacagggtatggactatctgtctggtggccccgtttctggtggtctgttggcaggctggccagtccaggctgccgtggggccgccgcctctttcaagcagtcgtgcctgtgtccatgcgctcagggccatgctgaggcctgggccgctgccacgttggagaagcccgtgtgagaagtgaatgctgggactcagccttcagacagagaggactgtagggagggcggcaggggcctggagatcctcctgcagaccacgcccgtcctgcctgtggcgccgtctccaggggctgcttcctcctggaaattgacgaggggtgtcttgggcagagctggctctgagcgcctccatccaaggccaggttctccgttagctcctgtggccccaccctgggccctgggctggaatcaggaatattttccaaagagtgatagtcttttgcttttggcaaaactctacttaatccaatgggtttttccctgtacagtagattttccaaatgtaataaactttaatataaagtagtcctgtgaatgccactgccttcgcttcttgcctctgtgctgtgtgtgacgtgaccggacttttctgcaaacaccaacatgttgggaaacttggctcgaatctctgtgccttcgtctttcccatggggagggattctggttccagggtccctctgtgtatttgcttttttgttttggctgaaattctcctggaggtcggtaggttcagccaaggttttataaggctgatgtcaatttctgtgttgccaagctccaagccccatcttctaaatggcaaaggaaggtggatggccccagcacagcttgacctgaggctgtggtcacagcggaggtgtggagccgaggcctaccccgcagacaccttggacatcctcctcccacccggctgcagaggccagaggcccccagcccagggctcctgcacttacttgcttatttgacaacgtttcagcgactccgttggccactccgagaggtgggccagtctgtggatcagagatgcaccaccaagccaagggaacctgtgtccggtattcgatactgcgactttctgcctggagtgtatgactgcacatgactcgggggtggggaaaggggtcggctgaccatgctcatctgctggtccgtgggacggtgcccaagccagaggctgggttcatttgtgtaacgacaataaacggtacttgtcatttcgggcaa

SEQ ID NO: 112 - DNA, 3006 bp, Homo sapiens
ctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 113 - DNA, 2201 bp, Homo sapiens
aacttggaggcagagacccaagcagctggaggctctgtgtgtgggtcgctgatttcttggagcctgaaaagaaggagcagcgactggacccagagccatgtggctgtgccctctggccctcaccctcatcttgatggcagcctctggtgctgcgtgcgaagtgaaggacgtttgtgttggaagccctggtatccccggcactcctggatcccacggcctgccaggcagggacgggagagatggtgtcaaaggagaccctggccctccaggccccatgggtccgcctggagaaacaccatgtcctcctgggaataatgggctgcctggagcccctggtgtccctggagagcgtggagagaagggggaggctggcgagagaggccctccagggcttccagctcatctagatgaggagctccaagccacactccacgacttcagacatcaaatcctgcagacaaggggagccctcagtctgcagggctccataatgacagtaggagagaaggtcttctccagcaatgggcagtccatcacttttgatgccattcaggaggcatgtgccagagcaggcggccgcattgctgtcccaaggaatccagaggaaaatgaggccattgcaagcttcgtgaagaagtacaacacatatgcctatgtaggcctgactgagggtcccagccctggagacttccgctactcagatgggacccctgtaaactacaccaactggtaccgaggggagcctgcaggtcggggaaaagagcagtgtgtggagatgtacacagatgggcagtggaatgacaggaactgcctgtactcccgactgaccatctgtgagttctgagaggcatttaggccatgggacagggaggatcctgtctggccttcagtttccatccccaggatccacttggtctgtgagatgctagaactccctttcaacagaattcacttgtggctattagagctggaggcacccttagccacttcattcccctgatgggccctgactcttccccataatcactgaccagccttgacactccccttgcaaaccatcccagcactgcaccccaggcagccactcctagccttggcctttggcatgagatggaggcctccttattccccatctggtccagttccttcacttacagatggcagcagtgaggccttggggtagaaggatcctccaaagtcacacagagtgcctgcctcctggtcccctcagctctgcctctgcagcccactgcctgcccagtgccatcaggatgagcagtaccggccaagcataatgacagagagaggcagatttcagggaagccctgactgtgtggagctaaggacacagtggagattctctggcactctgaggtctctgtggcaggcctggtcaggctctccaggtggtcagagggcccagtggtgccccagcacggtggtgcccaagccaaccctgtgactgacatgtacgattcactcctttgagtctttggatgccaactcagccccctgacctggaggcagccggccaaggcctctagggaagagccccccactgcagacatgacccgagtaactttctgctgatgaacaaatctgcaccccacttcagacctcggtgggcattcacaccaccccccatgccaccggctccactttccccttttattaatacattcacccagataatcattaaaattaacatgtgccaggtcttaggatgtgtcttggggtgggcacagtacccggtgactcttggggatatttatttattttccctgagcctatatcttcatctgtgaaatggggataaaaatacttgttgctgtcacaattattaccatctctccagctagcaaaattactaccagagccgttactacacacaaaggctattgaccgagcacataccatgtgccacacaccttgacaaaatcttttaatacagtttattatgtactattcaatctttacacaatgtcacgggaccagtattgtttacccaattttttataaggacactgaagcttagaggagtgaaatgttttgagtgttatttcagagagcaaatggcaaagactggatccaaacccatcttcctggacctgaagttcatgctcccagccaccccacccctgagctgaataaagatgatttaagcataataaatcgttagtgtgttcacatgagtttccata

SEQ ID NO: 114 - DNA, 1672 bp, Homo sapiens
gcaggttctcttacatcgaccgcctaagagtcgcgctgtaagaagcaacaacctctcctcttcgtctccgccatcagctcggcagtcgcgaagcagcaaccatgcgtgagtgcatctccatccacgttggccaggctggtgtccagattggcaatgcctgctgggagctctactgcctggaacacggcatccagcccgatggccagatgccaagtgacaagaccattgggggaggagatgattccttcaacaccttcttcagtgagacgggggctggcaagcatgtgccccgggcagtgtttgtagacttggaacccacagtcattgatgaagttcgcactggcacctaccgccagctcttccaccctgagcaacttatcacaggcaaagaagatgctgccaataactatgcccgagggcactacaccattggcaaggagatcattgacctcgtgttggaccgaattcgcaagctggccgaccagtgcacgggtctccagggcttcttggttttccacagctttggtgggggaactggttctgggttcacctcgctgctcatggaacgtctctcagttgattatggcaagaagtccaagctggagttctctatttacccggcgccccaggtttccacagctgtagttgagccctacaactccatcctcaccacccacaccaccctggagcactctgattgtgccttcatggtagacaatgaggccatctatgacatctgtcgtagaaacctcgatattgagcgtccaacctatactaacctgaataggttaataggtcaaattgtgtcctccatcactgcttccctgagatttgatggagccctgaatgttgacctgacagaattccagaccaacctggtgccctatccccgcatccacttccctctggccacatatgcccctgtcatctctgctgagaaagcctaccatgaacagctttctgtagcagagatcaccaatgcttgctttgagccagccaaccagatggtgaaatgtgaccctcgccatggtaaatacatggcttgctgcctgttgtaccgtggtgacgtggttcccaaagatgtcaatgctgccattgccaccatcaagaccaagcgtaccatccagtttgtggattggtgccccactggcttcaaggttggcatcaactaccagcctcccactgtggtgcctggtggagacctggccaaggtacagagagctgtgtgcatgctgagcaacaccacagccattgctgaggcctgggctcgcctggaccacaagtttgacctgatgtatgccaaacgtgcctttgttcactggtacgttggggaggggatggaggaaggtgagttttcagaggcccgtgaggacatggctgcccttgagaaggattatgaggaggttggtgtggattctgttgaaggagagggtgaggaagaaggagaggaatactaaagttaaaacgtcacaaaggtgctgcttttacagggaagcttattctgttttaaacattgaaaagttgtggtctgatcagttaatttgtatgtagcagtgtatgctctcatatacaattactgacctatgctctaaaacatgaatgctttgttacagacccaagctgtccatttctgtgatgggttttgaataaagtattccctgtcttaaatgaa

SEQ ID NO: 115 - DNA, 2515 bp, Homo sapiens
ctctcagaaccttcctgccgtcgcgtttgcacctcgctgctccagcctctggggcgcattccaaccttccagcctgcgacctgcggagaaaaaaaattacttattttcttgccccatacataccttgaggcgagcaaaaaaattaaattttaaccatgagggaaatcgtgcacatccaggctggtcagtgtggcaaccagatcggtgccaagttctgggaggtgatcagtgatgaacatggcatcgaccccaccggcacctaccacggggacagcgacctgcagctggaccgcatctctgtgtactacaatgaagccacaggtggcaaatatgttcctcgtgccatcctggtggatctagaacctgggaccatggactctgttcgctcaggtccttttggccagatctttagaccagacaactttgtatttggtcagtctggggcaggtaacaactgggccaaaggccactacacagagggcgccgagctggttgattctgtcctggatgtggtacggaaggaggcagagagctgtgactgcctgcagggcttccagctgacccactcactgggcgggggcacaggctctggaatgggcactctccttatcagcaagatccgagaagaataccctgatcgcatcatgaataccttcagtgtggtgccttcacccaaagtgtctgacaccgtggtcgagccctacaatgccaccctctccgtccatcagttggtagagaatactgatgagacctattgcattgacaacgaggccctctatgatatctgcttccgcactctgaagctgaccacaccaacctacggggatctgaaccaccttgtctcagccaccatgagtggtgtcaccacctgcctccgtttccctggccagctcaatgctgacctccgcaagttggcagtcaacatggtccccttcccacgtctccatttctttatgcctggctttgcccctctcaccagccgtggaagccagcagtatcgagctctcacagtgccggaactcacccagcaggtcttcgatgccaagaacatgatggctgcctgtgacccccgccacggccgatacctcaccgtggctgctgtcttccgtggtcggatgtccatgaaggaggtcgatgagcagatgcttaacgtgcagaacaagaacagcagctactttgtggaatggatccccaacaatgtcaagacagccgtctgtgacatcccacctcgtggcctcaagatggcagtcaccttcattggcaatagcacagccatccaggagctcttcaagcgcatctcggagcagttcactgccatgttccgccggaaggccttcctccactggtacacaggcgagggcatggacgagatggagttcaccgaggctgagagcaacatgaacgacctcgtctctgagtatcagcagtaccaggatgccaccgcagaagaggaggaggatttcggtgaggaggccgaagaggaggcctaaggcagagcccccatcacctcaggcttctcagttcccttagccgtcttactcaactgcccctttcctctccctcagaatttgtgtttgctgcctctatcttgttttttgttttttcttctggggggggtctagaacagtgcctggcacatagtaggcgctcaataaatacttgtttgttgaatgtctcctctctctttccactctgggaaacctaggtttctgccattctgggtgaccctgtatttctttctggtgcccattccatttgtccagttaatacttcctcttaaaaatctccaagaagctgggtctccagatcccatttagaaccaaccaggtgctgaaaacacatgtagataatggccatcatcctaagcccaaagtagaaaatggtagaaggtagtgggtagaagtcactatataaggaaggggatgggattttccattctaaaagttttggagagggaaatccaggctattaaagtcactaaatttctaagtatgtccatttcccatctcagcttcaagggaggtgtcagcagtattatctccactttcaatctccctccaagctctactctggaggagtctgtcccactctgtcaagtggaatccttccctttccaactctacctccctcactcagctcctttcccctgatcagagaaagggatcaagggggttgggaggggggaaagagaccagccttggtccctaagcctccagaaacgtcttcttaatccccaccttttcttactcccaaaaaagaatgaacacccctgactctggagtggtgtatactgccacatcagtgtttgagtcagtccccagaggagaggggaaccctcctccatcttttttgcaacatctcatttcttccttttgctgttgcttcccccctcacacacttggttttgttctatcctacatttgagatttctattttatgttgaacttgctgctttttttcatattgaaaagatgacatcgccccaagagccaaaaataaatgggaattgaaaaaa

SEQ ID NO: 116 - DNA, 933 bp, Homo sapiens
atttaggggcggttggctttgttgggtgagcttgtttgtgtccctgtgggtggacgtggttggtgattggcaggatcctggtatccgctaacaggtcaaaatgcagatcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggtatgcagatcttcgtgaagaccctgaccggcaagaccatcaccctggaagtggagcccagtgacaccatcgaaaatgtgaaggccaagatccaggataaagaaggcatccctcccgaccagcagaggctcatctttgcaggcaagcagctggaagatggccgcactctttctgactacaacatccagaaggagtcgaccctgcacctggtcctgcgtctgagaggtggtatgcagatcttcgtgaagaccctgaccggcaagaccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtcctgcgcctgaggggtggctgttaattcttcagtcatggcattcgcagtgcccagtgatggcattactctgcactatagccatttgccccaacttaagtttagaaattacaagtttcagtaatagctgaacctgttcaaaatgttaataaaggtttcgttgcatggta

SEQ ID NO: 117 - DNA, 2193 bp, Homo sapiens
agttccgtcgcagccgggatttgggtcgcagttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtcgagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcaaatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaa

SEQ ID NO: 118 - DNA, 2154 bp, Homo sapiens
gggaggcccacgtatggcgcctctccaaaggctgcagaagtttcttgctaacaaaaagtccgcacattcgagcaaagacaggctttagcgagttattaaaaacttaggggcgctcttgtcccccacagggcccgaccgcacacagcaaggcgatggcccagctgtaagttggtagcactgagaactagcagcgcgcgcggagcccgctgagacttgaatcaatctggtctaacggtttcccctaaaccgctaggagccctcaatcggcgggacagcagggcgcgtcctctgccactctcgctccgaggtccccgcgccagagacgcagccgcgctcccaccacccacacccaccgcgccctcgttcgcctcttctccgggagccagtccgcgccaccgccgccgcccaggccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacacttctgattaagacggttgaaactagagatggacaggttatcaacgaaacttctcagcatcacgatgaccttgaataaaaattgcacacactcagtgcagcaatatattaccagcaagaataaaaaagaaatccatatcttaaagaaacagctttcaagtgcctttctgcagtttttcaggagcgcaagatagatttggaataggaataagctctagttcttaacaaccgacactcctacaagatttagaaaaaagtttacaacataatctagtttacagaaaaatcttgtgctagaatactttttaaaaggtattttgaataccattaaaactgcttttttttttccagcaagtatccaaccaacttggttctgcttcaataaatctttggaaaaactc

SEQ ID NO: 119 - DNA, 2052 bp, Homo sapiens
ggattgaggcgccgccatttttgctgcccggacgcggagcgagaggctgagagagtcggagacactatccgcttccatccgtcgcgcagaccctgccggagccgctgccgctatggatgatcgagaggatctggtgtaccaggcgaagctggccgagcaggctgagcgatacgacgaaatggtggagtcaatgaagaaagtagcagggatggatgtggagctgacagttgaagaaagaaacctcctatctgttgcatataagaatgtgattggagctagaagagcctcctggagaataatcagcagcattgaacagaaagaagaaaacaagggaggagaagacaagctaaaaatgattcgggaatatcggcaaatggttgagactgagctaaagttaatctgttgtgacattctggatgtactggacaaacacctcattccagcagctaacactggcgagtccaaggttttctattataaaatgaaaggggactaccacaggtatctggcagaatttgccacaggaaacgacaggaaggaggctgcggagaacagcctagtggcttataaagctgctagtgatattgcaatgacagaacttccaccaacgcatcctattcgcttaggtcttgctctcaatttttccgtattctactacgaaattcttaattcccctgaccgtgcctgcaggttggcaaaagcagcttttgatgatgcaattgcagaactggatacgctgagtgaagaaagctataaggactctacacttatcatgcagttgttacgtgataatctgacactatggacttcagacatgcagggtgacggtgaagagcagaataaagaagcgctgcaggacgtggaagacgaaaatcagtgagacataagccaacaagagaaaccatctctgaccaccccctcctccccatcccaccctttggaaactccccattgtcactgagaaccaccaaatctgacttttacatttggtctcagaatttaggttcctgccctgttggttttttttttttttttttaaacagttttcaaaagttcttaaaggcaagagtgaatttctgtggattttactggtcccagcttttaggttctttaagacactaacaggactacatagaggctttttcagcattactgtgtcgtctccgtgccagatgtggcaagatcaccattagcaaatggaaattacatttgaaagccattagacttataggtgatgcaagcatctaagagagaggttaatcacactatagaggcataagtggtatcagttttcatttttctaattgtttaaactgtgttttataccagtgtttgcaagtaattgggtgttagcttgagatggttaaaggtggtttggggagggacttcgttgtaatggttttgctgtaaaaaatgtttccaactccgctgaaatgttgctgaaaagcatggtgctggtaacagttcaacaatccgtggctgctcattcttgcctactttactctcccactgaagcaggttagcgttgaaggtggtatggaaaagcctgcatgcctgttcaattcttttgtttcttctccttccccctccccctacctccttcccctcactcctcccctccttcgctcgctcaacctcttttgttcagtatgtgtaacttgaagctaatttgtactactggatatctgactggagccacagatacagaatctgtattgttcttactgaaacacagcatggaattaacattaaacttaaataaaacaaacctaaattaaaaatgccaaatatcgcgcctccatcctttatacttctaaaataaaatctctagtccaaacacactgacagtattatgtttgcctgaatgtgaaacattcctccttttttcttccataaataatttgtttgcttcccaaagatgtatgttttggaaaaaaattgcctgtagtctggttgtgaagaacactgagtcagttgatggtgtgtcttgtcctccagaccaggcagcaacatggtatttattttaatcattaaagtacaacatgagtagta

SEQ ID NO: 120 - DNA, 3501 bp, Homo sapiens; identical to SEQ ID NO: 72